(19) United States
(12) Patent Application Publication
(10) Pub. No.: US 2022/0387488 A1
(43) Pub. Date: Dec. 8, 2022

(54) GENETICALLY ENGINEERED IMMUNE CELLS TARGETING CD70 FOR USE IN TREATING HEMATOPOIETIC MALIGNANCIES

(71) Applicant: CRISPR THERAPEUTICS AG, Zug (CH)

(72) Inventors: Jonathan Alexander TERRETT, Cambridge, MA (US); Mary-Lee DEQUÉANT, Cambridge, MA (US); Matthias WILL, Cambridge, MA (US)

(21) Appl. No.: 17/742,940

(22) Filed: May 12, 2022

Related U.S. Application Data
(60) Provisional application No. 63/187,619, filed on May 12, 2021.

(51) Int. Cl. A61K 35/17; C07K 16/28; C07K 14/725; C12N 5/0783

(52) U.S. Cl. CPC A61K 35/17; C07K 16/2803; C07K 16/2866; C07K 14/7051; C12N 5/0636; A61K 2039/505

(57) ABSTRACT

Aspects of the present disclosure relate to compositions comprising a population of genetically engineered T cells that expresses a chimeric antigen receptor (CAR) that binds CD70, and methods of using such for the treatment of T cell and B cell malignancies.

### CROSS REFERENCE TO RELATED APPLICATIONS

This application claims the benefit of the filing date of U.S. Provisional Application No. 63/187,619, filed May 12, 2021, the entire contents of which are incorporated by reference herein.

### SEQUENCE LISTING

The instant application contains a Sequence Listing which has been filed electronically in ASCII format and is hereby incorporated by reference in its entirety. Said ASCII copy, created on May 5, 2022, is named 095136-0671-047WO1_SEQ.txt and is 72,922 bytes in size.

### BACKGROUND

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

### SUMMARY

The present disclosure is based, at least in part, on the surprising discovery that anti-CD70 CAR+ T cells, such as CTX130 cells disclosed herein, provided long-term tumor elimination in a subcutaneous T cell lymphoma xenograft model. For example, anti-CD70 CAR+ T cells described herein (e.g., CTX130 cells) provided complete tumor elimination for at least 90 days following administration. Significant reductions in tumor burden were also observed in an additional subcutaneous T cell lymphoma xenograft model. Further, CTX130 cell distribution, expansion, and persistence were observed in human subjects receiving the CAR-T cells. Superior treatment efficacy was also observed in human lymphoma patients who received the CTX130 cell treatment.

In some aspects, the present disclosure features a method for treating a hematopoietic cancer, the method comprising: (i) administering to a human patient (e.g., a human adult patient, for example, ≥18) having a hematopoietic cancer, which optionally is a CD70+ hematopoietic cancer, one or more doses of an anti-CD38 antibody, (ii) performing a lymphodepletion treatment to the human patient after the first dose of the anti-CD38 antibody; and (iii) administering to the human patient an effective amount of a population of genetically engineered T cells, which expresses a chimeric antigen receptor (CAR) that binds CD70 and is deficient in MHC Class I expression (e.g., have a substantially reduced level of MHC Class I expression as relative to a wild-type counterpart or no detectable level of MHC Class I expression). In some embodiments, the hematopoietic cancer is a T cell malignancy or a B cell malignancy.

In some embodiments, the population of genetically engineered T cells may comprise a disrupted β2M gene. For example, the population of genetically engineered T cells may comprise T cells having a disrupted TRAC gene, and a disrupted β2M gene. In some examples, the population of genetically engineered T cells comprises T cells having a disrupted TRAC gene, a disrupted β2M gene, and a disrupted CD70 gene. In some examples, a nucleotide sequence encoding the CAR is inserted into the disrupted TRAC gene.

In any of the methods disclosed herein, step (i) may comprise administering to the human patient a first dose of the anti-CD38 antibody at least 12 hours prior to the lymphodepletion treatment in step (ii) and within 10 days of the administration of the genetically engineered T cells in step (iii). In some instances, step (i) may further comprise administering to the human patient a second dose of the anti-CD38 antibody about three weeks after the first dose of the anti-CD70 CAR-T cells. In some instances, step (i) may further comprise administering to the human patient a third dose of the anti-CD83 antibody about six weeks after the first dose of the anti-CD70 CAR-T cells.

In any of the methods disclosed herein, step (iii) may further comprise administering to the human patient a second dose of the population of anti-CD70 CAR-T cells. In some instances, the second dose of the anti-CD70 CAR-T cells is performed about 4-15 days (e.g., 4-6 days or 5-7 days) after the first dose of the anti-CD70 CAR-T cells. In some instances, the second dose of the anti-CD70 CAR-T cells is not accompanied with a second lymphodepletion treatment.

In some embodiments, any of the methods disclosed herein may further comprise repeating steps (ii)-(iii), optionally step (i), when the human patient (a) loses complete response within 2 years after the first dose of the anti-CD70 CAR-T cells, or (b) show partial response, stable disease or progressive disease with clinical benefit. In some instances, steps (ii)-(iii), optionally step (i), are repeated once. Alternatively, steps (ii)-(iii), optionally step (i), are repeated twice.

In some embodiments, the second dose of the anti-CD70 CAR-T cells may be performed about 4-8 weeks after the first dose of the anti-CD70 CAR-T cells in step (iii). In some instances, the second dose of the anti-CD70 CAR-T cells is accompanied with a second lymphodepletion treatment, and optionally treatment with the anti-CD83 antibody. In some instances, the human patient achieves complete response, partial response, stable disease, or progressive disease with clinical benefit about 4 weeks after the first dose of the anti-CD70 CAR-T cells. In other instances, the second dose of the anti-CD70 CAR-T cells is not accompanied with a second lymphodepletion treatment when the human patient experiences significant cytopenia.

In some embodiments, any of the methods disclosed herein may further comprise (iv) administering to the human patient a third dose of the anti-CD70 CAR-T cells when the human patient (a) loses complete response within 2 years after the first dose of the anti-CD70 CAR-T cells, or (b) show partial response, stable disease, or progressive disease with clinical benefit. In some instances, the third dose of the anti-CD70 CAR-T cells is greater than or equal to the first dose and/or the second dose of the anti-CD70 CAR-T cells. In some instances, the third dose of the anti-CD70 CAR-T cells is accompanied with a third lymphodepletion treatment, and optionally a further treatment with the anti-CD38 antibody. In other instances, the third dose of the anti-CD70 CAR-T cells is not accompanied with a third lymphodepletion treatment when the human patient experiences significant cytopenia.

In some embodiments, the anti-CD38 antibody is daratumumab. In some embodiments, the one or more doses of the anti-CD38 antibody may be about 8 mg/kg to about 16 mg/kg via intravenous infusion or 1800 mg via subcutaneous injection. In some instances, the first dose, the second dose, or both of the anti-CD38 antibody may be 16 mg/kg via intravenous infusion. Such a dose may split evenly into two portions (e.g., 8 mg/kg each), which can be administered to the human patient in two consecutive days. In other instances, the first dose, the second dose, or both of the anti-CD38 antibody may be 8 mg/kg via intravenous infusion.

In some embodiments, the lymphodepletion treatment in step (ii) may comprise co-administering to the human patient fludarabine at 30 mg/m² and cyclophosphamide at 500 mg/m² intravenously per day for three days. Prior to the lymphodepletion treatment of step (ii), the human patient does not show one or more of the following features: (a) change in performance status to ECOG>1, (b) significant worsening of clinical status, (c) requirement for supplemental oxygen to maintain a saturation level of greater than 92%, (d) uncontrolled cardiac arrhythmia, (e) hypotension requiring vasopressor support, (f) active infection, (g) any acute neurological toxicity, and (h) platelet count≤25,000/mm³ and/or absolute neutrophil count≤500/mm³.

In some instances, the lymphodepletion treatment of (ii) can be performed about 2-7 days prior to step (iii).

In any of the methods disclosed herein, the effective amount of the genetically engineered T cells in step (ii) may range from about 1×10⁶ CAR+ T cells to about 1.8×10⁹ CAR+ T cells. In some examples, the effective amount of the genetically engineered T cells in step (ii) may range from about 1×10⁶ CAR+ T cells to about 9×10⁸ CAR+ T cells. In some examples, the effective amount of the genetically engineered T cells in step (ii) may range from about 3×10⁷ CAR+ T cells to about 1×10⁸ CAR+ T cells. In some examples, the effective amount of the genetically engineered T cells in step (ii) may range from about 1×10⁸ CAR+ T cells to about 3×10⁸ CAR+ T cells. In some examples, the effective amount of the genetically engineered T cells in step (ii) may range from about 3×10⁸ CAR+ T cells to about 4.5×10⁸ CAR+ T cells. In some examples, the effective amount of the genetically engineered T cells in step (ii) may range from about 4.5×10⁸ CAR+ T cells to about 6×10⁸ CAR+ T cells. In some examples, the effective amount of the genetically engineered T cells in step (ii) may range from about 6×10⁸ CAR+ T cells to about 7.5×10⁸ CAR+ T cells. In some examples, the effective amount of the genetically engineered T cells in step (ii) may range from about 7.5×10⁸ CAR+ T cells to about 9×10⁸ CAR+ T cells. In some examples, the effective amount of the genetically engineered T cells in step (ii) may range from about 9×10⁸ CAR+ T cells to about 1.8×10⁹ CAR+ T cells. In specific examples, the effective amount of the genetically engineered T cells in step (ii) may be about 3×10⁷, 1×10⁸, 3×10⁸, 4.5×10⁸, 6×10⁸, 7.5×10⁸, 9×10⁸, or 1.8×10⁹ CAR+ T cells.

In some embodiments, prior to step (iii) and after step (ii), the human patient does not show one or more of the following features: (a) change in performance status to Eastern Cooperative Oncology Group (ECOG)>1, (b) active uncontrolled infection, (c) significant worsening of clinical status, and (d) any acute neurological toxicity.

In some embodiments, the methods disclosed herein may comprise repeating steps (i)-(iii) up to three times when the human patient show: (a) loss of response within the first 2 years after last dose of the genetically engineered T cells, or (b) stable disease or progressive disease with significant clinical benefit after the last dose of the genetically engineered T cells (e.g., after four weeks of the last dose of the genetically engineered T cells). In some instances, a subsequent dose of the genetically engineered T cells is about 28 days after the preceding dose of the genetically engineered T cells. In some instances, a human patient who is subjecting to repeated doses of the anti-CD70 CAR T cells may not show one or more of the following prior to a subsequent dose of the genetically engineered T cells: (a) dose-limiting toxicity (DLT), (b) CRS≥3 that does not resolve to grade 2 within 72 hours following the last dose of the genetically engineered T cells, (c) grade>1 GvHD, and (d) grade≥2 ICAN.

In some embodiments, the methods disclosed herein may further comprise confirming presence of CD70+ cancer cells in the human patient prior to a subsequent dose of the genetically engineered T cells.

A human patient to be treated by any of the methods disclosed herein may be free of one or more of the following prior to a subsequent dose of the anti-CD38 antibody: (a) severe or unmanageable toxicity with prior doses of the anti-CD38 antibody, (b) disease progression, (c) ongoing uncontrolled infection, (d) grade≥3 thrombocytopenia; (e) CD4+ T cell count<100/μl; and (f) platelet count<25,000 cells/μl.

In some aspects, the present disclosure features a method for treating a hematopoietic cancer, the method comprising: (i) performing a first lymphodepletion treatment to a human patient (e.g., a human adult patient, for example, ≥18) having a hematopoietic cancer, which optionally is a CD70+ hematopoietic cancer, (ii) administering to the human patient a first dose of a population of genetically engineered T cells, which expresses a chimeric antigen receptor (CAR) that binds CD70 (anti-CD70 CAR-T cells); and (iii) administering to the human patient a second dose of the anti-CD70 CAR-T cells. In some embodiments, the hematopoietic cancer is a T cell malignancy or a B cell malignancy.

In some embodiments, the population of genetically engineered T cells may comprise a disrupted β2M gene. For example, the population of genetically engineered T cells may comprise T cells having a disrupted TRAC gene, and a disrupted β2M gene. In some examples, the population of genetically engineered T cells comprises T cells having a disrupted TRAC gene, a disrupted β2M gene, and a disrupted CD70 gene. In some examples, a nucleotide sequence encoding the CAR is inserted into the disrupted TRAC gene.

In some embodiments, the second dose of the anti-CD70 CAR-T cells in step (iii) is performed about 4-15 days (e.g., 4-6 days or 5-7 days) after the first dose of the anti-CD70 CAR-T cells in step (ii). In some instances, the second dose of the anti-CD70 CAR-T cells is not accompanied with a second lymphodepletion treatment.

In some embodiments, the methods disclosed herein may further comprise repeating steps (i)-(iii), when the human patient (a) loses complete response within 2 years after the first dose of the anti-CD70 CAR-T cells, or (b) show partial response, stable disease or progressive disease with clinical benefit. In some instances, steps (i)-(iii) are repeated once. In other instances, steps (i)-(iii) are repeated twice.

In some embodiments, the second dose of the anti-CD70 CAR-T cells in step (iii) is performed about 4-8 weeks after the first dose of the anti-CD70 CAR-T cells in step (ii). In some instances, the second dose of the anti-CD70 CAR-T cells is accompanied with a second lymphodepletion treatment. In some instances, the human patient achieves complete response, partial response, stable disease, or progressive disease with clinical benefit about 4 weeks after the first dose of the anti-CD70 CAR-T cells. In other instances, the second dose of the anti-CD70 CAR-T cells is not accompanied with a second lymphodepletion treatment when the human patient experiences significant cytopenia.

In some embodiments, the methods disclosed herein may further comprise (iv) administering to the human patient a third dose of the anti-CD70 CAR-T cells when the human patient (a) loses complete response within 2 years after the first dose of the anti-CD70 CAR-T cells, or (b) show partial response, stable disease or progressive disease with clinical benefit. In some instances, the third dose of the anti-CD70 CAR-T cells is greater than or equal to the first dose and/or the second dose of the anti-CD70 CAR-T cells. In some instances, the third dose of the anti-CD70 CAR-T cells is accompanied with a third lymphodepletion treatment. In other instances, the third dose of the anti-CD70 CAR-T cells is not accompanied with a third lymphodepletion treatment when the human patient experiences significant cytopenia.

In some embodiments, the first, second, and/or third lymphodepletion treatment in step (ii) may comprise co-administering to the human patient fludarabine at 30 mg/m² and cyclophosphamide at 500 mg/m² intravenously per day for three days. Prior to the lymphodepletion treatment of step (ii), the human patient does not show one or more of the following features: (a) change in performance status to ECOG>1, (b) significant worsening of clinical status, (c) requirement for supplemental oxygen to maintain a saturation level of greater than 92%, (d) uncontrolled cardiac arrhythmia, (e) hypotension requiring vasopressor support, (f) active infection, (g) any acute neurological toxicity, and (h) platelet count≤25,000/mm³ and/or absolute neutrophil count≤500/mm³. In some instances, the lymphodepletion treatment is performed about 2-7 days prior to the subsequent administration of the anti-CD70 CAR-T cells.

In the methods disclosed herein, the effective amount of the genetically engineered T cells in step (ii) may range from about 1×10⁶ CAR+ T cells to about 1.8×10⁹ CAR+ T cells. In some examples, the effective amount of the genetically engineered T cells in step (ii) may range from about 1×10⁶ CAR+ T cells to about 9×10⁸ CAR+ T cells. In some examples, the effective amount of the genetically engineered T cells in step (ii) may range from about 3×10⁷ CAR+ T cells to about 1×10⁸ CAR+ T cells. In some examples, the effective amount of the genetically engineered T cells in step (ii) may range from about 1×10⁸ CAR+ T cells to about 3×10⁸ CAR+ T cells. In some examples, the effective amount of the genetically engineered T cells in step (ii) may range from about 3×10⁸ CAR+ T cells to about 4.5×10⁸ CAR+ T cells. In some examples, the effective amount of the genetically engineered T cells in step (ii) may range from about 4.5×10⁸ CAR+ T cells to about 6×10⁸ CAR+ T cells. In some examples, the effective amount of the genetically engineered T cells in step (ii) may range from about 6×10⁸ CAR+ T cells to about 7.5×10⁸ CAR+ T cells. In some examples, the effective amount of the genetically engineered T cells in step (ii) may range from about 7.5×10⁸ CAR+ T cells to about 9×10⁸ CAR+ T cells. In some examples, the effective amount of the genetically engineered T cells in step (ii) may range from about 9×10⁸ CAR+ T cells to about 1.8×10⁹ CAR+ T cells. In specific examples, the effective amount of the genetically engineered T cells in step (ii) may be about 3×10⁷, 1×10⁸, 3×10⁸, 4.5×10⁸, 6×10⁸, 7.5×10⁸, 9×10⁸, or 1.8×10⁹ CAR+ T cells.

In some embodiments, prior to step (iii) and after step (ii), the human patient does not show one or more of the following features: (a) change in performance status to Eastern Cooperative Oncology Group (ECOG)>1, (b) active uncontrolled infection, (c) significant worsening of clinical status, and (d) any acute neurological toxicity.

In some embodiments, the methods disclosed herein may comprise repeating steps (i)-(iii) up to three times when the human patient show: (a) loss of response within the first 2 years after last dose of the genetically engineered T cells, or (b) stable disease or progressive disease with significant clinical benefit after the last dose of the genetically engineered T cells (e.g., after four weeks of the last dose of the genetically engineered T cells). In some instances, a subsequent dose of the genetically engineered T cells is about 28 days after the preceding dose of the genetically engineered T cells. In some instances, a human patient who is subjecting to repeated doses of the anti-CD70 CAR T cells may not show one or more of the following prior to a subsequent dose of the genetically engineered T cells: (a) dose-limiting toxicity (DLT), (b) CRS≥3 that does not resolve to grade 2 within 72 hours following the last dose of the genetically engineered T cells, (c) grade>1 GvHD, and (d) grade≥2 ICAN.

In some embodiments, the methods disclosed herein may further comprise confirming presence of CD70+ cancer cells in the human patient prior to a subsequent dose of the anti-CD70 CAR-T cells.

In some aspects, the present disclosure features a method for treating a hematopoietic cancer, the method comprising (i) performing a lymphodepletion treatment to the human patient; and (ii) administering to the human patient an effective amount of a population of genetically engineered T cells, which expresses a chimeric antigen receptor (CAR) that binds CD70 (anti-CD70 CAR-T cells), wherein the effective amount of the anti-CD70 CAR-T cell ranges from about 9×10⁸ CAR+ T cells to about 1.8×10⁹ CAR+ T cells. In some examples, the effective amount of the anti-CD70 CAR-T cell is about 1.8×10⁹ CAR+ T cells.

In some embodiments, the lymphodepletion treatment in step (i) may comprise co-administering to the human patient fludarabine at 30 mg/m² and cyclophosphamide at 500 mg/m² intravenously per day for three days. In some embodiments, step (i) may be performed about 2-7 days prior to step (ii). In some embodiments, steps (i)-(ii) may be repeated up to two times when the human patient (a) loses complete response within 2 years after the first dose of the anti-CD70 CAR-T cells, or (b) show partial response, stable disease or progressive disease with clinical benefit.

In some embodiments, prior to any of the lymphodepletion treatments, the human patient does not show one or more of the following features: (a) change in performance status to ECOG>1, (b) significant worsening of clinical status, (c) requirement for supplemental oxygen to maintain a saturation level of greater than 92%, (d) uncontrolled cardiac arrhythmia, (e) hypotension requiring vasopressor support, (f) active infection, (g) any acute neurological toxicity, and (h) platelet count≤25,000/mm³ and/or absolute neutrophil count≤500/mm³.

In some embodiments, prior to administration of the anti-CD70 CAR-T cells and after the lymphodepletion treatment, the human patient does not show one or more of the following features: (a) change in performance status to Eastern Cooperative Oncology Group (ECOG)>1, (b) active uncontrolled infection, (c) significant worsening of clinical status, and (d) any acute neurological toxicity. In some instances, the human patient may have undergone a prior anti-cancer therapy. In some instances, the human patient has relapsed or refractory hematopoietic cell malignancies. In some embodiments, the human patient has a T cell malignancy, which is T cell lymphoma. Examples include, but are not limited to, cutaneous T-cell lymphoma (CTCL), peripheral T-cell lymphoma (PTCL), and T cell leukemia/lymphoma. In some examples, the CTCL is Sezary Syndrome (SS). In another example, the CTCL is mycosis fungoides (MF). In specific examples, the human patient may have Stage IIb or higher MF, optionally transformed large cell lymphoma. In some examples, the PTCL is angioimmunoblastic T cell lymphoma (AITL). In some examples, the PTCL is anaplastic large cell lymphoma (ALCL). In some examples, the PTCL is adult T cell leukemia or lymphoma (ATLL). In some examples, the PTCL is PTCL not otherwise (PTCL-NOS).

In some embodiments, the human patient has received up to 4 lines of prior anti-cancer therapy, which optionally is systemic therapy.

In some instances, the human patient has PTCL, ATLL, which optionally is a leukemic and lymphomatous subtype, or AITL and has failed at least one line of systemic therapy. For example, the human patient may have ALCL and has failed a combined therapy comprising brentuximab vedotin. In another example, the human patient may have ALK⁺ ALCL and has failed two prior lines of therapy, one of which comprises brentuximab vedotin.

In some instances, the human patient has ALK⁻ ALCL and has failed one prior line of therapy. For example, the human patient has MF or SS and has failed a prior systemic therapy or a prior mogamulizumab therapy. In some instances, the human patient has failed two of the following: brentuximab vedotin, a histone deacetylase inhibitor, which optionally is romidepsin, pralatrexate, mogamulizumab, total skin electron beam therapy (TSEBT), and pembrolizumab.

In some embodiments, the human patient has a B cell malignancy. Examples include, but are not limited to, diffuse large B cell lymphoma (DLBCL), follicular lymphoma, or mantle cell lymphoma (MCL). In some examples, the human patient has DLBCL and has received up to 4 lines of prior anti-cancer therapy, one line of which is a systemic therapy. In some examples, the human patient has DLBCL and has failed a prior anti-CD19 CAR-T cell therapy. In some examples, the human patient has a myeloid cell malignancy, for example, acute myeloid leukemia (AML).

In some instances, the human patient is free of mogamulizumab treatment at least 50 days prior to the first dose of the population of genetically modified T cells.

In some embodiments, the human patient has at least 10% CD70+ tumor cells in a biological sample obtained from the human patient. For example, the biological sample can be a tumor tissue sample. The level of CD70+ tumor cells may be measured by immunohistochemistry (IHC). In other examples, the biological sample is a blood sample or a bone marrow sample. The level of CD70+ tumor cells may be determined by flow cytometry.

Any of the methods disclosed herein may further comprise, prior to step (i), identifying a human patient having CD70+ tumor cells involved in a hematopoietic cell malignancy, e.g., a T cell malignancy, a B cell malignancy, or a myeloid cell malignancy.

A human patient to be treated by any of the methods disclosed herein may have one or more of the following features: (a) adequate organ function, (b) measurable disease, peripheral blood tumor burden, or last one measurable lesion by imaging, (c) free of a prior stem cell transplantation (SCT), (d) free of a prior anti-CD70 agent or adoptive T cell or NK cell therapy, (e) free of known contraindication to a lymphodepletion therapy, (f) free of T cell or B cell lymphomas with a present or a past malignant effusion that is or was symptomatic, (g) free of hemophagocytic lymphohistiocytosis (HLH), (h) free of central nervous system malignancy or disorders, (i) free of unstable angina, arrhythmia, and/or myocardial infarction, (j) free of diabetes mellitus, (k) free of uncontrolled infections, (1) free of immunodeficiency disorders or autoimmune disorders that require immunosuppressive therapy, and (m) free of solid organ transplantation.

In some embodiments, the method disclosed herein may further comprise monitoring development of acute toxicity after each administration of the population of genetically engineered T cells. Exemplary acute toxicity includes cytokine release syndrome (CRS), ICAN, tumor lysis syndrome, GvHD, on target off-tumor toxicity, viral encephalitis, and/or uncontrolled T cell proliferation. The method may further comprise subjecting the human patient to toxicity management when acute toxicity is observed.

In any of the methods disclosed herein, the anti-CD70 CAR-T cells may comprise a disrupted β2M gene, a disrupted TRAC gene, a disrupted CD70 gene, or a combination thereof. In some instances, the anti-CD70 CAR-T cells comprise a disrupted TRAC gene, and wherein a nucleotide sequence encoding the anti-CD70 CAR is inserted into the disrupted TRAC gene. In some instances, the anti-CD70 CAR-T cells comprise a disrupted TRAC gene, a disrupted β2M gene, and a disrupted CD70 gene, and wherein a nucleotide sequence encoding the CAR is inserted into a genetic site of the anti-CD70 CAR-T cells, optionally wherein the genetic site is the disrupted TRAC gene.

In any of the methods disclosed herein, the CAR that binds CD70 comprises an extracellular domain, a CD8 transmembrane domain, a 4-1BB co-stimulatory domain, and a CD3ζ cytoplasmic signaling domain, and wherein the extracellular domain is a single-chain antibody fragment (scFv) that binds CD70. In some instances, the scFv comprises a heavy chain variable domain (V_{H}) comprising SEQ ID NO: 49, and a light chain variable domain (V_{L}) comprising SEQ ID NO: 50. In one example, the scFv comprises SEQ ID NO: 48. In one specific example, the CAR comprises SEQ ID NO: 46 or SEQ ID NO: 81.

In some embodiments, the disrupted TRAC gene is produced by a CRISPR/Cas9 gene editing system, which comprises a guide RNA comprising a spacer sequence of SEQ ID NO: 8 or 9. In some examples, the disrupted TRAC gene has a deletion of the region targeted the spacer sequence of SEQ ID NO: 8 or 9, or a portion thereof. Alternatively or in addition, the disrupted β2M gene is produced by a CRISPR/Cas9 gene editing system, which comprises a guide RNA comprising a spacer sequence of SEQ ID NO: 12 or 13. In some examples, the disrupted CD70 gene is produced by a CRISPR/Cas9 gene editing system, which comprises a guide RNA comprising a spacer sequence of SEQ ID NO: 4 or 5.

Any of the population of genetically engineered T cells for use in the method disclosed herein may comprise ≥30% CAR+ T cells, ≤0.5% TCR+ T cells, ≤30% B2M+ T cells, and ≤20% CD70+ T cells.

Also within the scope of the present disclosure are any of the anti-CD70 CAR T cells disclosed herein (e.g., the CTX130 cells) for use in treating a hematopoietic malignancy as also disclosed herein, either taken alone or in combination with daratumumab, using any of the treatment regimens disclosed herein. Further, the present disclosure provides uses of any of the anti-CD70 CAR T cells disclosed herein (e.g., the CTX130 cells), either alone or in combination with daratumumab, for manufacturing a medicament for treatment of the target hematopoietic malignancy by any of the treatment regimens disclosed herein.

### BRIEF DESCRIPTION OF THE DRAWINGS

FIG. 1 includes graphs showing efficient multiple gene editing in TRAC⁻/β2M⁻/CD70⁻/anti-CD70 CAR⁺ (i.e., 3× KO (CD70), CD70 CAR⁺) T cells.

FIG. 2 includes a graph showing that normal proportions of CD4+ and CD8+ T cells are maintained among the TRAC⁻/β2M⁻/CD70⁻/anti-CD70 CAR⁺ T cell population.

FIG. 3 includes a graph showing robust cell expansion in TRAC⁻/β2M⁻/CD70⁻/anti-CD70 CAR⁺ T cells. The total number of viable cells was quantified in 3× KO (TRAC−/β2M−/CD70−) and 2× KO (TRAC−/β2M−) anti-CD70 CAR T cells. 3× KO cells were generated with either CD70 sgRNA T7 or T8.

FIGS. 4A-4K includes graphs showing relative CD70 expression in various cancer cell lines. FIG. 4A: graph showing relative CD70 expression in nine different cancer cell lines. FIG. 4B: a graph showing cell kill activity using the triple knockout TRAC⁻/β2M⁻/CD70⁻/anti-CD70 CAR⁺ T cells (3KO (CD70), CD70 CAR⁺) against CD70-deficient chronic myelogenous leukemia (K562) cells at various effector:target ratios. FIG. 4C: a graph showing cell kill activity of the same triple knockout TRAC⁻/β2M⁻/CD70⁻/anti-CD70 CAR⁺ T cells (3KO (CD70), CD70 CAR⁺) against CD70-expressing multiple myeloma (MM.1S) cells at various effector:target ratios. FIG. 4D: a graph showing cell kill activity of the same triple knockout TRAC⁻/β2M⁻/CD70⁻/anti-CD70 CAR⁺ T cells (3KO (CD70), CD70 CAR+) against CD70− expressing T cell lymphoma (HuT78) cells at various effector:target ratios. FIG. 4E: a graph showing cell kill activity of the same triple knockout TRAC⁻/β2M⁻/CD70⁻/anti-CD70 CAR⁺ T cells (3KO (CD70), CD70 CAR⁺) against high CD70-expressing T cell lymphoma cells (MJ), lower CD70-expressing T cell lymphoma cells (HuT78), and non-CD70 expressing negative control cells (K562) at various effector:target ratios. FIGS. 4F-4K: graphs showing cell kill activity of TRAC⁻/β2M⁻/CD70⁻/anti-CD70 CAR⁺ T cells (e.g.: CTX130) in various types of acute myeloid leukemia cell lines, including MV411 (FIG. 4F), EOL-1 (FIG. 4G), HL60 (FIG. 4H), Kasumi-1 (FIG. 4I), KG1 (FIG. 4J), and THP-1 cells (FIG. 4K).

FIGS. 5A-5B include graphs showing anti-tumor activity of anti-CD70 CAR+ T cells, e.g., CTX130 cells. FIG. 5A: graph showing tumor volume reduction in a human T-cell lymphoma xenograft model (e.g., HuT78 tumor cells) exposed to TRAC−/B2M−/CD70− anti-CD70 CAR+ T cells, e.g., CTX130 cells. FIG. 5B: graph showing tumor volume reduction in a human T-cell lymphoma xenograft model (e.g., Hh tumor cells) exposed to TRAC−/B2M−/CD70− anti-CD70 CAR+ T cells, e.g., CTX130 cells.

FIGS. 6A-6D are graphs showing the effect of daratumumab (Dara) on normal immune cells (PBMCs) collected from a healthy donor 96 hours after culture in either media alone or media supplemented with 10% complement. Daratumumab was used at doses of 0.01, 0.1, or 1 μg/mL. Some cells were treated with control isotype mAb (Hu IgG1k). FIG. 6A shows the frequency of NK cells after these treatments. FIG. 6B shows the number of NK cells after these treatments. FIG. 6C shows the frequency of T cells after these treatments. FIG. 6D shows the number of T cells after these treatments.

FIG. 7 is a schematic depicting an exemplary clinical study design to evaluate the safety of a single escalating dose of anti-CD70 CAR+ T cells (e.g., CTX130 cells) administered to to adult subjects with relapsed or refractory T cell or B cell malignancies. The first course of treatment for subjects in this study will include LD chemotherapy (co-administration of fludarabine 30 mg/m2 and cyclophosphamide 500 mg/m2 IV daily for 3 days), followed by a single infusion of CTX130 48 hours to 7 days after LD chemotherapy. The subjects may be considered for up to 2 additional courses of treatment after 1) loss of CR within the first 2 years after initial infusion of CTX130, or 2) PR, SD, or PD with clinical benefit. CR: complete response; D: Day; DLT: dose-limiting toxicity; IV: intravenously; LD: lymphodepleting; M: month; Max: maximum; Min: minimum; PD: progressive disease; PR: partial response; SD: stable disease. The DLT evaluation period is the first 28 days after first CTX130 infusion.

FIG. 8 is a schematic depicting an exemplary clinical study design to evaluate the safety of a single escalating dose of anti-CD70 CAR+ T cells (e.g., CTX130 cells) administered, in combination with daratumumab added to the lymphodepletion regimen, to adult subjects with relapsed or refractory T cell or B cell malignancies. During the first course of treatment, subjects in this study will receive an infusion of daratumumab (single dose of 16 mg/kg IV or 1800 mg SC) followed by LD chemotherapy (co-administration of fludarabine 30 mg/m2 and cyclophosphamide 500 mg/m2 IV daily for 3 days). Daratumumab will be administered at least 12 hours prior to starting LD chemotherapy and within 10 days prior to CTX130 infusion. A single infusion of CTX130 will be administered 48 hours to 7 days after LD chemotherapy. The subjects may be considered for up to 2 additional courses of treatment after 1) loss of CR within the first 2 years after initial infusion of CTX130, or 2) PR, SD, or PD with clinical benefit. * Daratumumab administration at 16 mg/kg IV or 1800 mg SC will be repeated at Day 21 and Day 42. CR: complete response; D: day; Dara: daratumumab; DLT: dose-limiting toxicity; h: hours; IV: intravenously; LD: lymphodepleting; M: month; PD: progressive disease; PR: partial response; SC: subcutaneously; SD: stable disease. Note: DLT evaluation period=28 days.

FIG. 9 is a schematic depicting an exemplary clinical study design to evaluate the safety of an initial infusion of anti-CD70 CAR+ T cells (e.g., CTX130 cells) on Day 1 followed by an additional infusion of CTX130 on Day 5, administered to adult subjects with relapsed or refractory T cell or B cell malignancies. During the first course of treatment, subjects in this study will receive LD chemotherapy (coadministration of fludarabine 30 mg/m2 and cyclophosphamide 500 mg/m2 IV daily for 3 days). The first infusion of CTX130 will be administered 48 hours to 7 days after LD chemotherapy. The second CTX130 infusion on Day 5 (+2 days) will be administered without LD chemotherapy. The subjects may be considered for up to 2 additional courses of treatment after 1) loss of CR within the first 2 years after initial infusion of CTX130, or 2) PR, SD, or PD with clinical benefit. CR: complete response; D: day; DLT: dose-limiting toxicity; IV: intravenously; LD: lymphodepleting; M: month; PD: progressive disease; PR: partial response; SD: stable disease. Note: DLT evaluation period=28 days.

FIG. 10 is a schematic depicting an exemplary clinical study design to evaluate the safety of an initial infusion of anti-CD70 CAR+ T cells (e.g., CTX130 cells) with daratumumab added to the lymphodepletion regimen on Day 1 followed by an additional infusion of CTX130 on Day 5, administered to adult subjects with relapsed or refractory T cell or B cell malignancies. During the first course of treatment, subjects in this study will receive an infusion of daratumumab (single dose of 16 mg/kg IV or 1800 mg SC) followed by LD chemotherapy (co-administration of fludarabine 30 mg/m2 and cyclophosphamide 500 mg/m2 IV daily for 3 days). Daratumumab will be administered at least 12 hours prior to starting LD chemotherapy and within 10 days prior to CTX130 infusion. The first infusion of CTX130 will be administered 48 hours to 7 days after LD chemotherapy. The second CTX130 infusion on Day 5 (+2 days) will be administered without prior daratumumab or LD chemotherapy. The subjects may be considered for up to 2 additional courses of treatment after 1) loss of CR within the first 2 years after initial infusion of CTX130, or 2) PR, SD, or PD with clinical benefit. * Daratumumab administration at 16 mg/kg IV or 1800 mg SC will be repeated at Day 21 and Day 42. CR: complete response; D: day; Dara: daratumumab; DLT: dose-limiting toxicity; h: hours; IV: intravenously; LD: lymphodepleting; M: month; PD: progressive disease; PR: partial response; SC: subcutaneously; SD: stable disease. Note: DLT evaluation period=28 days.

FIG. 11 is a schematic depicting an exemplary clinical study design to evaluate an initial infusion of CTX130 followed by a second infusion of CTX130 on Day 35 (−7 days/+21 days) for subjects who achieve CR, PR, SD, or PD with clinical benefit. During the first course of treatment, subjects in this study will receive LD chemotherapy (coadministration of fludarabine 30 mg/m2 and cyclophosphamide 500 mg/m2 IV daily for 3 days). CTX130 will be administered 48 hours to 7 days after LD chemotherapy. Subjects who achieve CR, PR, SD, or PD with clinical benefit at Day 28 scan (based on Lugano or Olsen criteria as appropriate) will receive a second infusion of CTX130 on Day 35 (−7 days/+21 days) with prior LD chemotherapy (or without LD chemotherapy if the subject is experiencing significant cytopenia). After the first course of treatment, an optional single additional infusion of CTX130 can be administered with prior LD chemotherapy after 1) loss of CR within the first 2 years after initial infusion of CTX130, or 2) PR, SD, or PD with clinical benefit. CR: complete response; D: day; DLT: dose-limiting toxicity; IV: intravenously; LD: lymphodepleting; M: month; PD: progressive disease; PR: partial response; SD: stable disease. Note: DLT evaluation period=28 days.

FIG. 12 is a schematic depicting an exemplary clinical study design to evaluate an initial infusion of CTX130 with Daratumumab Added to the Lymphodepletion Regimen followed by a second infusion of CTX130 on Day 35 (−7 days/+21 days) for subjects who achieve CR, PR, SD, or PD with clinical benefit. During the first course of treatment, subjects in this study will receive an infusion of daratumumab (single dose of 16 mg/kg IV or 1800 mg SC) followed by LD chemotherapy (co-administration of fludarabine 30 mg/m2 and cyclophosphamide 500 mg/m2 IV daily for 3 days). Daratumumab will be administered at least 12 hours prior to starting LD chemotherapy and within 10 days prior to CTX130 infusion. CTX130 will be administered 48 hours to 7 days after LD chemotherapy. Subjects who achieve CR, PR, SD, or PD with clinical benefit at Day 28 scan (based on Lugano or Olsen criteria as appropriate) will receive a second infusion of CTX130 on Day 35 (−7 days/+21 days) with prior daratumumab and LD chemotherapy (or without LD chemotherapy if the subject is experiencing significant cytopenia). After the first course of treatment, an optional single additional infusion of CTX130 can be administered with prior daratumumab and LD chemotherapy after 1) loss of CR within the first 2 years after initial infusion of CTX130, or 2) PR, SD, or PD with clinical benefit. CR: complete response; D: day; Dara: daratumumab; DLT: dose-limiting toxicity; h: hours; IV: intravenously; LD: lymphodepleting; M: month; PD: progressive disease; PR: partial response; SC: subcutaneously; SD: stable disease. Note: DLT evaluation period=28 days.

The details of one or more embodiments of the invention are set forth in the description below. Other features or advantages of the present invention will be apparent from the following drawings and detailed description of several embodiments, and also from the appended claims.

### DETAILED DESCRIPTION

CD70 is a type II membrane protein and ligand for the tumor necrosis factor receptor (TNFR) superfamily member CD27 with a healthy tissue expression distribution limited to activated lymphocytes and subsets of dendritic and thymic epithelial cells and in both humans and mice.

In contrast to its tightly controlled normal tissue expression, CD70 is commonly expressed at elevated levels in multiple T cell and B cell malignancies including peripheral T cell lymphoma not otherwise specified (PTCL-NOS), anaplastic large cell lymphoma (ALCL), Sézary syndrome (SS) including mycosis fungoides (MF), non-smoldering acute adult T cell leukemia/lymphoma (ATLL), angioimmunoblastic T cell lymphoma (AITL; also known as PTCL-AITL), and diffuse large B cell lymphoma (DLBCL). CD70 is also expressed in other hematopoietic malignancies such as myeloid malignancies.

Although hematopoietic cell malignancies such as T cell and B cell malignancies may be treated using conventional treatments, such as chemotherapy and/or checkpoint inhibitors (CPIs), patients may respond poorly or not at all, or relapse after treatment. Such patients have no treatment options with established life-prolonging benefit and are in need of new treatment alternatives.

Surprisingly, the anti-CD70 CAR+ T cells disclosed herein such as CTX130 cells successfully reduced tumor burden in a subcutaneous T cell lymphoma xenograft model and displayed long-term in vivo efficacy that eliminated tumor growth for an extended period (e.g., 90 days after treatment). See also International Patent Application No. PCT/IB2020/060718, the relevant discloses of which are incorporated by reference for the subject matter and purpose referenced herein.

Without wishing to be bound by theory, it is believed that CAR T cells with disrupted MHC Class I are not able to provide the required MHC Class I-NK KIR receptor binding that prevents NK-cells from eliminating MHC-Class I sufficient cells, i.e., self-cells. Thus, allogeneic CAR T cells with disrupted MHC Class I are susceptible to elimination by NK cell-mediated immune surveillance. It was discovered that the administration of an NK cell inhibitor, such as anti-CD38 monoclonal antibody daratumumab, resulted in a reduction of NK cell numbers. The depletion of NK cells, in turn, protects the allogeneic CAR T cell from host NK-mediated cell lysis. The combination of CAR T cell therapy and NK cell inhibitors such as daratumumab thus presents an improvement over the existing CAR T cell therapy.

It was demonstrated that T cells isolated from PBMCs also express CD38 protein on the cell surface. Surprisingly, the addition of an anti-CD38 monoclonal antibody at doses that depleted NK cells did not affect T cell numbers, even after multi-day culture with an anti-CD38 monoclonal antibody. Nor does the addition of anti-CD38 monoclonal antibody at doses that depleted NK cell numbers induce CAR T cell activation. Accordingly, without wishing to be bound by theory, it is believed that anti-CD38 monoclonal antibody treatment is NK cell-specific, and induces reduction of NK cells without causing undesirable non-specific CAR T cell activation or elimination. The addition of an NK cell inhibitor, such as an anti-CD38 monoclonal antibody (e.g., daratumumab), could suppress specific T cells, B cells, and/or NK cells to mitigate potential host immune responses to the allogenic CAR T cells. The NK cell inhibitor may also allow increased expansion and persistence of the CAR T cells. It therefore represents an improvement to existing CAR T cell therapy. See also WO2020/261219, the relevant discloses of which are incorporated by reference for the subject matter and purpose referenced herein.

Accordingly, the present disclosure provides, in some aspects, therapeutic uses of anti-CD70 CAR+ T cells (e.g., CTX130 cells), either taken alone or in combination with an NK cell inhibitor such as an inhibitor of CD38 (e.g., anti-CD38 antibody such as Daratumumab) for treating T cell, B cell, and myeloid cell malignancies. The anti-CD70 CAR+ T cells may be given to a patient as a single dose. Alternatively, multiple doses (e.g., up to 3 doses) may be given to a patient, either taken alone or in combination with the NK cell inhibitor (e.g., Daratumumab). The anti-CD70 CAR T cells, methods of producing such (e.g., via the CRISPR approach), as well as components and processes (e.g., the CRISPR approach for gene editing and components used therein) for making the anti-CD70 CAR+ T cells disclosed herein are also within the scope of the present disclosure.

### I. Anti-CD70 Allogteneic CAR T Cells

Disclosed herein are anti-CD70 CAR T cells (e.g., CTX130 cells) for use in treating a hematopoietic cell malignancy, such as a T cell malignancy, a B cell malignancy, or a myeloid cell malignancy. In some embodiments, the anti-CD70 CAR T cells are allogeneic T cells having a disrupted TRAC gene, a disrupted B2M gene, a disrupted CD70 gene, or a combination thereof. In specific examples, the anti-CD70 CAR T cells express an anti-CD70 CAR and have endogenous TRAC, B2M, and CD70 genes disrupted. Any suitable gene editing methods known in the art can be used for making the anti-CD70 CAR T cells disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9).

Exemplary genetic modifications of the anti-CD70 CAR T cells include targeted disruption of T cell receptor alpha constant (TRAC), β2M, CD70, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection. The disruption of CD70 results in loss of expression of CD70, which prevents possible cell-to-cell fratricide prior to insertion of the CD70 CAR. The addition of the anti-CD70 CAR directs the modified T cells towards CD70-expressing tumor cells.

The anti-CD70 CAR may comprise an anti-CD70 single-chain variable fragment (scFv) specific for CD70, followed by hinge domain and transmembrane domain (e.g., a CD8 hinge and transmembrane domain) that is fused to an intracellular co-signaling domain (e.g., a 4-1BB co-stimulatory domain) and a CD3ζ signaling domain.
(i) Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include MLLLVTSLLLCELPHPAFLLIP (SEQ ID NO: 52) and MALPVTALLLPLALLLHAARP (SEQ ID NO: 53). Other signal peptides may be used.
(a) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen such as a tumor antigen. In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

In some examples, the CAR constructs disclosed herein comprise a scFv extracellular domain capable of binding to CD70. An example of an anti-CD70 CAR is provided in Examples below.
(b) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of FVPVFLPAKPTTTPAPRPPTPAPTIAS QPLSLRPEACRPAAGG AVHTRGLDFACDIYIWAPLAGTCGVLLLSLVITLYCNHRNR (SEQ ID NO: 54) or IYIWAPLAGTCGVLLLSLVITLY (SEQ ID NO: 55). Other transmembrane domains may be used.
(c) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.
(d) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

It should be understood that methods described herein encompasses more than one suitable CAR that can be used to produce genetically engineered T cells expressing the CAR, for example, those known in the art or disclosed herein. Examples can be found in, e.g., WO 2019/097305A2, and WO2019/215500, the relevant disclosures of each of the prior applications are incorporated by reference herein for the purpose and subject matter referenced herein.

For example, the CAR binds CD70 (also known as a “CD70 CAR” or an “anti-CD70 CAR”). The amino acid sequence of an exemplary CAR that binds CD70 is provided in SEQ ID NO: 46 or SEQ ID NO: 81. See also amino acid sequences and coding nucleotide sequences of components in an exemplary anti-CD70 CAR construct in Table 1 below.

TABLE 1
Sequences of Exemplary Anti-CD70 CAR Construct Components.

| | | SEQ |
|---|---|---|
| Description | Sequence | ID NO: |
| CD70 | CCTGCAGGCAGCTGCGCGCTCGCTCGCTCACTGAGGCCGCCCGGGCGTCGG | 43 |
| rAA V | GCGACCTTTGGTCGCCCGGCCTCAGTGAGCGAGCGAGCGCGCAGAGAGGGA | |
| (CD70B scFV | GTGGCCAACTCCATCACTAGGGGTTCCTGCGGCCGCACGCGTGAGATGTAA | |
| with 41BB) | GGAGCTGCTGTGACTTGCTCAAGGCCTTATATCGAGTAAACGGTAGTGCTG | |
| | GGGCTTAGACGCAGGTGTTCTGATTTATAGTTCAAAACCTCTATCAATGAG | |
| | AGAGCAATCTCCTGGTAATGTGATAGATTTCCCAACTTAATGCCAACATAC | |
| | CATAAACCTCCCATTCTGCTAATGCCCAGCCTAAGTTGGGGAGACCACTCC | |
| | AGATTCCAAGATGTACAGTTTGCTTTGCTGGGCCTTTTTCCCATGCCTGCC | |
| | TTTACTCTGCCAGAGTTATATTGCTGGGGTTTTGAAGAAGATCCTATTAAA | |
| | TAAAAGAATAAGCAGTATTATTAAGTAGCCCTGCATTTCAGGTTTCCTTGA | |
| | GTGGCAGGCCAGGCCTGGCCGTGAACGTTCACTGAAATCATGGCCTCTTGG | |
| | CCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCC | |
| | AGCCCCACAGAGCCCCGCCCTTGTCCATCACTGGCATCTGGACTCCAGCCT | |
| | GGGTTGGGGCAAAGAGGGAAATGAGATCATGTCCTAACCCTGATCCTCTTG | |
| | TCCCACAGATATCCAGAACCCTGACCCTGCCGTGTACCAGCTGAGAGACTC | |
| | TAAATCCAGTGACAAGTCTGTCTGCCTATTCACCGATTTTGATTCTCAAAC | |
| | AAATGTGTCACAAAGTAAGGATTCTGATGTGTATATCACAGACAAAACTGT | |
| | GCTAGACATGAGGTCTATGGACTTCAGGCTCCGGTGCCCGTCAGTGGGCAG | |
| | AGCGCACATCGCCCACAGTCCCCGAGAAGTTGGGGGGAGGGGTCGGCAATT | |
| | GAACCGGTGCCTAGAGAAGGTGGCGCGGGGTAAACTGGGAAAGTGATGTCG | |
| | TGTACTGGCTCCGCCTTTTTCCCGAGGGTGGGGGAGAACCGTATATAAGTG | |
| | CAGTAGTCGCCGTGAACGTTCTTTTTCGCAACGGGTTTGCCGCCAGAACAC | |
| | AGGTAAGTGCCGTGTGTGGTTCCCGCGGGCCTGGCCTCTTTACGGGTTATG | |
| | GCCCTTGCGTGCCTTGAATTACTTCCACTGGCTGCAGTACGTGATTCTTGA | |
| | TCCCGAGCTTCGGGTTGGAAGTGGGTGGGAGAGTTCGAGGCCTTGCGCTTA | |
| | AGGAGCCCCTTCGCCTCGTGCTTGAGTTGAGGCCTGGCCTGGGCGCTGGGG | |
| | CCGCCGCGTGCGAATCTGGTGGCACCTTCGCGCCTGTCTCGCTGCTTTCGA | |
| | TAAGTCTCTAGCCATTTAAAATTTTTGATGACCTGCTGCGACGCTTTTTTT | |
| | CTGGCAAGATAGTCTTGTAAATGCGGGCCAAGATCTGCACACTGGTATTTC | |
| | GGTTTTTGGGGCCGCGGGCGGCGACGGGGCCCGTGCGTCCCAGCGCACATG | |
| | TTCGGCGAGGCGGGGCCTGCGAGCGCGGCCACCGAGAATCGGACGGGGGTA | |
| | GTCTCAAGCTGGCCGGCCTGCTCTGGTGCCTGGCCTCGCGCCGCCGTGTAT | |
| | CGCCCCGCCCTGGGCGGCAAGGCTGGCCCGGTCGGCACCAGTTGCGTGAGC | |
| | GGAAAGATGGCCGCTTCCCGGCCCTGCTGCAGGGAGCTCAAAATGGAGGAC | |
| | GCGGCGCTCGGGAGAGCGGGCGGGTGAGTCACCCACACAAAGGAAAAGGGC | |
| | CTTTCCGTCCTCAGCCGTCGCTTCATGTGACTCCACGGAGTACCGGGCGCC | |
| | GTCCAGGCACCTCGATTAGTTCTCGAGCTTTTGGAGTACGTCGTCTTTAGG | |
| | TTGGGGGGAGGGGTTTTATGCGATGGAGTTTCCCCACACTGAGTGGGTGGA | |
| | GACTGAAGTTAGGCCAGCTTGGCACTTGATGTAATTCTCCTTGGAATTTGC | |
| | CCTTTTTGAGTTTGGATCTTGGTTCATTCTCAAGCCTCAGACAGTGGTTCA | |
| | AAGTTTTTTTCTTCCATTTCAGGTGTCGTGACCACCATGGCGCTTCCGGTG | |
| | ACAGCACTGCTCCTCCCCTTGGCGCTGTTGCTCCACGCAGCAAGGCCGCAG | |
| | GTCCAGTTGGTGCAAAGCGGGGCGGAGGTGAAAAAACCCGGCGCTTCCGTG | |
| | AAGGTGTCCTGTAAGGCGTCCGGTTATACGTTCACGAACTACGGGATGAAT | |
| | TGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACT | |
| | ATGACGCGCGATACCAGCATATCCACCGCATACATGGAGCTGTCCCGACTC | |
| | CGGTCAGACGACACGGCTGTCTACTATTGTGCTCGGGACTATGGCGATTAT | |
| | GGCATGGACTACTGGGGTCAGGGTACGACTGTAACAGTTAGTAGTGGTGGA | |
| | GGCGGCAGTGGCGGGGGGGGAAGCGGAGGAGGGGGTTCTGGTGACATAGTT | |
| | ATGACCCAATCCCCAGATAGTTTGGCGGTTTCTCTGGGCGAGAGGGCAACG | |
| | ATTAATTGTCGCGCATCAAAGAGCGTTTCAACGAGCGGATATTCTTTTATG | |
| | CATTGGTACCAGCAAAAACCCGGACAACCGCCGAAGCTGCTGATCTACTTG | |
| | GCTTCAAATCTTGAGTCTGGGGTGCCGGACCGATTTTCTGGTAGTGGAAGC | |
| | GGAACTGACTTTACGCTCACGATCAGTTCACTGCAGGCTGAGGATGTAGCG | |
| | GTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCA | |
| | GCCAAACCGACCACGACTCCCGCCCCGCGCCCTCCGACACCCGCTCCCACC | |
| | ATCGCCTCTCAACCTCTTAGTCTTCGCCCCGAGGCATGCCGACCCGCCGCC | |
| | GGGGGTGCTGTTCATACGAGGGGCTTGGACTTCGCTTGTGATATTTACATT | |
| | TGGGCTCCGTTGGCGGGTACGTGCGGCGTCCTTTTGTTGTCACTCGTTATT | |
| | ACTTTGTATTGTAATCACAGGAATCGCAAACGGGGCAGAAAGAAACTCCTG | |
| | TATATATTCAAACAACCATTTATGAGACCAGTACAAACTACTCAAGAGGAA | |
| | GATGGCTGTAGCTGCCGATTTCCAGAAGAAGAAGAAGGAGGATGTGAACTG | |
| | CGAGTGAAGTTTTCCCGAAGCGCAGACGCTCCGGCATATCAGCAAGGACAG | |
| | AATCAGCTGTATAACGAACTGAATTTGGGACGCCGCGAGGAGTATGACGTG | |
| | CTTGATAAACGCCGGGGGAGAGACCCGGAAATGGGGGGTAAACCCCGAAGA | |
| | AAGAATCCCCAAGAAGGACTCTACAATGAACTCCAGAAGGATAAGATGGCG | |
| | GAGGCCTACTCAGAAATAGGTATGAAGGGCGAACGACGACGGGGAAAAGGT | |
| | CACGATGGCCTCTACCAAGGGTTGAGTACGGCAACCAAAGATACGTACGAT | |
| | GCACTGCATATGCAGGCCCTGCCTCCCAGATAATAATAAAATCGCTATCCA | |
| | TCGAAGATGGATGTGTGTTGGTTTTTTGTGTGTGGAGCAACAAATCTGACT | |
| | TTGCATGTGCAAACGCCTTCAACAACAGCATTATTCCAGAAGACACCTTCT | |
| | TCCCCAGCCCAGGTAAGGGCAGCTTTGGTGCCTTCGCAGGCTGTTTCCTTG | |
| | CTTCAGGAATGGCCAGGTTCTGCCCAGAGCTCTGGTCAATGATGTCTAAAA | |
| | CTCCTCTGATTGGTGGTCTCGGCCTTATCCATTGCCACCAAAACCCTCTTT | |
| | TTACTAAGAAACAGTGAGCCTTGTTCTGGCAGTCCAGAGAATGACACGGGA | |
| | AAAAAGCAGATGAAGAGAAGGTGGCAGGAGAGGGCACGTGGCCCAGCCTCA | |
| | GTCTCTCCAACTGAGTTCCTGCCTGCCTGCCTTTGCTCAGACTGTTTGCCC | |
| | CTTACTGCTCTTCTAGGCCTCATTCTAAGCCCCTTCTCCAAGTTGCCTCTC | |
| | CTTATTTCTCCCTGTCTGCCAAAAAATCTTTCCCAGCTCACTAAGTCAGTC | |
| | TCACGCAGTCACTCATTAACCCACCAATCACTGATTGTGCCGGCACATGAA | |
| | TGCACCAGGTGTTGAAGTGGAGGAATTAAAAAGTCAGATGAGGGGTGTGCC | |
| | CAGAGGAAGCACCATTCTAGTTGGGGGAGCCCATCTGTCAGCTGGGAAAAG | |
| | TCCAAATAACTTCAGATTGGAATGTGTTTTAACTCAGGGTTGAGAAAACAG | |
| | CTACCTTCAGGACAAAAGTCAGGGAAGGGCTCTCTGAAGAAATGCTACTTG | |
| | AAGATACCAGCCCTACCAAGGGCAGGGAGAGGACCCTATAGAGGCCTGGGA | |
| | CAGGAGCTCAATGAGAAAGGTAACCACGTGCGGACCGAGGCTGCAGCGTCG | |
| | TCCTCCCTAGGAACCCCTAGTGATGGAGTTGGCCACTCCCTCTCTGCGCGC | |
| | TCGCTCGCTCACTGAGGCCGGGCGACCAAAGGTCGCCCGACGCCCGGGCTT | |
| | TGCCCGGGCGGCCTCAGTGAGCGAGCGAGCGCGCAGCTGCCTGCAGG | |
| CD70 | GAGATGTAAGGAGCTGCTGTGACTTGCTCAAGGCCTTATATCGAGTAAACG | 44 |
| LHA to RHA | GTAGTGCTGGGGCTTAGACGCAGGTGTTCTGATTTATAGTTCAAAACCTCT | |
| (CD70B scFV | ATCAATGAGAGAGCAATCTCCTGGTAATGTGATAGATTTCCCAACTTAATG | |
| with 41BB) | CCAACATACCATAAACCTCCCATTCTGCTAATGCCCAGCCTAAGTTGGGGA | |
| | GACCACTCCAGATTCCAAGATGTACAGTTTGCTTTGCTGGGCCTTTTTCCC | |
| | ATGCCTGCCTTTACTCTGCCAGAGTTATATTGCTGGGGTTTTGAAGAAGAT | |
| | CCTATTAAATAAAAGAATAAGCAGTATTATTAAGTAGCCCTGCATTTCAGG | |
| | TTTCCTTGAGTGGCAGGCCAGGCCTGGCCGTGAACGTTCACTGAAATCATG | |
| | GCCTCTTGGCCAAGATTGATAGCTTGTGCCTGTCCCTGAGTCCCAGTCCAT | |
| | CACGAGCAGCTGGTTTCTAAGATGCTATTTCCCGTATAAAGCATGAGACCG | |
| | TGACTTGCCAGCCCCACAGAGCCCCGCCCTTGTCCATCACTGGCATCTGGA | |
| | CTCCAGCCTGGGTTGGGGCAAAGAGGGAAATGAGATCATGTCCTAACCCTG | |
| | ATCCTCTTGTCCCACAGATATCCAGAACCCTGACCCTGCCGTGTACCAGCT | |
| | GAGAGACTCTAAATCCAGTGACAAGTCTGTCTGCCTATTCACCGATTTTGA | |
| | TTCTCAAACAAATGTGTCACAAAGTAAGGATTCTGATGTGTATATCACAGA | |
| | CAAAACTGTGCTAGACATGAGGTCTATGGACTTCAGGCTCCGGTGCCCGTC | |
| | AGTGGGCAGAGCGCACATCGCCCACAGTCCCCGAGAAGTTGGGGGGAGGGG | |
| | TCGGCAATTGAACCGGTGCCTAGAGAAGGTGGCGCGGGGTAAACTGGGAAA | |
| | GTGATGTCGTGTACTGGCTCCGCCTTTTTCCCGAGGGTGGGGGAGAACCGT | |
| | ATATAAGTGCAGTAGTCGCCGTGAACGTTCTTTTTCGCAACGGGTTTGCCG | |
| | CCAGAACACAGGTAAGTGCCGTGTGTGGTTCCCGCGGGCCTGGCCTCTTTA | |
| | CGGGTTATGGCCCTTGCGTGCCTTGAATTACTTCCACTGGCTGCAGTACGT | |
| | GATTCTTGATCCCGAGCTTCGGGTTGGAAGTGGGTGGGAGAGTTCGAGGCC | |
| | TTGCGCTTAAGGAGCCCCTTCGCCTCGTGCTTGAGTTGAGGCCTGGCCTGG | |
| | GCGCTGGGGCCGCCGCGTGCGAATCTGGTGGCACCTTCGCGCCTGTCTCGC | |
| | TGCTTTCGATAAGTCTCTAGCCATTTAAAATTTTTGATGACCTGCTGCGAC | |
| | GCTTTTTTTCTGGCAAGATAGTCTTGTAAATGCGGGCCAAGATCTGCACAC | |
| | TGGTATTTCGGTTTTTGGGGCCGCGGGCGGCGACGGGGCCCGTGCGTCCCA | |
| | GCGCACATGTTCGGCGAGGCGGGGCCTGCGAGCGCGGCCACCGAGAATCGG | |
| | ACGGGGGTAGTCTCAAGCTGGCCGGCCTGCTCTGGTGCCTGGCCTCGCGCC | |
| | GCCGTGTATCGCCCCGCCCTGGGCGGCAAGGCTGGCCCGGTCGGCACCAGT | |
| | TGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAG | |
| | GAAAAGGGCCTTTCCGTCCTCAGCCGTCGCTTCATGTGACTCCACGGAGTA | |
| | CCGGGCGCCGTCCAGGCACCTCGATTAGTTCTCGAGCTTTTGGAGTACGTC | |
| | GTCTTTAGGTTGGGGGGAGGGGTTTTATGCGATGGAGTTTCCCCACACTGA | |
| | GTGGGTGGAGACTGAAGTTAGGCCAGCTTGGCACTTGATGTAATTCTCCTT | |
| | GGAATTTGCCCTTTTTGAGTTTGGATCTTGGTTCATTCTCAAGCCTCAGAC | |
| | AGTGGTTCAAAGTTTTTTTCTTCCATTTCAGGTGTCGTGACCACCATGGCG | |
| | CTTCCGGTGACAGCACTGCTCCTCCCCTTGGCGCTGTTGCTCCACGCAGCA | |
| | AGGCCGCAGGTCCAGTTGGTGCAAAGCGGGGCGGAGGTGAAAAAACCCGGC | |
| | GCTTCCGTGAAGGTGTCCTGTAAGGCGTCCGGTTATACGTTCACGAACTAC | |
| | GGGATGAATTGGGTTCGCCAAGCGCCGGGGCAGGGACTGAAATGGATGGGG | |
| | TGGATAAATACCTACACCGGCGAACCTACATACGCCGACGCTTTTAAAGGG | |
| | CGAGTCACTATGACGCGCGATACCAGCATATCCACCGCATACATGGAGCTG | |
| | TCCCGACTCCGGTCAGACGACACGGCTGTCTACTATTGTGCTCGGGACTAT | |
| | GGCGATTATGGCATGGACTACTGGGGTCAGGGTACGACTGTAACAGTTAGT | |
| | AGTGGTGGAGGCGGCAGTGGCGGGGGGGGAAGCGGAGGAGGGGGTTCTGGT | |
| | GACATAGTTATGACCCAATCCCCAGATAGTTTGGCGGTTTCTCTGGGCGAG | |
| | AGGGCAACGATTAATTGTCGCGCATCAAAGAGCGTTTCAACGAGCGGATAT | |
| | TCTTTTATGCATTGGTACCAGCAAAAACCCGGACAACCGCCGAAGCTGCTG | |
| | ATCTACTTGGCTTCAAATCTTGAGTCTGGGGTGCCGGACCGATTTTCTGGT | |
| | AGTGGAAGCGGAACTGACTTTACGCTCACGATCAGTTCACTGCAGGCTGAG | |
| | GATGTAGCGGTCTATTATTGCCAGCACAGTAGAGAAGTCCCCTGGACCTTC | |
| | GGTCAAGGCACGAAAGTAGAAATTAAAAGTGCTGCTGCCTTTGTCCCGGTA | |
| | TTTCTCCCAGCCAAACCGACCACGACTCCCGCCCCGCGCCCTCCGACACCC | |
| | GCTCCCACCATCGCCTCTCAACCTCTTAGTCTTCGCCCCGAGGCATGCCGA | |
| | CCCGCCGCCGGGGGTGCTGTTCATACGAGGGGCTTGGACTTCGCTTGTGAT | |
| | ATTTACATTTGGGCTCCGTTGGCGGGTACGTGCGGCGTCCTTTTGTTGTCA | |
| | CTCGTTATTACTTTGTATTGTAATCACAGGAATCGCAAACGGGGCAGAAAG | |
| | AAACTCCTGTATATATTCAAACAACCATTTATGAGACCAGTACAAACTACT | |
| | CAAGAGGAAGATGGCTGTAGCTGCCGATTTCCAGAAGAAGAAGAAGGAGGA | |
| | TGTGAACTGCGAGTGAAGTTTTCCCGAAGCGCAGACGCTCCGGCATATCAG | |
| | CAAGGACAGAATCAGCTGTATAACGAACTGAATTTGGGACGCCGCGAGGAG | |
| | TATGACGTGCTTGATAAACGCCGGGGGAGAGACCCGGAAATGGGGGGTAAA | |
| | CCCCGAAGAAAGAATCCCCAAGAAGGACTCTACAATGAACTCCAGAAGGAT | |
| | AAGATGGCGGAGGCCTACTCAGAAATAGGTATGAAGGGCGAACGACGACGG | |
| | GGAAAAGGTCACGATGGCCTCTACCAAGGGTTGAGTACGGCAACCAAAGAT | |
| | ACGTACGATGCACTGCATATGCAGGCCCTGCCTCCCAGATAATAATAAAAT | |
| | CGCTATCCATCGAAGATGGATGTGTGTTGGTTTTTTGTGTGTGGAGCAACA | |
| | AATCTGACTTTGCATGTGCAAACGCCTTCAACAACAGCATTATTCCAGAAG | |
| | ACACCTTCTTCCCCAGCCCAGGTAAGGGCAGCTTTGGTGCCTTCGCAGGCT | |
| | GTTTCCTTGCTTCAGGAATGGCCAGGTTCTGCCCAGAGCTCTGGTCAATGA | |
| | TGTCTAAAACTCCTCTGATTGGTGGTCTCGGCCTTATCCATTGCCACCAAA | |
| | ACCCTCTTTTTACTAAGAAACAGTGAGCCTTGTTCTGGCAGTCCAGAGAAT | |
| | GACACGGGAAAAAAGCAGATGAAGAGAAGGTGGCAGGAGAGGGCACGTGGC | |
| | CCAGCCTCAGTCTCTCCAACTGAGTTCCTGCCTGCCTGCCTTTGCTCAGAC | |
| | TGTTTGCCCCTTACTGCTCTTCTAGGCCTCATTCTAAGCCCCTTCTCCAAG | |
| | TTGCCTCTCCTTATTTCTCCCTGTCTGCCAAAAAATCTTTCCCAGCTCACT | |
| | AAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAG | |
| | GGGTGTGCCCAGAGGAAGCACCATTCTAGTTGGGGGAGCCCATCTGTCAGC | |
| | TGGGAAAAGTCCAAATAACTTCAGATTGGAATGTGTTTTAACTCAGGGTTG | |
| | AGAAAACAGCTACCTTCAGGACAAAAGTCAGGGAAGGGCTCTCTGAAGAAA | |
| | TGCTACTTGAAGATACCAGCCCTACCAAGGGCAGGGAGAGGACCCTATAGA | |
| | GGCCTGGGACAGGAGCTCAATGAGAAAGG | |
| CD70 CAR | ATGGCGCTTCCGGTGACAGCACTGCTCCTCCCCTTGGCGCTGTTGCTCCACG | 45 |
| nucleotide | CAGCAAGGCCGCAGGTCCAGTTGGTGCAAAGCGGGGCGGAGGTGAAAAAACC | |
| sequence | CGGCGCTTCCGTGAAGGTGTCCTGTAAGGCGTCCGGTTATACGTTCACGAAC | |
| (CD70B scFv | TACGGGATGAATTGGGTTCGCCAAGCGCCGGGGCAGGGACTGAAATGGATGG | |
| with 41BB) | GGTGGATAAATACCTACACCGGCGAACCTACATACGCCGACGCTTTTAAAGG | |
| | GCGAGTCACTATGACGCGCGATACCAGCATATCCACCGCATACATGGAGCTG | |
| | TCCCGACTCCGGTCAGACGACACGGCTGTCTACTATTGTGCTCGGGACTATG | |
| | GCGATTATGGCATGGACTACTGGGGTCAGGGTACGACTGTAACAGTTAGTAG | |
| | TGGTGGAGGCGGCAGTGGCGGGGGGGGAAGCGGAGGAGGGGGTTCTGGTGAC | |
| | ATAGTTATGACCCAATCCCCAGATAGTTTGGCGGTTTCTCTGGGCGAGAGGG | |
| | CAACGATTAATTGTCGCGCATCAAAGAGCGTTTCAACGAGCGGATATTCTTT | |
| | TATGCATTGGTACCAGCAAAAACCCGGACAACCGCCGAAGCTGCTGATCTAC | |
| | TTGGCTTCAAATCTTGAGTCTGGGGTGCCGGACCGATTTTCTGGTAGTGGAA | |
| | GCGGAACTGACTTTACGCTCACGATCAGTTCACTGCAGGCTGAGGATGTAGC | |
| | GGTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCAG | |
| | CCAAACCGACCACGACTCCCGCCCCGCGCCCTCCGACACCCGCTCCCACCAT | |
| | CGCCTCTCAACCTCTTAGTCTTCGCCCCGAGGCATGCCGACCCGCCGCCGGG | |
| | GGTGCTGTTCATACGAGGGGCTTGGACTTCGCTTGTGATATTTACATTTGGG | |
| | CTCCGTTGGCGGGTACGTGCGGCGTCCTTTTGTTGTCACTCGTTATTACTTT | |
| | GTATTGTAATCACAGGAATCGCAAACGGGGCAGAAAGAAACTCCTGTATATA | |
| | TTCAAACAACCATTTATGAGACCAGTACAAACTACTCAAGAGGAAGATGGCT | |
| | GTAGCTGCCGATTTCCAGAAGAAGAAGAAGGAGGATGTGAACTGCGAGTGAA | |
| | GTTTTCCCGAAGCGCAGACGCTCCGGCATATCAGCAAGGACAGAATCAGCTG | |
| | TATAACGAACTGAATTTGGGACGCCGCGAGGAGTATGACGTGCTTGATAAAC | |
| | GCCGGGGGAGAGACCCGGAAATGGGGGGTAAACCCCGAAGAAAGAATCCCCA | |
| | AGAAGGACTCTACAATGAACTCCAGAAGGATAAGATGGCGGAGGCCTACTCA | |
| | GAAATAGGTATGAAGGGCGAACGACGACGGGGAAAAGGTCACGATGGCCTCT | |
| | ACCAAGGGTTGAGTACGGCAACCAAAGATACGTACGATGCACTGCATATGCA | |
| | GGCCCTGCCTCCCAGATAA | |
| CD70 CAR | MALPVTALLLPLALLLHAARPQVQLVQSGAEVKKPGASVKVSCKASGYTFTN | 46 |
| amino acid | YGMNWVRQAPGQGLKWMGWINTYTGEPTYADAFKGRVTMTRDTSISTAYMEL | |
| sequence | SRLRSDDTAVYYCARDYGDYGMDYWGQGTTVTVSSGGGGSGGGGSGGGGSGD | |
| (CD70B scFv | IVMTQSPDSLAVSLGERATINCRASKSVSTSGYSFMHWYQQKPGQPPKLLIY | |
| with 41BB) | LASNLESGVPDRFSGSGSGTDFTLTISSLQAEDVAVYYCQHSREVPWTFGQG | |
| | TKVEIKSAAAFVPVFLPAKPTTTPAPRPPTPAPTIASQPLSLRPEACRPAAG | |
| | GAVHTRGLDFACDIYIWAPLAGTCGVLLLSLVITLYCNHRNRKRGRKKLLYI | |
| | FKQPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| CD70 CAR | QVQLVQSGAEVKKPGASVKVSCKASGYTFTNYGMNWVRQAPGQGLKWMGWIN | 81 |
| amino acid | TYTGEPTYADAFKGRVTMTRDTSISTAYMELSRLRSDDTAVYYCARDYGDYG | |
| sequence | MDYWGQGTTVTVSSGGGGSGGGGSGGGGSGDIVMTQSPDSLAVSLGERATIN | |
| (without signal | CRASKSVSTSGYSFMHWYQQKPGQPPKLLIYLASNLESGVPDRFSGSGSGTD | |
| peptide) | FTLTISSLQAEDVAVYYCQHSREVPWTFGQGTKVEIKSAAAFVPVFLPAKPT | |
| (CD70B scFv | TTPAPRPPTPAPTIASQPLSLRPEACRPAAGGAVHTRGLDFACDIYIWAPLA | |
| with 41BB) | GTCGVLLLSLVITLYCNHRNRKRGRKKLLYIFKQPFMRPVQTTQEEDGCSCR | |
| | FPEEEEGGCELRVKFSRSADAPAYQQGQNQLYNELNLGRREEYDVLDKRRGR | |
| | DPEMGGKPRRKNPQEGLYNELQKDKMAEAYSEIGMKGERRRGKGHDGLYQGL | |
| | STATKDTYDALHMQALPPR | |
| CD70B | CAGGTCCAGTTGGTGCAAAGCGGGGCGGAGGTGAAAAAACCCGGCGCTTCCG | 47 |
| scFv nucleotide | TGAAGGTGTCCTGTAAGGCGTCCGGTTATACGTTCACGAACTACGGGATGAA | |
| sequence | TTGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACTA | |
| | TGACGCGCGATACCAGCATATCCACCGCATACATGGAGCTGTCCCGACTCCG | |
| | GTCAGACGACACGGCTGTCTACTATTGTGCTCGGGACTATGGCGATTATGGC | |
| | ATGGACTACTGGGGTCAGGGTACGACTGTAACAGTTAGTAGTGGTGGAGGCG | |
| | GCAGTGGCGGGGGGGGAAGCGGAGGAGGGGGTTCTGGTGACATAGTTATGAC | |
| | CCAATCCCCAGATAGTTTGGCGGTTTCTCTGGGCGAGAGGGCAACGATTAAT | |
| | TGTCGCGCATCAAAGAGCGTTTCAACGAGCGGATATTCTTTTATGCATTGGT | |
| | ACCAGCAAAAACCCGGACAACCGCCGAAGCTGCTGATCTACTTGGCTTCAAA | |
| | TCTTGAGTCTGGGGTGCCGGACCGATTTTCTGGTAGTGGAAGCGGAACTGAC | |
| | TTTACGCTCACGATCAGTTCACTGCAGGCTGAGGATGTAGCGGTCTATTATT | |
| | GCCAGCACAGTAGAGAAGTCCCCTGGACCTTCGGTCAAGGCACGAAAGTAGA | |
| | AATTAAA | |
| CD70B | QVQLVQSGAEVKKPGASVKVSCKASGYTFTNYGMNWVRQAPGQGLKWMGWIN | 48 |
| scFv amino acid | TYTGEPTYADAFKGRVTMTRDTSISTAYMELSRLRSDDTAVYYCARDYGDYG | |
| sequence | MDYWGQGTTVTVSSGGGGSGGGGSGGGGSGDIVMTQSPDSLAVSLGERATIN | |
| (linker | CRASKSVSTSGYSFMHWYQQKPGQPPKLLIYLASNLESGVPDRFSGSGSGTD | |
| underlined) | FTLTISSLQAEDVAVYYCQHSREVPWTFGQGTKVEIK | |
| CD70 V_{H} | QVQLVQSGAEVKKPGASVKVSCKASGYTFTNYGMNWVRQAPGQGLKWMGWIN | 49 |
| | TYTGEPTYADAFKGRVTMTRDTSISTAYMELSRLRSDDTAVYYCARDYGDYG | |
| | MDYWGQGTTVTVSS | |
| CD70 V_{L} | DIVMTQSPDSLAVSLGERATINCRASKSVSTSGYSFMHWYQQKPGQPPKLLI | 50 |
| | YLASNLESGVPDRFSGSGSGTDFTLTISSLQAEDVAVYYCQHSREVPWTFGQ | |
| | GTKVEIK | |
| Linker | GGGGSGGGGSGGGGSG | 51 |
| signal peptide | MLLLVTSLLLCELPHPAFLLIP | 52 |
| signal peptide | MALPVTALLLPLALLLHAARP | 53 |
| CD8a | FVPVFLPAKPTTTPAPRPPTPAPTIASQPLSLRPEACRPAAGGAVHTRGLDF | 54 |
| transmembrane | ACDIYIWAPLAGTCGVLLLSLVITLYCNHRNR | |
| domain | | |
| CD8a | IYIWAPLAGTCGVLLLSLVITLY | 55 |
| transmembrane | | |
| 4-1BB | AAACGGGGCAGAAAGAAACTCCTGTATATATTCAAACAACCATTTATGAGAC | 56 |
| nucleotide | CAGTACAAACTACTCAAGAGGAAGATGGCTGTAGCTGCCGATTTCCAGAAGA | |
| sequence | AGAAGAAGGAGGATGTGAACTG | |
| 4-1BB amino | KRGRKKLLYIFKQPFMRPVQTTQEEDGCSCRFPEEEEGGCEL | 57 |
| acid sequence | | |
| CD28 nucleotide | TCAAAGCGGAGTAGGTTGTTGCATTCCGATTACATGAATATGACTCCTCGCC | 58 |
| sequence | GGCCTGGGCCGACAAGAAAACATTACCAACCCTATGCCCCCCCACGAGACTT | |
| | CGCTGCGTACAGGTCC | |
| CD28 amino | SKRSRLLHSDYMNMTPRRPGPTRKHYQPYAPPRDFAAYRS | 59 |
| acid sequence | | |
| CD3ζ nucleotide | CGAGTGAAGTTTTCCCGAAGCGCAGACGCTCCGGCATATCAGCAAGGACAGA | 60 |
| sequence | ATCAGCTGTATAACGAACTGAATTTGGGACGCCGCGAGGAGTATGACGTGCT | |
| | TGATAAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGAGG | |
| | CCTACTCAGAAATAGGTATGAAGGGCGAACGACGACGGGGAAAAGGTCACGA | |
| | TGGCCTCTACCAAGGGTTGAGTACGGCAACCAAAGATACGTACGATGCACTG | |
| | CATATGCAGGCCCTGCCTCCCAGA | |
| CD3ζ amino acid | RVKFSRSADAPAYQQGQNQLYNELNLGRREEYDVLDKRRGRDPEMGGKPRRK | 61 |
| sequence | NPQEGLYNELQKDKMAEAYSEIGMKGERRRGKGHDGLYQGLSTATKDTYDAL | |
| | HMQALPPR | |
| TRAC-LHA | GAGATGTAAGGAGCTGCTGTGACTTGCTCAAGGCCTTATATCGAGTAAACGG | 62 |
| | TAGTGCTGGGGCTTAGACGCAGGTGTTCTGATTTATAGTTCAAAACCTCTAT | |
| | CAATGAGAGAGCAATCTCCTGGTAATGTGATAGATTTCCCAACTTAATGCCA | |
| | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCA | |
| EF1α promoter | GGCTCCGGTGCCCGTCAGTGGGCAGAGCGCACATCGCCCACAGTCCCCGAGA | 63 |
| | AGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCGCA | |
| | ACGGGTTTGCCGCCAGAACACAGGTAAGTGCCGTGTGTGGTTCCCGCGGGCC | |
| | TGGCCTCTTTACGGGTTATGGCCCTTGCGTGCCTTGAATTACTTCCACTGGC | |
| | TGCAGTACGTGATTCTTGATCCCGAGCTTCGGGTTGGAAGTGGGTGGGAGAG | |
| | TTCGAGGCCTTGCGCTTAAGGAGCCCCTTCGCCTCGTGCTTGAGTTGAGGCC | |
| | TGGCCTGGGCGCTGGGGCCGCCGCGTGCGAATCTGGTGGCACCTTCGCGCCT | |
| | GTCTCGCTGCTTTCGATAAGTCTCTAGCCATTTAAAATTTTTGATGACCTGC | |
| | TGCGACGCTTTTTTTCTGGCAAGATAGTCTTGTAAATGCGGGCCAAGATCTG | |
| | CACACTGGTATTTCGGTTTTTGGGGCCGCGGGCGGCGACGGGGCCCGTGCGT | |
| | CCCAGCGCACATGTTCGGCGAGGCGGGGCCTGCGAGCGCGGCCACCGAGAAT | |
| | CGGACGGGGGTAGTCTCAAGCTGGCCGGCCTGCTCTGGTGCCTGGCCTCGCG | |
| | CCGCCGTGTATCGCCCCGCCCTGGGCGGCAAGGCTGGCCCGGTCGGCACCAG | |
| | TTGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAGG | |
| | AAAAGGGCCTTTCCGTCCTCAGCCGTCGCTTCATGTGACTCCACGGAGTACC | |
| | GGGCGCCGTCCAGGCACCTCGATTAGTTCTCGAGCTTTTGGAGTACGTCGTC | |
| | TTTAGGTTGGGGGGAGGGGTTTTATGCGATGGAGTTTCCCCACACTGAGTGG | |
| | GTGGAGACTGAAGTTAGGCCAGCTTGGCACTTGATGTAATTCTCCTTGGAAT | |
| | TTGCCCTTTTTGAGTTTGGATCTTGGTTCATTCTCAAGCCTCAGACAGTGGT | |
| | TCAAAGTTTTTTTCTTCCATTTCAGGTGTCGTGA | |
| Synthetic | AATAAAATCGCTATCCATCGAAGATGGATGTGTGTTGGTTTTTTGTGTG | 64 |
| poly(A) signal | | |
| TRAC-RHA | TGGAGCAACAAATCTGACTTTGCATGTGCAAACGCCTTCAACAACAGCATTA | 65 |
| | TTCCAGAAGACACCTTCTTCCCCAGCCCAGGTAAGGGCAGCTTTGGTGCCTT | |
| | CGCAGGCTGTTTCCTTGCTTCAGGAATGGCCAGGTTCTGCCCAGAGCTCTGG | |
| | TCAATGATGTCTAAAACTCCTCTGATTGGTGGTCTCGGCCTTATCCATTGCC | |
| | ACCAAAACCCTCTTTTTACTAAGAAACAGTGAGCCTTGTTCTGGCAGTCCAG | |
| | AGAATGACACGGGAAAAAAGCAGATGAAGAGAAGGTGGCAGGAGAGGGCACG | |
| | TGGCCCAGCCTCAGTCTCTCCAACTGAGTTCCTGCCTGCCTGCCTTTGCTCA | |
| | GACTGTTTGCCCCTTACTGCTCTTCTAGGCCTCATTCTAAGCCCCTTCTCCA | |
| | AGTTGCCTCTCCTTATTTCTCCCTGTCTGCCAAAAAATCTTTCCCAGCTCAC | |
| | TAAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAGG | |
| | GGTGTGCCCAGAGGAAGCACCATTCTAGTTGGGGGAGCCCATCTGTCAGCTG | |
| | GGAAAAGTCCAAATAACTTCAGATTGGAATGTGTTTTAACTCAGGGTTGAGA | |
| | AAACAGCTACCTTCAGGACAAAAGTCAGGGAAGGGCTCTCTGAAGAAATGCT | |
| | ACTTGAAGATACCAGCCCTACCAAGGGCAGGGAGAGGACCCTATAGAGGCCT | |
| | GGGACAGGAGCTCAATGAGAAAGG | |

(ii) Knock-Out of TRAC, B2M, and/or CD70 Genes

The anti-CD70 CAR-T cells disclosed herein may further have a disrupted TRAC gene, a disrupted B2M gene, a disrupted CD70 gene, or a combination thereof. The disruption of the TRAC locus results in loss of expression of the T cell receptor (TCR) and is intended to reduce the probability of Graft versus Host Disease (GvHD), while the disruption of the #2M locus results in lack of expression of the major histocompatibility complex type I (MHC I) proteins and is intended to improve persistence by reducing the probability of host rejection. The disruption of the CD70 gene would minimize the fratricide effect in producing the anti-CD70 CAR-T cells. Further, disruption of the CD70 gene unexpectedly increased healthy and activity of the resultant engineered T cells. The addition of the anti-CD70 CAR directs the modified T cells towards CD70-expressing tumor cells.

As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region.

In some instances, the disrupted TRAC gene in the anti-CD70 CAR-T cells disclosed herein may comprise a deletion, for example, a deletion of a fragment in Exon 1 of the TRAC gene locus. In some examples, the disrupted TRAC gene comprises a deletion of a fragment comprising the nucleotide sequence of SEQ ID NO: 17, which is the target site of TRAC guide RNA TA-1. See sequence tables below. In some examples, the fragment of SEQ ID NO: 17 may be replaced by a nucleic acid encoding the anti-CD70 CAR. Such a disrupted TRAC gene may comprise the nucleotide sequence of SEQ ID NO: 44.

The disrupted B2M gene in the anti-CD70 CAR-T cells disclosed herein may be generated using the CRISPR/Cas technology. In some examples, a B2M gRNA provided in the sequence table below can be used. The disrupted B2M gene may comprise a nucleotide sequence of any one of SEQ ID Nos: 31-36. See Table 4 below.

Alternatively or in addition, the disrupted CD70 gene in the anti-CD70 CAR-T cells disclosed herein may be generated using the CRISPR/Cas technology. In some examples, a CD70 gRNA provided in the sequence table below can be used. The disrupted CD70 gene may comprise a nucleotide sequence of any one of SEQ ID NOs:37-42. See Table 5 below.

(iii) Exemplary Anti-CD70 CAR T Cells

In some examples, the anti-CD70 CAR T cells are CTX130 cells, which are CD70− directed T cells having disrupted TRAC gene, B2M gene, and CD70 gene. CTX130 cells can be produced via ex vivo genetic modification using CRISPR/Cas9 (Clustered Regularly Interspaced Short Palindromic Repeats/CRISPR associated protein 9) gene editing components (sgRNAs and Cas9 nuclease).

Also within the scope of the present disclosure are populations of anti-CD70 CAR T cells (e.g., a population of CTX130 cells), which comprises genetically engineered cells (e.g., CRISPR-Cas9-mediated gene edited) expressing the anti-CD70 CAR disclosed herein and disrupted TRAC, B2M, and CD70 genes; and the nucleotide sequence encoding the anti-CD70 CAR is inserted into the TRAC locus.

It should be understood that gene disruption encompasses gene modification through gene editing (e.g., using CRISPR/Cas gene editing to insert or delete one or more nucleotides). As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In specific instances, the anti-CD70 CAR+ T cells are CTX130 cells, which are produced using CRISPR technology to disrupt targeted genes, and adeno-associated virus (AAV) transduction to deliver the CAR construct. CRISPR-Cas9-mediated gene editing involves three guide RNAs (sgRNAs): CD70−7 sgRNA (SEQ ID NO: 2) which targets the CD70 locus, TA-1 sgRNA (SEQ ID NO: 6) which targets the TRAC locus, and B2M-1 sgRNA (SEQ ID NO: 10) which targets the β2M locus. The anti-CD70 CAR of CTX130 cells is composed of an anti-CD70 single-chain antibody fragment (scFv) specific for CD70, followed by a CD8 hinge and transmembrane domain that is fused to an intracellular co-signaling domain of 4-1BB and a CD3ζ signaling domain. As such, CTX130 is a CD70-directed T cell immunotherapy comprised of allogeneic T cells that are genetically modified ex vivo using CRISPR/Cas9 gene editing components (sgRNA and Cas9 nuclease).

In some embodiments, at least 50% of a population of CTX130 cells may not express a detectable level of β2M surface protein. For example, at least 55%, at least 60%, at least 70%, at least 75%, at least 80%, at least 85%, at least 90%, or at least 95% of the engineered T cells of a population may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of CTX130 cells may not express a detectable level of TRAC surface protein. For example, at least 55%, at least 60%, at least 70%, at least 75%, at least 80%, at least 85%, at least 90%, or at least 95% of the engineered T cells of a population may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein.

In some embodiments, at least 50% of a population of CTX130 cells may not express a detectable level of CD70 surface protein. For example, at least 55%, at least 60%, at least 70%, at least 75%, at least 80%, at least 85%, at least 90%, at least 95%, or at least 98% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, or 95%-100% of the engineered T cells of a population does not express a detectable level of CD70 surface protein.

In some embodiments, a substantial percentage of the population of CTX130 cells may comprise more than one gene edit, which results in a certain percentage of cells not expressing more than one gene and/or protein.

For example, at least 50% of a population of CTX130 cells may not express a detectable level of two surface proteins, e.g., does not express a detectable level of β2M and TRAC proteins, β2M and CD70 proteins, or TRAC and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of two surface proteins. In another example, at least 50% of a population of the CTX130 cells may not express a detectable level of all of the three target surface proteins β2M, TRAC, and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M, TRAC, and CD70 surface proteins.

In some embodiments, the population of CTX130 cells may comprise more than one gene edit (e.g., in more than one gene), which may be an edit described herein. For example, the population of CTX130 cells may comprise a disrupted TRAC gene via the CRISPR/Cas technology using guide RNA TA-1 (see also Table 2, SEQ ID NOS: 6-7). Alternatively or in addition, the population of CTX130 cells may comprise a disrupted 32M gene via CRISPR/Cas9 technology using the guide RNA of B2M-1 (see also Table 2, SEQ ID NOS: 10-11). Such CTX130 cells may comprise Indels in the β2M gene, which comprise one or more of the nucleotide sequences listed in Table 4. For example, the population of CTX130 cells may comprise a disrupted CD70 gene via the CRISPR/Cas technology using guide RNA CD70-7 (see also Table 2, SEQ ID NOS: 2-3). Further, the population of the CTX130 cells may comprise Indels in the CD70 gene, which may comprise one or more nucleotide sequences listed in Table 5.

In some embodiments, the CTX130 cells may comprise a deletion in the TRAC gene relative to unmodified T cells. For example, the CTX130 cells may comprise a deletion of the fragment AGAGCAACAGTGCTGTGGCC (SEQ ID NO: 17) in the TRAC gene, or a portion of thereof, e.g., a fragment of SEQ ID NO: 17 comprising 1, 2, 3, 4, 5, 6, 7, 8, 9, 10, 11, 12, 13, 14, 15, 16, 17, 18, or 19 consecutive base pairs. In some embodiments, the CTX130 cells include a deletion comprising the fragment of SEQ ID NO: 17 in the TRAC gene. In some embodiments, an engineered T cell comprises a deletion of SEQ ID NO: 17 in the TRAC gene relative to unmodified T cells. In some embodiments, an engineered T cell comprises a deletion comprising SEQ ID NO: 17 in the TRAC gene relative to unmodified T cells.

Further, the population of CTX130 cells may comprise cells expressing an anti-CD70 CAR such as those disclosed herein (e.g., SEQ ID NO: 46 or SEQ ID NO: 81). The coding sequence of the anti-CD70 CAR may be inserted into the TRAC locus, e.g., at the region targeted by guide RNA TA-1 (see also Table 2, SEQ ID NOS: 6-7). In such instances, the amino acid sequence of the exemplary anti-CD70 CAR comprises the amino acid sequence of SEQ ID NO:46.

In some embodiments, at least 30% at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% of the CTX130 cells are CAR+ cells, which express the anti-CD70 CAR. See also WO 2019/097305A2, and WO2019/215500, the relevant disclosures of each of which are incorporated by reference for the subject matter and purpose referenced herein.

In specific examples, the anti-CD70 CAR-T cells disclosed herein (e.g., CTX130 cells) is a population of T cells having ≥30% CAR+ T cells, ≤0.4% TCR+ T cells, ≤30% B2M+ T cells, and ≤2% CD70+ T cells.

### (v) Pharmaceutical Compositions

In some aspects, the present disclosure provides pharmaceutical compositions comprising any of the populations of genetically engineered anti-CD70 CAR T cells as disclosed herein, for example, CTX130 cells, and a pharmaceutically acceptable carrier. Such pharmaceutical compositions can be used in cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered anti-CD70 CAR-T cells (e.g., CTX130 cells) suspended in a cryopreservation solution (e.g., CryoStor® C55). The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, postassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of genetically engineered anti-CD70 CAR-T cells such as the CTX130 cells suspended in a cryopreservation solution (e.g., substantially free of serum) may be placed in storage vials.

Any of the pharmaceutical compositions disclosed herein, comprising a population of genetically engineered anti-CD70 CAR T cells as also disclosed herein (e.g., CTX130 cells), which optionally may be suspended in a cryopreservation solution as disclosed herein may be stored in an environment that does not substantially affect viability and bioactivity of the T cells for future use, e.g., under conditions commonly applied for storage of cells and tissues. In some examples, the pharmaceutical composition may be stored in the vapor phase of liquid nitrogen at ≤−135° C. No significant changes were observed with respect to appearance, cell count, viability, % CAR⁺ T cells, % TCR⁺ T cells, % B2M⁺ T cells, and % CD70⁺ T cells after the cells have been stored under such conditions for a period of time.

In some embodiments, the pharmaceutical composition disclosed herein can be a suspension for infusion, comprising the anti-CD70 CAR T cells disclosed herein such as the CTX130 cells. In some examples, the suspension may comprise about 25-85×10⁶ cells/ml (e.g., 50×10⁶ cells/ml) with ≥30% CAR+ T cells, ≤0.4% TCR+ T cells, ≤30% B2M+ T cells, and ≤2% CD70+ T cells. In some examples, the suspension may comprise about 25×10⁶ CAR+ cells/mL. In specific examples, the pharmaceutical composition may be placed in a vial, each comprising about 1.5×10⁸ CAR+ T cells such as CTX130 cells (e.g., viable cells). In other examples, the pharmaceutical composition may be placed in a vial, each comprising about 3×10⁸ CAR+ T cells such as CTX130 cells (e.g., viable cells).

### II. Preparation of Anti-CD70 CAR T Cells

Any suitable gene editing methods known in the art can be used for making the genetically engineered immune cells (e.g., T cells such as CTX130 cells) disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9). In specific examples, the genetically engineered immune cells such as CTX130 cells are produced by the CRISPR technology in combination with homologous recombination using an adeno-associated viral vector (AAV) as a donor template.
(i) Sources of T Cells

In some embodiments, primary T cells isolated from one or more donors may be used for making the genetically engineered anti-CD70 CAR-T cells. For example, primary T cells may be isolated from a suitable tissue of one or more healthy human donors, e.g., peripheral blood mononuclear cells (PBMCs), bone marrow, lymph nodes tissue, cord blood, thymus issue, tissue from a site of infection, ascites, pleural effusion, spleen tissue, or a combination thereof. In some embodiments, a subpopulation of primary T cells expressing TCRαβ, CD3, CD4, CD8, CD27 CD28, CD38, CD45RA, CD45RO, CD62L, CD127, CD122, CD95, CD197, CCR7, KLRG1, MHC-I proteins, MHC-II proteins, or a combination thereof may be further enriched, using a positive or negative selection technique, which is known in the art. In some embodiments, the T cell subpopulation express TCRαβ, CD4, CD8, or a combination thereof. In some embodiments, the T cell subpopulation express CD3, CD4, CD8, or a combination thereof. In some embodiments, the primary T cells for use in making the genetic edits disclosed herein may comprise at least 40%, at least 50%, or at least 60% CD27+CD45RO− T cells.

In other embodiments, the T cells for use in generating the genetically engineered T cells disclosed herein may be derived from a T cell bank. A T cell bank may comprise T cells with genetic editing of certain genes (e.g., genes involved in cell self renewal, apoptosis, and/or T cell exhaustion or replicative senescence) to improve T cell persistence in cell culture. A T cell bank may be produced from bonafide T cells, for example, non-transformed T cells, terminally differentiated T cells, T cells having stable genome, and/or T cells that depend on cytokines and growth factors for proliferation and expansion. Alternatively, such a T cell bank may be produced from precursor cells such as hematopoietic stem cells (e.g., iPSCs), e.g., in vitro culture. In some examples, the T cells in the T cell bank may comprise genetic editing of one or more genes involved in cell self-renewal, one or more genes involved in apoptosis, and/or one or more genes involved in T cell exhaustion, so as to disrupt or reduce expression of such genes, leading to improved persistence in culture. Examples of the edited genes in a T cell bank include, but are not limited to, Tet2, Fas, CD70, Reg1, or a combination thereof. Compared with the non-edited T counterpart, T cells in a T cell bank may have enhanced expansion capacity in culture, enhanced proliferation capacity, greater T cell activation, and/or reduced apoptosis levels. Additional information of T cell bank may be found in International Application No. PCT/IB2020/058280, the relevant disclosures of which are incorporated by reference for the subject matter and purpose referenced herein.

In some embodiments, parent T cells for use in making the genetically engineered CAR T cells (e.g., any of the T cells derived from primary T cell sources) may be undergone one or more rounds of stimulation, activation, expansion, or a combination thereof. In some embodiments, the parent T cells are activated and stimulated to proliferate in vitro before gene editing. In some embodiments, the T cells are activated, expanded, or both, before or after gene editing. In some embodiments, the T cells are activated and expanded at the same time as gene editing. In some embodiments, the T cells are activated and expanded for about 1-4 days, e.g., about 1-3 days, about 1-2 days, about 2-3 days, about 2-4 days, about 3-4 days, about 1 day, about 2 days, about 3 days, or about 4 days. In some embodiments, the allogeneic T cells are activated and expanded for about 4 hours, about 6 hours, about 12 hours, about 18 hours, about 24 hours, about 36 hours, about 48 hours, about 60 hours, or about 72 hours. Non-limiting examples of methods to activate and/or expand T cells are described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041.
(ii) CRISPR-Cas9-Mediated Gene Editing System for Genetic Engineering of T Cells

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.
(a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 1.

Amino acid sequence of Cas9 nuclease (SEQ ID NO:
1):
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

(b) Guide RNAs (gRNAs)

CRISPR-Cas9-mediated gene editing as described herein includes the use of a guide RNA or a gRNA. As used herein, a “gRNA” refers to a genome-targeting nucleic acid that can direct the Cas9 to a specific target sequence within a CD70 gene or a TRAC gene or a β2M gene for gene editing at the specific target sequence. A guide RNA comprises at least a spacer sequence that hybridizes to a target nucleic acid sequence within a target gene for editing, and a CRISPR repeat sequence.

An exemplary gRNA targeting a CD70 gene is provided in SEQ ID NO: 2. See also WO2019/215500, the relevant disclosures of which are incorporated by reference herein for the subject matter and purpose referenced herein. Other gRNA sequences may be designed using the CD70 gene sequence located on chromosome 19 (GRCh38: chromosome 19: 6,583,183-6,604,103; Ensembl; ENSG00000125726). In some embodiments, gRNAs targeting the CD70 genomic region and Cas9 create breaks in the CD70 genomic region resulting Indels in the CD70 gene disrupting expression of the mRNA or protein.

An exemplary gRNA targeting a TRAC gene is provided in SEQ ID NO: 6. See WO2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein.

An exemplary gRNA targeting a β2M gene is provided in SEQ ID NO: 10. See also WO 2019/097305A2, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

TABLE 2
sgRNA Sequences and Target Gene Sequences.

| | | | SEQ ID |
|---|---|---|---|
| sgRNA Sequences | | | NO: |
| CD70 | Modified | G*C*U*UUGGUCCCAUUGGUCGCguuuuagagcuagaaauagc | 2 |
| sgRNA | | aaguuaaaauaaggcuaguccguuaucaacuugaaaaaguggc | |
| (CD70-7) | | accgagucggugcU*U*U*U | |
| | Unmodified | GCUUUGGUCCCAUUGGUCGCguuuuagagcuagaaauagcaag | 3 |
| | | uuaaaauaaggcuaguccguuaucaacuugaaaaaguggcacc | |
| | | gagucggugcUUUU | |
| CD70 | Modified | G*C*U*UUGGUCCCAUUGGUCGC | 4 |
| spacer | Unmodified | GCUUUGGUCCCAUUGGUCGC | 5 |
| sgRNA | | | |
| TRAC | Modified | A*G*A*GCAACAGUGCUGUGGCCguuuuagagcuagaaauagc | 6 |
| sgRNA | | aaguuaaaauaaggcuaguccguuaucaacuugaaaaaguggc | |
| (TA-1) | | accgagucggugcU*U*U*U | |
| | Unmodified | AGAGCAACAGUGCUGUGGCCguuuuagagcuagaaauagcaag | 7 |
| | | uuaaaauaaggcuaguccguuaucaacuugaaaaaguggcacc | |
| | | gagucggugcUUUU | |
| TRAC | Modified | A*G*A*GCAACAGUGCUGUGGCC | 8 |
| spacer | Unmodified | AGAGCAACAGUGCUGUGGCC | 9 |
| sgRNA | | | |
| β2M | Modified | G*C*U*ACUCUCUCUUUCUGGCCguuuuagagcuagaaauagc | 10 |
| sgRNA | | aaguuaaaauaaggcuaguccguuaucaacuugaaaaaguggc | |
| (B2M-1) | | accgagucggugcU*U*U*U | |
| | Unmodified | GCUACUCUCUCUUUCUGGCCguuuuagagcuagaaauagcaag | 11 |
| | | uuaaaauaaggcuaguccguuaucaacuugaaaaaguggcacc | |
| | | gagucggugcUUUU | |
| β2M | Modified | G*C*U*ACUCUCUCUUUCUGGCC | 12 |
| spacer | Unmodified | GCUACUCUCUCUUUCUGGCC | 13 |
| sgRNA | | | |

Target Sequences (PAM)

| | | |
|---|---|---|
| CD70 | GCTTTGGTCCCATTGGTCGC (GGG) | 14 |
| target | | |
| sequence | | |
| with | | |
| (PAM) | | |
| CD70 | GCTTTGGTCCCATTGGTCGC | 15 |
| target | | |
| sequence | | |
| TRAC | AGAGCAACAGTGCTGTGGCC (TGG) | 16 |
| target | | |
| sequence | | |
| with | | |
| (PAM) | | |
| TRAC | AGAGCAACAGTGCTGTGGCC | 17 |
| target | | |
| sequence | | |
| β2M target | GCTACTCTCTCTTTCTGGCC (TGG) | 18 |
| sequence | | |
| with | | |
| (PAM) | | |
| β2M target | GCTACTCTCTCTTTCTGGCC | 19 |
| sequence | | |

Exemplary sgRNA Formulas

| | | |
|---|---|---|
| sgRNA | nnnnnnnnnnnnnnnnnnnnguuuuagagcuagaaauagcaaguuaaaauaagg | 20 |
| sequence | cuaguccguuaucaacuugaaaaaguggcaccgagucggugcuuuu | |
| sgRNA | nnnnnnnnnnnnnnnnnnnnguuuuagagcuagaaauagcaaguuaaaauaagg | 21 |
| sequence | cuaguccguuaucaacuugaaaaaguggcaccgagucggugc | |
| sgRNA | n(17- | 22 |
| sequence | 30)guuuuagagcuagaaauagcaaguuaaaauaaggcuaguccguuaucaacu | |
| | ugaaa aaguggcaccgagucggugcu(1-8) | |

*indicates a nucleotide with a 2′-O-methyl phosphorothioate modification, “n” refers to the spacer sequence at the 5′ end.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the CD70 target sequence is 5′-GCTTTGGTCCCATTGGTCGC-3′ (SEQ ID NO: 15), then the gRNA spacer sequence is 5′-GCUUUGGUCCCAUUGGUCGC-3′ (SEQ ID NO: 5). In another example, if the TRAC target sequence is 5′-AGAGCAACAGTGCTGTGGCC-3′ (SEQ ID NO: 17), then the gRNA spacer sequence is 5′-AGAGCAACAGUGCUGUGGCC-3′ (SEQ ID NO: 9). In yet another example, if the β2M target sequence is 5′-GCTACTCTCTCTTTCTGGCC-3′ (SEQ ID NO: 19), then the gRNA spacer sequence is 5′-GCUACUCUCUCUUUCUGGCC-3′ (SEQ ID NO: 13). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

A spacer sequence in a gRNA is a sequence (e.g., a 20 nucleotide sequence) that defines the target sequence (e.g., a DNA target sequences, such as a genomic target sequence) of a target gene of interest. An exemplary spacer sequence of a gRNA targeting a CD70 gene is provided in SEQ ID NO: 4. An exemplary spacer sequence of a gRNA targeting a TRAC gene is provided in SEQ ID NO: 8. An exemplary spacer sequence of a gRNA targeting a β2M gene is provided in SEQ ID NO: 12.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO2019/215500, the relevant disclosures of each of the prior applications are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of the prior applications are herein incorporated by reference for the purposes and subject matter referenced herein.

In some embodiments, gRNAs targeting the TRAC genomic region create Indels in the TRAC gene comprising at least one nucleotide sequence selected from the sequences in Table 3. In some embodiments, the gRNA (e.g., SEQ ID NO: 6) targeting the TRAC genomic region creates Indels in the TRAC gene comprising at least one nucleotide sequence selected from the sequences in Table 3.

TABLE 3
Edited TRAC Gene Sequence.

| | Sequence (Deletions indicated by dashes (-); | |
|---|---|---|
| Description | insertions indicated by bold) | SEQ ID NO: |
| TRAC gene edit | A---------------------AGAGCAACAAATCTGACT | 23 |
| TRAC gene edit | AAGAGCAACAGTGCTGT-GCCTGGAGCAACAAATCTGACT | 24 |
| TRAC gene edit | AAGAGCAACAGTG-------CTGGAGCAACAAATCTGACT | 25 |
| TRAC gene edit | AAGAGCAACAGT------GCCTGGAGCAACAAATCTGACT | 26 |
| TRAC gene edit | AAGAGCAACAGTG---------------------CTGACT | 27 |
| TRAC gene edit | AAGAGCAACAGTGCTGTGGGCCTGGAGCAACAAATCTGACT | 28 |
| TRAC gene edit | AAGAGCAACAGTGC--TGGCCTGGAGCAACAAATCTGACT | 29 |
| TRAC gene edit | AAGAGCAACAGTGCTGTGTGCCTGGAGCAACAAATCTGACT | 30 |

In some embodiments, gRNAs targeting the β2M genomic region create Indels in the β2M gene comprising at least one nucleotide sequence selected from the sequences in Table 4. In some embodiments, the gRNA (e.g., SEQ ID NO: 10) targeting the β2M genomic region creates Indels in the β2M gene comprising at least one nucleotide sequence selected from the sequences in Table 4.

TABLE 4
Edited β2M Gene Sequence.

| | Sequence (Deletions indicated by dashes (-); | SEQ ID |
|---|---|---|
| Description | insertions indicated by bold) | NO: |
| β2M gene-edit | CGTGGCCTTAGCTGTGCTCGCGCTACTCTCTCTTTCT- | 31 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 32 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT----- | 33 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATAGCCTG | 34 |
| | GAGGCTATCCAGCGTGAGTCTCTCCTACCCTCCCGCT | |
| β2M gene-edit | CGTGGCCTTAGCTGTGCTCGC------------------------- | 35 |
| | GCTATCCAGCGTGAGTCTCTCCTACCCTCCCGCT | |
| β2M gene-edit | CGTGGCCTTAGCTGTGCTCGCGCTACTCTCTCTTTCTGTGGCCTGGA | 36 |
| | GGCTATCCAGCGTGAGTCTCTCCTACCCTCCCGCT | |

In some embodiments, gRNAs targeting the CD70 genomic region create Indels in the CD70 gene comprising at least one nucleotide sequence selected from the sequences in Table 5. In some embodiments, the gRNA (e.g., SEQ ID NO: 2) targeting the CD70 genomic region creates Indels in the CD70 gene comprising at least one nucleotide sequence selected from the sequences in Table 5.

TABLE 5
Edited CD70 Gene Sequence.

| | Sequence (Deletions indicated by dashes (-); | |
|---|---|---|
| Description | insertions indicated by bold) | SEQ ID NO: |
| CD70 gene-edit | CACACCACGAGGCAGATCACCAAGCCCGCG-- | 37 |
| | CAATGGGACCAAAGCAGCCCGCAGGACG | |
| CD70 gene-edit | CACACCACGAGGCAGATCACCAAGCCCGCGAACCAATGGGACCAAAG | 38 |
| | CAGCCCGCAGGACG | |
| CD70 gene-edit | CACACCACGAGGCAGATC------------ | 39 |
| | ACCAATGGGACCAAAGCAGCCCGCAGGACG | |
| CD70 gene-edit | CACACCACGAGGCAGATCACCAAGCCCGCG- | 40 |
| | CCAATGGGACCAAAGCAGCCCGCAGGACG | |
| CD70 gene-edit | CACACCACGAGGCAGATCACCAAGCCCGC- | 41 |
| | ACCAATGGGACCAAAGCAGCCCGCAGGACG | |
| CD70 gene-edit | CACACCACGAGGCAGATCACCA------------------------- | 42 |
| | AGCCCGCAGGACG | |

(iii) AAV Vectors for Delivery of CAR Constructs to T Cells

A nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids, which confer AAV serotype, which determines which target organs the capsids primarily binds and thus what cells the AAV most efficiently infects. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions and inserting a CAR coding segment into the TRAC gene.

A donor template as disclosed herein can contain a coding sequence for a CAR. In some examples, the CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EFlu promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

### III. NK Cell Inhibitors

NK cells play an important role in both innate and adaptive immunity—including mediating anti-tumor and anti-viral responses. Because NK cells do not require prior sensitization or priming to mediate its cytotoxic function, they are the first line of defense against virus-infected and malignant cells that have missing or nonfunctioning MHC class I (e.g., disrupted MHC class I, or disrupted MCH Class I subunits). NK cells recognize “non-self” cells without the need for antibodies and antigen-priming. MHC class I-specific inhibitory receptors on NK cells negatively regulate NK cell function. Engagement of NK cell inhibitory receptors with their MHC class I ligand checks NK cell-mediated lysis. When MHC class I-disrupted cells fail to bind inhibitory NK receptors (e.g., KIRs), the cells become susceptible to NK cell-mediated lysis. This phenomenon is also referred to as the “missing self recognition.” See e.g., Malmberg K J et al., Immunogenetics (2017), 69:547-556; Cruz-Munoz M E et al., J. Leukoc. Biol. (2019), 105:955-971.

Therefore, engineered human CAR T cells comprising disrupted MHC class I as described herein are susceptible to NK cell-mediated lysis, thus reducing the persistence and subsequent efficacy of the engineered human CAR T cells. Accordingly, in some embodiments the present disclosure provides NK cell inhibitors for use in combination with CAR T cell therapy comprising a population of engineered human CAR T cells as described herein.

The NK cell inhibitor to be used in the methods described herein can be a molecule that blocks, suppresses, or reduces the activity or number of NK cells, either directly or indirectly. The term “inhibitor” implies no specific mechanism of biological action whatsoever, and is deemed to expressly include and encompass all possible pharmacological, physiological, and biochemical interactions with NK cells whether direct or indirect. For the purpose of the present disclosure, it will be explicitly understood that the term “inhibitor” encompasses all the previously identified terms, titles, and functional states and characteristics whereby the NK cell itself, a biological activity of the NK cell (including but not limited to its ability to mediate cell killing), or the consequences of the biological activity, are substantially nullified, decreased, or neutralized in any meaningful degree, e.g., by at least 20%, 50%, 70%, 85%, 90%, 100%, 150%, 200%, 300%, or 500%, or by 10-fold, 20-fold, 50-fold, 100-fold, 1000-fold, or 10⁴-fold.

NK cell inhibitors may be a small molecule compound, a peptide or polypeptide, a nucleic acid, etc. Such NK cell inhibitors may be found in, for example, in International Patent Application No. PCT/IB2020/056085, the relevant discloses of which are incorporated by reference for the subject matter and purpose referenced herein. In some embodiments, the NK cell inhibitor disclosed herein is an antibody specific to CD38.

A. Antibodies that Bind CD38 (Anti-CD38 Antibodies)

In some embodiments, the present disclosure provides antibodies that specifically bind CD38 (anti-CD38 antibodies) for use in the methods described herein. CD38, also known as cyclic ADP ribose hydrolase, is a 46-kDa type II transmembrane glycoprotein that synthesizes and hydrolyzes cyclic adenosine 5′-diphosphate-ribose, an intracellular calcium ion mobilizing messenger. A multifunctional protein, CD38 is also involved in receptor-mediated cell adhesion and signaling. An amino acid sequence of an exemplary human CD38 protein is provided in SEQ ID NO: 70 (NCBI Reference Sequence: NP001766.2). See Table 6 below. Methods for generating antibodies that specifically bind human CD38 are known to those of ordinary skill in the art.

An antibody (interchangeably used in plural form) as used herein is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv, single chain variable fragment (scFv)), mutants thereof, fusion proteins comprising an antibody portion, humanized antibodies, chimeric antibodies, diabodies, linear antibodies, single chain antibodies, single domain antibodies (e.g., camel or llama VHH antibodies), multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

A typical antibody molecule comprises a heavy chain variable region (VH) and a light chain variable region (VL), which are usually involved in antigen binding. These regions/residues that are responsible for antigen-binding can be identified from amino acid sequences of the VH/VL sequences of a reference antibody (e.g., an anti-CD38 antibody as described herein) by methods known in the art. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. As used herein, a CDR may refer to the CDR defined by any method known in the art. Two antibodies having the same CDR means that the two antibodies have the same amino acid sequence of that CDR as determined by the same method. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used as provided herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC).

In some embodiments, an antibody of the present disclosure is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Other forms of humanized antibodies have one or more CDRs (one, two, three, four, five, and/or six) which are altered with respect to the original antibody, which are also termed one or more CDRs “derived from” one or more CDRs from the original antibody. Humanized antibodies may also involve affinity maturation.

In some embodiments, an antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure specifically binds a target antigen (e.g., human CD38). An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a CD38 epitope or is an antibody that binds this epitope with greater affinity, avidity, more readily, and/or with greater duration than it binds to other epitopes of the same antigen or a different antigen. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

Also within the scope of the present disclosure are functional variants of any of the exemplary antibodies as disclosed herein. A functional variant may contain one or more amino acid residue variations in the VH and/or VL, or in one or more of the HC CDRs and/or one or more of the VL CDRs as relative to a reference antibody, while retaining substantially similar binding and biological activities (e.g., substantially similar binding affinity, binding specificity, inhibitory activity, anti-tumor activity, or a combination thereof) as the reference antibody.

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) A→G, S; (b) R→K, H; (c) N→Q, H; (d) D→E, N; (e) C→S, A; (f) Q→N; (g) E→D, Q; (h) G→A; (i) H→N, Q; j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

Anti-CD38 antibodies have been tested in various pre-clinical and clinical studies, e.g., for NK/T cell lymphoma, or T-cell acute lymphoblastic leukemia. Exemplary anti-CD38 antibodies tested for anti-tumor properties include SAR650984 (also referred to as isatuximab, chimeric mAb), which is in phase I clinical trials in patients with CD38+ B-cell malignancies (Deckert J. et al., Clin. Cancer. Res. (2014): 20(17):4574-83), MOR202 (also referred to as MOR03087, fully human mAb), and TAK-079 (fully human mAb).

In some embodiments, an anti-CD38 antibody for use in the present disclosure includes SAR650984 (Isatuximab), MOR202, Ab79, Ab10, HM-025, HM-028, HM-034; as well as antibodies disclosed in U.S. Pat. Nos. 9,944,711, 7,829,673, WO2006/099875, WO 2008/047242, WO2012/092612, and EP 1 720 907 B1, herein incorporated by reference. In some embodiments, the anti-CD38 antibody disclosed herein may be a functional variant of any of the reference antibodies disclosed herein. Such a functional variant may comprise the same heavy chain and light chain complementary determining regions as the reference antibody. In some examples, the functional variant may comprise the same heavy chain variable region and the same light chain variable region as the reference antibody.

In some embodiments, the anti-CD38 antibody for use in the present disclosure is daratumumab. Daratumumab (also referred to as Darzalex®, HuMax-CD38, or IgG1-005) is a fully human IgGκ monoclonal antibody that targets CD38 and has been approved for treating multiple myeloma. It is used as a monotherapy or as a combination therapy for treating newly diagnosed or previously treated multiple myeloma patients. Daratumumab is described in U.S. Pat. No. 7,829,673 and WO2006/099875.

Daratumumab binds an epitope on CD38 that comprises two O-strands located at amino acids 233-246 and 267-280 of CD38. Experiments with CD38 mutant polypeptides show that the S274 amino acid residue is important for daratumumab binding. (van de Donk NWCJ et al., Immunol. Rev. (2016) 270:95-112). Daratumumab's binding orientation to CD38 allows for Fc-receptor mediated downstream immune processes.

Mechanisms of action attributed to Daratumumab as a lymphoma and multiple myeloma therapy includes Fc-dependent effector mechanisms such as complement-dependent cytotoxicity (CDC), natural killer (NK)-cell mediated antibody-dependent cellular cytotoxicity (ADCC) (De Weers M, et al., J. Immunol. (2011) 186:1840-8), antibody-mediated cellular phagocytosis (ADCP) (Overdijk M B et al., MAbs (2015), 7(2):311-21), and apoptosis after cross-linking (van de Donk NWCJ and Usmani S Z, Front. Immunol. (2018), 9:2134).

The full heavy chain amino acid sequence of daratumumab is set forth in SEQ ID NO: 71 and the full light chain amino acid sequence of daratumumab is set forth in SEQ ID NO: 73. The amino acid sequence of the heavy chain variable region of daratumumab is set forth in SEQ ID NO: 64 and the amino acid sequence of the light chain variable region of daratumumab is set forth in SEQ ID NO: 74. Daratumumab includes the heavy chain complementary determining regions (HCDRs) 1, 2, and 3 (SEQ ID NOS: 75, 76, and 77, respectively), and the light chain CDRs (LCDRs) 1, 2, and 3 (SEQ ID NOS. 78, 79, and 80, respectively). See Table 6 below. In some embodiments, these sequences can be used to produce a monoclonal antibody that binds CD38. For example, methods for making daratumumab are described in U.S. Pat. No. 7,829,673 (incorporated herein by reference for the purpose and subject matter referenced herein).

TABLE 6
Amino Acid Sequences of Daratumumab and CD38

| Name | | SEQ |
|---|---|---|
| Description | Amino Acid Sequences | ID NO |
| CD38 | MANCEFSPVSGDKPCCRLSRRAQLCLGVSILVLILVVVLAVVVPRWRQ | 70 |
| | QWSGPGTTKRFPETVLARCVKYTEIHPEMRHVDCQSVWDAFKGAFISK | |
| | HPCNITEEDYQPLMKLGTQTVPCNKILLWSRIKDLAHQFTQVQRDMFTL | |
| | EDTLLGYLADDLTWCGEFNTSKINYQSCPDWRKDCSNNPVSVFWKTVS | |
| | RRFAEAACDVVHVMLNGSRSKIFDKNSTFGSVEVHNLQPEKVQTLEAW | |
| | VIHGGREDSRDLCQDPTIKELESIISKRNIQFSCKNIYRPDKFLQCVKNPE | |
| | DSSCTSEI | |
| Daratumumab | EVQLLESGGGLVQPGGSLRLSCAVSGFTFNSFAMSWVRQAPGKGLE | 71 |
| heavy chain | WVSAISGSGGGTYYADSVKGRFTISRDNSKNTLYLQMNSLRAEDTA | |
| full sequence | VYFCAKDKILWFGEPVFDYWGQGTLVTVSSASTKGPSVFPLAPSSKST | |
| | SGGTAALGCLVKDYFPEPVTVSWNSGALTSGVHTFPAVLQSSGLYSLS | |
| | SV VTVPSSSLGTQTYICNVNHK PSNTKVDKRVEPKSCDKTHTCPPCP | |
| | APELLGGPSVFLFPPKPKDTLMISRTPEVTCVVVDVSHEDPEVKFNWYV | |
| | DGVEVHNAKTKPREEQYNSTYRVVSVLTVLHQDWLNGKEYKCKVSN | |
| | KALPAPIEKTISKAKGQPREPQVYTLPPSREEMTKNQVSLTCLVKGFYPS | |
| | DIAVEWESNGQPENNYKTTPPVLDSDGSFFLYSKLTVDKSRWQQGNV | |
| | FSC SVMHEALHNHYTQKSLSLSP GK | |
| Daratumumab | EVQLLESGGGLVQPGGSLRLSCAVSGFTFNSFAMSWVRQAPGKGLEW | 72 |
| heavy chain | VSAISGSGGGTYYADSVKGRFTISRDNSKNTLYLQMNSLRAEDTAVYF | |
| variable | CAKDKILWFGEPVFDYWGQGTLVTVSSAS | |
| region | | |
| Daratumumab | EIVLTQSPAT LSLSPGERAT LSCRASQSVS SYLAWYQQKPQAPRLLIY | 73 |
| light chain full | DASNRATGIPARFSGSGSGTDFTLTISSLEPEDFAVYYCQQRSNWPPTFG | |
| sequence | QGTKVEIKRTVAAPSVFIFPPSDEQLKSGTASVVCLLNNFYPREAKVQW | |
| | KVDNALQSGNSQESVTEQDSKDSTYSLSSTLTLSKADYEKHKVYACE | |
| | VTHQGLSSPVTKSFNRGEC | |
| Daratumumab | EIVLTQSPATLSLSPGERATLSCRASQSVSSYLAWYQQKPGQAPRLLIY | 74 |
| light chain | DASNRATGIPARFSGSGSGTDFTLTISSLEPEDFAVYYCQQRSNWPPTF | |
| variable | GQGTKVEIK | |
| region | | |
| Daratumumab | SFAMS | 75 |
| heavy chain | | |
| CDR1 | | |
| Daratumumab | AISGSGGGTY YADSVKG | 76 |
| heavy chain | | |
| CDR2 | | |
| Daratumumab | DKILWFGEPV FDY | 77 |
| heavy chain | | |
| CDR3 | | |
| Daratumumab | RASQSVSSYL A | 78 |
| light chain | | |
| CDR1 | | |
| Daratumumab | DASNRAT | 79 |
| light chain | | |
| CDR2 | | |
| Daratumumab | QQRSNWPPT | 80 |
| light chain | | |
| CDR3 | | |

In some embodiments, an anti-CD38 antibody for use in the present disclosure is daratumumab, an antibody having the same functional features as daratumumab, or an antibody which binds to the same epitope as daratumumab or competes against daratumumab from binding to CD38.

In some embodiments, the anti-CD38 antibody comprises: (a) an immunoglobulin heavy chain variable region and (b) an immunoglobulin light variable region, wherein the heavy chain variable region and the light chain variable region defines a binding site (paratope) for CD38. In some embodiments, the heavy chain variable region comprises an HCDR1 comprising the amino acid sequence set forth in SEQ ID NO: 75, an HCDR2 comprising the amino acid sequence set forth in SEQ ID NO: 76; and an HCDR3 comprising the amino acid sequence in SEQ ID NO: 77. The HCDR1, HCDR2, and HCDR3 sequences are separated by the immunoglobulin framework (FR) sequences.

In some embodiments, the anti-CD38 antibody comprises: (a) an immunoglobulin light chain variable region and (b) an immunoglobulin heavy chain variable region, wherein the light chain variable region and the heavy chain variable region defines a binding site (paratope) for CD38. In some embodiments, the light chain variable region comprises an LCDR1 comprising the amino acid sequence set forth in SEQ ID NO: 78, an LCDR2 comprising the amino acid sequence set forth in SEQ ID NO: 79; and an LCDR3 comprising the amino acid sequence in SEQ ID NO: 80. The LCDR1, LCDR2, and LCDR3 sequences are separated by the immunoglobulin framework (FR) sequences.

In some embodiments, the anti-CD38 antibody comprises an immunoglobulin heavy chain variable region (VH) comprising the amino acid sequence set forth in SEQ ID NO: 72, and an immunoglobulin light chain variable region (VL). In some embodiments, the anti-CD38 antibody comprises an immunoglobulin light chain variable region (VL) comprising the amino acid sequence set forth in SEQ ID NO: 74, and an immunoglobulin heavy chain variable region (VH). In some embodiments, the anti-CD38 antibody comprises a VH comprising an amino acid sequence that is at least 70%, 75%, 70%, 85%, 90%, 95%, 96%, 97%, 98%, and 99% identical to the amino acid sequence set forth in SEQ ID NO: 72, and comprises an VL comprising an amino acid sequence that is at least 70%, 75%, 70%, 85%, 90%, 95%, 96%, 97%, 98%, and 99% identical to the amino acid sequence set forth in SEQ ID NO: 74.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

CD38 is expressed on NK cells and infusion of daratumumab results in a reduction of NK cells in peripheral blood and bone marrow. The reduction of NK cells is due to NK-cell killing via ADCC, in which NK cells mediate cytotoxic killing of neighboring NK cells. Administration of daratumumab has also been shown to decrease cell numbers of myeloid derived suppressor cells, regulatory T cells, and regulatory B cells. The elimination of regulatory immune cells results in increased T cell responses and increased T cell numbers (J Krejcik et al., Blood (2016), 128(3):384-394.

Accordingly, in some embodiments, the anti-CD38 antibody (e.g., daratumumab) reduces absolute NK cell numbers. In some embodiments, the anti-CD38 antibody reduces NK cell percentage in PBMCs. In some embodiments, the anti-CD38 antibody inhibits NK cell activity through Fc-mediated mechanisms. In other embodiments, the anti-CD38 antibody mediates the killing of NK cells through CDC. In other embodiments, the anti-CD38 antibody mediates the killing of NK cells through ADCC. In other embodiments, the anti-CD38 antibody enhances phagocytosis of NK cells. In other embodiments, the anti-CD38 antibody enhances apoptosis induction after FcγR-mediated cross-linking.

In some embodiments, the anti-CD38 antibody is daratumumab or an antibody having the same functional features as daratumumab, for example, a functional variant of daratumumab. In some examples, a functional variant comprises substantially the same V_{H} and V_{L} CDRs as daratumumab. For example, it may comprise only up to 8 (e.g., 8, 7, 6, 5, 4, 3, 2, or 1) amino acid residue variations in the total CDR regions of the antibody and binds the same epitope of CD38 with substantially similar affinity (e.g., having a KD value in the same order) as daratumumab. In some instances, the functional variants may have the same heavy chain CDR3 as daratumumab, and optionally the same light chain CDR3 as daratumumab. Alternatively or in addition, the functional variants may have the same heavy chain CDR2 as daratumumab. Such an anti-CD38 antibody may comprise a V_{H} fragment having CDR amino acid residue variations in only the heavy chain CDR1 as compared with the V_{H} of daratumumab. In some examples, the anti-CD38 antibody may further comprise a V_{L} fragment having the same V_{L} CDR3, and optionally same V_{L} CDR1 or V_{L} CDR2 as daratumumab. Alternatively or in addition, the amino acid residue variations can be conservative amino acid residue substitutions (see above disclosures).

In some embodiments, the anti-CD38 antibody may comprise heavy chain CDRs that are at least 80% (e.g., 85%, 90%, 95%, or 98%) sequence identity, individually or collectively, as compared with the V_{H} CDRs of daratumumab. Alternatively or in addition, the anti-CD38 antibody may comprise light chain CDRs that are at least 80% (e.g., 85%, 90%, 95%, or 98%) sequence identity, individually or collectively, as compared with the V_{L} CDRs as daratumumab. As used herein, “individually” means that one CDR of an antibody shares the indicated sequence identity relative to the corresponding CDR of daratumumab. “Collectively” means that three V_{H} or V_{L} CDRs of an antibody in combination share the indicated sequence identity relative the corresponding three V_{H} or V_{L} CDRs of daratumumab.

In some embodiments, the anti-CD38 antibody binds to the same epitope bound by daratumumab on human CD38. In some embodiments, the anti-CD38 antibody competes with daratumumab for binding to human CD38.

Competition assays for determining whether an antibody binds to the same epitope as daratumumab, or competes with daratumumab for binding to CD38, are known in the art. Exemplary competition assays include immunoassays (e.g., ELISA assay, RIA assays), surface plasmon resonance, (e.g., BIAcore analysis), bio-layer interferometry, and flow cytometry.

A competition assay typically involves an immobilized antigen (e.g., CD38), a test antibody (e.g., CD38-binding antibody) and a reference antibody (e.g., daratumumab). Either one of the reference or test antibody is labeled, and the other unlabeled. In some embodiments, competitive binding is determined by the amount of a reference antibody bound to the immobilized antigen in increasing concentrations of the test antibody. Antibodies that compete with a reference antibody include antibodies that bind the same or overlapping epitopes as the reference antibody. In some embodiments, the test antibodies bind to adjacent, non-overlapping epitopes such that the proximity of the antibodies causes a steric hindrance sufficient to affect the binding of the reference antibody to the antigen.

A competition assay can be conducted in both directions to ensure that the presence of the label or steric hindrance does not interfere or inhibit binding to the epitope. For example, in the first direction, the reference antibody is labeled and the test antibody is unlabeled. In the second direction, the test antibody is labeled, and the reference antibody is unlabeled. In another embodiment, in the first direction, the reference antibody is bound to the immobilized antigen, and increasing concentrations of the test antibody are added to measure competitive binding. In the second direction, the test antibody is bound to the immobilized antigen, and increasing concentrations of the reference antibody are added to measure competitive binding.

In some embodiments, two antibodies can be determined to bind to the same epitope if essentially all amino acid mutations in the antigen that reduce or eliminate the binding of one antibody reduce or eliminate binding of the other. Two antibodies can be determined to bind to overlapping epitopes if only a subset of the mutations that reduce or eliminate the binding of one antibody reduces or eliminates the binding of the other.

In some embodiments, the heavy chain of any of the anti-CD38 antibodies as described herein (e.g., daratumumab) may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-CD38 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

Any of the anti-CD38 antibodies, including human antibodies or humanized antibodies, can be prepared by conventional approaches, for example, hybridoma technology, antibody library screening, or recombinant technology. See, for example, Harlow and Lane, (1998) Antibodies: A Laboratory Manual, Cold Spring Harbor Laboratory, New York, WO 87/04462, Morrison et al., (1984) Proc. Nat. Acad. Sci. 81:6851, and Queen et al., Proc. Natl. Acad. Sci. USA, 86:10029-10033 (1989).

It should be understood that the described antibodies are only exemplary and that any anti-CD38 antibodies can be used in the compositions and methods disclosed herein. Methods for producing antibodies are known to those of skill in the art.

### IV. Treatment of Hematopoietic Cell Malignancies

In some aspects, provided herein are methods for treating a human patient having a hematopoietic cell malignancy (e.g., a T cell or B cell malignancy, or a myeloid cell malignancy) using a population of any of the anti-CD70 CAR T cells such as the CTX130 cells as disclosed herein, either taken alone or in combination with an NK cell inhibitor such as an anti-CD38 antibody (e.g., Daratumumab), either by a single dose or by multiple doses. Such an allogeneic anti-CD70 CAR T cell therapy may comprise two stages of treatment (i) a conditioning regimen (lymphodepleting treatment), which comprises giving one or more doses of one or more lymphodepleting agents to a suitable human patient, and (ii) a treatment regimen (anti-CD70 CAR T cell therapy), which comprises administration of the population of anti-CD70 CAR T cells such as the CTX130 cells as disclosed herein to the human patient. When applicable, multiple doses of the anti-CD70 CAR T cells may be given to the human patient and a lymphodepletion treatment can be applied to the human patient prior to each dose of the anti-CD70 CAR T cells. Alternatively, the treatment regimen in the second stage may further comprise administering to the human patient one or more doses of an NK cell inhibitor such as Daratumumab.
(i) Patient Population

A human patient may be any human subject for whom diagnosis, treatment, or therapy is desired. A human patient may be of any age. In some embodiments, the human patient is an adult (e.g., a person who is at least 18 years old). In some embodiments, the human patient is a child. In some embodiments, the human patient has a body weight≥40 kg (e.g., ≥60 kg).

A human patient to be treated by the methods described herein can be a human patient having, suspected of having, or a risk for having a hematopoietic cell malignancy (e.g., comprising CD70+ disease cells). In some examples, the human patient has, is suspected of having, or is at risk for a T cell malignancy. In some examples, the human patient has, is suspected of having, or is at risk for a B cell malignancy. In some examples, the human patient has, is suspected of having, or is at risk for a myeloid cell malignancy. A subject suspected of having a hematopoietic cell malignancy might show one or more symptoms of the hematopoietic cell malignancy, e.g., unexplained weight loss, fatigue, night sweats, shortness of breath, or swollen glands. A subject at risk for a hematopoietic cell malignancy can be a subject having one or more of the risk factors for a hematopoietic cell malignancy, e.g., a weakened immune system, age, male, or infection (e.g., Epstein-Barr virus infection). A human patient who needs the anti-CD70 CAR T cell (e.g., CTX130 cell) treatment may be identified by routine medical examination, e.g., physical examination, laboratory tests, biopsy (e.g., bone marrow biopsy and/or lymph node biopsy), magnetic resonance imaging (MRI) scans, or ultrasound exams.

In some embodiments, the human patient has a T cell malignancy, e.g., a relapsed or refractory T cell malignancy. Such a human patient may carry CD70+ disease T cells. Examples include, but are not limited to, cutaneous T-cell lymphoma (CTCL), peripheral T-cell lymphoma (PTCL), and T cell leukemia. In some instances, the T cell malignancy can be CTCL, which may include mycosis fungoides (MF), for example, stage IIb or higher, including transformed large cell lymphoma, or Sezary Syndrome (SS).

In some instances, the T cell malignancy is PTCL. Examples include, but are not limited to, angioimmunoblastic T cell lymphoma (AITL), anaplastic large cell lymphoma (ALCL), which may be Alk positive or Alk negative, adult T cell leukemia or lymphoma (ATLL), which may exclude the smoldering subtype (non-smoldering ATLL); and peripheral T-cell lymphoma not otherwise (PTCL-NOS).

In some embodiments, the human patient may have a B cell malignancy, for example, a relapsed or refractory B cell malignancy. Such a human patient may carry CD70+ disease B cells. In some examples, the human patient has diffused large B cell lymphoma (DLBCL). Such a human patient may have failed a prior anti-CD19 CAR-T cell therapy. In other examples, the human patient has mantle cell lymphoma (MCL), which is an aggressive type of B-cell non-Hodgkin lymphoma (NHL) associated with poor prognosis.

In yet other embodiments, the human patient may have a myeloid cell malignancy, for example, a relapsed or refractory myeloid cell malignancy. In some examples, the human patient has acute myeloid leukemia (AML, also referred to as acute myelogenous leukemia).

In some embodiments, the human patient has a CD70+ leukemia. In some embodiments, the human patient has a CD70+ T cell leukemia. In some embodiments, the human patient has a CD70+ lymphoma. In some embodiments, the human patient has a CD70+ T cell lymphoma.

In some embodiments, the human patient to be treated by the methods described herein can be a human patient having a tumor comprising CD70-expressing tumor cells (CD70− expressing tumor), which may be identified by any method known in the art. For example, a CD70-expressing tumor may be identified by immunohistochemistry (IHC) in tissue collected by excisional or core biopsy of a representative tumor. In another example, a CD70-expressing tumor may be identified by flow cytometry in tumor cells defined by immunophenotyping collected in the peripheral blood or bone marrow. In specific examples, the human patient to be treated by the method disclosed herein may have a tumor comprising at least 10% CD70+ tumor cells in the total cancer cells in a biological sample (e.g., a tissue sample such as a lymph node sample, a blood sample or a bone marrow sample).

Any of the methods disclosed herein may further comprise a step of identifying a human patient suitable for the allogeneic anti-CD70 CAR T therapy based on presence and/or level of CD70+ tumor cells in the patient. The identifying step can be performed by determining presence and/or level of CD70+ tumor cells in a biopsy sample obtained from a candidate patient via, e.g., IHC. Alternatively, the identifying step can be performed by determining presence and/or level of CD70+ tumor cells in a blood sample or a bone marrow sample obtained from the candidate patient via, e.g., flow cytometry.

A human patient to be treated by methods described herein may be a human patient that has relapsed following a treatment and/or that has been become resistant to a treatment and/or that has been non-responsive to a treatment. Non-limiting examples include a patient that has: (a) relapsed or refractory hematopoietic cell malignancy (e.g., T cell or B cell malignancies, or myeloid cell malignancy), (b) SS or mycosis fungoides (MF)≥Stage IIB, who may be in need of transplant, (c) diffuse large B cell lymphoma (DLBCL), who may be non-responsive to anti-CD19 CAR T cell therapy, (d) PTCL, ATLL (e.g., leukemic ATLL, lymphomatous ATLL), or AITL and has failed a first line systemic therapy, (e) ALCL and has failed a combined therapy comprising breutuximab vedotin, (f) ALK+ ALCL and has failed two prior lines of therapy (for example, one of such may comprise brentuximab vedotin), (g) ALK− ALCL and has failed one prior line of therapy, or (h) MF or SS and has failed one or more (e.g., at least two) prior systemic therapies, which, in some instances, may comprise a prior mogamulizumab therapy. In some instances, the human patient may have received up to four lines of priority anti-cancer therapy, one or more of which may be systemic therapy.

A human patient to be treated by methods described herein may be a human patient that has had recent prior treatment or a patient that is free of prior treatment. For example, a human patient to be treated as described herein may be free of mogamulizumab treatment at least three months prior to the first dose of the population of genetically modified T cells.

Any of the human patients treated using a method disclosed herein may receive subsequent treatment. For example, the human patient is subject to an anti-cytokine therapy. In another example, the human patient is subject to autologous or allogeneic hematopoietic stem cell transplantation after treatment with the population of genetically engineered T cells.

A human patient may be screened to determine whether the patient is eligible to undergo a conditioning regimen (lymphodepleting treatment) and/or a treatment regimen (anti-CD70 CAR T cell therapy). For example, a human patient who is eligible for lymphodepletion treatment does not show one or more of the following features: (a) change in performance status to Eastern Cooperative Oncology Group (ECOG)>1, (b) significant worsening of clinical status (e.g., significant worsening of clinical status that may increase the potential risk of AEs associated with the conditioning regimen and/or the treatment regimen), (c) requirement for supplemental oxygen to maintain a saturation level of greater than 92%, (d) uncontrolled cardiac arrhythmia, (e) hypotension requiring vasopressor support, (f) active infection, (g) any acute neurological toxicity (e.g., ≥2 acute neurological toxicity), and (h) platelet count≤25,000/mm³ and/or absolute neutrophil count≤500/mm³.

In another example, a human patient who is eligible for a treatment regimen does not show one or more of the following features: (a) change in performance status to Eastern Cooperative Oncology Group (ECOG)>1, (b) active uncontrolled infection, (c) significant worsening of clinical status (e.g., significant worsening of clinical status that may increase the potential risk of AEs associated with allogenic CAR T cell infusion), (d) any acute neurological toxicity (e.g., ≥2 acute neurological toxicity).

Significant worsening of clinical status that may increase the potential risk of AEs associated with the conditioning regimen and/or the treatment regimen may include, but is not limited to, clinically significant worsening of cytopenia, clinically significant increase of transaminase levels (e.g., >3×ULN), clinically significant increase of total bilirubin (e.g., >2×ULN), and clinically significant increase in serum creatinine.

A human patient may be screened and excluded from the conditioning regimen and/or treatment regimen based on such screening results. For example, a human patient may be excluded from a conditioning regimen and/or a treatment regimen if the patient meets any of the following exclusion criteria: (a) prior allogeneic stem cell transplant (SCT), (b) less than 60 days from autologous SCT at time of screening and with unresolved serious complications, (c) prior treatment with any anti-CD70 targeting agents, (d) prior treatment with any CAR T cells or any other modified T or natural killer (NK) cells except autologous CD19 CAR T cells, and the patient has DLBCL, (e) known contraindication to any lymphodepletion treatment or any of the excipients of any treatment regimen, (f) T cell or B cell lymphomas with a present or past malignant effusion that is or was symptomatic, (g) clinical signs of hemophagocytic lymphohistiocytosis (HLH), (h) detectable malignant cells from cerebrospinal fluid (CSF) or magnetic resonance imaging (MRI) indicating brain metastases, (i) history or presence of clinically relevant CNS pathology, (j) unstable angina, arrhythmia, or myocardial infarction within 6 months prior to screening, (k) previous or concurrent malignancy, except those treated with a curative approach who have been in remission for >12 months without requiring systemic therapy (in some instances, basal cell or squamous cell skin carcinoma, adequately resected and in situ carcinoma of cervix, or a previous malignancy that was completely resected and has been in remission for greater than 3 years may be allowed), and (1) uncontrolled, acute life-threatening bacterial, viral, or fungal infection.

A human patient subjected to lymphodepletion treatment may be screened for eligibility to receive one or more doses of the anti-CD70 CAR T cells disclosed herein such as the CTX130 cells. For example, a human patient subjected to lymphodepletion treatment that is eligible for an anti-CD70 CAR T cell treatment does not show one or more of the following features: (a) change in performance status to Eastern Cooperative Oncology Group (ECOG)>1, (b) active uncontrolled infection, (c) significant worsening of clinical status (e.g., significant worsening of clinical status that may increase the potential risk of AEs associated with allogenic CAR T cell infusion), (d) any acute neurological toxicity (e.g., ≥2 acute neurological toxicity).

Following each dosing of anti-CD70 CAR T cells, a human patient may be monitored for acute toxicities such as cytokine release syndrome (CRS), tumor lysis syndrome (TLS), neurotoxicity (e.g., immune effector cell-associated neurotoxicity syndrome or ICANS), and graft versus host disease (GvHD). In addition, one or more of the following adverse effects may be monitored: hypotension, renal insufficiency (which may be caused, e.g., by suppression of renal tubular-like epithelium cells), hemophagocytic lymphohistiocytosis (HLH), prolonged cytopenias, and/or suppression of osteoblasts. After each dose of anti-CD70 CAR T cells, a human patient may be monitored for at least 28 days for development of toxicity.

When a human patient exhibits one or more symptoms of acute toxicity, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CAR T cells.

Alternatively, or in addition to, when a human patient exhibits one or more symptoms of acute toxicity, treatment of the human patient may be terminated. Patient treatment may also be terminated if the patient exhibits one or more signs of an adverse event (AE), e.g., the patient has an abnormal laboratory finding and/or the patient shows signs of disease progression.
(ii) Conditioning Regimen (Lymphodepleting Therapy)

Any human patients suitable for the treatment methods disclosed herein may receive a lymphodepleting therapy to reduce or deplete the endogenous lymphocyte of the subject.

Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

In some embodiments, the method described herein involves a conditioning regimen that comprises one or more lymphodepleting agents, for example, fludarabine and cyclophosphamide. A human patient to be treated by the method described herein may receive multiple doses of the one or more lymphodepleting agents for a suitable period (e.g., 1-5 days) in the conditioning stage. The patient may receive one or more of the lymphodepleting agents once per day during the lymphodepleting period. In one example, the human patient receives fludarabine at about 20-50 mg/m² (e.g., 20 mg/m² or 30 mg/m²) per day for 2-4 days (e.g., 3 days) and cyclophosphamide at about 300-600 mg/m² (e.g., 500 mg/m²) per day for 2-4 days (e.g., 3 days). In another example, the human patient receives fludarabine at about 20-30 mg/m² (e.g., 25 mg/m²) per day for 2-4 days (e.g., 3 days) and cyclophosphamide at about 300-500 mg/m² (e.g., 300 mg/m² or 400 mg/m²) per day for 2-4 days (e.g., 3 days). If needed, the dose of cyclophosphamide may be increased, for example, to up to 1,000 mg/m².

The human patient may then be administered any of the anti-CD70 CAR T cells such as CTX130 cells within a suitable period after the lymphodepleting therapy as disclosed herein. For example, a human patient may be subject to one or more lymphodepleting agent about 2-7 days (e.g., for example, 2, 3, 4, 5, 6, 7 days) before administration of the anti-CD70 CAR+ T cells (e.g., CTX130 cells).

Since the allogeneic anti-CD70 CAR-T cells such as CTX130 cells can be prepared in advance, the lymphodepleting therapy as disclosed herein may be applied to a human patient having a T cell or B cell malignancy within a short time window (e.g., within 2 weeks) after the human patient is identified as suitable for the allogeneic anti-CD70 CAR-T cell therapy disclosed herein.

Methods described herein encompass redosing a human patient with anti-CD70 CAR+ T cells. In such instances, the human patient is subjected to lymphodepletion treatment prior to redosing. For example, a human patient may be subject to a first lymphodepletion treatment and a first dose of CTX130 followed by a second lymphodepletion treatment and a second dose of CTX130. In another example, a human patient may be subject to a first lymphodepletion treatment and a first dose of CTX130, a second lymphodepletion treatment and a second dose of CTX130, and a third lymphodepletion treatment and a third dose of CTX130.

Prior to any of the lymphodepletion steps (e.g., prior to the initial lymphodepletion step or prior to any follow-on lymphodepletion step in association with a re-dosing of the anti-CD70 CAR T cells such as CTX130 cells), a human patient may be screened for one or more features to determine whether the patient is eligible for lymphodepletion treatment. For example, prior to lymphodepletion, a human patient eligible for lymphodepletion treatment does not show one or more of the following features: (a) change in performance status to Eastern Cooperative Oncology Group (ECOG)>1, (b) significant worsening of clinical status (e.g., significant worsening of clinical status that may increase the potential risk of AEs associated with lymphodepletion treatment), (c) requirement for supplemental oxygen to maintain a saturation level of greater than 92%, (d) uncontrolled cardiac arrhythmia, (e) hypotension requiring vasopressor support, (f) active infection, (g) any acute neurological toxicity (e.g., ≥2 acute neurological toxicity), and (h) platelet count≤25,000/mm³ and/or absolute neutrophil count≤500/mm³.

In some examples, significant worsening of clinical status that may increase potential risk of adverse events associated with lymphodepletion treatment includes, but is not limited to, clinically significant worsening of any cytopenia, clinically significant increase of transaminase levels (e.g., >3×ULN), clinically significant increase of total bilirubin (e.g., >2×ULN), and/or clinically significant increase in serum creatinine.

In some instances, when the LD chemotherapy cannot be completed in 3 consecutive days, the LD chemotherapy may restart.

Following lymphodepletion, a human patient may be screened for one or more features to determine whether the patient is eligible for treatment with anti-CD70 CAR T cells. For example, prior to anti-CD70 CAR T cell treatment and after lymphodepletion treatment, a human patient eligible for anti-CD70 CAR T cells treatment does not show one or more of the following features: (a) change in performance status to Eastern Cooperative Oncology Group (ECOG)>1, (b) active uncontrolled infection, (c) significant worsening of clinical status (e.g., significant worsening of clinical status that may increase the potential risk of AEs associated with allogenic CAR T cell infusion), and (d) any acute neurological toxicity (e.g., ≥2 acute neurological toxicity).
(iii) Administration of Anti-CD70 CAR T Cells

After receiving the lymphodepletion treatment disclosed herein (e.g., within 2-7 days after the lymphodepletion treatment), a human patient may be given an effective amount of a population of genetically engineered T cells described herein (e.g., CTX130 cells) via a suitable route (e.g., intravenous infusion).

Administering anti-CD70 CAR T cells may include placement (e.g., transplantation) of a genetically engineered T cell population into a human patient by a method or route that results in at least partial localization of the genetically engineered T cell population at a desired site, such as a tumor site, such that a desired effect(s) can be produced. The genetically engineered T cell population can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of the genetically engineered T cell population can be administered via a systemic route of administration, such as an intraperitoneal or intravenous route.

In some embodiments, the genetically engineered T cell population is administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

An effective amount refers to the amount of a genetically engineered T cell population needed to prevent or alleviate at least one or more signs or symptoms of a medical condition (e.g., a T cell or B cell malignancy), and relates to a sufficient amount of a genetically engineered T cell population to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

An effective amount of a genetically engineered T cell population (e.g., one dose) may comprise about 1×10⁶ to about 1.8×10⁹ CAR+ T cells (e.g., 1×10⁹ CAR+ T cells), for example, about 1×10⁷ CAR+ cells to about 1.8×10⁹ CAR+ cells, e.g., 1×10⁷ CAR+ cells to about 9×10⁸ CAR+ cells, about 3×10⁷ cells to about 9×10⁸ cells that express a CAR that binds CD70, or about 9×10⁸ CAR+ cells to about 1.8×10⁹ CAR+ T cells (e.g., CAR⁺ CTX130 cells).

In some instances, an effective amount of a genetically engineered T cell population may comprise about 3.0×10⁷ cells to about 1.8×10⁹ cells (e.g., about 3.0×10⁷ cells to about 9×10⁸ cells) that express an anti-CD70 CAR (CAR⁺ cells), for example, CAR⁺ CTX130 cells. In some instances, an effective amount of a genetically engineered T cell population may comprise about 3.0×10⁷ cells to about 7.5×10⁸ cells that express an anti-CD70 CAR (CAR⁺ cells), for example, CAR⁺ CTX130 cells. In some instances, an effective amount of a genetically engineered T cell population may comprise about 3.0×10⁷ cells to about 6×10⁸ cells that express an anti-CD70 CAR (CAR⁺ cells), for example, CAR⁺ CTX130 cells. In some instances, an effective amount of a genetically engineered T cell population may comprise about 3.0×10⁷ cells to about 4.5×10⁸ cells that express an anti-CD70 CAR (CAR⁺ cells), for example, CAR⁺ CTX130 cells. In some instances, an effective amount of a genetically engineered T cell population may comprise about 3.0×10⁷ cells to about 1×10⁸ cells that express an anti-CD70 CAR (CAR⁺ cells), for example, CAR⁺ CTX130 cells. In some instances, an effective amount of a genetically engineered T cell population may comprise about 1.0×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 3.0×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 4.5×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 6.0×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 7.5×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 3.0×10⁸ cells to about 4.5×10⁸ cells that express an anti-CD70 CAR (CAR⁺ cells), for example, CAR⁺ CTX130 cells. In some instances, an effective amount of a genetically engineered T cell population may comprise about 4.5×10⁸ cells to about 6×10⁸ cells that express an anti-CD70 CAR (CAR⁺ cells), for example, CAR⁺ CTX130 cells. In some instances, an effective amount of a genetically engineered T cell population may comprise about 6×10⁸ cells to about 7.5×10⁸ cells that express an anti-CD70 CAR (CAR⁺ cells), for example, CAR⁺ CTX130 cells. In some instances, an effective amount of a genetically engineered T cell population may comprise about 9×10⁸ cells to about 1.8×10⁹ cells that express an anti-CD70 CAR (CAR⁺ cells), for example, CAR⁺ CTX130 cells.

In some embodiments, an effective amount of a genetically engineered T cell population may comprise at least 3.0×10⁷ CAR⁺ CTX130 cells, at least 1×10⁸ CAR⁺ CTX130 cells, at least 3.0×10⁸ CAR⁺ CTX130 cells, at least 4×10⁸ CAR⁺ CTX130 cells, at least 4.5×10⁸ CAR⁺ CTX130 cells, at least 5×10⁸ CAR⁺ CTX130 cells, at least 5.5×10⁸ CAR⁺ CTX130 cells, at least 6×10⁸ CAR⁺ CTX130 cells, at least 6.5×10⁸ CAR⁺ CTX130 cells, at least 7×10⁸ CAR⁺ CTX130 cells, at least 7.5×10⁸ CAR⁺ CTX130 cells, at least 8×10⁸ CAR⁺ CTX130 cells, at least 8.5×10⁸ CAR+ CTX130 cells, or at least 9×10⁸ CAR⁺ CTX130 cells.

In some examples, an effective amount of the genetically engineered T cell population as disclosed herein (e.g., the CTX130 cells) may be about 3.0×10⁷ CAR⁺ CTX130 cells. In some examples, an effective amount of the genetically engineered T cell population as disclosed herein (e.g., the CTX130 cells) may be about 1.0×10⁸ CAR⁺ CTX130 cells. In some examples, an effective amount of the genetically engineered T cell population as disclosed herein (e.g., the CTX130 cells) may be about 3.0×10⁸ CAR⁺ CTX130 cells. In some examples, an effective amount of the genetically engineered T cell population as disclosed herein (e.g., the CTX130 cells) may be about 4.5×10⁸ CAR⁺ CTX130 cells. In some examples, an effective amount of the genetically engineered T cell population as disclosed herein (e.g., the CTX130 cells) may be about 6.0×10⁸ CAR⁺ CTX130 cells. In some examples, an effective amount of the genetically engineered T cell population as disclosed herein (e.g., the CTX130 cells) be about 7.5×10⁸ CAR+ CTX130 cells. In some examples, an effective amount of the genetically engineered T cell population as disclosed herein (e.g., the CTX130 cells) may be about 9.0×10⁸ CAR⁺ CTX130 cells. In some examples, an effective amount of the genetically engineered T cell population as disclosed herein (e.g., the CTX130 cells) may be about 1.8×10⁹ CAR⁺ CTX130 cells.

In some examples, a patient having CTCL, for example mycosis fungoides (MF) with large cell transformation, may be given a suitable dose of CTX130 cells, for example, about 3×10⁷ to about 6×10⁸ CAR⁺ CTX130 cells. Such an MF patient may be administered about 3×10⁷ CAR⁺ CTX130 cells. Alternatively, the MF patient may be administered about 1×10⁸ CAR⁺ CTX130 cells. In another example, the MF patient may be administered about 3×10⁸ CAR⁺ CTX130 cells. In another example, the MF patient may be administered about 4.5×10⁸ CAR⁺ CTX130 cells. In another example, the MF patient may be administered about 6×10⁸ CAR⁺ CTX130 cells. In another example, the MF patient may be administered about 7.5×10⁸ CAR⁺ CTX130 cells. In another example, the MF patient may be administered about 9×10⁸ CAR+ CTX130 cells. In yet another example, the MF patient may be administered about 1.8×10⁹ CAR+ CTX130 cells.

In some examples, a patient having CTCL, for example mycosis fungoides (MF) with large cell transformation, may be given a suitable dose of CTX130 cells, for example, about 9×10⁹ to about 1×10⁹ CAR⁺ CTX130 cells. Such an MF patient may be administered about 9×10⁹ CAR⁺ CTX130 cells. Alternatively, the MF patient may be administered about 1×10⁹ CAR⁺ CTX130 cells.

In some embodiments, a human patient (e.g., ≥18) having a body wight of 40-70 kg may start with a dose of 3×10⁷ CAR⁺ CTX130 cells, a dose of 1×10⁸ CAR⁺ CTX130 cells, or a dose of 3×10⁸ CAR⁺ CTX130 cells. In some embodiments, a human patient (e.g., ≥18) having a body weight≥70 kg may start with a dose of 9×10⁸ CAR⁺ CTX130 cells or a dose of 1.8×10⁹ CAR+ CTX130 cells.

In some instances, the amount of the anti-CD70 CAR T cells such as CTX130 cells administered to a human patient does not exceed 1×105 TCR⁺ cells/kg. In some examples, the amount of the anti-CD70 CAR T cells such as CTX130 cells administered to a human patient does not exceed 7×10⁴ TCR⁺ cells/kg.

In some embodiments, a suitable dose of CTX130 cells administered from one or more vials of the pharmaceutical composition, each vial comprising about 1.5×10⁸ CAR+ CTX130 cells. In some embodiments, a suitable dose of CTX130 cells is administered from one or more vials of the pharmaceutical composition, each vial comprising about 3×10⁸ CAR⁺ CTX130 cells. In some embodiments, a suitable dose of CTX130 cells is administered to a subject in one or more folds of 1.5×10⁸ CAR+ CTX130 cells, e.g., 1-fold, 2-fold, 3-fold, 4-fold, 5-fold, or 6-fold of 1.5×10⁸ CAR+ CTX130 cells. In some embodiments, a suitable dose of CTX130 cells is administered from one or more full or partial vials of the pharmaceutical composition.

The efficacy of anti-CD70 CAR T cell therapy described herein can be determined by the skilled clinician. An anti-CD70 CAR T cell therapy is considered “effective”, if any one or all of the signs or symptoms of, as but one example, levels of CD70 are altered in a beneficial manner (e.g., decreased by at least 10%), or other clinically accepted symptoms or markers of a T cell or B cell malignancy are improved or ameliorated. Efficacy can also be measured by failure of a subject to worsen as assessed by hospitalization or need for medical interventions (e.g., progression of the T cell or B cell malignancy is halted or at least slowed). Methods of measuring these indicators are known to those of skill in the art and/or described herein. Treatment includes any treatment of a T cell or B cell malignancy in a human patient and includes: (1) inhibiting the disease, e.g., arresting, or slowing the progression of symptoms; or (2) relieving the disease, e.g., causing regression of symptoms; and (3) preventing or reducing the likelihood of the development of symptoms.

Treatment methods described herein encompass redosing of anti-CD70 CAR T cells (e.g., CTX130 cells), for example, up to 2 additional doses. Prior to each redosing of anti-CD70 CAR T cells, the patient is subjected to another lymphodepletion treatment. The doses of anti-CD70 CAR T cells may be the same for the first, second, and third doses. For example, each of the first, second, and third doses can be 1×10⁷ CAR⁺ cells, 3×10⁷ CAR⁺ cells, 1×10⁸ CAR⁺ cells, 1.5×10⁸ CAR⁺ cells, 3×10⁸ CAR⁺ cells, 4.5×10⁸ CAR⁺ cells, 6×10⁸ CAR⁺ cells, 7.5×10⁸ CAR+ cells, or 9×10⁸ CAR⁺ cells. In other instances, the doses of anti-CD70 CAR T cells may increase in number of CAR+ cells as the number of doses increases. For example, the first dose is 1×10⁷ CAR+ cells, the second dose is 1×10⁸ CAR+ cells, and the third dose is 1×10⁹ CAR+ cells. Alternatively, the first dose of CAR+ cells is lower than the second and/or third dose of CAR+ cells, e.g., the first dose is 1×10⁷ CAR+ cells and the second and the third doses are 1×10⁹ CAR+ cells. In some examples, the dose of anti-CD70 CAR T cells may increase by 1.5×10⁸ CAR+ cells for each subsequent dose.

Patients may be assessed for re-dosing following each administration of anti-CD70 CAR T cells. For example, following a first dose of anti-CD70 CAR T cells, a human patient may be eligible for receiving a second dose of anti-CD70 CAR T cells if the patient does not show one or more of the following: (a) dose-limiting toxicity (DLT), (b) grade 4 CRS that does not resolve to grade 2 within 72 hours, (c) grade>1 GvHD, (d) grade≥3 neurotoxicity, (e) active infection, (f) hemodynamically unstable, and (g) organ dysfunction. In another example, following a second dose of anti-CD70 CAR T cells, a human patient may be eligible for receiving a third dose of CTX130 if that patient does not show one or more of the following: (a) dose-limiting toxicity (DLT), (b) grade 4 CRS that does not resolve to grade 2 within 72 hours, (c) grade>1 GvHD, (d) grade≥3 neurotoxicity, (e) active infection, (f) hemodynamically unstable, and (g) organ dysfunction.

In some embodiments, a human patient as disclosed herein may be given multiple doses of the anti-CD70 CAR T cells (e.g., the CTX130 cells as disclosed herein), i.e., re-dosing. The human patient may be given up to three doses in total (i.e., re-dosing for no more than 2 times). The interval between two consecutive doses may be about 8 weeks to about 2 years. In some instances, two consecutive doses of the anti-CD70 CAR T cells disclosed herein such as the CTX130 cells may be about 28 days apart.

In some examples, a human patient may be re-dosed if the patient achieved a partial response (PR) or complete response (CR) after a first dose (or a second dose) and subsequently progressed within 2 years of last dose. In other examples, a human patient may be re-dosed when the patient achieved PR (but not CR) or stable disease (SD) after the most recent dose.

In some examples, a human patient may be re-dosed for up to 2 additional doses, each of which is preceded with the LD treatment disclosed herein, when the patient shows loss of response within the first 2 years after last dose of the anti-CD70 CAR T cells. Alternatively, re-dosing may be performed when a patient shows stable disease or progressive disease with significant clinical benefit after the last dose (e.g., at least 28 days after the last dose) as determined by a medical practioner.

In some instances, re-dosing of anti-CD70 CAR T cells may take place up to 12 weeks after the first dose of anti-CD70 CAR T cells. A human patient may be re-dosed for up to two times at 12 weeks. When a patient is administered two doses, the second dose may be administered 3-6 weeks or 9-12 weeks after the first dose. When a patient is administered three doses, the third dose may be administered 9-12 weeks after the first dose, and the second dose may be administered 3-6 weeks after the first dose.

Following each dosing of anti-CD70 CAR T cells, a human patient may be monitored for acute toxicities such as cytokine release syndrome (CRS), tumor lysis syndrome (TLS), neurotoxicity, graft versus host disease (GvHD), viral encephalitis, and/or on target off-tumor toxicities (e.g., due to the activity of the anti-CD70 CAR T cells against activated T lymphocytes, B lymphocytes, dendritic cells, osteoblasts, and/or renal tubular-like epithelium) and/or uncontrolled T cell proliferation. In addition, one or more of the following adverse effects may be monitored: hypotension, renal insufficiency (which may be caused, e.g., by suppression of renal tubular-like epithelium cells), hemophagocytic lymphohistiocytosis (HLH), prolonged cytopenias, and/or suppression of osteoblasts. After each dose of anti-CD70 CAR T cells, a human patient may be monitored for at least 28 days for development of toxicity. If development of toxicity is observed, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CAR T cells.

Anti-CD70 CAR T cell treatment methods described herein may be used on a human patient that has undergone a prior anti-cancer therapy such as a prior anti-CD19 CAR T cell therapy, a prior first line systemic therapy, a prior combined therapy, or a prior mogamulizumab therapy.

Anti-CD70 CAR T cells treatment methods described herein may also be used in combination therapies. For example, anti-CD70 CAR T cells treatment methods described herein may be co-used with other therapeutic agents, for treating a T cell or a B cell malignancy, or for enhancing efficacy of the genetically engineered T cell population and/or reducing side effects of the genetically engineered T cell population.
(iv) NK Cell Inhibitor Treatment

In some embodiments, any of the anti-CD70 CAR T cells such as the CTX130 cells disclosed herein are used in combination with an NK cell inhibitor, such as a CD38 inhibitor. In some instances, the CD38 inhibitor is an anti-CD38 antibody. In one specific example, the anti-CD38 antibody is daratumumab.

An NK cell inhibitor such as daratumumab may be formulated in a pharmaceutical composition and given to a suitable subject as disclosed herein at a suitable time point relative to the LD and/or allogeneic anti-CD70 CAR-T cell (e.g., CTX130) therapy. A pharmaceutical composition comprising daratumumab and one or more pharmaceutically acceptable carriers may be administered to the subject via a suitable route, for example, orally, parenterally, by inhalation spray, rectally, nasally, buccally, vaginally or via an implanted reservoir.

In some embodiments, the pharmaceutical composition comprising daratumumab is to be administered by injection, for example, intravenous infusion or subcutaneous injection. A sterile injectable composition, e.g., a sterile injectable aqueous or oleaginous suspension, can be formulated according to techniques known in the art using suitable dispersing or wetting agents (such as Tween® 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

The pharmaceutical compositions as described herein can comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some embodiments, an effective amount of daratumumab (e.g., about 10-20 mg/kg such as about 16 mg/kg) may be given to the subject via a suitable route (e.g., intravenous infusion). The effective amount of daratumumab may split into two parts (e.g., equally) and be administered to the subject on two consecutive days. In some embodiments, a reduced dose of daratumumab (e.g., 8 mg/kg) may be administered to a patient. In other embodiments, an effective amount of daratumumab may be about 1500-2000 mg (e.g., 1800 mg) by subcutaneous injection.

In some examples, administration of daratumumab may be performed prior to the LD therapy. In specific examples, administration of daratumumab may be performed within 3 days (e.g., at least 12 hours) prior to the LD therapy. Alternatively or in addition, administration of daratumumab may be performed no more than 10 days prior to the treatment with the anti-CD70 CAR-T cells such as CTX130 cells. In one example, administration of daratumumab may be performed at least 12 hours prior to starting the LD treatment and within 10 days of the administration of the anti-CD70 CAR-T cells such as CTX130 cells.

In some instances, daratumumab treatment may be repeated once every 2-4 weeks. In some examples, daratumumab treatment may be repeated once every 3 weeks. For example, a patient may be given a second dose of daratumumab about 3 weeks after the first dose. A subsequent dose of daratumumab may be the same as the preceding dose of daratumumab given to the patient, for example, 16 mg/kg, via intravenous infusion, which may split into two parts as disclosed herein. In other embodiments, an effective amount of daratumumab may be about 1500-2000 mg (e.g., 1800 mg) by subcutaneous injection. Alternatively, the subsequent doses of daratumumab may be lower than that of the preceding dose. The additional doses of daratumumab may vary as determined by a medical practitioner. If the subject exhibits disease progress or severe toxicity, the additional daratumumab treatment may be terminated. In some embodiments, a lower daratumumab dose, for example, 8 mg/kg, may be used.

NK cell inhibitors such as anti-CD38 antibodies (e.g., daratumumab) were found to suppress potential host immune responses to allogenic CAR T cells, for example, immune responses mediated by NK cells against allogenic CAR T cells that are deficient in MHC Class I expression. The NK cell inhibitor may also allow increased expansion and persistence of the CAR T cells. Accordingly, the NK cell inhibitors as disclosed herein, such as anti-CD38 antibodies (e.g., daratumumab), could be co-used with CAR T cells that express an anti-CD70 CAR and are deficient in MHC Class I expression. In some instances, the anti-CD70 CAR T cells that are deficient in MHC Class I expression may have a level of MHC Class I expression at least 50% (e.g., at least 60%, at least 70%, at least 80%, or at least 90%) lower than the anti-CD70 CAR T cell counterpart that is not deficient in MHC Class I expression (i.e., having the same genetic editings except for MHC Class I). In some examples, the anti-CD70 CAR T cells that are deficient in MHC Class I expression may have no detectable level of MHC Class I expression as measured by a conventional assay.

In some embodiments, the deficience in MHC Class I expression may be caused by gene editing of one or more genes coding for components of the MHC Class I complex to disrupte the expression thereof. Such gene editing may be achieved by a conventional method. In some examples, the one or more genes coding for MHC Class I components may be disrupted by a CRISPR/Cas gene ediging system. In some examples, the β2M gene can be disrupted via a gene editing method, for example, CRISPR. More details for disrupting the #2M gene via a CRISPR/Cas gene editing system are provided elsewhere herein.

The combined therapy of an NK cell inhibitor such as an anti-CD38 antibody (e.g., daratumumab) and anti-CD70 CAR T cells deficient in MHC Class I expression for treating a target CD70+ hematopoietic malignancies such as a T cell or B cell malignancy is also within the scope of the present disclosure. Such a combined therapy may involve any of the treatment regimens as also disclosed herein.
(v) Exemplary Treatment Regimens

In some embodiments, a treatment method as provided herein may be performed as follows. A suitable human patient having one of the target diseases as disclosed herein may be identified via routine medical practice or as disclosed herein (see Example 6 below). Such a human patient may meet the inclusion and/or exclusion criteria disclosed in Example 6 below. An LD chemotherapy can be performed to the human patient. Such an LD chemotherapy may comprise co-administration (e.g., intravenous injection) of fludarabine at 30 mg/m² and cyclophosphamide at 500 mg/m² daily for three days. Within about 2-7 days, the human patient can be administered the anti-CD70 CAR T cells disclosed herein (e.g., CTX130) via intravenous infusion at one of the following doses: 3.0×10⁷ CAR+ cells, 1×10⁸ CAR⁺ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ Car+ cells Optionally, the human patient may be administered up to two additional doses of the anti-CD70 CAR T cells, each accompanied with the LD therapy, when the patient shows 1) loss of response within the first 2 years after the last dose of the anti-CD70 CAR T cells, or 2) stable disease or progressive disease with significant clinical benefit after the last dose of the anti-CD70 CAR T cells (e.g., at least 28 days after the treatment). Significant clinical benefit can be assessed by a medical practioner. The additional dose(s) may be the same as the initial dose. Alternatively, the subsequent dose(s) may be adjusted according to the patient's response to the initial dose, which can be determined by a medical practioner.

In other embodiments, a treatment method as provided herein may be performed as follows. A suitable human patient having one of the target diseases as disclosed herein may be identified via routine medical practice or as disclosed herein (see Example 6 below). Such a human patient may meet the inclusion and/or exclusion criteria disclosed in Example 6 below. A first dose of darabumumab (e.g., 16 mg/kg via intravenous infusion or 1800 mg via subcutaneous injection) may be administered to the human patient. In some instances, the dose of daratumumab may be split into two parts evenly (e.g., 8 mg/kg each i.v.), which can be administered to the patient on two consecutive days. An LD chemotherapy can be performed to the human patient at a suitable time point after the daratumumab treatment, for example, at least 12 hours after the daratumumab treatment. Such an LD chemotherapy may comprise co-administration (e.g., intravenous injection) of fludarabine at 30 mg/m² and cyclophosphamide at 500 mg/m² daily for three days. Within about 2-7 days after the LD therapy and within 10 days after the daratumumab treatment, the human patient can be administered the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. Following the anti-CD70 CAR T cell therapy, a second dose of daratumumab may be administered to the human patient, for example, about three weeks after the first dose of the anti-CD70 CAR-T cells. The second dose of daratumumab may be the same as the first dose of daratumumab. Alternatively, the second dose of daratumumab may be lower than the first dose. About 6 weeks after the first dose of the anti-CD70 CAR-T cells, a third dose of daratumumab may be administered to the human patient. The third dose of daratumumab may be the same as the first and/or second dose of daratumumab. Alternatively, the third dose of daratumumab may be lower than the first and/or second dose.

Optionally, the above treatment cycle may be repeated for multiple times (e.g., up to two times) when the patient shows 1) loss of response within the first 2 years after the last dose of the anti-CD70 CAR T cells, or 2) stable disease or progressive disease with significant clinical benefit after the last dose of the anti-CD70 CAR T cells (e.g., at least 28 days after the treatment). Significant clinical benefit can be assessed by a medical practioner. The additional dose(s) of the anti-CD70 CAR T cells and/or daratumumab may be the same as the initial dose. Alternatively, the subsequent dose(s) may be adjusted according to the patient's response to the initial dose, which can be determined by a medical practioner. In some instances, a lower dose of daratumumab (e.g., 8 mg/kg) may be used in the initial treatment cycle, or in the subsequent treatment cycles.

In some embodiments, a treatment method as provided herein may be performed as follows. A suitable human patient having one of the target diseases as disclosed herein may be identified via routine medical practice or as disclosed herein (see Example 6 below). Such a human patient may meet the inclusion and/or exclusion criteria disclosed in Example 6 below. An LD chemotherapy can be performed to the human patient. Such an LD chemotherapy may comprise co-administration (e.g., intravenous injection) of fludarabine at 30 mg/m² and cyclophosphamide at 500 mg/m² daily for three days. Within about 2-7 days, the human patient can be administered a first dose of the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. Within 4-15 days (e.g., 4-6 days or 5-7 days), the human patient can be administered a second dose of the CD70 CAR T cells disclosed herein (e.g., CTX130) via intravenous infusion at one of the following doses: 3.0×10⁷ CAR⁺ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. The second dose of the anti-CD70 CAR-T cells may not be accompanied with a lymphodepletion treatment.

The above-described course of treatment may be repeated multiple times, for example, two times or three times. In some instances, a second course of the treatment may be performed with LD chemotherapy after the patient losses of CR within the first 2 years after the initial infusion of the anti-CD70 CAR-T cells or the patient has achieved PR, SD, or PD with clinical benefit as determined by a medical practitioner. In some instances, a third course of treatment, which may be identical to the first and/or second course, may be performed ot the patient after loss of CR within the first 2 years after the initial anti-CD70 CAR-T cell infusion or after assessment of PR, SD, or PD with clinical benefit.

In other embodiments, a treatment method as provided herein may be performed as follows. A suitable human patient having one of the target diseases as disclosed herein may be identified via routine medical practice or as disclosed herein (see Example 6 below). Such a human patient may meet the inclusion and/or exclusion criteria disclosed in Example 6 below. A first dose of daratumumab (e.g., 16 mg/kg via intravenous infusion or 1800 mg via subcutaneous injection) may be administered to the human patient. In some instances, the dose of daratumumab may be split into two parts evenly (e.g., 8 mg/kg each i.v.), which can be administered to the patient on two consecutive days. The first dose of daratumumab can be administered at least 12 hours prior to the starting of An LD chemotherapy and within 10 days prior to the first infusion of the anti-CD70 CAR-T cells such as CTX130 cells. Daratumumab administration can be repeated about 3 weeks and about 6 weeks after the first infusion of the anti-CD70 CAR-T cells.

The LD chemotherapy may comprise co-administration (e.g., intravenous injection) of fludarabine at 30 mg/m² and cyclophosphamide at 500 mg/m² daily for three days. Within about 2-7 days after the LD therapy and within 10 days after the daratumumab treatment, the human patient can be administered the anti-CD70 CAR T cells disclosed herein (e.g., CTX130) via intravenous infusion at one of the following doses: 3.0×10⁷ CAR⁺ cells, 1×10⁸ CAR⁺ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. Within 4-15 days (e.g., 4-6 days or 5-7 days), the human patient can be administered a second dose of the CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. The second dose of the anti-CD70 CAR-T cells may not be accompanied with a lymphodepletion treatment and/or the daratumumab treatment.

The above-described course of treatment may be repeated multiple times, for example, two times or three times. In some instances, a second course of the treatment may be performed with LD chemotherapy after the patient loses CR within the first 2 years after the initial infusion of the anti-CD70 CAR-T cells or the patient has achieved PR, SD, or PD with clinical benefit as determined by a medical practitioner. In some instances, a third course of treatment, which may be identical to the first and/or second course, may be performed ot the patient after loss of CR within the first 2 years after the initial anti-CD70 CAR-T cell infusion or after assessment of PR, SD, or PD with clinical benefit.

In some embodiments, a treatment method as provided herein may be performed as follows. A suitable human patient having one of the target diseases as disclosed herein may be identified via routine medical practice or as disclosed herein (see Example 6 below). Such a human patient may meet the inclusion and/or exclusion criteria disclosed in Example 6 below. An LD chemotherapy can be performed to the human patient. Such an LD chemotherapy may comprise co-administration (e.g., intravenous injection) of fludarabine at 30 mg/m² and cyclophosphamide at 500 mg/m² daily for three days. Within about 2-7 days, the human patient can be administered a first dose of the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. A second dose of the anti-CD70 CAR-T cells, accompanied with a second LD chemotherapy, can be performed to the human patient about 4-8 weeks (e.g., 5 weeks) after the first dose of the anti-CD70 CAR-T cells. The second dose of the CD70 CAR T cells disclosed herein (e.g., CTX130) can be administered via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. The second dose of the anti-CD70 CAR-T cells may not be accompanied with a lymphodepletion treatment. Human patients suitable for the second dose of the anti-CD70 CAR-T cells may achieve CR, PR, SD, or PD with clinical benefit about 4 weeks after the first dose of the anti-CD70 CAR-T cells (e.g., based on Lugano or Olsen criteria as appropriate). In some instances, the second dose may not be accompanied with the second LD chemotherapy, e.g., when the patient experiences significant cytopenia.

After the above-described course of treatment, the human patient may be given an optional single additional dose of the anti-CD70 CAR-T cells, which can be accompanied with an additional LD chemotherapy. Patients suitable for this additional single dose may lose CR within the first 2 years after the initial infusion of the anti-CD70 CAR-T cells or may achieve PR, SD, or PD with clinical benefit as determined by a medical practitioner. The additional dose may be greater than or equal to the doses used in the first course of treatment.

In yet embodiments, a treatment method as provided herein may be performed as follows. A suitable human patient having one of the target diseases as disclosed herein may be identified via routine medical practice or as disclosed herein (see Example 6 below). Such a human patient may meet the inclusion and/or exclusion criteria disclosed in Example 6 below. A first dose of daratumumab (e.g., 16 mg/kg via intravenous infusion or 1800 mg via subcutaneous injection) may be administered to the human patient. In some instances, the dose of daratumumab may be split into two parts evenly (e.g., 8 mg/kg each i.v.), which can be administered to the patient on two consecutive days. The first dose of daratumumab can be administered at least 12 hours prior to the starting of An LD chemotherapy and within 10 days prior to the first infusion of the anti-CD70 CAR-T cells such as CTX130 cells.

The LD chemotherapy may comprise co-administration (e.g., intravenous injection) of fludarabine at 30 mg/m² and cyclophosphamide at 500 mg/m² daily for three days. Within about 2-7 days after the LD therapy and within 10 days after the daratumumab treatment, the human patient can be administered the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. A second dose of the anti-CD70 CAR-T cells, accompanied with a second LD chemotherapy, can be performed to the human patient about 4-8 weeks (e.g., 5 weeks) after the first dose of the anti-CD70 CAR-T cells. The second dose of the CD70 CAR T cells disclosed herein (e.g., CTX130) can be administered via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, 9.0×10⁸ CAR+ cells, or 1.8×10⁹ CAR+ cells. The second dose of the anti-CD70 CAR-T cells may not be accompanied with a lymphodepletion treatment. Human patients suitable for the second dose of the anti-CD70 CAR-T cells may achieve CR, PR, SD, or PD with clinical benefit about 4 weeks after the first dose of the anti-CD70 CAR-T cells (e.g., based on Lugano or Olsen criteria as appropriate). In some instances, the second dose may not be accompanied with the second LD chemotherapy, e.g., when the patient experiences significant cytopenia.

After the above-described course of treatment, the human patient may be given an optional single additional dose of the anti-CD70 CAR-T cells, which can be accompanied with an additional LD chemotherapy. Patients suitable for this additional single dose may lose CR within the first 2 years after the initial infusion of the anti-CD70 CAR-T cells or may achieve PR, SD, or PD with clinical benefit as determined by a medical practitioner. The additional dose may be greater than or equal to the doses used in the first course of treatment.

### V. Kit for Treating CD70 Positive Hematopoietic Maligancies

The present disclosure also provides kits for use of a population of anti-CD70 CAR T cells such as CTX130 T cells and optionally an NK cell inhibitor such as an anti-CD38 antibody (e.g., daratumumab) as described herein in methods for treating hematopoietic maligancy, such as those disclosed herein. Such kits may include a first container comprising a first pharmaceutical composition that comprises any of the populations of genetically engineered anti-CD70 CAR T cells (e.g., those described herein such as CTX130 cells), and a pharmaceutically acceptable carrier, and optionally a second container comprising a second pharmaceutical composition comprising the NK cell inhibitor such as daratumumab. The anti-CD70 CAR-T cells may be suspended in a cryopreservation solution such as those disclosed herein. Optionally, the kit may further comprise a third container comprising a third pharmaceutical composition that comprises one or more lymphodepleting agents.

In some embodiments, the kit can comprise instructions for use in any of the methods described herein. The included instructions can comprise a description of administration of the anti-CD70 CAR T cells, and optionally daratumumab and any additional therapeutic agents to a subject to achieve the intended activity in a human patient having a hematopoietic malignancy such as those disclosed herein. The kit may further comprise a description of selecting a human patient suitable for treatment based on identifying whether the human patient is in need of the treatment.

The instructions relating to the use of a population of anti-CD70 CAR-T cells such as CTX130 T cells described herein generally include information as to dosage, dosing schedule, and route of administration for the intended treatment. The instructions may also include information relating to the use of daratumumab, for example, dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a symptom of the hematopoietic maligancy in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the anti-CD70 CAR-T cells such as the CTX130 T cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (lRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Example 1: Generation of T Cells with Multiple Gene Knockouts

This example describes the use of CRISPR/Cas9 gene editing technology to produce human T cells that lack expression of two or three genes simultaneously. Specifically, the T cell receptor (TCR) gene (gene edited in the TCR Alpha Constant (TRAC) region), the β2-microglobulin (β2M) gene, and the Cluster of Differentiation 70 (CD70) gene were edited by CRISPR/Cas9 gene editing to produce T cells deficient in two or more of the listed genes. The following abbreviations are used in for brevity and clarity:
2× KO: TRAC⁻/β2M⁻
3× KO (CD70): TRAC⁻/β2M⁻/CD70⁻

Activated primary human T cells were electroporated with Cas9:gRNA RNP complexes. The nucleofection mix contained the Nucleofector™ Solution, 5×10⁶ cells, 1 μM Cas9, and 5 μM gRNA (as described in Hendel et al., Nat Biotechnol. 2015; 33(9):985-989, PMID: 26121415). For the generation of double knockout T cells (2× KO), the cells were electroporated with two different RNP complexes, each containing Cas9 protein and one of the following sgRNAs: TRAC (SEQ ID NO: 6) and β2M (SEQ ID NO: 10) at the concentrations indicated above. For the generation of triple knockout T cells (3× KO), the cells were electroporated with three different RNP complexes, each RNA complex containing Cas protein and one of the following sgRNAs: (a) TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOs: 3, 7, 11, and/or 67). See also sequences in Table 7.

TABLE 7
gRNA Sequences/Target Sequences.

| Name | Unmodified Sequence | Modified Sequence |
|---|---|---|
| TRAC sgRNA | AGAGCAACAGUGCUGUGG | A*G*A*GCAACAGUGCUGU |
| | CCguuuuagagcuagaaauagcaagu | GGCCguuuuagagcuagaaauagca |
| | uaaaauaaggcuaguccguuaucaacu | aguuaaaauaaggcuaguccguuauca |
| | ugaaaaaguggcaccgagucggugcU | acuugaaaaaguggcaccgagucggug |
| | UUU | cU*U*U*U (SEQ ID NO: 6) |
| | (SEQ ID NO: 7) | |
| TRAC sgRNA spacer | AGAGCAACAGUGCUGUGG | A*G*A*GCAACAGUGCUGU |
| | CC (SEQ ID NO: 9) | GGCC (SEQ ID NO: 8) |
| β2M sgRNA | GCUACUCUCUCUUUCUGG | g*c*u*acucucucuuucu |
| | CCguuuuagagcuagaaauagcaagu | GGCCguuuuagagcuagaaauagca |
| | uaaaauaaggcuaguccguuaucaacu | aguuaaaauaaggcuaguccguuauca |
| | ugaaaaaguggcaccgagucggugcU | acuugaaaaaguggcaccgagucggug |
| | UUU | cU*U*U*U |
| | (SEQ ID NO: 11) | (SEQ ID NO: 10) |
| β2M sgRNA spacer | GCUACUCUCUCUUUCUGG | G*C*U*ACUCUCUCUUUCU |
| | CC (SEQ ID NO: 13) | GGCC (SEQ ID NO: 12) |
| CD70 sgRNA; also referred to | GCUUUGGUCCCAUUGGUC | G*C*U*UUGGUCCCAUUGG |
| as: T7 | GCguuuuagagcuagaaauagcaagu | UCGCguuuuagagcuagaaauagca |
| | uaaaauaaggcuaguccguuaucaacu | aguuaaaauaaggcuaguccguuauca |
| | ugaaaaaguggcaccgagucggugcU | acuugaaaaaguggcaccgagucggug |
| | UUU | cU*U*U*U (SEQ ID NO: 2) |
| | (SEQ ID NO: 3) | |
| CD70 sgRNA spacer; also | GCUUUGGUCCCAUUGGUC | G*C*U*UUGGUCCCAUUGG |
| referred to as: T7 | GC (SEQ ID NO: 5) | UCGC (SEQ ID NO: 4) |
| CD70 sgRNA; also referred to | GCCCGCAGGACGCACCCA | G*C*C*CGCAGGACGCACC |
| as: T8 | UAguuuuagagcuagaaauagcaagu | CAUAguuuuagagcuagaaauagca |
| | uaaaauaaggcuaguccguuaucaacu | aguuaaaauaaggcuaguccguuauca |
| | ugaaaaaguggcaccgagucggugcU | acuugaaaaaguggcaccgagucggug |
| | UUU | cU*U*U*U (SEQ ID NO: 66) |
| | (SEQ ID NO: 67) | |
| CD70 sgRNA spacer; also | GCCCGCAGGACGCACCCA | G*C*C*CGCAGGACGCACC |
| referred to as: T8 | UA (SEQ ID NO: 69) | CAUA (SEQ ID NO: 68) |

About one (1) week post electroporation, cells were either left untreated or treated with phorbol myristate acetate (PMA)/ionomycin overnight. The next day cells were processed for flow cytometry (see, e.g., Kalaitzidis D et al., J Clin Invest 2017; 127(4): 1405-1413) to assess TRAC, β2M, and CD70 expression levels at the cell surface of the edited cell population. The following primary antibodies were used (Table 8):

TABLE 8
Antibodies.

| Antibody | Clone | Fluor | Catalogue # | Dilution | For 1 |
|---|---|---|---|---|---|
| TCR | BW242/412 | PE | 130-091-236 | 1:100 | 1 μL |
| | | | (Miltenyi) | | |
| β2M | 2M2 | PE-Cy7 | 316318 (Biolegend) | 1:100 | 1 μL |
| CD70 | 113-16 | FITC | 355105 (Biolegend) | 1:100 | 1 μL |

Table 9 shows highly efficient multiple gene editing. For the triple knockout cells, 80% of viable cells lacked expression of TCR, β2M, and CD70 (Table 9).

TABLE 9
Percent of viable cells lacking expression in 3KO cell populations.

| | TRAC KO | β2M KO | CD70 KO | 3KO |
|---|---|---|---|---|
| 3KO (CD70) | 99% | 79% | 99% | 80% |

To assess whether triple gene editing in T cells affects cell expansion, cell numbers were enumerated among double and triple gene edited T cells (unedited T cells were used as a control) over a two-week period of post editing. 5×10⁶ cells were generated and plated for each genotype of T cells.

Cell proliferation (expansion) continued over the post-electroporation window test. Similar cell proliferation was observed among the double (β2M−/TRAC−) and triple β2M−/TRAC−/CD70−), knockout T cells, as indicated by the number of viable cells (data not shown). These data suggest that multiple gene editing does not impact T cell health as measured by T cell proliferation.

### Example 2: Generation of Anti-CD70 CAR T Cells with Multiple Knockouts

This example describes the production of allogeneic human T cells that lack expression of the TCR gene, β2M gene, and/or CD70 gene, and express a chimeric antigen receptor (CAR) targeting CD70. These cells are designated TCR⁻/β2M⁻/CD70⁻/anti-CD70 CAR⁺ or 3×KO (CD70) CD70 CAR⁺.

A recombinant adeno-associated adenoviral vector, serotype 6 (AAV6) (MOI 50,000) comprising the nucleotide sequence of SEQ ID NO: 43 (comprising the donor template in SEQ ID NO: 44, encoding anti-CD70 CAR comprising the amino acid sequence of SEQ ID NO: 46 or SEQ ID NO: 81) was delivered with Cas9:sgRNA RNPs (1 μM Cas9, 5 μM gRNA) to activated allogeneic human T cells. The following sgRNAs were used: TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOS: 3, 7, 11, and/or 67). About one (1) week post electroporation, cells were processed for flow cytometry to assess TRAC, β2M, and CD70, expression levels at the cell surface of the edited cell population. The following primary antibodies were used (Table 10):

TABLE 10
Antibodies.

| Antibody | Clone | Fluor | Catalogue # | Dilution |
|---|---|---|---|---|
| TCR | BW242/412 | PE | 130-091-236 (Miltenyi) | 1:100 |
| β2M | 2M2 | PE-Cy7 | 316318 (Biolegend) | 1:100 |
| CD70 | 113-16 | FITC | 355105 (Biolegend) | 1:100 |

T cell Proportion Assay. The proportions of CD4+ and CD8+ cells were then assessed in the edited T cell populations by flow cytometry using the following antibodies (Table 11):

TABLE 11
Antibodies.

| Antibody | Clone | Fluor | Catalogue # | Dilution |
|---|---|---|---|---|
| CD4 | RPA-T4 | BV510 | 300545 (Biolegend) | 1:100 |
| CD8 | SK1 | BV605 | 344741 (Biolegend) | 1:100 |

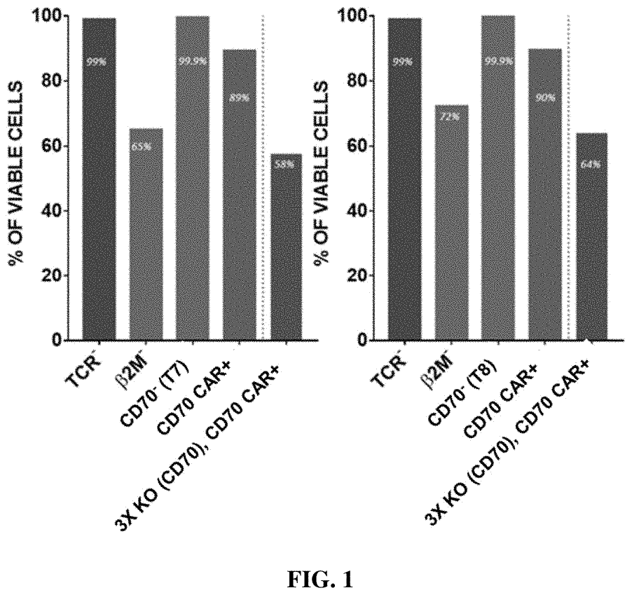

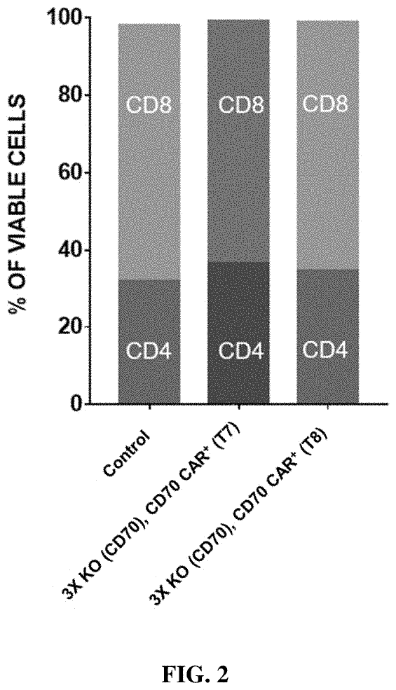

High efficiency gene editing and CAR expression was achieved in the edited anti-CD70 CAR T cell populations. In addition, editing did not adversely alter CD4/CD8 T cell populations. FIG. 1 shows highly efficient gene editing and anti-CD70 CAR expression in the triple knockout CAR T cell. More than 55% of viable cells lacked expression of TCR, β2M, and CD70, and also expressed the anti-CD70 CAR. FIG. 2 shows that normal proportions of CD4/CD8 T cell subsets were maintained in the TRAC−/β2M−/CD70−/anti-CD70 CAR+ cells, suggesting that these multiple gene edits do not affect T cell biology as measured by the proportion of CD4/CD8 T cell subsets.

### Example 3: Effect of CD70 KO on Cell Proliferation and Cytotoxicity of Anti-CD70 CAR T Cells In Vitro

(A) Cell Proliferation

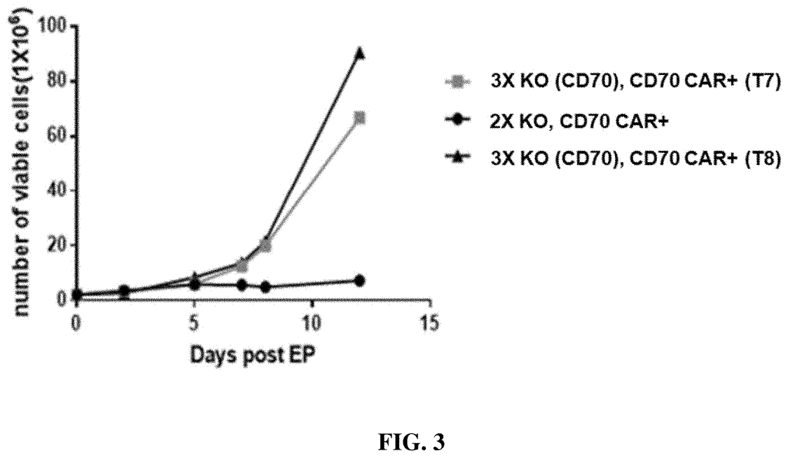

To further assess the impact of disrupting the CD70 gene in CAR T cells, anti-CD70 CAR T cells were generated as described in Example 2. Specifically, TRAC−/β2M−/CD70− anti-CD70 CAR+ T cells were generated using two different gRNAs (T7 (SEQ ID NO: 2 and T8 (SEQ ID NO: 66)). After electroporation, cell expansion was assessed by enumerating double or triple gene edited T cells over a two week period of post editing. 5×10⁶ cells were generated and plated for each genotype of T cells. Proliferation was determined by counting the number of viable cells. FIG. 3 shows that triple knockout TRAC⁻/β2M⁻/CD70⁻/anti-CD70 CAR⁺ T cells generated with either T7 or T8 gRNAs exhibited greater cell expansion relative to double knockout TRAC⁻/β2M⁻/anti-CD70 CAR⁺ T cells. These data suggest that knocking-out the CD70 gene gives a cell proliferation advantage to anti-CD70 CAR+ T cells.
(B) CD70 Expression in Various Cancer Cell Lines

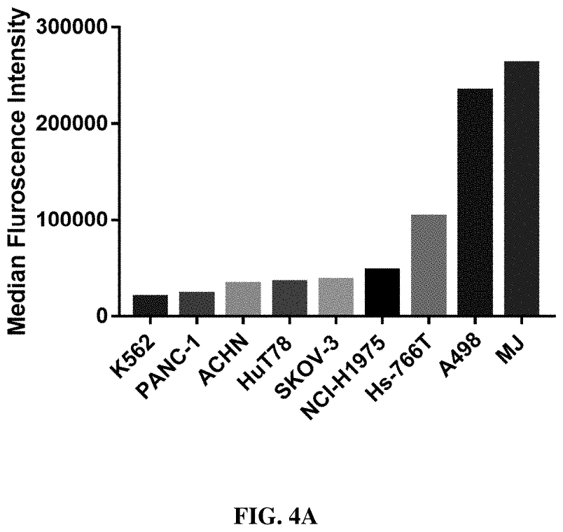

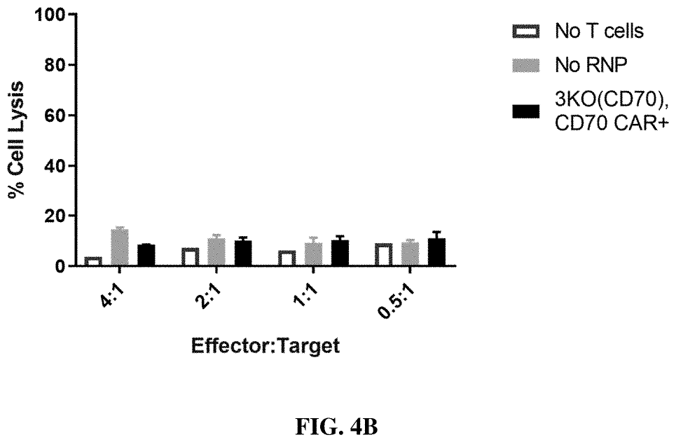

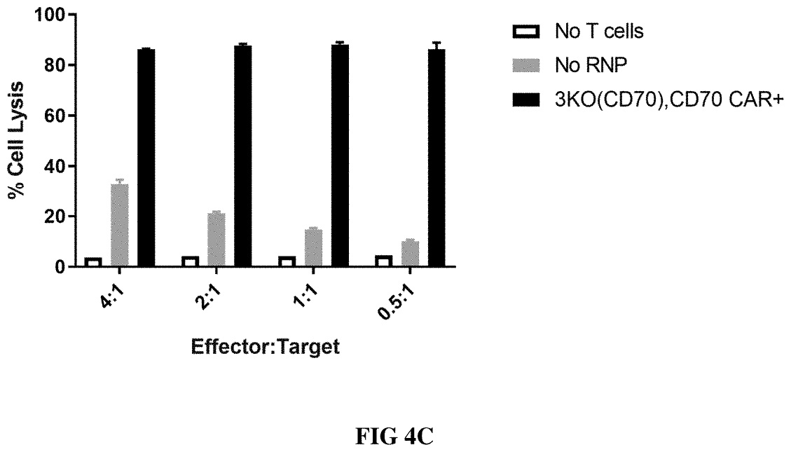

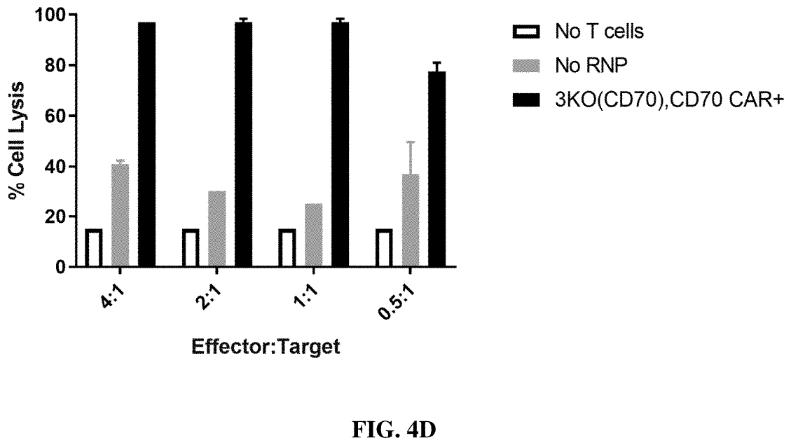

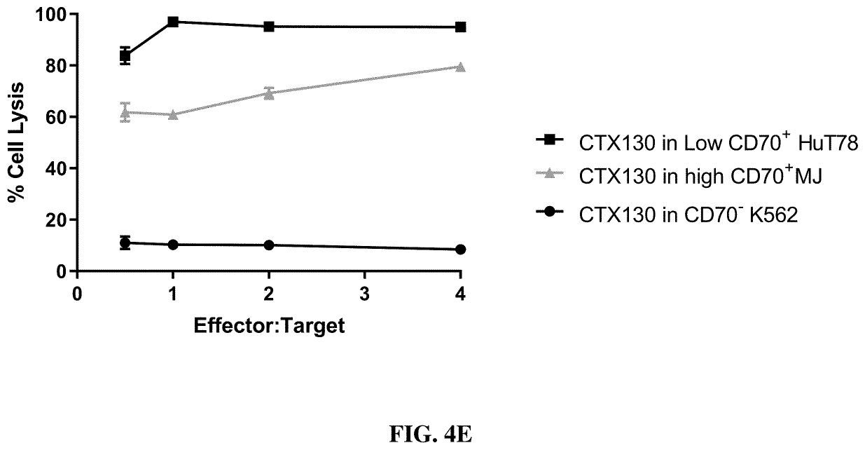

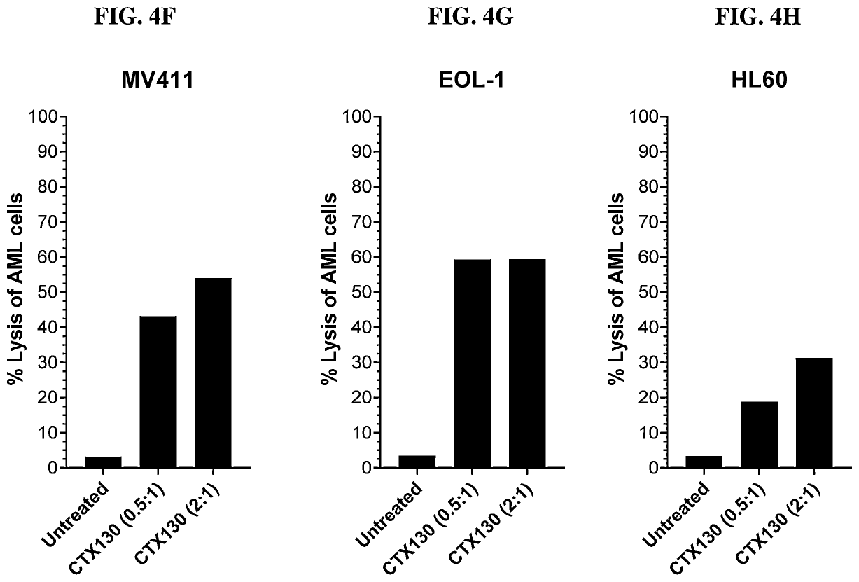

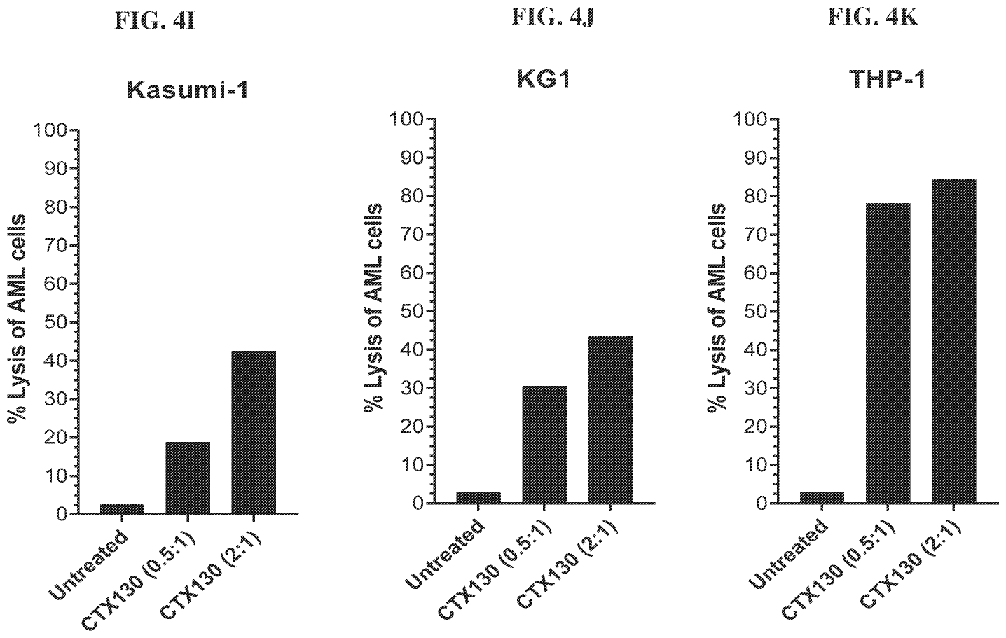

Relative CD70 expression was measured in various cancer cell lines to further evaluate the ability of anti-CD70 CAR⁺ T cells to kill various cancer types. CD70 expression was measured by flow cytometric analysis using Alexa Fluor 647 anti-human CD70 antibody (BioLegend Cat. No. 355115). Cancer cell lines were evaluated for CD70 expression by flow cytometric analysis (Table 12, FIG. 4A) using a FITC anti-human CD70 antibody (BioLegend Cat. No. 355105) in FIG. 4A. SKOV-3 (ovarian), HuT78 (lymphoma), NCI-H1975 (lung) and Hs-766T (pancreatic) cell lines exhibited levels of CD70 expression that were similar or higher than ACHN but lower than A498 (Table 11, FIG. 4A).

Acute myeloid Leukemia (AML) can express high levels of CD70. CD70 expression was measured in several acute myeloid leukemia cell lines by flow cytometric analysis: THP-1, MV-4-11, EOL-1, HL-60, Kasumi-1, and KG1. Table 12 shows that these cells express CD70 and can all be targeted by anti-CD70 CAR T cells, as demonstrated by the cell killing data described herein.

TABLE 12
CD70 Expression in Cancer Cell Lines.

| Cell Line | Cancer type | Relative CD70 expression |
|---|---|---|
| A498 | Kidney Carcinoma | High |
| ACHN | Kidney (derived from metastasis) | Medium-Low |
| SK-OV-3 | Ovarian Adenocarcinoma | Medium |
| NCI-H1975 | Lung Adenocarcinoma (NSCLC) | Medium |
| Calu-1 | Lung Carcinoma | Low |
| DU 145 | Prostate Carcinoma | Low |
| SNU-1 | Gastric Carcinoma | High |
| Hs 766T | Pancreatic Carcinoma | Medium |
| MJ | Cutaneous T cell Lymphoma | High |
| | (CTCL) | |
| HuT78 | Cutaneous T cell Lymphoma | Medium-Low |
| | (CTCL; Sézary syndrome) | |
| HuT102 | Cutaneous T cell Lymphoma | Medium |
| | (CTCL) | |
| HH | Cutaneous T cell Lymphoma | Medium-Low |
| | (CTCL) | |
| PANC-1 | Pancreatic Carcinoma | Low |
| U937 | AML: acute myeloid leukemia | No expression |
| K562 | chronic myelogenous leukemia | No expression (Negative |
| | | Control) |

TABLE 13
CD70 Expression in Leukemic Cell Lines.

| Exemplary AML Cell Lines | CD70 expression by Flow Cytometry |
|---|---|
| THP-1 | 88.9% |
| MV-4-11 | 99.9% |
| EOL-1 | 70.1% |
| HL-60 | 12.5% |
| Kasumi-1 | 19.4% |
| KG1 | 14.8% |

(C) CAR T Cell Cytotoxicity

The ability of anti-CD70 CAR+ T cells to selectively kill CD70-expressing cells was determined. A flow cytometry assay was designed to test killing of cancer cell suspension lines (e.g., K562, MM.1S, HuT78 and MJ cancer cells that are referred to as “target cells”) by 3× KO (CD70) (TRAC⁻/B2M⁻/CD70⁻) anti-CD70 CAR+ T cells. Three of the target cell lines that were used were CD70-expressing cancer cells (e.g., MM.1S, HuT78, and MJ), while a third that was used as negative control cancer cells lack CD70 expression (e.g., K562). The TRAC⁻/B2M⁻/CD70⁻/anti-CD70 CAR+ T cells were co-cultured with either the CD70-expressing MM.1S, HuT78 or MJ cell lines or the CD70-negative K562 cell line. The target cells were labeled with 5 μM efluor670 (eBiosciences), washed and seeded at a density of 50,000 target cells per well in a 96-well U-bottom plate. The target cells were co-cultured with TRAC⁻/B2M⁻/CD70⁻ anti-CD70 CAR+ T cells at varying ratios (0.5:1, 1:1, 2:1 and 4:1 CAR+ T cells to target cells) and incubated overnight. Target cell killing was determined following a 24 hour co-culture. The cells were washed and 200 μL of media containing a 1:500 dilution of 5 mg/mL DAPI (Molecular Probes) (to enumerate dead/dying cells) was added to each well. Cells were then analyzed by flow cytometry and the amount of remaining live target cells was quantified.

FIG. 4B, FIG. 4C, FIG. 4D, and FIG. 4E demonstrate selective target cell killing by TRAC−/B2M−/CD70− anti-CD70 CAR+ T cells (e.g., CTX130). A 24 hour co-culture with 3× KO (CD70) CAR+ T cells resulted in nearly complete killing of T cell lymphoma cells (HuT78), even at a low CAR+ T cell to CD70-expressing target cell ratio of 0.5:1 (FIG. 4D). Likewise, a 24 hour co-culture resulted in nearly complete killing of multiple myeloma cells (MM.1S) at all CAR+ T cell to target cell ratios tested (FIG. 4C). Similarly, a 24 hour co-culture resulted in effective cell lysis of high CD70 expressing T cell lymphoma cells (MJ) at all CAR⁺ T cell to target cell ratios tested. FIG. 4E shows cell lysis relative to a lower expressing CD70 T cell lymphoma cells (HuT78). Killing of target cells was found to be selective in that TRAC−/B2M-CD70−/anti-CD70 CAR+ T cells induced no killing of CD70− deficient K562 cells that was above the level of control samples (e.g., either cancer cells alone or co-culture with no RNP T cells) at any effector:target cell ratio tested (FIG. 4B). FIGS. 4F-4K demonstrate that TRAC−/B2M−/CD70− anti-CD70 CAR+ T cells (e.g., CTX130) are capable of effectively killing various CD70 expressing AML cell lines. Specifically, a 24 hour co-culture resulted in effective killing of the various acute myeloid leukemia cell lines, including MV411 (FIG. 4F), EOL-1 (FIG. 4G), HL60 (FIG. 4H), Kasumi-1 (FIG. 4I), KG1 (FIG. 4J), and THP-1 cells (FIG. 4K). In addition, the data demonstrate that the killing effect of anti-CD70 CAR T cells on acute myeloid leukemia cells increases with an increase dose of the anti-CD70 CAR T cells.

### Example 4: Efficacy of CTX130 Cells: Treatment in the Cutaneous T-Cell Lymphoma Tumor Xenograft Model

The ability of T cells expressing an anti-CD70 CAR to eliminate T cell lymphoma was evaluated in in vivo using a subcutaneous T-cell lymphoma (Hu T78 or Hh) tumor xenograft model in mice.

CRISPR/Cas9 and AAV6 were used as above (see for example, Example 2) to create human anti-CD70 CAR+ T cells that lack expression of the TCR, β2M, CD70 with concomitant expression from the TRAC locus using a CAR construct targeting CD70 (SEQ ID NO: 46 or SEQ ID NO: 81). In this example activated T cells were first electroporated with 3 distinct Cas9:sgRNA RNP complexes containing sgRNAs targeting TRAC (SEQ ID NO: 6), β2M (SEQ ID NO: 10), and CD70 (SEQ ID NO: 2). The DNA double stranded break at the TRAC locus was repaired by homology directed repair with an AAV6-delivered DNA template (SEQ ID NO: 43) (encoding anti-CD70 CAR comprising the amino acid sequence of SEQ ID NO: 46 or SEQ ID NO: 81) containing right and left homology arms to the TRAC locus flanking a chimeric antigen receptor cassette (−/+ regulatory elements for gene expression).

The resulting modified T cells are TRAC−/β2M−/CD70− anti-CD70 CAR+ T cells (CTX130). The ability of these anti-CD70 CAR+ T cells to ameliorate disease caused by a CD70+ T-cell lymphoma cell line was evaluated in NOG mice using methods employed by Translational Drug Development, LLC (Scottsdale, Ariz.). In brief, 12, 5-8 week old female, CIEA NOG (NOD.Cg-Prkdc^{scid}I12rg^{tm1Sug}/JicTac) mice were individually housed in ventilated microisolator cages, maintained under pathogen-free conditions, 5-7 days prior to the start of the study. Mice received a subcutaneous inoculation of 3×10⁶ T-cell lymphoma cells (HuT78 or Hh) in the right hind flank. When mean tumor size reached 25-75 mm³ (target of ˜50 mm³), the mice were further divided into 2 treatment groups as shown in Table 14. On Day 1, treatment group 2 received a single 200 μl intravenous dose of anti-CD70 CAR+ T cells according to Table 14.

TABLE 14
Treatment groups.

| | | | CAR+ T cell | |
|---|---|---|---|---|
| Group | CAR-T | Tumor cells | treatment (i.v.) | N |
| 1 | None | 3 × 10⁶ cells/mouse | None | 5 |
| 2 | CTX130 CAR | 3 × 10⁶ cells/mouse | 1 × 10⁷ cells/ | 5 |
| | T cells | | mouse | |

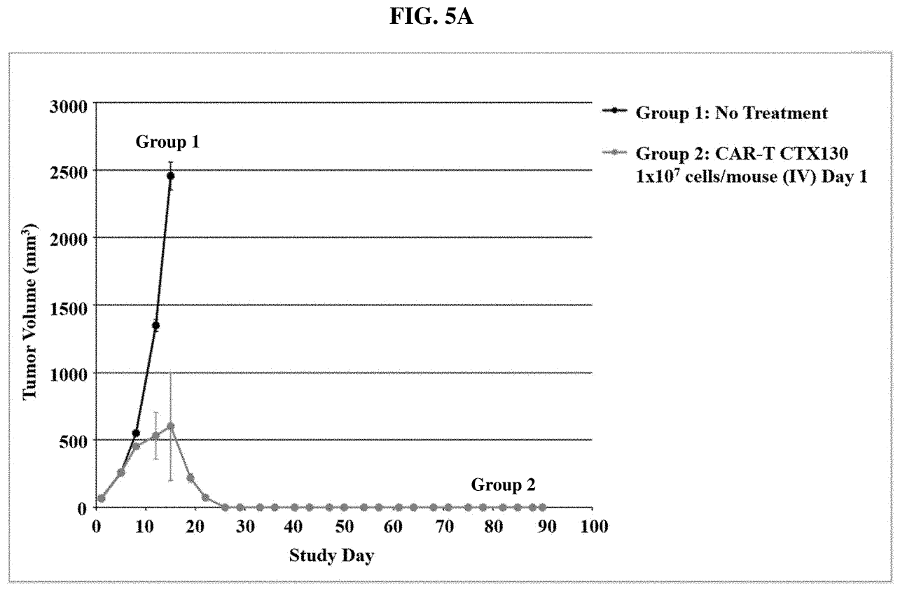

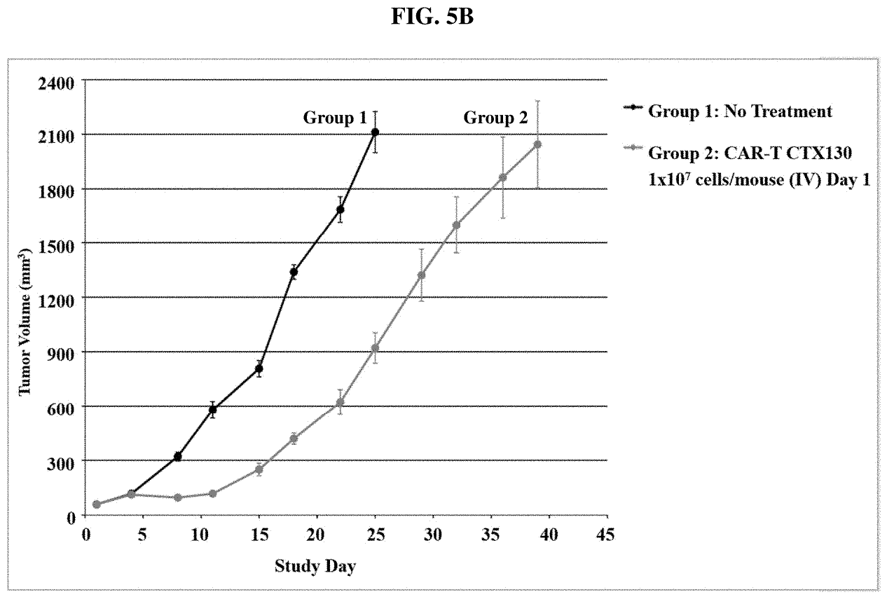

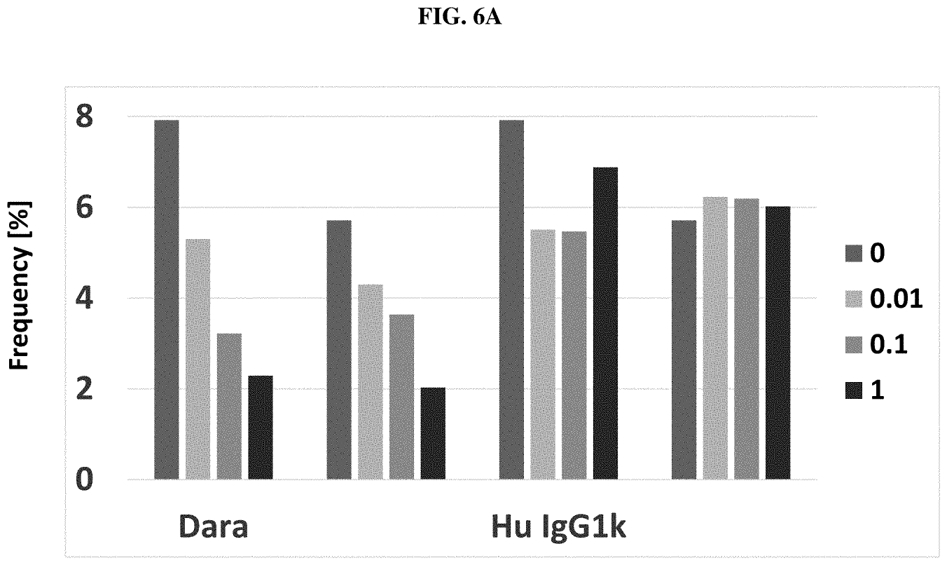

Tumor volume was measured 2 times weekly from day of treatment initiation. By Day 12 post-injection, HuT78 tumors treated with anti-CD70 CAR T cells began to show a decrease in tumor volume in 4 of the 5 treated mice (FIG. 5A). Further tumors were eliminated by day 30 and for the remainder of the study (FIG. 5A). HuT78 tumor growth was significantly inhibited over a 90 day study period (FIG. 5A). Treatment with anti-CD70 CAR+ T cells effectively slowed tumor growth of the Hh T-cell lymphoma tumors in all mice tested over a 45 day period (FIG. 5B).

These data demonstrate that anti-CD70 CAR+ cells (CTX130) inhibited growth of human CD70+ T-cell lymphoma tumors in vivo, with potent activity against established HuT78 and Hh T-cell lymphoma xenografts.

### Example 5: Daratumumab Treatment Depleted NK Cells while T Cell Numbers Remained Unaffected

Based on the expression levels of CD38 on NK and T cells, the effect of an anti-CD38 antibody, daratumumab (TAB-236, Creative Biolabs), on such cells was assessed. PBMCs from a healthy donor were cultured for 96 hours in media containing 0.01, 0.1, or 1 μg/mL of daratumumab. The effect of 10% complement on the cell cultures was also tested. Untreated cells and cells treated with 0.01, 0.1 or 1 μg/mL isotype control mAb (human IgGlk)(cat #403501, BioLegend) were used as controls. After 96 hours of culture, NK and T cell frequency and numbers were measured.

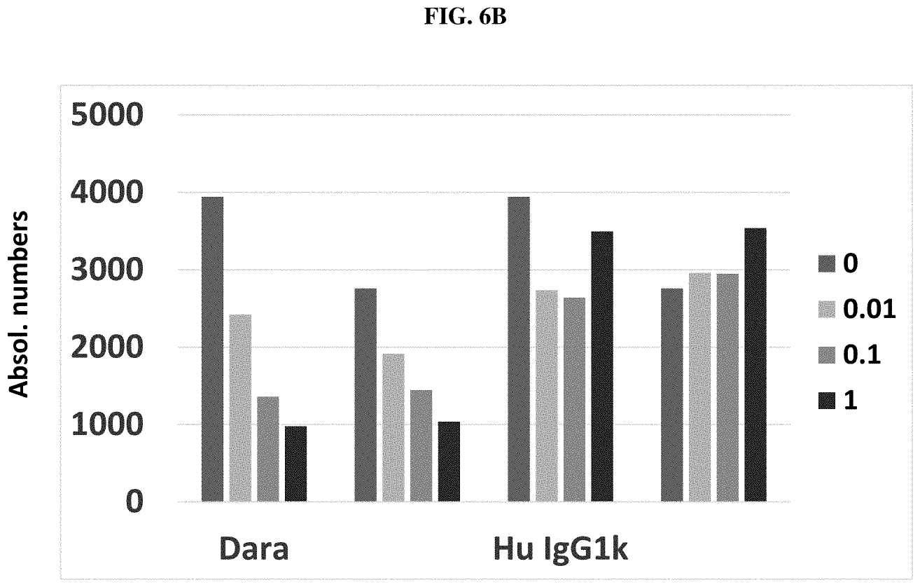

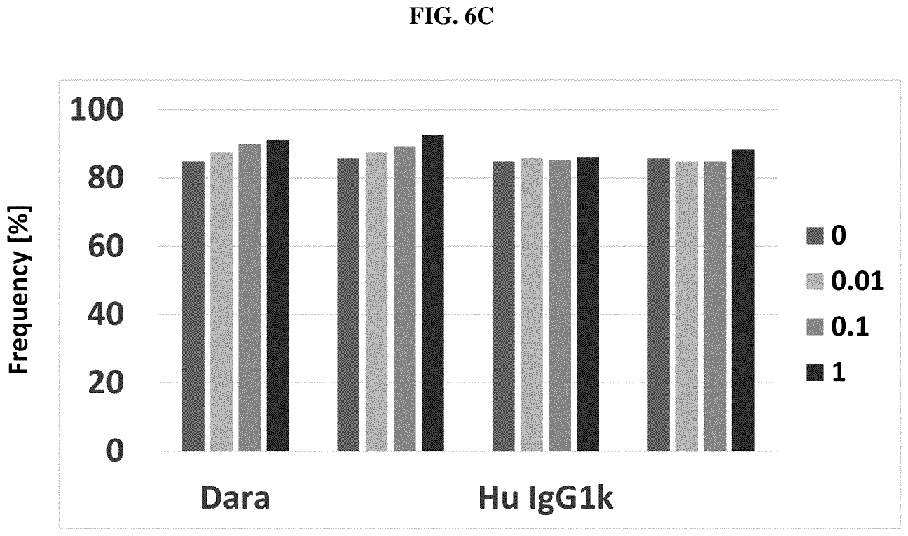

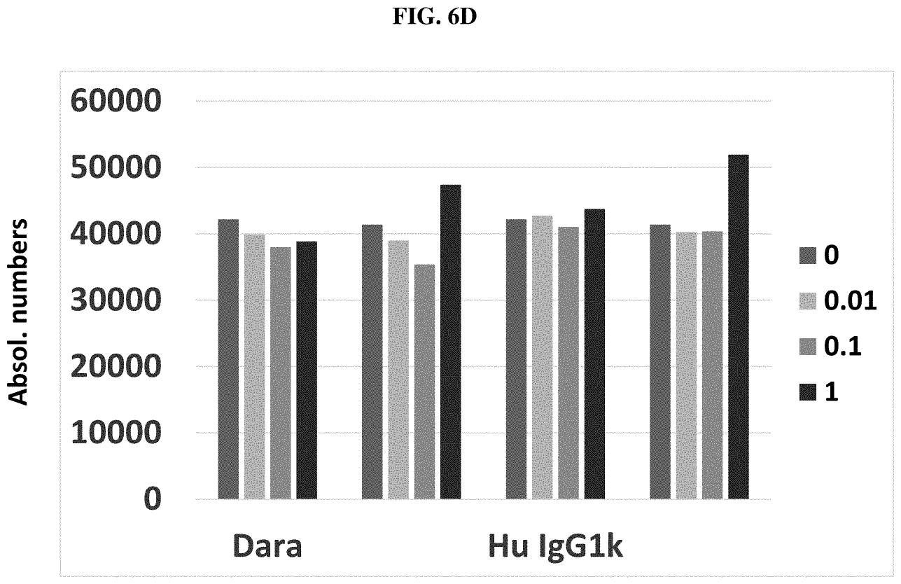

In vitro culture of daratumumab resulted in a dose-dependent decrease of NK cell frequency and numbers (FIGS. 6A-6B). At the highest dose tested, 1 μg/mL, daratumumab reduced NK cell numbers by approximately 75% after 96 hours. This effect is specific to daratumumab, as treatment with an isotype control mAb did not affect NK cell numbers. The reduction in NK cells is not complement dependent under these culture conditions, as the addition of 10% complement to the cell culture did not alter daratumumab's effect of NK cells.

In a second experiment PBMCs from a different donor, daratumumab reduced NK cell numbers ˜57% after only 72 hours (data not shown). These data demonstrate that daratumumab has similar effects on NK cells from different donor populations.

Contrary to its effect on NK cells, daratumumab did not affect T cell numbers or frequency (FIGS. 6C-6D). Although CD38 expression was detected on T cells and in vitro culture of PBMC resulted in upregulation of CD38 surface expression in T cells, T cell numbers were surprisingly unaffected by the addition of daratumumab to the culture media.

### Example 6: A Phase 1, Open-Label, Multicenter, Dose Escalation and Cohort Expansion Study of the Safety and Efficacy of Anti-CD70 Allogeneic CRISPR-Cas9-Engineered T Cells (CTX130) in Adult Subjects with Relapsed or Refractory T or B Cell Malignancies

CTX130 is a CD70-directed allogeneic T cell immunotherapy comprised of T cells that are genetically modified using CRISPR-Cas9 gene editing components (sgRNA and Cas9 nuclease) to knock out the T cell receptor alpha constant (TRAC) and beta 2-microglobulin (β2M) genes, which contribute to graft versus host and host versus graft reactions, respectively.

Simultaneously, an anti-CD70 CAR is inserted at the TCR locus using an AAV vector. The CAR is comprised of a scFv specific for CD70, followed by a CD8 hinge and transmembrane region that is fused to the intracellular co-signaling domain of CD137 (i.e., 4-1BB) and the signaling domain of CD3ζ. The target for CTX130 (i.e., the CD70 protein) is also removed from the final CTX130 product using the CRISPR-Cas9 system by using an sgRNA that targets the CD70 loci. This creates a functional knockout of the CD70 gene and protein in cells in which both copies of the CD70 gene have been edited.

The drug product can be prepared from healthy donor peripheral blood mononuclear cells obtained via a standard leukapheresis procedure. The product is stored onsite and thawed immediately prior to administration.

### 1. Study Population

Part A (single dose escalation) includes adult subjects with the following relapsed/refractory T or B cell malignancies: (a) peripheral T cell lymphoma, not otherwise specified (PTCL-NOS), (b) anaplastic large cell lymphoma (ALCL), (c) Sézary syndrome (SS) or mycosis fungoides (MF), (d) adult T cell leukemia/lymphoma (ATLL), leukemic and lymphomatous subtypes, (e) angioimmunoblastic T cell lymphoma (AITL), and (f) diffuse large B cell lymphoma (DLBCL).

### 2. Study Purpose and Rationale

There is an unmet medical need in subjects with the selected and described T cell lymphomas as well as DLBCL after failed autologous CD19-directed CAR T cell therapy. The selected T or B cell malignancies are reported to have a high expression of CD70 and, therefore, are a potential target for CD70-directed CAR T cell therapies (Baba et al., J Virol, 2008; Lens et al., Br J Hematol, 1999; McEarchern et al., Blood, 2007; Shaffer et al., Blood, 2011).

Although CAR T cell therapy has led to tremendous clinical success, the approved products are autologous and require patient-specific cell collection and manufacturing. These challenges have led to a significant proportion (approximately 30% in 1 study) of subjects enrolled that never received the autologous CAR T cell product (Schuster et al., N Engl J Med, 2019). In addition, the heterogenous nature of each autologous product has made it challenging to demonstrate correlation between CAR T cell dose, toxicity, and/or response in most of the disease indications studied (Mueller et al., Blood, 2017). Recent data suggest that the starting material, specifically the immunophenotype of isolated T cells, may have an impact on disease response (Fraietta et al., Nat Med, 2018). These findings underpin the benefit of an allogeneic CAR T treatment approach for those patients when in need of an urgent, cytoreductive therapy.

CTX130 is manufactured from the T cells of healthy donors, which is intended to result in consistent CAR expression and immunophenotypes across manufacturing runs. Additionally, the manufacturing process initiated from healthy donor cells greatly diminishes the risk of unintentionally transducing malignant T cells during treatment. The recently reported case of a subject with ALL who relapsed with malignant B cells transduced with CAR T cells further underscores this potential risk of a lentiviral approach in which CAR insertion is not coupled to TCR disruption (Ruella et al., Nat Med, 2018). Individual subject manufacturing failures, scheduling complexities, toxicity associated with bridging chemotherapy, and the risks of leukapheresis to the subject do not apply to allogeneic CAR T cell products. The ability to administer CTX130 immediately allows for subjects to receive the product in a timely fashion and helps subjects avoid the need for bridging chemotherapy.

Autologous CAR T cells generated from patients with advanced, relapsed malignancies might be prone to early exhaustion (Fraietta et al., Nat Med, 2018; Mackall, Cancer Res, 2019; Riches et al., Blood, 2013). The use of healthy donor T lymphocytes as the basis for multi-edited allogeneic CAR T cells becomes possible due to the highly precise editing tool CRISPR-Cas9.

The 4 editing steps applied to CTX130 address the safety and efficacy in the following manner:
Safety: Deletion of the TRAC locus to disrupt the endogenous TCR and its interactions with the host MHC system to suppress GvHD.
T cell activity: Insertion of the CD70-targeting CAR construct, deletion of the B2M locus, and deletion of the CD70 locus.

CRISPR-Cas9 allows the coupling of the introduction of the CAR construct as the locus of the deleted through homologous recombination. The delivery and precise insertion of the CAR at the TRAC genomic locus using an AAV-delivered DNA donor template and HDR contrasts with the random insertion of genetic material using other common transduction methods such as lentiviral and retroviral transduction. CAR gene insertion at the TRAC locus results in elimination of TCR in nearly all cells expressing the CAR. While CRISPR-Cas9-mediated disruption of the endogenous TCR can significantly reduce or eliminate the risk of GvHD, the disruption of MHC class I proteins is hypothesized to increase CAR T cell persistence. Deletion of the CD70 locus is intended to increase the persistence of CTX130 and to reduce potential fraternization through elevated expression on activated CAR T cells.

CD70 is a promising target in T or B cell malignancies, (Chahlavi et al., Cancer Res, 2005; Shaffer et al., Blood, 2011). The CAR construct targeting CD70 with its fusion to the costimulatory domains of CD137 (i.e., 4-1BB) and the signaling domain of CD3ζ has been associated with a strong stimulatory signal for the allogeneic cytotoxic T lymphocytes.

This first-in-human trial with CTX130 in subjects with relapsed or refractory T or B cell malignancies evaluates the safety and efficacy of this CRISPR-Cas9-modified allogeneic CAR T cell approach.

### 3. Study Objectives

Primary Objective, Part A (Dose escalation): To assess the safety of escalating doses and/or dosing regimens of CTX130 in subjects with relapsed/refractory T or B cell malignancies and to determine one or more recommended Part B dose (RPBD) regimens.

Primary Objective, Part B (Cohort expansion): To assess the efficacy of CTX130 as measured by objective response rate (ORR) in the following 2 expansion arms, according to Lugano response criteria (Cheson et al., 2014) Appendix A) or International Society for Cutaneous Lymphomas (ISCL) response criteria (Olsen et al., 2011); MF/SS; PTCL.

Secondary Objectives, Parts A and B: To assess activity of CTX130: time to response (TTR), duration of response (DOR), duration of response by best overall response (DOR by BOR), duration of clinical benefit (DOCB), treatment-failure-free survival (TFFS), progression-free survival (PFS), overall survival (OS), MF/SS disease response by compartment; to describe and assess adverse events of special interest (AESIs), including CRS and GvHD; to characterize PK (expansion and persistence) of CTX130 in blood; to describe the effect of CTX130 on patient-reported outcomes.

Exploratory Objectives, Parts A and B: To identify biomarkers that are associated with disease, clinical response, resistance, or safety; to characterize pharmacodynamic activity potentially related to clinical response; to further describe the kinetics of efficacy of CTX130; to evaluate the activity of CTX130: time to complete response (TTCR), best duration of response (BDOR), disease control rate (DCR), time to progression (TTP), PTCL disease response by compartment.

### 4. Study Eligibility

Inclusion Criteria

To be considered eligible to participate in this study, a subject must meet all the inclusion criteria listed below:
1. ≥18 years of age.
2. Able to understand and comply with protocol-required study procedures and voluntarily sign a written informed consent document.
3. For subjects with T cell lymphoma, only the following subtypes are enrolled:
   PTCL-NOS
   ALCL
   SS or MF≥Stage IIB with ≥2-compartment disease or single compartment disease with large cell transformation
   Leukemic and lymphomatous subtypes of ATLL
   AITL
   Subjects with PTCL-NOS, leukemic and lymphomatous ATLL, or AITL should have failed≥1 lines of systemic therapy.
   Subjects with ALCL should have failed, be ineligible for, or have refused combination chemotherapy and therapy with brentuximab vedotin in combination or as single agent.
      Subjects with ALK⁻ ALCL should have failed a minimum of 1 prior line of therapy
      Subjects with ALK⁺ ALCL should have failed a minimum of 2 prior lines of therapy
   Subjects with MF or SS must have failed at least 2 of the following systemic or total body directed therapies: brentuximab vedotin, romidepsin (or other indicated histone deacetylase inhibitors), pralatrexate, mogamulizumab, total skin electron beam therapy (TSEBT), pembrolizumab, or other systemic chemotherapy. If mogamulizumab was the last therapy prior to enrollment, there must be at least 50 days between the last dose of mogamulizumab and the infusion of CTX130.
4. For subjects with B cell lymphoma: DLBCL in subjects who have received up to 4 lines of prior systemic therapy, including autologous CD19-directed CAR T cell therapy unless CD19-directed CAR T cell therapy was refused or attempted and failed manufacturing.
5. Subjects must have CD70-expressing tumors as determined by laboratories meeting applicable local requirements (e.g., Clinical Laboratory Improvement Amendments or equivalent for non-US locations) by either:
   CD70 positivity (≥10% of cells) by IHC in tissue collected by excisional or core biopsy of a representative tumor lesion. In cases where more than one sample is submitted, a single sample testing positive is sufficient for eligibility.
   CD70 positivity (≥10% of cells) by flow cytometry in tumor cells defined by immunophenotyping collected in the peripheral blood or bone marrow at screening
6. Be willing to provide tissue from a newly obtained core or excisional biopsy of a tumor lesion at screening unless a biopsy performed within 3 months prior to enrollment and after the last systemic or targeted therapy post progression is available.
7. Eastern Cooperative Oncology Group (ECOG) performance status of 0-1

TABLE 15
ECOG Performance Status Scale

| Grade | Description |
|---|---|
| 0 | Fully active, able to carry on all pre-disease performance |
| | without restriction |
| 1 | Restricted in physically strenuous activity but ambulatory and |
| | able to carry out work of a light or sedentary nature, e.g., light |
| | house work, office work |
| 2 | Ambulatory and capable of all self-care but unable to carry |
| | out any work activities; up and about more than 50% of |
| | waking hours |
| 3 | Capable of only limited self-care; confined to bed or chair |
| | more than 50% of waking hours |
| 4 | Completely disabled; cannot carry on any self-care; totally |
| | confined to bed or chair |
| 5 | Dead |

Developed by the Eastern Cooperative Oncology Group, Robert L. Comis, MD, Group Chair (Oken et al., 1982).

8. Meets protocol-specified criteria to undergo daratumamab infusion (Parts A2, A4, and A6 only), LD chemotherapy, and CAR T cell infusion
9. Adequate organ function:
   Renal: creatinine clearance (CrCl) ≥50 mL/min
   Liver:
      Aspartate aminotransferase and alanine aminotransferase <3× upper limit of normal (ULN)
      Total bilirubin <2×ULN (for Gilbert's syndrome: total bilirubin <3 mg/dL and normal conjugated bilirubin)
   Cardiac: Hemodynamically stable and left ventricular ejection fraction (LVEF)≥45% by echocardiogram
   Pulmonary: Oxygen saturation level on room air >92% per pulse oximetry
   Hematologic: Platelet count >25,000/mm3 and absolute neutrophil count >500/mm³
10. Female subjects of childbearing potential (postmenarcheal, has an intact uterus and at least 1 ovary, and is less than 1 year postmenopausal) must agree to use a highly effective method of contraception from enrollment through at least 12 months after last CTX130 infusion.
11. Male subjects must agree to use acceptable effective method(s) of contraception from enrollment through at least 12 months after last CTX130 infusion.
12. Subjects must have measurable disease per mSWAT or peripheral blood tumor burden, or at least 1 measurable lesion by imaging (PET-CT or CT) according to Lugano criteria; lesion cannot have been biopsied or irradiated.

Exclusion Criteria

To be eligible for entry into the study, the subject must not meet any of the exclusion criteria listed below:
1. Prior allogeneic SCT.
2. Less than 60 days from autologous SCT at time of screening and with unresolved serious complications.
3. Prior treatment with anti-CD70 targeting agents.
4. For subjects with DLBCL, prior treatment with CAR T cells or other modified T or NK cells except autologous CD19-directed CAR T cells.
5. Known contraindication to daratumumab (Parts A2, A4, and A6 only), any LD chemotherapy agent(s), or any of the excipients of CTX130 product.
6. T cell or B cell lymphomas with a present or past malignant effusion that is or was symptomatic.
7. Clinical signs of HLH: A combination of fever, bicytopenia, hypertriglyceridemia or hypofibrinogenemia and ferritin >500 μg/L.
8. Active central nervous system (CNS) manifestation of underlying disease in screening imaging (i.e., brain MRI).
9. History or presence of clinically relevant CNS pathology such as seizure, stroke, severe brain injury, cerebellar disease, myelopathy (e.g., tropical spastic paraparesis), history of posterior reversible encephalopathy syndrome with prior therapy, or another condition that may increase CAR T-related toxicities.
10. Unstable angina, arrhythmia, or myocardial infarction within 6 months prior to screening.
11. Ongoing bacterial, viral, or fungal infection requiring systemic anti-infectives.
12. Positive for presence of human immunodeficiency virus type 1 or 2 (HIV-1 or HIV-2), or active hepatitis B virus or hepatitis C virus infection. Subjects with prior history of hepatitis B or C infection who have documented undetectable viral load (by quantitative polymerase chain reaction (PCR) or nucleic acid testing) are permitted.
13. Previous or concurrent malignancy, except for the following:
   Those treated with curative approach who have been in remission for >12 months without requiring systemic therapy (antihormonal therapy accepted)
   Adequately treated non-melanoma skin cancer or lentigo maligna without evidence of disease.
   Adequately treated cervical carcinoma in situ without evidence of disease.
   Adequately treated breast ductal carcinoma in situ without evidence of disease.
   Prostatic intraepithelial neoplasia without evidence of prostate cancer.
   Adequately treated urothelial papillary noninvasive carcinoma or carcinoma in situ
14. Primary immunodeficiency disorder or active autoimmune disease requiring steroids and/or other immunosuppressive therapy.
15. Prior solid organ transplantation.
16. Prior use of antitumor agents, except palliative radiotherapy, 14 days prior to LD chemotherapy. Washout time needs to be discussed with the medical monitor. Use of physiological doses of steroids (≤10 mg/day of prednisone or equivalent doses of other corticosteroids) is permitted for subjects previously on steroids. Intrathecal prophylaxis for subjects with ATLL is permitted if indicated. Subjects with ATLL receiving the receptor activator of nuclear factor kappa-B ligand (RANKL) inhibitor denosumab should be on therapy for at least 4 weeks and must have stabilized corrected serum calcium levels; and are excluded if serum calcium level is >11.5 mg/dL or >2.9 mmol/L, or ionized calcium level is >1.5 mmol/L. Use of CCR-4-directed antibodies is prohibited 3 months prior to CTX130 infusion, except for mogamulizumab, which is prohibited 50 days prior to CTX130 infusion.
17. Diagnosis of significant psychiatric disorder that could seriously impede the subject's ability to participate in the study.
18. Received live vaccines or herbal medicines as part of traditional Chinese medicine or non-over-the-counter herbal remedies within 28 days prior to enrollment.
19. Pregnant or breastfeeding females.

### 5. Study Design

### Investigational Plan

This is an open-label, multicohort, multicenter, dose escalation Phase 1 study in subjects ≥18 years of age with relapsed or refractory T or B cell malignancies. The study is divided into 2 parts: dose escalation (Part A) followed by cohort expansion (Part B). Part A establishes one or more recommended Part B dosing regimens for expansion.

Part A includes 6 subparts (Parts A1 through A6; details on the treatment regimen specific to each subpart can be found in Table 2):
Part A1 (dose escalation) Part A2 (dose escalation with daratumumab added to the lymphodepletion regimen)
Part A3 (dose escalation with a second CTX130 infusion on Day 5 [+2 days])
Part A4 (dose escalation with daratumumab added to the lymphodepletion regimen and with additional CTX130 infusion on Day 5 [+2 days])
Part A5 (dose escalation with Day 35 [−7 days/+21 days] CTX130 consolidation)
Part A6 (dose escalation with daratumumab added to the lymphodepletion regimen and with Day 35 [−7 days/+21 days] CTX130 consolidation)

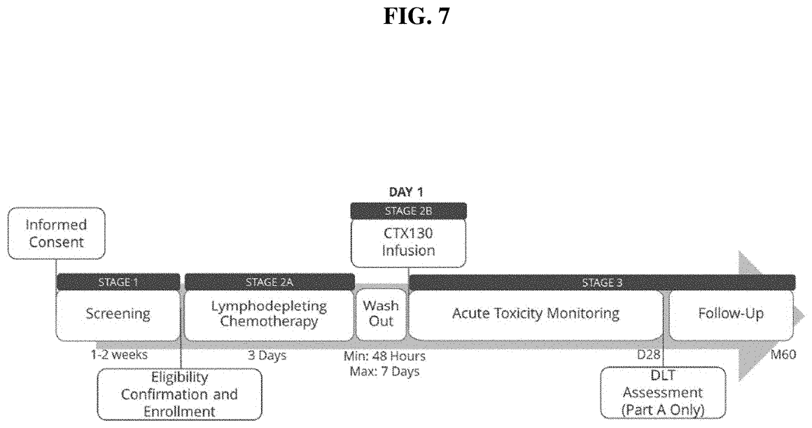

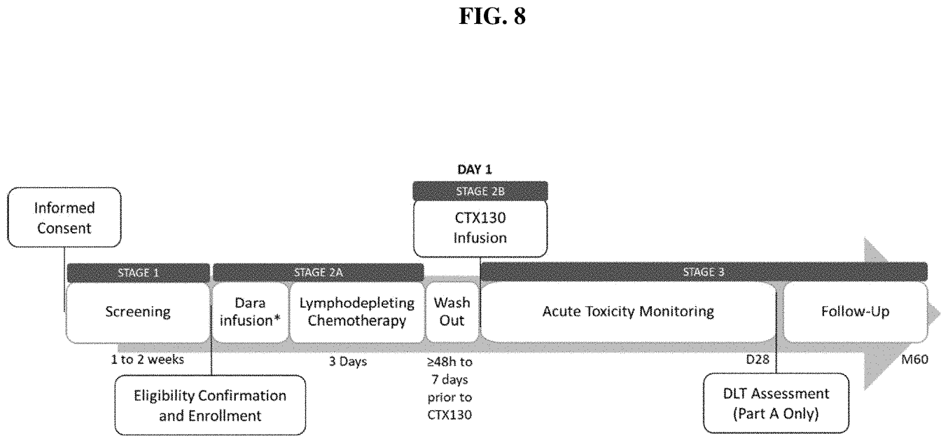

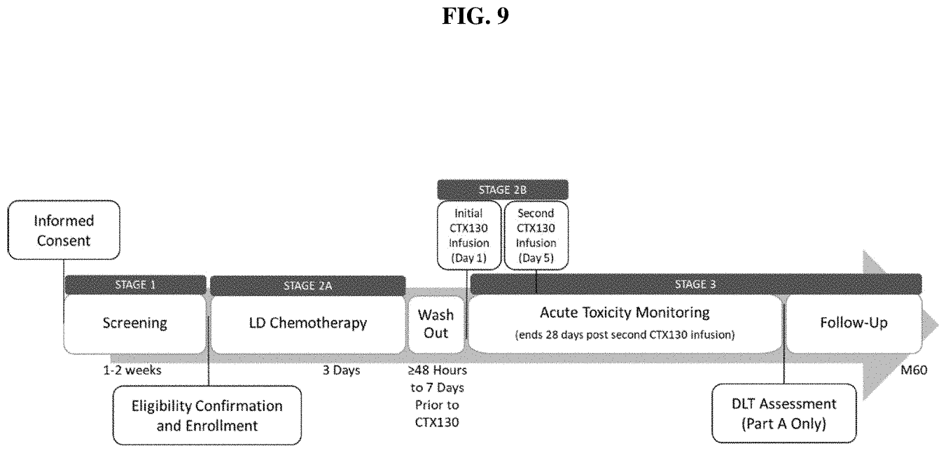

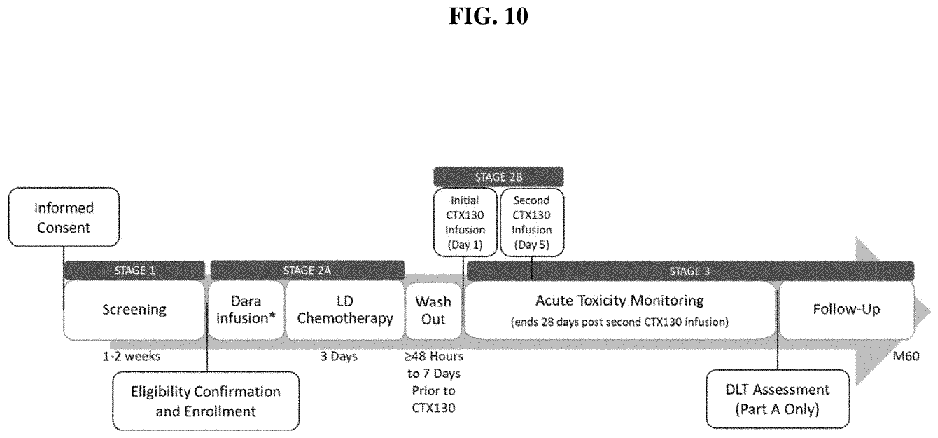

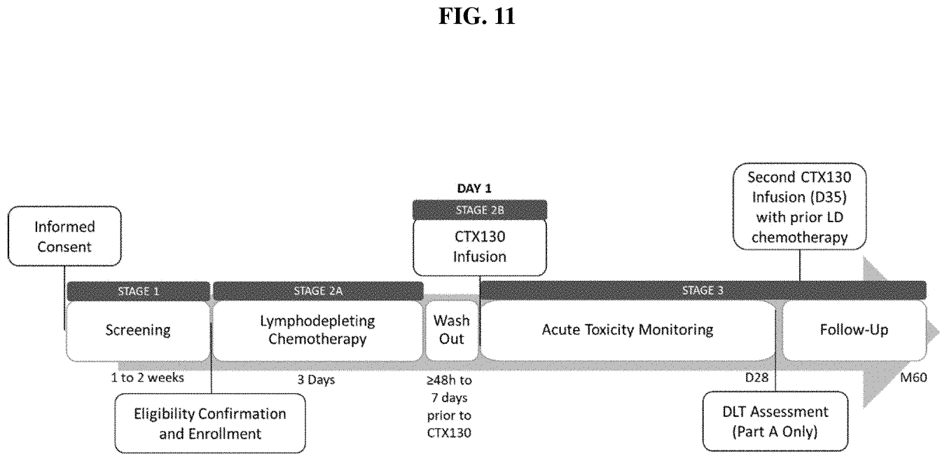

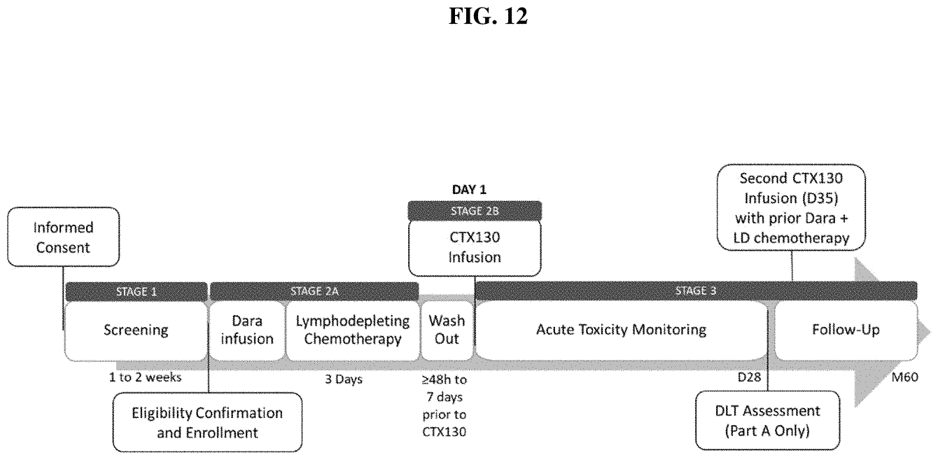

Parts A1 (FIG. 7) and A2 (FIG. 8) evaluate the safety of a single escalating dose of CTX130, Parts A3 (FIG. 9) and A4 (FIG. 10) evaluate the safety of an initial infusion of CTX130 on Day 1 followed by an additional infusion of CTX130 on Day 5, and Parts A5 (FIG. 11) and A6 (FIG. 12) evaluate an initial infusion of CTX130 followed by a second infusion of CTX130 on Day 35 (−7 days/+21 days) for subjects who achieve CR, PR, SD, or PD with clinical benefit. Part B assesses the safety and efficacy of one or more recommended dosing regimens of CTX130 in cohort expansion.

In Parts A1 through A4 of this study, subjects may be considered for up to 2 additional courses of treatment with CTX130 based on the investigator's decision in consultation with the sponsor's medical monitor. Courses of treatment are performed as follows:
For Parts A1 and A2, the first course of treatment encompasses a single infusion of CTX130 on Day 1 with respective LD regimen. For Parts A3 and A4, the first course of treatment encompasses an initial infusion of CTX130 on Day 1 with respective LD regimen and a second CTX130 infusion without LD regimen on Day 5.
For Parts A1 through A4, an optional second course of treatment can be administered with LD chemotherapy (and with daratumumab if applicable) after 1) loss of complete response (CR) within the first 2 years after initial infusion of CTX130, or 2) partial response (PR), stable disease (SD), or progressive disease (PD) with clinical benefit as determined by the investigator. The second course of treatment includes a single infusion of CTX130 with respective LD regimen for Parts A1 and A2, and a CTX130 infusion with respective LD regimen followed by a second CTX130 infusion 4 days later without LD regimen for Parts A3 and A4.
An optional third course of treatment is also available for Parts A1 through A4 that is identical to the second course of treatment and can be administered after loss of CR within the first 2 years after initial infusion of CTX130 or after assessment of PR, SD, or PD with clinical benefit.

In Parts A5 and A6 of this study, the first course of treatment includes an initial infusion of CTX130 on Day 1 with respective LD regimen, and for subjects who achieve CR, PR, SD, or PD with clinical benefit, a second CTX130 infusion on Day 35 with respective LD regimen (see Table 2 below for details).

In Parts A5 and A6, after the first course of treatment, an optional single additional infusion of CTX130 can be administered with LD chemotherapy (and with daratumumab if applicable), based on the investigator's decision in consultation with the sponsor's medical monitor, after 1) loss of complete response (CR) within the first 2 years after initial infusion of CTX130, or 2) partial response (PR), stable disease (SD), or progressive disease (PD) with clinical benefit as determined by the investigator. The first day of LD chemotherapy prior to the single additional infusion of CTX130 must be at least 28 days after the last day of LD chemotherapy in the first course of treatment.

For all subjects in the study, the first day of LD chemotherapy prior to a CTX130 infusion must be at least 28 days after the last day of LD chemotherapy for the previous infusion of CTX130.

For subjects experiencing significant cytopenia, the LD regimen may be omitted prior to:
additional courses of treatment in Parts A1 through A4
a single additional infusion of CTX130 after the first course of treatment in Parts A5 and A6
the Day 35 CTX130 infusion during the first course of treatment in Parts A5 and A6

In Part A, dose escalation begins in adult subjects with 1 of the following:

1. T Cell Malignancies:
Subjects with PTCL-NOS, leukemic and lymphomatous ATLL, or AITL should have failed ≥1 lines of systemic therapy.
Subjects with ALCL should have failed, be ineligible for, or have refused combination chemotherapy and/or therapy with brentuximab vedotin.
   Subjects with ALK− ALCL should have failed 1 prior line of therapy.
   Subjects with ALK+ ALCL should have failed 2 prior lines of therapy.
Subjects with MF or SS must have failed 2 of the following systemic or total body directed therapies: brentuximab vedotin, romidepsin (or other indicated histone deacetylase inhibitors), pralatrexate, mogamulizumab, total skin electron beam therapy (TSEBT), pembrolizumab, or other systemic chemotherapy. If mogamulizumab was the last therapy prior to enrollment, there must be a period of at least 50 days between the last dose of mogamulizumab and the infusion of CTX130.

2. B Cell Malignancy:
DLBCL in subjects who have received up to 4 lines of prior systemic therapy, including autologous CD19-directed CAR T cell therapy unless autologous CD19-directed CAR T cell therapy was refused or attempted and failed manufacturing. Dose escalation is performed according to the criteria described herein.

Subjects in Part A2, A4, and A6 receive daratumumab for IV use (Darzalex®, USPI 2019) or daratumumab and hyaluronidase-fihj for subcutaneous use (FASPRO, USPI 2020), Janssen; a human immunoglobulin G1 monoclonal antibody that targets CD38 surface antigen) prior to LD chemotherapy to achieve depletion of CD38-positive immune suppressor and effector cells (e.g., natural killer (NK) cells). CTX130 is an allogeneic CAR T cell with disruption of the B2M locus resulting in elimination of major histocompatibility complex (MHC) class I expression on the cell surface. NK cells can potentially detect and clear these “non-self” MHC class 1 negative cells. Rapid NK cell recovery after LD chemotherapy coincides with peak CTX130 expansion. Based on these observations, the suppression of specific NK cell subpopulations with daratumumab in addition to LD chemotherapy may reduce the potential host immune response to an allogeneic CAR T cell product, and therefore allow increased expansion and persistence of CTX130.

Dosing of CTX130 at any dose level in Parts A2, A3 and A5 will not begin unless the dose level has been deemed safe by the SRC in Part A1, and dosing of CTX130 at any dose level in Parts A4 and A6 will not begin unless the dose level has been deemed safe by the SRC in Part A2. Dose escalation/de-escalation is allowed according to the 3+3 design. In Parts A2 and A4, daratumumab administration at 16 mg/kg IV or 1800 mg SC is repeated at Day 21 and Day 42.

In Part B, an expansion cohort is initiated to further assess the safety and efficacy of CTX130 at the RPBD regimen in subjects with the following T cell lymphoma subtypes:
Advanced and transformed MF and SS with ≥2 prior systemic therapies
PTCL with ≥1 prior systemic therapy, including:
PTCL-NOS
AITL
ALCL (with prior brentuximab vedotin therapy)
Leukemic and lymphomatous subtypes of ATLL

Each arm will have an interim analysis to assess futility and early efficacy after approximately 50% of subjects have been enrolled and have completed at least their Month 3 visit or discontinued earlier, followed by a final analysis.

### Study Design

This is an open-label, multicohort, multicenter, dose escalation Phase 1 study in subjects ≥18 years of age with relapsed or refractory T or B cell malignancies. The study is divided into 2 parts: dose escalation (Part A, which includes Parts A1 through A6) followed by cohort expansion (Part B).

Both Parts A and B of the study consist of the following 3 main stages:
Stage 1—Screening to determine eligibility for treatment (up to 14 days).
Stage 2—Treatment (Stage 2A and Stage 2B); see Table 16 for treatment in each Part of the study.
Stage 3—Follow-up (up to 5 years after last CTX130 infusion)

Administration of daratumumab (Parts A2, A4, and A6 only), initiation of LD chemotherapy, or infusion of CTX130, is delayed if subjects do not meet the protocol-specified criteria described herein.

Lymphodepletion regimens and CTX130 dosing in Part A are summarized in Table 16.

TABLE 16
Lymphodepletion Regimens and CTX130 Dosing
Part A

| | |
|---|---|
| A1 | Stage 2A |
| (Dose | LD chemotherapy: co-administration of fludarabine 30 mg/m² and |
| Escalation) | cyclophosphamide 500 mg/m² IV daily for 3 days. |
| | Stage 2B |
| | A single infusion of CTX130 starting at DL1, administered at least 48 hours |
| | (but no more than 7 days) after completion of LD chemotherapy. |
| A2 | Stage 2A |
| (Dose | One dose of daratumumab (16 mg/kg IV or 1800 mg SC injection) |
| escalation with | administered at least 12 hours prior to starting LD chemotherapy and within |
| addition of | 10 days prior to CTX130 infusion. To facilitate administration, the 16 |
| daratumumab | mg/kg IV dose may be split (to 8 mg/kg IV) over 2 consecutive days per |
| to | daratumumab prescribing information. Daratumumab administration at 16 |
| lymphodepletion | mg/kg IV or 1800 mg SC is repeated at Day 21 and Day 42. |
| regimen) | LD chemotherapy: Co-administration of fludarabine 30 mg/m2 + |
| | cyclophosphamide 500 mg/m2 IV daily for 3 days. |
| | Stage 2B |
| | A single infusion of CTX130 will start at a dose level that has been deemed |
| | safe by the SRC in Part A1. CTX130 is administered at least 48 hours (but |
| | no more than 7 days) after completion of LD chemotherapy. |
| A3 | Stage 2A |
| (Dose | LD chemotherapy: co-administration of fludarabine 30 mg/m2 and |
| escalation with | cyclophosphamide 500 mg/m2 IV daily for 3 days. |
| second | Stage 2B |
| CTX130 | Initial CTX130 infusion on Day 1 will start at a dose level that has been |
| infusion on | deemed safe by the SRC in Part A1. CTX130 is administered at least 48 |
| Day 5 [+2 | hours (but no more than 7 days) after completion of LD chemotherapy. |
| days]) | A second infusion of CTX130 on Day 5 (+2 days) is administered without |
| | LD chemotherapy for subjects meeting safety parameters |
| A4 (Dose | Stage 2A |
| escalation with | One dose of daratumumab (16 mg/kg IV or 1800 mg SC injection) |
| daratumumab | administered at least 12 hours prior to starting LD chemotherapy and within |
| added to | 10 days prior to CTX130 infusion. To facilitate administration, the 16 |
| lymphodepletion | mg/kg IV dose may be split (to 8 mg/kg IV) over 2 consecutive days per |
| regimen and | daratumumab prescribing information. Daratumumab administration at 16 |
| with second | mg/kg IV or 1800 mg SC is repeated at Day 21 and Day 42. |
| CTX130 | LD chemotherapy: Co-administration of fludarabine 30 mg/m2 + |
| infusion on | cyclophosphamide 500 mg/m2 IV daily for 3 days. |
| Day 5 [+2 | Stage 2B |
| days]) | Initial CTX130 infusion on Day 1 will start at a dose level that has been |
| | deemed safe by the SRC in Part A2. CTX130 is administered at least 48 |
| | hours (but no more than 7 days) after completion of LD chemotherapy. |
| | A second infusion of CTX130 on Day 5 (+2 days) is administered without |
| | daratumumab and LD chemotherapy for subjects meeting safety parameters. |
| A5 (Dose | Stage 2A |
| escalation with | LD chemotherapy: co-administration of fludarabine 30 mg/m2 and |
| Day 35 | cyclophosphamide 500 mg/m2 IV daily for 3 days. |
| CTX130 | Stage 2B |
| consolidation) | An initial infusion of CTX130 will start at a dose level that has been |
| | deemed safe by the SRC in Part A1. CTX130 is administered at least 48 |
| | hours (but no more than 7 days) after completion of LD chemotherapy. |
| | A second infusion of CTX130 on Day 35 (−7 days/+21 days) is administered |
| | with LD chemotherapy for subjects who achieve CR, PR, SD, or PD with |
| | clinical benefit at Day 28 scan (based on Lugano or Olsen criteria as |
| | appropriate). The second infusion may be administered without LD |
| | chemotherapy if subject is experiencing significant cytopenia. |
| A6 (Dose | Stage 2A |
| escalation with | One dose of daratumumab (16 mg/kg IV or 1800 mg SC injection) |
| daratumumab | administered at least 12 hours prior to starting LD chemotherapy and within |
| added to | 10 days prior to CTX130 infusion. To facilitate administration, the 16 |
| lymphodepletion | mg/kg IV dose may be split (to 8 mg/kg IV) over 2 consecutive days per |
| regimen and | daratumumab prescribing information. |
| with Day 35 | LD chemotherapy: Co-administration of fludarabine 30 mg/m2 + |
| CTX130 | cyclophosphamide 500 mg/m2 IV daily for 3 days. |
| consolidation) | Stage 2B |
| | An initial infusion of CTX130 will start at a dose level that has been |
| | deemed safe by the SRC in Part A2. CTX130 is administered at least 48 |
| | hours (but no more than 7 days) after completion of LD chemotherapy. |
| | A second infusion of CTX130 on Day 35 (−7 days/+21 days) is administered |
| | with daratumumab and LD chemotherapy for subjects who achieve CR, PR, |
| | SD, or PD with clinical benefit at Day 28 scan (based on Lugano or Olsen |
| | criteria as appropriate). The second infusion may be administered without |
| | LD chemotherapy if subject is experiencing significant cytopenia. |
| Part B | Dosing regimen determined in Part A |
| (Cohort | |
| Expansion) | |

CR: complete response; DL: dose level; IV: intravenously; LD: lymphodepleting; PD: progressive disease; PR: partial response; SC: subcutaneous; SD: stable disease; SRC: Safety Review Committee Note: In Parts A1 through A4, a second course of treatment can be administered with LD chemotherapy (and with daratumumab if applicable) after 1) loss of CR within the first 2 years after initial infusion of CTX130, or 2) PR, SD, or PD with clinical benefit as determined by the investigator. A third course of treatment is also available for Parts A1 through A4 that is identical to the second course of treatment and can be administered after loss of CR within the first 2 years after initial infusion of CTX130 or after assessment of PR, SD, or PD with clinical benefit. These additional courses of treatment is allowed at a CTX130 dose level that has been deemed safe by the SRC and that is greater than or equal to the CTX130 dose level administered during the first course of treatment.
Note: In Parts A5 and A6, after the first course of treatment, an optional single additional infusion of CTX130 can be administered with LD chemotherapy (and with daratumumab if applicable) after 1) loss of complete response (CR) within the first 2 years after initial infusion of CTX130, or 2) partial response (PR), stable disease (SD), or progressive disease (PD) with clinical benefit as determined by the investigator. The additional CTX130 infusion is allowed at a CTX130 dose level that has been deemed safe by the SRC and that is greater than or equal to the CTX130 dose level administered during the first course of treatment.
Note: prior to additional courses of treatment in Parts A1 through A4, or prior to a single additional infusion of CTX130 after the first course of treatment in Parts A5 and A6, the LD regimen may be omitted if subject is experiencing significant cytopenia.

For Parts A2, A4, and A6, after at least 3 subjects are treated at a specific CTX130 dose with addition of daratumumab to the lymphodepletion regimen, the total safety and efficacy data is reviewed and may additional subjects may be enrolled at a specific dose level with a lower dose of daratumumab (8 mg/kg IV).

During the post-CTX130 infusion period, subjects are monitored for all acute toxicities (Days 1-28), including CRS, immune effector cell-associated neurotoxicity syndrome (ICANS), GvHD, and other adverse events (AEs). Toxicity management guidelines are provided herein. During Part A (dose escalation), all subjects are hospitalized for the first 7 days following each CTX130 infusion, or longer if required by local regulation or site practice. In both Part A and Part B, subjects must remain within proximity of the investigative site (i.e., 1-hour transit time) for 28 days after each CTX130 infusion.

After the acute toxicity observation period, subjects are subsequently followed for up to 5 years after last CTX130 infusion with physical exams, regular laboratory and imaging assessments, and AE assessments. After completion of this study, subjects are asked to participate in a separate long-term follow-up study for an additional 10 years to assess long-term safety and survival.

See Tables 20, 21, 40, and 41 for the schedule of study assessments.

Subjects who are discontinued from the regular schedule of assessments due to disease progression, investigator decision/start of new anticancer therapy, adverse events, protocol violation, or pregnancy will attend annual visits to collect safety information for up to 5 years.

### CTX130 Dose Escalation

Dose levels evaluated in this study are presented in Table 17. There is a dose limit of 7×10⁴ TCR+ cells/kg imposed for all dose levels.

TABLE 17
Dose Escalation of CTX130

| Dose Level | Total CAR+ T Cell Dose |
|---|---|
| −1 (de-escalation) | 1 × 10⁶ |
| 1 | 3 × 10⁷ |
| 2 | 1 × 10⁸ |
| 3 | 3 × 10⁸ |
| 4 | 9 × 10⁸ * |
| 5 | 1.8 × 10⁹ |

CAR: chimeric antigen receptor; DL: dose level.
* An intermediate dose level between DL3 and DL4, i.e., 4.5 × 10⁸, 6 × 10⁸, or 7.5 × 10⁸ CAR⁺ T cells, may be administered based on review of DL4 safety data.

Dose escalation in Part A is performed using a standard 3+3 design in which 3 to 6 subjects are treated at each dose level depending on the occurrence of DLTs The DLT evaluation period begins with the initial CTX130 infusion and last for 28 days. For Parts A3 and A4, the DLT evaluation period will last for 28 days after the second infusion (Day 5).

Subjects who receive a subsequent CTX130 infusion is monitored for frequency and severity of AEs and adverse events of special interest during the immediate 28-day period after each additional CTX130 infusion in addition to the assessment of safety per the DLT criteria defined in the protocol.

For Part A1 (dose escalation): In all Dose Levels (−1 to 5), subjects 1 through 3 are treated in a staggered manner, such that a subject will only receive CTX130 once the previous subject has completed the DLT evaluation period (i.e., staggered by at least 28 days). Dosing between each dose level will also be staggered by at least 28 days; for expansion of the same dose level, cohorts of up to 3 subjects may be enrolled and dosed concurrently.

For Part A2 (dose escalation with daratumumab added to the lymphodepletion regimen): Dosing of CTX130 at any dose level in Part A2 will not begin unless the dose level has been deemed safe by the SRC in Part A1. Dose escalation/de-escalation is allowed according to the 3+3 design (see dose escalation rules below). Sentinel dosing is implemented for the starting dose level only, i.e., the first subject will complete the DLT evaluation period before the second and third subjects are dosed. The second and third subjects may be dosed concurrently. In subsequent dose levels or expansion of the same dose level, cohorts of up to 3 subjects may be enrolled and dosed concurrently. Daratumumab administration at 16 mg/kg IV or 1800 mg SC is repeated at Day 21 and Day 42.

For Part A3 (dose escalation with additional CTX130 infusion on Day 5 [+2 days]) and Part A5 (dose escalation with Day 35 [−7 days/+21 days] CTX130 consolidation): Dosing of CTX130 will start at a dose level that has been deemed safe by the SRC in Part A1. Sentinel dosing is implemented for the starting dose level only, i.e., the first 2 subjects are treated in a staggered manner, such that the second subject will only receive CTX130 after the previous subject has completed the DLT evaluation period. The second and third subjects may be dosed concurrently. In subsequent dose levels or expansion of the same dose level, cohorts of up to 3 subjects may be enrolled and dosed concurrently.

For Part A4 (dose escalation with daratumumab added to the lymphodepletion regimen and with second CTX130 infusion on Day 5 [+2 days]) and Part A6 (dose escalation with daratumumab added to the lymphodepletion regimen and with Day 35 [−7 days/+21 days] CTX130 consolidation): Dosing of CTX130 will start at a dose level that has been deemed safe by the SRC in Part A2. Sentinel dosing is implemented for the starting dose level only, i.e., the first 2 subjects are treated in a staggered manner, such that the second subject will only receive CTX130 after the previous subject has completed the DLT evaluation period. The second and third subjects may be dosed concurrently. In subsequent dose levels or expansion of the same dose level, cohorts of up to 3 subjects may be enrolled and dosed concurrently. In Part A4, daratumumab administration at 16 mg/kg IV or 1800 mg SC is repeated at Day 21 and Day 42. In Part A6, daratumumab is administered only as part of the LD regimen prior to CTX130 infusion.

For Part A: Subjects must receive CTX130 to be evaluated for DLT. If a subject discontinues the study any time prior to the initial CTX130 infusion, the subject is deemed nonevaluable for DLT and is replaced. If a DLT-evaluable subject (i.e., a subject that has been administered CTX130) has signs or symptoms of a potential DLT, the DLT evaluation period is extended according to the protocol-defined window to allow for improvement or resolution before a DLT is declared.

Dose escalation is performed according to the following rules:
If 0 of 3 subjects experience a DLT, escalate to the next dose level.
If 1 of 3 subjects experiences a DLT, expand the current dose level to 6 subjects.
If 1 of 6 subjects experiences a DLT, escalate to the next dose level.
If ≥2 of 6 subjects experience a DLT:
   If in DL-1, evaluate alternative dosing schema or declare inability to determine recommended dose for Part B cohort expansion.
   If in DL1, de-escalate to DL-1.
If in DLs 2-5, declare previous dose level the maximum tolerated dose (MTD).
If ≥2 of 3 subjects experience a DLT:
   If in DL-1, evaluate alternative dosing schema or declare inability to determine the recommended dose for Part B cohort expansion.
   If in DL1, decrease to DL-1.
   If in DLs 2-5, declare previous dose level the MTD. If this is the starting dose level, de-escalate to a dose previously cleared in Part A1.
No dose escalation beyond highest dose listed in Table 17.

At least 6 subjects are administered CTX130 before an RPBD is declared.

### Dose Limiting Toxicity (DLT) Definitions

Toxicities are graded and documented according to NCI Common Terminology Criteria for Adverse Events version 5.0 (CTCAE v5.0) except for CRS (American Society for Transplantation and Cellular Therapy [ASTCT] criteria; (Lee et al., Blood, 2014), neurotoxicity (ICANS criteria and CTCAE v5.0; (Lee et al., Biol Blood Marrow Transplant, 2019), and GvHD (Mount Sinai Acute GvHD International Consortium [MAGIC] criteria; Harris et al., Biol Blood Marrow Transplant, 2016). AEs that have no plausible causal relationship with CTX130 are not considered DLTs.

DLTs are defined as:
(a) Grade 4 CRS
(b) Grade ≥2 GvHD that is steroid-refractory (e.g., progressive disease after 3 days of steroid treatment [e.g., 1 mg/kg/day], or having no response after 7 days of treatment). GvHD that is not steroid-refractory and resolves to grade 1 within 14 days is not defined as a DLT (GvHD grading is provided in Table 38).
(c) Grade 3 or 4 neurotoxicity (based on ICANS criteria).
(d) Death during the DLT period (except due to disease progression).
(e) Any grade 4 hematologic toxicity that does not recover to grade≤2 within 28 days.
(f) Any grade≥3 CTX130-treatment emergent vital organ toxicity (e.g., pulmonary, cardiac) of any duration that is not related to the underlying malignancy or its progression is considered a DLT with the following exceptions in Table 18:

TABLE 18
Exception Criteria

| Exceptions | Criteria |
|---|---|
| #1 | Any grade 3 CRS according to the CRS Grading System (Table 33) |
| | that improves to grade ≤2 with appropriate medical intervention |
| | within 72 hours. |
| #2 | Grade 3 or 4 fever resolving within 72 hours with appropriate |
| | medical intervention. |
| #3 | Grade 3 fatigue lasting <7 days. |
| #4 | Any grade 3 or 4 abnormal liver function tests that improve to |
| | grade ≤2 within 7 days. |
| #5 | Grade 3 or 4 renal insufficiency that improves to grade ≤2 within |
| | 7 days. |
| #6 | Death due to disease progression. |
| #7 | Grade 3 or 4 thrombocytopenia or neutropenia is assessed |
| | retrospectively. After at least 6 subjects are infused, if ≥50% of |
| | subjects have prolonged cytopenias (i.e., lasting more than 28 days |
| | postinfusion), dose escalation is suspended. Grade ≥3 cytopenias |
| | that were present at the start of LD chemotherapy may not be |
| | considered a DLT and identification of another etiology. |

If a subject has a potential DLT for which the protocol definition allows time for improvement or resolution, the DLT evaluation period is extended accordingly before a DLT is declared.

### 6. Study Procedures

Both the dose escalation and expansion parts of the study consist of 3 distinct stages:
(1) screening and eligibility confirmation,
(2) treatment, including daratumumab administration (Parts A2, A4, and A6), LD chemotherapy and CTX130 infusion, and
(3) follow-up.

During the screening period, subjects are assessed according to the eligibility criteria outlined herein. After enrollment, subjects in Part A1 receive LD chemotherapy, followed by a single infusion of CTX130. Subjects in Part A2 will receive daratumumab followed by LD chemotherapy and then a single infusion of CTX130. Subjects in Part A3 will receive LD chemotherapy, an initial CTX130 infusion on Day 1 and a second CTX130 infusion without LD regimen on Day 5. Subjects in Part A4 will receive daratumumab followed by LD chemotherapy, then an initial CTX130 infusion on Day 1 and a second CTX130 infusion without LD regimenon Day 5. Subjects in Part A5 will receive LD chemotherapy followed by an initial infusion of CTX130 on Day 1, and for subjects who achieve CR, PR, SD, or PD with clinical benefit, a second CTX130 infusion on Day 35 with prior LD chemotherapy. Subjects in Part A6 will receive daratumumab followed by LD chemotherapy, then an initial infusion of CTX130 on Day 1, and for subjects who achieve CR, PR, SD, or PD with clinical benefit, a second CTX130 infusion on Day 35 with prior daratumumab and LD chemotherapy.

In Parts A2 and A4, a second dose of daratumumab (16 mg/kg IV or 1800 mg SC) is administered on Day 21 and a third dose on Day 42. After CTX130 infusion, subjects are assessed for disease response, disease progression, and survival. Throughout all study periods, subjects are regularly monitored for safety.

A complete schedule of assessments is provided in Tables 20, 21, 40, and 41. Descriptions of all required study procedures are provided in this section. In addition to protocol-mandated assessments, subjects are followed per institutional guidelines, and unscheduled assessments are performed when clinically indicated. Missed evaluations are rescheduled and performed as close to the originally scheduled date as possible. An exception is made when rescheduling becomes medically unnecessary or unsafe because it is too close in time to the next scheduled evaluation. In that case, the missed evaluation is abandoned.

For the purposes of this protocol, there is no Day 0. All visit dates and windows are calculated using Day 1 as the date of CTX130 infusion.

### Immune Effector Cell-Associated Encephalopathy (ICE) Assessment

Neurocognitive assessment is performed using ICE assessment. The ICE assessment tool is a slightly modified version of the CARTOX-10 screening tool, which now includes a test for receptive aphasia (Neelapu et al., J Clin Oncol, 2018). ICE assessment examines various areas of cognitive function: orientation, naming, following commands, writing, and attention (Table 19).

TABLE 19
ICE Assessment

| Domain | Assessment | Maximum Score |
|---|---|---|
| Orientation | Orientation to year, month, city, hospital | 4 points |
| Naming | Name 3 objects (e.g., point to clock, pen, | 3 points |
| | button) | |
| Following | Ability to follow commands (e.g., “Show me | 1 point |
| command | 2 fingers” or “Close your eyes and stick out | |
| | your tongue”) | |
| Writing | Ability to write a standard sentence (includes | 1 point |
| | a noun and verb) | |
| Attention | Ability to count backward from 100 by 10 | 1 point |

ICE score is reported as the total number of points (0-10) across all assessments.

ICE assessment is performed at screening, before administration of CTX130 on Day 1, and then as per applicable schedule of assessments. If a subject experiences CNS symptoms, ICE assessment is continued to be performed approximately every 2 days until resolution of symptoms to grade 1 or baseline. To minimize variability, whenever possible the assessment is performed by the same research staff member who is familiar with or trained in administration of the ICE assessment tool.

### Patient-Reported Outcomes

Four PRO surveys, the European Organisation for Research and Treatment of Cancer (EORTC) QLQ-C30 and EuroQol EQ-5D-5L questionnaires for all indications, and the Functional Assessment of Cancer Therapy-General (FACT-G) and the Skindex-29 questionnaires for SS, MF, and any subjects with skin lesions, is administered according to the schedules in Tables 20, 21, 40, and 41. Note that subjects who do not have SS or MF but have skin involvement should still fill out FACT-G and Skindex-29 questionnaires. Questionnaires should be completed (self-administered in the language the subject is most familiar) before clinical assessments are performed.

The EORTC QLQ-C30 is a questionnaire designed to measure quality of life in cancer. It is composed of 5 multi-item functioning scales (physical, role, social, emotional, and cognitive function), 3 symptom scales (fatigue, nausea, pain) and additional single symptom items (financial impact, appetite loss, diarrhea, constipation, sleep disturbance, and quality of life). The EORTC QLQ-C30 is validated and has been widely used among cancer patients (Wisloff et al., Br J Haematol, 1996; Wisloff et al., Br J Haematol, 1997). It is scored on a 4-point scale (1=not at all, 2=a little, 3=quite a bit, 4=very much). The EORTC QLQ-C30 instrument also contains 2 global scales that use 7-point scale scoring with anchors (1=very poor and 7=excellent).

The EQ-5D-5L is a generic measure of health status and contains a questionnaire that assesses 5 domains, including mobility, self-care, usual activities, pain/discomfort, and anxiety/depression, plus a visual analog scale. EQ-5D-5L has been used in conjunction with QLQ-C30 (Moreau et al., Leukemia, 2019).

The FACT-G is a validated 27-item instrument that measures the impacts of cancer therapy in 4 domains: physical, social/family, emotional, and functional well-being. The FACT-G total score is based on all 27 items and ranges from 0 to 108, with higher scores indicating better quality of life (Cella et al., J Clin Oncol, 1993).

The Skindex-29 is designed to measure the effects of skin disease on quality of life in 3 domains: symptoms (7 items), emotions (10 items), and functioning (12 items). All responses are transformed to a linear scale of 100, varying from 0 (no effect) to 100 (effect experienced all the time). Scores are reported as 3 scale scores, corresponding to the 3 domains; a scale score is the average of a patient's responses to items in a given domain (Chren, Dermatol Clin, 2012).

TABLE 20
Schedule of Assessments: Parts A1 and A2-Screening to Day 28; Parts A1 through A6-Day 35 to Month 24

| | | | | | Follow-Up | | | | | | | | | |
|---|---|---|---|---|---|---|---|---|---|---|---|---|---|---|
| | | | | | | | | | | | | | | Day 35 to |
| | | | | | | | | | | | | | | Month 24 |
| | | | | | | | | | | | | | | assess- |
| | | | | | | | | | | | | | | ments |
| | | | | | | | | | | | | | | for |
| | | | | | | | | | | | | | | Parts A1 |
| | | | | | | | | | | | | | | through |
| | | | | | | | | | | | | | | A6* |
| | | | | | | | | | | | | | | *Parts |
| | | | | | | | | | | | | | | A5/A6 |
| | | | | | | | | | | | | | | subjects |
| | | | | | | | | | | | | | | who |
| | Screen- | | | | | | | | | | | | | receive |
| | ing¹ | Treatment | | | | | | | | | | | | second |
| | Screening to Day 28 assessments for Parts A1 and A2 only | | | | | | | | | | | | | (D35) |
| | | D − 10 | | | | | | | | | | | | CTX130 |
| | | to | | | | | | | | | | | | infusion |
| | | 1 day | | | | | | | | | | | | begin |
| | | prior | | | | | | | | | | | | Table 20 |
| | | to LD | | | | | | | | | | | | assess- |
| | | chemo | | | | | | | | | | PartA2 | | ments at |
| | | Part | D − 7 | | | | | | | | | only | | M4 |
| Assessment | Screen- | A2 | to D − | | | | | D7 + | D10 ± | D14 ± | D21 ± | D25 ± | D28 ± | D35 ± |
| Day | ing¹ | only² | 3³ | D14 | D2 | D3 | D5 | 2 d | 1 d | 2 d | 2 d | 2 d | 2 d | 2 d |
| Eligibility | X | | X | X | | | | | | | | | | |
| confirmation⁵ | | | | | | | | | | | | | | |
| Informed | X | | | | | | | | | | | | | |
| consent | | | | | | | | | | | | | | |
| CD70 | X | | | | | | | | | | | | | |
| expression⁶ | | | | | | | | | | | | | | |
| Medical | X | | | | | | | | | | | | | |
| history⁷ | | | | | | | | | | | | | | |
| Physical | X | X | X | X | X | X | X | X | X | X | X | X | X | X |
| exam⁸ | | | | | | | | | | | | | | |
| Vital signs⁹ | X | X | 3X | X | X | X | X | X | X | X | X | X | X | X |
| Height, | X | X | X | X | | | | X | | X | | | X | X |
| weight¹⁰ | | | | | | | | | | | | | | |
| Pregnancy | X | X¹¹ | X¹¹ | | | | | | | | | | X | |
| test11 | | | | | | | | | | | | | | |
| ECOG status | X | X | 3X¹² | X | | | | | | | | | X | |
| Echo- | X | | | | | | | | | | | | | |
| cardiogram | | | | | | | | | | | | | | |
| 12-lead | X | X | X | X | | | | | | | | | X | |
| ECG¹³ | | | | | | | | | | | | | | |
| ICE | X | | | X | X | X | X | X | | | | | X | |
| assessment¹⁴ | | | | | | | | | | | | | | |
| PRO¹⁵ | X | | | X | | | | | | X | | | X | |
| Con meds¹⁶ | Continuous | | | | | | | | | | | | | |
| AEs¹⁷ | Continuous | | | | | | | | | | | | | |
| Hospital | | | | Continuous | | | | | | | | | | |
| utilization | | | | | | | | | | | | | | |

Treatment

| | | | | |
|---|---|---|---|---|
| Daratu- | X | | | X |
| mumab | | | | |
| Part A2 | | | | |
| only¹⁸ | | | | |
| LD chemo- | | 3X²¹ | | |
| therapy²⁰ | | | | |
| CTX130 | | | X²¹ | |
| infusion²² | | | | |

B and T Cell Lymphoma Disease/Response Assessments Assessed by the local investigators and/or a central review)

| | | | | |
|---|---|---|---|---|
| Whole body | X | | | X |
| PET/CT scan | | | | |
| or CT scan²³ | | | | |
| Global/ | | | | X |
| Overall | | | | |
| Response 24 | | | | |
| Brain MRI²³ | X | | | |
| Cutaneous | | X²⁶ | X | X |
| assessment | | | | |
| for | | | | |
| all T-cell | | | | |
| lymphomas | | | | |
| (mSWAT) ²⁵ | | | | |
| TNMB | X | | | |
| Assessment | | | | |
| (for all T cell | | | | |
| lymphomas) | | | | |
| Tumor | X | | X²⁸ | X |
| biopsy²⁷ | | | | |
| Peripheral | X | | X | X |
| blood tumor | | | | |
| burden/ | | | | |
| immuno- | | | | |
| pheno- | | | | |
| typing ²⁹ | | | | |
| BM aspirate/ | X³⁰ | | | |
| biopsy³⁰ | | | | |

Laboratory Assessments (Local)

| | | | | | | | | | | | | | | |
|---|---|---|---|---|---|---|---|---|---|---|---|---|---|---|
| CBC w/ | X | X | 3X | X | X | X | X | X | X | X | X | X | X³² | X |
| differential³¹ | | | | | | | | | | | | | | |
| Serum | X | X | 3X | X³³ | X³³ | X³³ | X³³ | X³³ | X³³ | X³³ | X³³ | X³³ | X³³ | X |
| chemistry³³ | | | | | | | | | | | | | | |
| Coagulation | X | X | X | X | X | X | X | X | X | X | | | X | |
| parameters³⁴ | | | | | | | | | | | | | | |
| Viral | X | | | | | | | | | | | | | |
| serology³⁵ | | | | | | | | | | | | | | |
| EBV | X | | | X | | | | X | | X | X | | X | |
| monitoring³⁶ | | | | | | | | | | | | | | |
| SARS- | X | | | | | | | | | | | | | |
| CoV-2 | | | | | | | | | | | | | | |
| 37 | | | | | | | | | | | | | | |
| Immuno- | X | | | X | | | | X | | X | X | | X | |
| globulins³⁸ | | | | | | | | | | | | | | |
| Lymphocyte | X | X³⁹ | X³⁹ | X | X | X | X | X | X | X | X | | X | |
| subsets | | | | | | | | | | | | | | |
| (TBNK)³⁹ | | | | | | | | | | | | | | |
| Ferritin, CRP | X | | | X | X | X | X | X | X | X | X | X | X | |
| sCD25⁴⁰ | X | X | | | | | | | | X | X | X | X | |
| Troponin, | X | | | | | | | | | | | | | |
| NT-proBNP, | | | | | | | | | | | | | | |
| BNP⁴¹ | | | | | | | | | | | | | | |

Biomarkers (Blood, Central)

| | | | | | | | | | |
|---|---|---|---|---|---|---|---|---|---|
| CTX130 | X | | X⁴² | X | X | X | X | X | X |
| levels⁴² | | | pre/ | | | | | | |
| | | | post | | | | | | |
| Cytokines43 | | X | X | X | X | X | X | X | X |
| BSAP, | X | | X | | X | | X | X | X |
| PINP44 | | | | | | | | | |
| Anti-CTX130 | X | | | | | | | | X |
| Ab | | | | | | | | | |
| Exploratory | X | X | X | | X | | X | X | X |
| biomarkers | | | | | | | | | |
| 45 | | | | | | | | | |

| | Follow-Up | | | | | | | | | | | | |
|---|---|---|---|---|---|---|---|---|---|---|---|---|---|
| | Day 35 to Month 24 assessments for Parts A1 through A6* | | | | | | | | | | | | |
| | *Parts A5/A6 subjects who receive second (D35) CTX130 infusion begin Table | | | | | | | | | | | | |
| | 20 assessments at M4 | | | | | | | | | | | | |
| | | | M2 | M3 | M4 | M5 | M6 | M9 | M12 | M15 | M18 | M21 | M24 |
| Assessment | D42 ± | D49 ± | D567 ± | D847 ± | D112 ± | D140 ± | D168 ± | D252 ± | D336 ± | D420 ± | D504 ± | D588 ± | D672 ± |
| Day | 2 d | 2 d | 7 d | 7 d | 7 d | 7 d | 7 d | 14 d | 14 d | 14 d | 14 d | 14 d | 14 d |
| Eligibility | | | | | | | | | | | | | |
| confirmation⁵ | | | | | | | | | | | | | |
| Informed | | | | | | | | | | | | | |
| consent | | | | | | | | | | | | | |
| CD70 | | | | | | | | | | | | | |
| expression⁶ | | | | | | | | | | | | | |
| Medical | | | | | | | | | | | | | |
| history⁷ | | | | | | | | | | | | | |
| Physical | X | X | X | X | X | X | X | X | X | X | X | X | X |
| exam⁸ | | | | | | | | | | | | | |
| Vital signs⁹ | X | X | X | X | X | X | X | X | X | X | X | X | X |
| Height, | X | X | | X | | | | | | | | | |
| weight¹⁰ | | | | | | | | | | | | | |
| Pregnancy | | | X | X | | | | | | | | | |
| test11 | | | | | | | | | | | | | |
| ECOG status | | | | X | | | | | | | | | |
| Echo- | | | | | | | | | | | | | |
| cardiogram | | | | | | | | | | | | | |
| 12-lead | | | | | | | | | | | | | |
| ECG¹³ | | | | | | | | | | | | | |
| ICE | | | | | | | | | | | | | |
| assessment¹⁴ | | | | | | | | | | | | | |
| PRO¹⁵ | | | X | X | | | X | X | X | | X | | X |
| Con meds¹⁶ | Continuous | | | | | | | | | | | | |
| AEs¹⁷ | Continuous | | | | | | | | | | | | |
| Hospital | Continuous | | | | | | | | | | | | |
| utilization | | | | | | | | | | | | | |

Treatment

| | |
|---|---|
| Daratu- | X¹⁹ |
| mumab | |
| Part A2 | |
| only¹⁸ | |
| LD chemo- | |
| therapy²⁰ | |
| CTX130 | |
| infusion²² | |

B and T Cell Lymphoma Disease/Response Assessments Assessed by the local investigators and/or a central review)

| | | | | | | |
|---|---|---|---|---|---|---|
| Whole body | X | X | X | X | X | X |
| PET/CT scan | | | | | | |
| or CT scan²³ | | | | | | |
| Global/ | X | X | X | X | X | X |
| Overall | | | | | | |
| Response 24 | | | | | | |
| Brain MRI²³ | | | | | | |
| Cutaneous | X | X | X | X | X | X |
| assessment | | | | | | |
| for | | | | | | |
| all T-cell | | | | | | |
| lymphomas | | | | | | |
| (mSWAT) ²⁵ | | | | | | |
| TNMB | | | | | | |
| Assessment | | | | | | |
| (for all T cell | | | | | | |
| lymphomas) | | | | | | |
| Tumor | | | | | | |
| biopsy²⁷ | | | | | | |
| Peripheral | X | X | X | X | X | X |
| blood tumor | | | | | | |
| burden/ | | | | | | |
| immuno- | | | | | | |
| pheno- | | | | | | |
| typing ²⁹ | | | | | | |
| BM aspirate/ | | | | | | |
| biopsy³⁰ | | | | | | |

Laboratory Assessments (Local)

| | | | | | | | | | | | | | |
|---|---|---|---|---|---|---|---|---|---|---|---|---|---|
| CBC w/ | X | X | X | X | X | X | X | X | X | X | X | X | X |
| differential³¹ | | | | | | | | | | | | | |
| Serum | X | X | X | X | X | X | X | X | X | X | X | X | X |
| chemistry³³ | | | | | | | | | | | | | |
| Coagulation | | | | | | | | | | | | | |
| parameters³⁴ | | | | | | | | | | | | | |
| Viral | | | | | | | | | | | | | |
| serology³⁵ | | | | | | | | | | | | | |
| EBV | | | | | | | | | | | | | |
| monitoring³⁶ | | | | | | | | | | | | | |
| SARS- | | | | | | | | | | | | | |
| CoV-2 | | | | | | | | | | | | | |
| 37 | | | | | | | | | | | | | |
| Immuno- | | | X | X | X | X | X | X | X | X | X | X | X |
| globulins³⁸ | | | | | | | | | | | | | |
| Lymphocyte | | | X | X | | | | | | | | | |
| subsets | | | | | | | | | | | | | |
| (TBNK)³⁹ | | | | | | | | | | | | | |
| Ferritin, CRP | | | | | | | | | | | | | |
| sCD25⁴⁰ | | | | | | | | | | | | | |
| Troponin, | | | | | | | | | | | | | |
| NT-proBNP, | | | | | | | | | | | | | |
| BNP⁴¹ | | | | | | | | | | | | | |

Biomarkers (Blood, Central)

| | | | | | | |
|---|---|---|---|---|---|---|
| CTX130 | X | X | X | X | X | X |
| levels⁴² | | | | | | |
| Cytokines43 | X | X | X | | | |
| BSAP, | | X | | | | |
| PINP44 | | | | | | |
| Anti-CTX130 | | X | | X | X | |
| Ab | | | | | | |
| Exploratory | X | X | | X | X | X |
| biomarkers | | | | | | |
| 45 | | | | | | |

Ab: antibody;
AE: adverse event;
AESI: adverse event of special interest;
ALT: alanine aminotransferase;
AST: aspartate aminotransferase;
ATLL: adult T cell leukemia/lymphoma;
BM: bone marrow;
BNP: B-type natriuretic peptide;
BSAP: bone-specific alkaline phosphatase;
BUN: blood urea nitrogen;
CBC: complete blood count;
chemo: chemotherapy;
CMV: cytomegalovirus;
CNS: central nervous system;
con meds: concomitant medications;
CR: complete response;
CRISPR: clustered regularly interspaced short palindromic repeats;
CRP: C-reactive protein;
CRS: cytokine release syndrome;
CT: computed tomography;
D or d: day;
DNA: deoxyribonucleic acid;
EBER: EBV-encoded small RNA;
EBV: Epstein-Barr virus;
ECG: electrocardiogram;
ECOG: Eastern Cooperative Oncology Group;
eGFR: estimated glomerular filtration rate;
EORTC QLQ-C30: European Organisation for Research and Treatment of Cancer QLQ-C30 and questionnaire;
FACT-G: Functional Assessment of Cancer Therapy-General;
FDG: fluorodeoxyglucose;
GvHD: graft versus host disease;
HBcAb: hepatitis B core antibody;
HBsAb: hepatitis B surface antibody;
HBsAg: hepatitis B surface antigen;
HBV: hepatitis B virus;
HCV: hepatitis C virus;
HHV-6: human herpesvirus 6;
HIV-1/-2: human immunodeficiency virus type 1 or 2;
HLH: hemophagocytic lymphohistiocytosis;
HTLV-1: human T cell leukemia virus type 1;
ICE: immune effector cell-associated encephalopathy;
Ig: immunoglobulin;
INR: international normalized ratio;
ISH: in situ hybridization;
LD: lymphodepleting;
LDH: lactate dehydrogenase;
LP: lumbar puncture;
M: month;
MF: mycosis fungoides;
MRD: minimal residual disease;
MRI: magnetic resonance imaging;
mSWAT: modified Severity Weighted Assessment Tool;
NK: natural killer;
NT-proBNP: N-terminal pro hormone B-type natriuretic peptide;
PET: positron emission tomography;
PINP: procollagen type 1N propeptide;
PRO: patient-reported outcomes;
PT: prothrombin time;
PTT: partial prothrombin time;
RNA: ribonucleic acid;
sCD25: soluble CD25;
SGOT: serum glutamic oxaloacetic transaminase;
SGPT: serum glutamic pyruvic transaminase;
SS: Sézary syndrome;
TBNK: T, B, and NK cells.
Note:
Assessments scheduled on CTX130 infusion days are to be performed pre-CTX130 infusion unless otherwise specified; for samples tested centrally, refer to Laboratory Manual.
Note:
For Parts A1 and A2, this study will allow for additional courses of treatment with CTX130 per the criteria described herein. Prior to an additional course of treatment, all screening assessments must be repeated, except for radiological (PET-CT or CT) disease assessments if performed within 28 days prior to next CTX130 infusion, bone marrow biopsy/aspirate unless clinically indicated, echocardiogram (unless new cardiac signs or symptoms), and brain MRI. In addition, not all central lab screening samples may be required. Subjects who undergo additional course(s) of treatment will receive daratumumab (Part A2 only), 3 days of LD chemotherapy, and should be followed per the schedule of assessments consistent with the first course of treatment (including the 7 days of hospitalization post CTX130 infusion), except that tumor biopsy will not be performed on Day 7 and Day 28 after CTX130 infusion. After an additional course of treatment and repeat of screening through D28 assessments, subjects should continue with the next scheduled visit assessments; if the next scheduled visit occurs within 28 days of previous disease response assessments, then disease response assessments for next scheduled visit do not need to be performed-note, however, that through Month 12 post-initial CTX130 infusion, there should not be more than a 3-month gap between disease response assessments, and after Month 12 (post-initial CTX130 infusion), there should be no more than a 6-month gap between disease response assessments.
Note:
CTX130 infusion during an additional course of treatment may be administered without prior LD chemotherapy if subject is experiencing significant cytopenia.
Note:
Certain assessments for visits after 7 days post-CTX130 infusion may be performed as in-home or alternate-site visits. Assessments may include hospital utilization, changes in health and/or changes in medications, vital signs, weight, PRO questionnaire distribution, and blood sample collections for local and central laboratory assessments.
¹Screening assessments to be completed within 14 days of informed consent. The screening period may be extended beyond 14 days to allow for COVID-19 testing only. Screening assessment of disease category/subtype is reviewed via central pathology examination of collected tissue representative of patient’s disease. Subjects are allowed a one-time rescreening, which may take place within 3 months of initial consent. Subjects who rescreen may use previous BM biopsy/aspirate samples for rescreening if no anticancer therapies have been administered in the interim. In addition, not all central lab samples may be required (see Laboratory Manual for details).
²All assessments must occur on the same day and prior to daratumumab administration.
³“X” indicates occurrence only on first day of LD chemo. “3X” indicates occurrence on each of the 3 days of LD chemo. Assessments scheduled on LD chemo days are to be performed pre-LD chemo unless otherwise specified.
⁴All baseline assessments on Day 1 are to be performed prior to CTX130 infusion unless otherwise specified; refer to the Laboratory Manual for details.
⁵Eligibility should be confirmed each time screening is completed. Eligibility should also be confirmed on day of daratumumab administration (Part A2 only), on first day of LD chemotherapy, and on day of CTX130 infusion. Eligibility should be reconfirmed after all assessments for that day are completed and before dosing.
⁶Subjects must have CD70-expressing tumors to receive first course or any additional course of CTX130 treatment. Tissue may be submitted and tested locally or centrally at any time prior to or during the 14-day window for screening, provided the subject has signed an appropriate consent. Tissue for CD70 testing should be representative of the subject’s disease but does not need to be collected within 3 months of CTX130 infusion or post progression after the last systemic therapy. Prior to any additional course of treatment with CTX130, subjects must have CD70 expression of tumors confirmed from a new tumor sample.
⁷Includes complete surgical, neurological, and cardiac history.
⁸Includes assessment for signs and symptoms of GvHD: skin, oral mucosa, sclera, hands, and feet.
⁹Includes sitting blood pressure, heart rate, respiratory rate, pulse oximetry, and temperature.
¹⁰Height at initial screening only.
¹¹For female subjects of childbearing potential. Serum pregnancy test required at screening, within 72 hours of beginning LD chemotherapy (Part A1) or initial daratumumab dose (Part A2), and at Day 28, Day 56, and Month 3 visit. Is assessed at a local laboratory.
¹²Prior to initiation of LD chemotherapy on each of the three days, ECOG performance status should be checked.
¹³2-lead ECG test should be conducted at screening, prior to initial daratumumab administration (Part A2 only), first day of LD chemotherapy, and CTX130 infusion, and on Day 28.
¹⁴On Day 1, prior to CTX130 administration. If CNS symptoms persist, ICE assessment should continue to be performed approximately every 2 days until symptom resolution to grade 1 or baseline.
¹⁵EORTC QLQ-30, EQ-5D-5L questionnaires for all indications; FACT-G, Skindex-29 questionnaire for SS and MF and for any subjects with skin lesions. PRO surveys should be administered before any visit-specific procedures are performed.
¹⁶All concomitant medications are collected up to 3 months after each CTX130 infusion, after which only select concomitant medications are collected.
¹⁷Collect all AEs from informed consent to 3 months after each CTX130 infusion and collect only SAEs and AESIs from 3 months after last CTX130 infusion through Month 24 visit. After Month 24 to Month 60 or after a subject starts a new anticancer therapy, only CTX130-related SAEs, CTX130-related AESIs, and new malignancies are reported.
¹⁸Part A2 only: All assessments on days of daratumumab administration must be completed prior to daratumumab dosing unless otherwise specified. One dose of daratumumab 16 mg/kg IV or 1800 mg SC administered at least 12 hours prior to starting LD chemotherapy and within 10 days prior to CTX130 infusion. Daratumumab administration at 16 mg/kg IV or 1800 mg SC is repeated at Day 21 and Day 42. To facilitate administration, the 16 mg/kg IV dose may be split (to 8 mg/kg IV) over 2 consecutive days per the daratumumab prescribing information. If a subject experiences disease progression or unacceptable adverse events related to daratumumab, repeat dosing with daratumumab will not be permitted.
¹⁹Subjects in Part A2 and in Part A4 will receive daratumumab at Day 42.
²⁰For first CTX130 infusion, start LD chemotherapy within 7 days of study enrollment. After completion of LD chemotherapy, ensure washout period of ≥ 48 hours (but ≤ 7 days) before CTX130 infusion. Physical exam, weight, and coagulation laboratories are performed prior to first dose of LD chemotherapy. Vital signs, CBC with differential, serum chemistry, ECOG status, and AEs/concomitant medications should be assessed and recorded daily (i.e., 3 times) during LD chemotherapy.
²¹This study will allow for up to 2 additional courses of treatment. Subjects should be followed per the schedule of assessments.
²²CTX130 is administered 48 hours to 7 days after completion of LD chemotherapy.
²³Whole body (including neck) PET/CT and MRI brain scan to be performed as part of or prior to screening (within 28 days prior to CTX130 infusion). Brain MRI is only required as part of screening for the first course of treatment. Non FDG-avid lymphomas may be followed post-baseline by CT as clinically indicated. Whole body (including neck if involved at baseline) PET/CT or CT, as clinically indicated, to be performed per the schedule of assessments and upon suspected CR. Postinfusion scans are performed per the schedule of assessments, per the protocol-defined response criteria, and as clinically indicated for all baseline FDG-avid lymphomas. MRI with contrast may be used for the CT portion when CT is clinically contraindicated or as required by local regulation. If PET cannot be performed with diagnostic quality CT, a separate diagnostic CT must be performed. PET/CT is evaluated locally and centrally.
24Global response by ISCL response criteria and Overall response by Lugano criteria.
²⁵ Cutaneous assessment (mSWAT) to be evaluated locally but may also be evaluated centrally if indicated (i.e., skin punch biopsy).
²⁶Skin photographs and modified severity weight assessment tool (mSWAT) to be performed post LD chemotherapy day 3 and prior to CTX130 infusion (Day 1 pre-infusion is best).
²⁷Biopsy (including skin punch biopsy) to be performed at screening if postprogression biopsy tissue is not available/acceptable, Day 7 (+2 days), and Day 28 (±2 days) after the initial infusion of CTX130. For subjects undergoing additional course(s) of treatment, tumor biopsy will not be performed on Day 7 and Day 28 after CTX130 infusion. Tumor biopsy to be evaluated locally and centrally. If a relapse occurs while a subject is on study, every attempt should be made to obtain biopsy of relapse tumor to send to central laboratory.
²⁸Day 7 tumor biopsy for Part A only
²⁹ Perform peripheral blood tumor burden assessments, e.g., Sezary cell counts, ATLL cell counts, per institutional guidelines.
³⁰Bone marrow biopsy and aspirate collection are performed if clinically indicated. For subjects with bone marrow involvement at screening, an additional bone marrow biopsy and aspirate collection are performed to confirm CR. In the event a bone marrow biopsy or aspirate is performed, samples should be sent to central lab.
³¹Hematocrit, hemoglobin, red blood cell count, white blood cell count, neutrophils, lymphocytes, monocytes, basophils, eosinophils, platelet count, absolute neutrophil count.
³²For subjects experiencing grade ≥ 3 neutropenia, thrombocytopenia, or anemia that has not resolved within 28 days of CTX130 infusion, a CBC with differential must be performed weekly until resolution to grade ≤ 2.
³³Serum chemistries to include ALT (SGPT), AST (SGOT), bilirubin (total), albumin, alkaline phosphatase, bicarbonate, BUN, calcium, chloride, creatinine, eGFR, glucose, LDH, phosphorus, potassium, sodium, total protein, uric acid (uric acid should be included in serum chemistry assessments through Day 28). If acute renal tubular damage is suspected, additional tests should be conducted including urine sediment analysis and fractional excretion of sodium in urine, and consultation by a nephrologist should be initiated.
³⁴Include PTT, fibrinogen, INR, and d-dimer.
³⁵Viral serologies for HIV-1, HIV-2, HBV (HBsAg, HBsAb, HBcAb), HCV (HCV antibody and RNA), EBV (see EBV monitoring line item in schedule of assessments table), HHV-6, HHV-7, CMV at screening; however, historical results obtained within 60 days of enrollment may be used to determine eligibility.
³⁶ Assessed using quantitative polymerase chain reaction (qPCR). Monitor as clinically indicated until CD4+ counts of 200/μl are reached. Suspicious or ambiguous lesions in the context of rising EBV DNA levels should be biopsied and examined by local pathology, EBER ISH.
³⁷ SARS-CoV-2 test is performed at screening. Screening test does not need to be repeated if within 3-4 days prior to start of lymphodepletion regimen (LD chemotherapy with or without daratumumab).
³⁸Include IgA, IgG, IgM.
³⁹ Lymphocyte subset assessment at screening, before start of first day of LD chemo (Part Al) or pre initial daratumumab dose (Part A2), before CTX130 infusion, then all listed time points is assessed at local laboratory and will include 6-color TBNK panel, or equivalent for T, B, and NK cells.
⁴⁰ sCD25 also to be assessed during suspected HLH.
⁴¹Troponin, NT-proBNP, and BNP assessed at screening and in the event of a grade ≥ 2 CRS on day 1, 3, and 7 of CRS event or as clinically indicated (Maus et al, 2020).
⁴²For CTX130 levels, 2 samples should be collected on Day 1: one pre-CTX130 infusion and one 20 minutes (±5 minutes) after the end of CTX130 infusion. If CRS occurs, samples for assessment of CTX130 levels are collected every 48 hours (±5 hours) between scheduled visits until CRS resolves. Samples for CTX130 levels should be collected from any lumbar puncture, blood samples, BM aspirate samples and tissue biopsies performed following CTX130 infusion, including unscheduled visits. In subjects experiencing signs or symptoms of neurotoxicity and suspected HLH, additional blood samples should be drawn at intervals outlined in the laboratory manual. Sponsor may discontinue testing and request discontinuation of sample collection if consecutive tests are negative. Continue sample collection for all listed time points until otherwise instructed by sponsor.
43In the event of grade ≥ 2 CRS, cytokine samples should be collected at the onset of symptoms. Additional cytokine samples should be collected every 24 hours (±5 hours) for the duration of any grade CRS. During neurotoxicity and suspected HLH, additional cytokine samples are collected (see Laboratory Manual for specific information).
44Samples are to be collected at the same time of day (±2 hours) on the specified collection days.
45Samples for exploratory biomarkers should be sent from any LP or BM biopsy performed following CTX130 infusion. Subsequent LP and BM aspirate should only be collected when a screening sample is positive, and when follow-up samples are collected. If CRS occurs, samples for assessment of exploratory biomarkers are collected every 48 hours (±5 hours) between scheduled visits until CRS resolves. If neurotoxicity or HLH occur, samples for assessment of exploratory biomarkers are collected as instructed in the laboratory manual.

TABLE 21
Schedule of Assesssments: Months 30-60

| | M 30 | M 36 | M 42 | M 48 | M 54 | M 60 | Progressive | Secondary |
|---|---|---|---|---|---|---|---|---|
| Assessments | (±21 days) | (±21 days) | (±21 days) | (±21 days) | (±21 days) | (±21 days) | Disease¹ | Follow-Up² |
| Physical exam | X | X | X | X | X | X | X | X |
| Vital signs³ | X | X | X | X | X | X | X | X |
| PRO⁴ | X | X | X | X | X | X | X | |
| Concomitant medications⁵ | X | X | X | X | X | X | X | X |
| AEs⁶ | X | X | X | X | X | X | X | X |
| Disease/response | X | X | X | X | X | X | X | |
| assessment⁷ | | | | | | | | |

Laboratory Assessments (Blood, Local)

| | | | | | | | | |
|---|---|---|---|---|---|---|---|---|
| CBC with differential⁸ | X | X | X | X | X | X | X | X |
| Serum chemistry⁸ | X | X | X | X | X | X | X | X |
| Lymphocyte subsets⁸ | | | | | | | X | |

Biomarkers (Blood, Central)

| | | |
|---|---|---|
| CTX130 levels⁹ | X | X |
| Anti-CTX130 Ab | X | |
| Exploratory biomarkers ¹⁰ | X | X |

Ab: antibody;
AE: adverse event;
AITL: angioimmunoblastic T cell lymphoma;
ALCL: anaplastic large cell lymphoma;
ATLL: adult T cell leukemia/lymphoma;
BM: bone marrow;
CBC: complete blood count;
CT: computed tomography;
DLBCL: diffuse large B cell lymphoma;
DNA: deoxyribonucleic acid;
EORTC QLQ-C30: European Organisation for Research and Treatment of Cancer QLQ-C30 questionnaire;
FACT-G: Functional Assessment of Cancer Therapy-General;
ISCL: International Society for Cutaneous Lymphomas;
LP: lumbar puncture;
M: month;
MF: mycosis fungoides;
PD: progressive disease;
PET: positron emission tomography;
PRO: patient-reported outcomes;
PTCL-NOS: peripheral T cell lymphoma, not otherwise specified;
SAE: serious adverse event;
SCT: stem cell transplantation;
SS: Sézary syndrome.
¹Subjects with PD will discontinue the normal schedule of assessments and undergo study assessments listed unless already collected in a scheduled visit where PD was documented. Subjects should transition to secondary follow-up with the initial annual follow-up visit occurring a year after PD is documented (see footnote 2).
²Subjects who are discontinued from the regular schedule of assessments due to disease progression, investigator decision/start of new anticancer therapy, AEs, protocol violation, or pregnancy will attend annual visits to collect safety information for up to 5 years.
³Includes temperature, blood pressure, heart rate, pulse oximetry, and respiratory rate.
⁴EORTC QLQ-30, EQ-5D-5L questionnaires for all indications; FACT-G, Skindex-29 questionnaire for SS and MF and for any subjects with skin lesions. PRO surveys should be administered before any visit-specific procedures are performed.
⁵Only select concomitant medications are collected
⁶If a subject begins new anticancer therapy, only events defined as AESIs that are possibly related or related to CTX130 should be reported.
⁷Disease evaluations are based on assessments in accordance with Lugano response criteria (Cheson et al., 2014) for subjects with PTCL-NOS, ALCL, leukemic and lymphomatous ATLL, AITL, and DLBCL, and ISCL response criteria (Olsen et al., 2011) for subjects with SS or MF, andwill include BM aspirate and biopsy if clinically indicated, whole body PET/CT, and cutaneous assessment.
⁸Assessed at local laboratory.
⁹In addition to time points listed, samples for analysis of CTX130 levels should be sent to the central laboratory from any unscheduled collection of blood, BM aspirate, or tissue biopsy performed following CTX130 infusion.
¹⁰ LP and BM aspirate should be sent to central lab when samples are collected in response to a clinical event or suspected AE (e.g., ICANS).

TABLE 40
Schedule of Unique Assessments for Parts A3 and A4: Second CTX130 Infusion on Day 5 to Day 28 Visit

| | | | | | | | Unique Assessments for Parts A3 and A4 | | | | | | | | | | |
|---|---|---|---|---|---|---|---|---|---|---|---|---|---|---|---|---|---|
| | | Treatment | | | | | | Follow-up After Second Infusion | | | | | | | | | |
| | | D − 10 | | | | | Second | | | | | | | | | | Continue |
| | | to | | | | | CTX130 | | | | | | | | | | Day 35 |
| | | 1 day | | | | | Infusion | | | | | | | | | | Follow- |
| | | prior | | | Same as | | on D5 | | | | | | | | | | up |
| | | to LD | | | Table 20 | | (+2 | | | | | | | | | Part | as in |
| | | chemo | | | Follow-up | | days)² | | | | | | | | | A4 | Table |
| | | Part | D − 7 | | After Initial | | (no LD | | | | | | | | | only | 20 |
| Study Stage | Screen- | A4 | to D − | | Infusion | | regimen) | | | | | D12 + | D14 ± | D18 ± | D21 | D25 ± | D28 ± |
| Day | ing¹ | only³ | 34 | D1⁵ | D2 | D3 | D5 ^{5,6} | D6 | D7 | D9 | D10 | 2d | 2d | 2d | +2d | 2d | 2d |
| Eligibility | X | | X | X | | | X | | | | | | | | | | |
| confirmation⁷ | | | | | | | | | | | | | | | | | |
| Informed | X | | | | | | | | | | | | | | | | |
| consent | | | | | | | | | | | | | | | | | |
| CD70 | X | | | | | | | | | | | | | | | | |
| expression⁸ | | | | | | | | | | | | | | | | | |
| Medical | X | | | | | | | | | | | | | | | | |
| history⁹ | | | | | | | | | | | | | | | | | |
| Physical | X | | X | X | X | X | X | X | X | X | X | X | X | X | X | X | X |
| exam¹⁰ | | | | | | | | | | | | | | | | | |
| Vital signs¹¹ | X | | 3X | X | X | X | X | X | X | X | X | X | X | X | X | X | X |
| Height, | X | | X | X | | | X | | | | | | X | | | | X |
| weight¹² | | | | | | | | | | | | | | | | | |
| Pregnancy test¹³ | X | X¹³ | X¹³ | | | | | | | | | | | | | | X |
| ECOG status | X | | 3X¹⁴ | X | | | X | | | | | | X | | | | X |
| Echocardiogram | X | | | | | | | | | | | | | | | | |
| 12-lead ECG¹⁵ | X | | X | X | | | X | | | | | | | | | | X |
| ICE | X | | | X | X | X | X | X | X | X | X | X | X | X | | | X |
| assessment¹⁶ | | | | | | | | | | | | | | | | | |
| PRO¹⁷ | X | | | X | | | X | | | | | | X | | | | X |

| Concomitant | Continuous | |
|---|---|---|
| meds¹⁸ | | |
| Adverse events¹⁹ | Continuous | |
| Hospital | | Continuous |
| utilization | | |

Treatment

| | | | | | |
|---|---|---|---|---|---|
| Daratumumab | X | | | | X |
| Part A4 only ²⁰ | | | | | |
| LD | | 3X²² | | | |
| chemotherapy²¹ | | | | | |
| CTX130 | | | X²² | X²⁴ | |
| infusion²³ | | | | | |

B and T cell Lymphoma Disease Response/Assessment (Central and Local)

| | | | | |
|---|---|---|---|---|
| Whole body | X | | | X |
| PET/CT scan | | | | |
| or CT scan²⁵ | | | | |
| Brain MRI²⁵ | X | | | |
| Global/Overall | | | | X |
| Response²⁶ | | | | |
| Cutaneous | | X²⁸ | X | X |
| assessment for | | | | |
| all T-cell | | | | |
| lymphomas | | | | |
| (mSWAT) ²⁷ | | | | |
| TNMB | X | | | |
| BM | X | | | |
| aspirate/biopsy²⁹ | | | | |
| Tumor biopsy³⁰ | X | | X³¹ | X |
| Peripheral blood | X | | X | X |
| tumor burden/ | | | | |
| immuno- | | | | |
| phenotyping ³² | | | | |

Laboratory Assessments (Local)

| | | | | | | | | | | | | | | | | | |
|---|---|---|---|---|---|---|---|---|---|---|---|---|---|---|---|---|---|
| CBC w/ | X | | 3X | X | X | X | X | X | X | X | X | X | X | X | X | X | X |
| differential^{33,34} | | | | | | | | | | | | | | | | | |
| Serum | X | | 3X | X³⁵ | X³⁵ | X³⁵ | X³⁵ | X³⁵ | X³⁵ | X³⁵ | X³⁵ | X³⁵ | X³⁵ | X³⁵ | X³⁵ | X³⁵ | X³⁵ |
| chemistry³⁵ | | | | | | | | | | | | | | | | | |
| Coagulation | X | | X | X | X | X | X | X | X | X | X | X | X | X | X | | X |
| parameters ³⁶ | | | | | | | | | | | | | | | | | |
| Viral serology³⁷ | X | | | | | | | | | | | | | | | | |
| EBV | X | | | | | | X | | | | | | X | | X | | X |
| monitoring ³⁸ | | | | | | | | | | | | | | | | | |
| SARS-CoV-2³⁹ | X | | | | | | | | | | | | | | | | |
| Immuno- | X | | | X | | | X | | | | | | X | | X | | X |
| globulins⁴⁰ | | | | | | | | | | | | | | | | | |
| Lymphocyte | X | X⁴¹ | X⁴¹ | X | X | X | X | | X | | X | | X | | X | | X |
| subsets | | | | | | | | | | | | | | | | | |
| (TBNK) ⁴¹ | | | | | | | | | | | | | | | | | |
| Ferritin, CRP | X | | | X | X | X | X | X | X | X | X | X | X | X | X | X | X |
| sCD25⁴² | X | X | | | | | | | | | | | X | | X | X | X |
| Troponin, NT- | X | | | | | | | | | | | | | | | | |
| proBNP, BNP⁴³ | | | | | | | | | | | | | | | | | |

Biomarkers (Blood, Central)

| | | | | | | | | | | | | | |
|---|---|---|---|---|---|---|---|---|---|---|---|---|---|
| CTX130 PK^{44,45} | X | | X⁴⁶ | X | X⁴⁶ | X | X | X | X | X | X | X | X |
| | | | pre/ | | pre/post | | | | | | | | |
| | | | post | | | | | | | | | | |
| Cytokines⁴⁷ | | X | X | X | X | X | X | X | X | X | X | X | X |
| Anti-CTX130 | X | | | | | | | | | | | | X |
| Ab | | | | | | | | | | | | | |
| BSAP, PINP⁴⁸ | X | | X | | | | X | | | X | | X | X |
| Exploratory | X | X | X | | X | | X | | | X | | X | X |
| biomarkers⁴⁹ | | | | | | | | | | | | | |

Ab: antibody;
AE: adverse event;
AESI: adverse event of special interest;
BM: bone marrow;
CBC: complete blood count;
CNS: central nervous system;
CRP: C-reactive protein;
CRS: cytokine release syndrome;
CT: computed tomography;
D or d: day;
EE ER: EBV-encoded small RNA;
EBV: Epstein-Barr virus;
ECG: electrocardiogram;
ECOG: Eastern Cooperative Oncology Group;
HBV: hepatitis B virus;
HCV: hepatitis C virus;
HIV-1/-2: human immunodeficiency virus type 1 or 2;
HLH: hemophagocytic lymphohistiocytosis;
ICE: immune effector cell-associated encephalopathy;
IPI: International Prognostic Index;
ISH: in situ hybridization;
IV: intravenously;
LD: lymphodepleting;
LP: lumbar puncture;
M: month;
MRI: magnetic resonance imaging;
NHL: non-Hodgkin lymphoma;
PBMC: peripheral blood mononuclear cell;
PCR: polymerase chain reaction;
PD: progressive disease;
PET: positron emission tomography;
PK: pharmacokinetic(s);
PRO: patient-reported outcome;
SAE: serious adverse event;
SC: subcutaneous;
SD: stable disease;
TBNK: T-, B-, natural killer (cells).
Note:
Assessments scheduled on CTX130 infusion days are to be performed pre-CTX130 infusion unless otherwise specified; for samples tested centrally, refer to Laboratory Manual.
Note:
For Parts A3 and A4, this study will allow for additional courses of treatment with CTX130. Prior to any additional course(s) of treatment, all screening assessments must be repeated, except for radiological (PET-CT or CT) disease assessments if performed within 28 days prior to next CTX130 infusion, bone marrow biopsy/aspirate unless clinically indicated, echocardiogram (unless new cardiac signs or symptoms), and brain MRI. In addition, not all central lab screening samples may be required (see Laboratory Manual for details). Subjects who undergo additional course(s) of treatment will receive daratumumab (Part A4 only), 3 days of LD chemotherapy, and should be followed per the schedule of assessments consistent with the first course of treatment (including the 7 days of hospitalization post CTX130 infusion), except that tumor biopsy will not be performed on Day 12 and Day 28 after CTX130 infusion. After an additional course of treatment and repeat of screening through D28 assessments, subjects should continue with the next scheduled visit assessments; if the next scheduled visit occurs within 28 days of previous disease response assessments, then disease response assessments for next scheduled visit do not need to be performed-note, however, that through Month 12 post-initial CTX130 infusion, there should not be more than a 3-month gap between disease response assessments, and after Month 12 (post-initial CTX130 infusion), there should be no more than a 6-month gap between disease response assessments.
Note:
CTX130 infusion during an additional course of treatment may be administered without prior LD chemotherapy if subject is experiencing significant cytopenia.
Note:
Certain assessments for visits after 7 days post-CTX130 infusion may be performed as in-home or alternate-site visits. Assessments include hospital utilization, changes in health and/or changes in medications, body system assessment, vital signs, weight, PRO questionnaire distribution, and blood sample collections for local and central laboratory assessments.
¹Screening assessments to be completed within 14 days of informed consent. The screening period may be extended beyond 14 days to allow for COVID-19 testing only. Screening assessment of disease category/subtype is reviewed via central pathology examination of collected tissue representative of patient’s disease. Subjects are allowed a one-time rescreening, which may take place within 3 months of initial consent. Subjects who rescreen may use previous BM biopsy/aspirate samples for rescreening if no anticancer therapies have been administered in the interim. In addition, not all central lab samples may be required
²If a subject receives the initial (Day 1) CTX130 infusion but does not receive the second (Day 5) CTX130 infusion, they can still receive an additional course(s) of treatment as long as they meet the requirements for an additional course of treatment
³All assessments must occur on the same day and prior to daratumumab administration.
⁴“X” indicates occurrence only on first day of LD chemo. “3X” indicates occurrence on each of the 3 days of LD chemo. Assessments scheduled on LD chemo days are to be performed pre-LD chemo unless otherwise specified.
⁵All baseline assessments on Day 1 and all Day 5 assessments are to be performed prior to CTX130 infusion unless otherwise specified ⁶Assessments on Days 6 and 7 are to be performed 1 and 2 days, respectively, after the second CTX130 infusion. If the second CTX130 infusion
occurs within the + 2 day time window of Day 5, the subsequent assessments should be adjusted accordingly.
⁷Eligibility should be confirmed each time screening is completed. Eligibility should also be confirmed prior to daratumumab administration (Part A4 only), on first day of LD chemotherapy, and on day of CTX130 infusion (on both Day 1 and Day 5). Eligibility should be reconfirmed after all assessments for that day are completed and before dosing.
⁸Subjects must have CD70-expressing tumors to receive first course or any additional course of CTX130 treatment. Tissue may be submitted and tested locally or centrally at any time prior to or during the 14-day window for screening, provided the subject has signed an appropriate consent. Tissue for CD70 testing should be representative of the subject’s disease but does not need to be collected within 3 months of CTX130 infusion or post progression after the last systemic therapy. Prior to any additional course of treatment with CTX130, subjects must have CD70 expression of tumors confirmed from a new tumor sample.
⁹Includes complete surgical, neurological, and cardiac history.
¹⁰Includes assessment for signs and symptoms of GvHD: skin, oral mucosa, sclera, hands, and feet.
¹¹Includes sitting blood pressure, heart rate, respiratory rate, pulse oximetry, and temperature.
¹²Height at initial screening only.
¹³For female subjects of childbearing potential. Serum pregnancy test at screening, within 72 hours of beginning LD chemotherapy (Part A3) or initial daratumumab dose (Part A4), at Day 28, and then as per schedule of assessments. Is assessed at a local laboratory.
¹⁴Prior to initiation of LD chemotherapy on each of the three days, ECOG performance status should be checked.
¹⁵12-lead ECG test should be conducted at screening, prior to daratumumab administration (Part A4 only), first day of LD chemotherapy, and CTX130 infusion, and on Day 28.
¹⁶Prior to CTX130 administration on Day 1 and Day 5. If CNS symptoms persist, ICE assessment should continue to be performed approximately every 2 days until symptom resolution to grade 1 or baseline.
¹⁷E0RTC QLQ-30, EQ-5D-5L questionnaires for all indications; FACT-G, Skindex-29 questionnaire for SS and MF and for any subjects with skin lesions. PRO surveys should be administered before any visit-specific procedures are performed.
¹⁸All concomitant medications are collected up to 3 months after each CTX130 infusion, after which only select concomitant medications are collected
¹⁹Collect all AEs from informed consent to 3 months after each CTX130 infusion and collect only SAEs and AESIs from 3 months after last CTX130 infusion through Month 24 visit. After Month 24 to Month 60 or after a subject starts a new anticancer therapy, only CTX130-related SAEs, CTX130-related AESIs, and new malignancies are reported.
²⁰Part A4 only: All assessments on days of daratumumab administration must be completed prior to daratumumab dosing unless otherwise specified. One dose of daratumumab 16 mg/kg IV or 1800 mg SC administered at least 12 hours prior to starting LD chemotherapy and within 10 days prior to CTX130 infusion. Daratumumab administration at 16 mg/kg IV or 1800 mg SC is repeated at Day 21 and Day 42 (see Table 11 for Day 42 daratumumab administration). To facilitate administration, the 16 mg/kg IV dose may be split (to 8 mg/kg IV) over 2 consecutive days per the daratumumab prescribing information. If a subject experiences disease progression or unacceptable adverse events related to daratumumab, repeat dosing with daratumumab will not be permitted.
²¹For first CTX130 infusion, start LD chemotherapy within 7 days of study enrollment. After completion of LD chemotherapy, ensure washout period of ≥ 48 hours (but ≤ 7 days) before CTX130 infusion. Physical exam, weight, and coagulation laboratories performed prior to first dose of LD chemotherapy. Vital signs, CBC with differential, serum chemistry, ECOG status, and AEs/concomitant medications should be assessed and recorded daily (i.e., 3 times) during LD chemotherapy.
²²This study will allow for up to 2 additional courses of treatment. For each course of treatment, subjects should be followed per the schedule of assessments in Table 13 from Screening through Day 28, and then per the schedule of assessments in Table 11 from Day 35 through Month 24
²³For first CTX130 infusion, CTX130 administered 48 hours to 7 days after completion of LD chemotherapy.
²⁴See eligibility for second (Day 5 [+2]) CTX130 infusion for subjects in Parts A3 and A4. CTX130 is administered without LD regimen at the same dose level as the first infusion. The second infusion is administered 4 days after the initial CTX130 infusion, with a time window of +2 days. In the event of a dose delay outside the + 2-day window, subjects should restart at Day 5 and follow visit schedule; the investigator may discuss the timing of the second dose with the medical monitor, with the second dose to occur no later than Day 15.
²⁵Whole body (including neck) PET/CT and MRI brain scan to be performed as part of or prior to screening (i.e., within 28 days prior to CTX130 infusion). Brain MRI is only required as part of screening for the first course of treatment. Non FDG-avid lymphomas may be followed post-baseline by CT as clinically indicated. Whole body (including neck if involved at baseline) PET/CT or CT, as clinically indicated, to be performed per the schedule of assessments and upon suspected CR. Postinfusion scans are performed per the schedule of assessments, per the protocol-defined response criteria, and as clinically indicated for all baseline FDG-avid lymphomas. MRI with contrast may be used for the CT portion when CT is clinically contraindicated or as required by local regulation. If PET cannot be performed with diagnostic quality CT, a separate diagnostic CT must be performed. PET/CT is evaluated locally and centrally.
²⁶Global response by ISCL response criteria and Overall response by Lugano criteria.
²⁷Cutaneous assessment (mSWAT) to be evaluated locally but may also be evaluated centrally if indicated (i.e., skin punch biopsy).
²⁸Skin photographs and modified severity weight assessment tool (mSWAT) to be performed post LD chemotherapy day 3 and prior to CTX130 infusion (Day 1 pre-infusion is best).
²⁹Bone marrow biopsy and aspirate collection is performed if clinically indicated. For subjects with bone marrow involvement at screening, an additional bone marrow biopsy and aspirate collection is performed to confirm CR. In the event a bone marrow biopsy or aspirate is performed, samples should be sent to central lab.
³⁰Biopsy (including skin punch biopsy) to be performed at screening if postprogression biopsy tissue is not available/acceptable, at Day 12 (+2 days), and at Day 28 (±2 days) after the initial infusion of CTX130. For subjects undergoing additional course(s) of treatment, tumor biopsy will not be performed on Day 12 and Day 28 after CTX130 infusion. Tumor biopsy to be evaluated locally and centrally. If a relapse occurs while a subject is on study, every attempt should be made to obtain biopsy of relapse tumor to send to central laboratory.
³¹Day 12 tumor biopsy for Part A only.
³²Perform peripheral blood tumor burden assessments, e.g., Sezary cell counts, ATLL cell counts, per institutional guidelines.
³³Hematocrit, hemoglobin, red blood cell count, white blood cell count, neutrophils, lymphocytes, monocytes, basophils, eosinophils, platelet count, absolute neutrophil count.
³⁴For subjects experiencing grade ≥ 3 neutropenia, thrombocytopenia, or anemia that has not resolved within 28 days of CTX130 infusion, a CBC with differential must be performed weekly until resolution to grade ≤ 2.
³⁵Serum chemistries to include ALT (SGPT), AST (SGOT), bilirubin (total), albumin, alkaline phosphatase, bicarbonate, BUN, calcium, chloride, creatinine, eGFR, glucose, LDH, phosphorus, potassium, sodium, total protein, uric acid (uric acid should be included in serum chemistry assessments through Day 28). If acute renal tubular damage is suspected, additional tests should be conducted including urine sediment analysis and fractional excretion of sodium in urine, and consultation by a nephrologist should be initiated.
³⁶Include PTT, fibrinogen, INR, and d-dimer.
³⁷Viral serologies for HIV-1, HIV-2, HBV (HBsAg, HBsAb, HBcAb), HCV (HCV antibody and RNA), EBV (see EBV monitoring line item in schedule of assessments table), HHV-6, HHV-7, CMV at screening; however, historical results obtained within 60 days of enrollment may be used to determine eligibility.
³⁸Assessed using quantitative polymerase chain reaction (qPCR). Monitor as clinically indicated until CD4+ counts of 200/μl are reached. Suspicious or ambiguous lesions in the context of rising EBV DNA levels should be biopsied and examined by local pathology, EBER ISH.
³⁹SARS-CoV-2 test is performed at screening. Screening test does not need to be repeated if within 3-4 days prior to start of lymphodepletion regimen (LD chemotherapy with or without daratumumab).
⁴⁰Include IgA, IgG, IgM
⁴¹Lymphocyte subset assessment at screening, before start of first day of LD chemo (Part A3) or pre initial daratumumab dose (Part A4), before CTX130 infusion, then all listed time points are assessed at local laboratory and will include 6-color TBNK panel, or equivalent for T, B, and NK cells.
⁴²sCD25 also to be assessed during suspected HLH.
⁴³Troponin, NT-proBNP, and BNP assessed at screening and in the event of a grade > 2 CRS on day 1, 3, and 7 of CRS event or as clinically indicated (Maus et al., 2020).
⁴⁴Samples for CTX130 levels should be collected from any lumbar puncture, blood samples, BM aspirate samples and tissue biopsies performed following CTX130 infusion, including unscheduled visits. If CRS occurs, samples for assessment of CTX130 levels are collected every 48 hours (±5 hours) between scheduled visits until CRS resolves. In subjects experiencing signs or symptoms of neurotoxicity and suspected HLH, additional blood samples should be drawn at intervals outlined in the laboratory manual.
⁴⁵Sponsor may discontinue testing and request discontinuation of sample collection if consecutive tests are negative. Continue sample collection for all listed time points until otherwise instructed by sponsor.
⁴⁶Two samples collected on Day 1 and Day 5: One pre-CTX130 infusion and one 20 (±5) minutes after the end of CTX130 infusion.
⁴⁷In the event of grade > 2 CRS, cytokine samples should be collected at the onset of symptoms. Additional cytokine samples should be collected every 24 hours (±5 hours) for the duration of any grade CRS. During neurotoxicity and suspected HLH, additional cytokine samples are collected
⁴⁸Samples are to be collected at the same time of day (±2 hours) on the specified collection days.
⁴⁹Samples for exploratory biomarkers should be sent from any LP or BM aspirate performed following CTX130 infusion. Subsequent LP and BM aspirate should only be collected when a screening sample is positive, and when follow-up samples are collected. If CRS occurs, samples for assessment of exploratory biomarkers are collected every 48 hours (±5 hours) between scheduled visits until CRS resolves. If neurotoxicity or HLH occur, samples for assessment of exploratory biomarkers are collected as instructed in the laboratory manual.

TABLE 41
Schedule of Assessments: Parts A5 and A6-Screening to Month 3

| | | | | | Follow-Up | | | | | | | | | | | |
|---|---|---|---|---|---|---|---|---|---|---|---|---|---|---|---|---|
| | | | | | | | | | | | | | | FOR | | |
| | | | | | | | | | | | | | | SUBJECTS | | |
| | | | | | | | | | | | | | | RECEIVING | | |
| | | | | | | | | | | | | | | DAY 35 | | |
| | | | | | | | | | | | | | | CTX130 | FOR | |
| | | | | | | | | | | | | | | INFUSION, | SUBJECTS | FOR |
| | | | | | | | | | | | | | | RESTART | WHO | SUBJECTS |
| | | | | | | | | | | | | | | AT | RECEIVED | WHO |
| | | | | | | | | | | | | | | SCREENING | DAY 35 | RECEIVED |
| | | | | | | | | | | | | | | AND | CTX130 | DAY 35 |
| | | | | | | | | | | | | | | FOLLOW | INFUSION: | CTX130 |
| | | | | | | | | | | | | | | VISIT | After | INFUSION: |
| | | | | | | | | | | | | | | SCHEDULE. | completing | Continue |
| | | Treatment | | | | | | | | | | | | For subjects | repeat | Month 4 |
| | | D - 10 | | | | | | | | | | | | NOT | of D28 | follow-up |
| | | to | | | | | | | | | | | | receiving | assessments, | as in |
| | | prior | | | | | | | | | | | | Day 35 | complete | Table 20 |
| | | to | | | | | | | | | | | | CTX130 | weekly | M3 |
| | | LD | D− | | | | | | | | | Part | | infusion, go | assessments | (D84 ± |
| | | chemo | 7 | | | | | | | | | A6 | | to Table | and go | 7d) |
| | | Part | to | | | | | | | | | only | | 20, | to Month 3 | post 1^{st} |
| Assessment | Screen- | A6 | D − | | | | | D7 + | D10 ± | D14 ± | D21 ± | D25 ± | D28 ± | Day 35 | Q7 | CTX130 |
| Day | ing¹ | only2 | 3³ | D1⁴ | D2 | D3 | D5 | 2 d | 1 d | 2 d | 2 d | 2 d | 2 d | assessments | days ± 2d | infusion |
| Eligibility | X | | X | X | | | | | | | | | | | | |
| confirmation⁵ | | | | | | | | | | | | | | | | |
| Informed | X | | | | | | | | | | | | | | | |
| consent | | | | | | | | | | | | | | | | |
| CD70 | X | | | | | | | | | | | | | | | |
| expression⁶ | | | | | | | | | | | | | | | | |
| Medical | X | | | | | | | | | | | | | | | |
| history⁷ | | | | | | | | | | | | | | | | |
| Physical | X | X | X | X | X | X | X | X | X | X | X | X | X | | X | X |
| exam⁸ | | | | | | | | | | | | | | | | |
| Vital signs⁹ | X | X | 3X | X | X | X | X | X | X | X | X | X | X | | X | X |
| Height, | X | X | X | X | | | | X | | X | | | X | | X | X |
| weight¹⁰ | | | | | | | | | | | | | | | | |
| Pregnancy | X | | X | | | | | | | | | | X | | | X |
| test¹¹ | | | | | | | | | | | | | | | | |
| ECOG status | X | X | 3X¹² | X | | | | | | | | | X | | | X |
| Echocardio- | X | | | | | | | | | | | | | | | |
| gram | | | | | | | | | | | | | | | | |
| 12-lead | X | X | X | X | | | | | | | | | X | | | |
| ECG¹³ | | | | | | | | | | | | | | | | |
| ICE | X | | | X | X | X | X | X | | | | | X | | | |
| assessment¹⁴ | | | | | | | | | | | | | | | | |
| PRO¹⁵ | X | | | X | | | | | | X | | | X | | | X |
| Con meds¹⁶ | Continous | | | | | | | | | | | | | | | |
| AEs¹⁷ | Continuous | | | | | | | | | | | | | | | |
| Hospital | | | | Continuous | | | | | | | | | | | | |
| Utilization | | | | | | | | | | | | | | | | |

Treatment

| | | | |
|---|---|---|---|
| Daratu- | X | | |
| mumab | | | |
| Part A6 | | | |
| only¹⁸ | | | |
| LD chemo- | | 3X | |
| herapy¹⁹ | | | |
| CTX130 | | | X |
| infusion²⁰-²¹ | | | |

B and T Cell Lymphoma Disease/Response Assessments

| | | | | | |
|---|---|---|---|---|---|
| Whole body | X | | | | |
| PET/CT scan | | | | | |
| or CT scan²² | | | | | |
| Global/ | | | | X | X |
| Overall | | | | | |
| Response²³ | | | | | |
| Brain MRI²² | X | | | | |
| Cutaneous | | X²⁵ | X | X | X |
| assessment | | | | | |
| for | | | | | |
| all T-cell | | | | | |
| lymphomas | | | | | |
| (mSWAT) ²⁴ | | | | | |
| TNMB | X | | | | |
| Tumor | X | | X²⁷ | X | |
| biopsy²⁶ | | | | | |
| Peripheral | X | | X | X | X |
| blood tumor | | | | | |
| burden/ | | | | | |
| immuno- | | | | | |
| pheno- | | | | | |
| typing²⁸ | | | | | |
| BM aspirate/ | X | | | | |
| biopsy²⁹ | | | | | |

Laboratory Assessments (Local)

| | | | | | | | | | | | | | | | |
|---|---|---|---|---|---|---|---|---|---|---|---|---|---|---|---|
| CBC w/ | X | X | 3X | X | X | X | X | X | X | X | X | | | | |
| differential³⁰ | | | | | | | | | | | | | | | |
| Serum | X | X | 3X | X³² | X³² | X³² | X³² | X³² | X³² | X³² | X³² | X³² | X³² | X | X |
| chemistry³² | | | | | | | | | | | | | | | |
| Coagulation | X | X | X | X | X | X | X | X | X | X | | | X | | |
| arameters33 | | | | | | | | | | | | | | | |
| Viral | X | | | | | | | | | | | | | | |
| serology³⁴ | | | | | | | | | | | | | | | |
| EBV | X | | | X | | | | X | | X | X | | X | | |
| nonitoring³⁵ | | | | | | | | | | | | | | | |
| SARS- | X | | | | | | | | | | | | | | |
| CoV-2³⁶ | | | | | | | | | | | | | | | |
| Immuno- | X | | | X | | | | X | | X | X | | X | | X |
| globulins³⁷ | | | | | | | | | | | | | | | |
| Lymphocyte | X | X³⁸ | X³⁸ | X | X | X | X | X | X | X | X | | X | | X |
| subsets | | | | | | | | | | | | | | | |
| (TBNK) ³⁸ | | | | | | | | | | | | | | | |
| Ferritin, CRP | X | | | X | X | X | X | X | X | X | X | X | X | | |
| CD25 ³⁹ | X | X | | | | | | | | X | X | X | X | | |
| Troponin, | X | | | | | | | | | | | | | | |
| NT-roBNP, | | | | | | | | | | | | | | | |
| BNP⁴⁰ | | | | | | | | | | | | | | | |

Biomarkers (Blood, Central)

| | | | | | | | | | | |
|---|---|---|---|---|---|---|---|---|---|---|
| CTX130 | X | | X⁴¹ | X | X | X | X | X | X | X |
| levels⁴¹ | | | pre/ | | | | | | | |
| | | | post | | | | | | | |
| Cytokines⁴² | | X | X | X | X | X | X | X | X | X |
| BSAP, | X | | X | | X | | X | X | X | X |
| PINP⁴³ | | | | | | | | | | |
| Anti- | X | | | | | | | | X | X |
| CTX130 | | | | | | | | | | |
| Ab | | | | | | | | | | |
| Exploratory | X | X | X | | X | | X | X | X | X |
| siomarkers⁴⁴ | | | | | | | | | | |

Ab: antibody;
AE: adverse event;
AESI: adverse event of special interest;
ALT: alanine aminotransferase;
AST: aspartate aminotransferase;
ATLL: adult T cell leukemia/lymphoma;
BM: bone marrow;
BNP: B-type natriuretic peptide;
BSAP: bone-specific alkaline phosphatase;
BUN: blood urea nitrogen;
CBC: complete blood count;
chemo: chemotherapy;
CMV: cytomegalovirus;
CNS: central nervous system;
con meds: concomitant medications;
CR: complete response;
CRISPR: clustered regularly interspaced short palindromic repeats;
CRP: C-reactive protein;
CRS: cytokine release syndrome;
CT: computed tomography;
D or d: day; DNA: deoxyribonucleic acid;
EBER: EBV-encoded small RNA;
EBV: Epstein-Barr virus;
ECG: electrocardiogram;
ECOG: Eastern Cooperative Oncology Group;
eGFR: estimated glomerular filtration rate;
EORTC QLQ-C30: European Organisation for Research and Treatment of Cancer QLQ-C30 and questionnaire;
FACT-G: Functional Assessment of Cancer Therapy-General;
FDG: fluorodeoxy glucose;
GvHD: graft versus host disease;
HBcAb: hepatitis B core antibody;
HBsAb: hepatitis B surface antibody;
HBsAg: hepatitis B surface antigen;
HBV: hepatitis B virus;
HCV: hepatitis C virus;
HHV-6: human herpesvirus 6;
HIV-1/-2: human immunodeficiency virus type 1 or 2;
HLH: hemophagocytic lymphohistiocytosis;
HTLV-1: human T cell leukemia virus type 1;
ICE: immune effector cell-associated encephalopathy;
Ig: immunoglobulin;
INR: international normalized ratio;
ISH: in situ hybridization;
LD: lymphodepleting;
LDH: lactate dehydrogenase;
LP: lumbar puncture;
M: month;
MF: mycosis fungoides;
MRD: minimal residual disease;
MRI: magnetic resonance imaging;
mSWAT: modified Severity Weighted Assessment Tool;
NK: natural killer;
NT-proBNP: N-terminal pro hormone B-type natriuretic peptide;
PET: positron emission tomography;
PINP: procollagen type 1N propeptide;
PRO: patient-reported outcomes;
PT: prothrombin time;
PTT: partial prothrombin time;
Q7 days: every 7 days;
RNA: ribonucleic acid;
sCD25: soluble CD25;
SGOT: serum glutamic oxaloacetic transaminase;
SGPT: serum glutamic pyruvic transaminase;
SS: Sézary syndrome;
TBNK: T, B, and NK cells.
Note:
Assessments scheduled on CTX130 infusion days are to be performed pre-CTX130 infusion unless otherwise specified; for samples tested centrally, refer to Laboratory Manual. Note: Subjects who achieve CR, PR, SD, or PD with clinical benefit at Day 28 scan (based on Lugano or Olsen criteria as appropriate) will receive a second infusion of CTX130 on Day 35 (−7 days/+21 days) with prior daratumumab (Part A6 only) and LD chemotherapy. In addition, after the first course of treatment, an optional single additional infusion of CTX130 can be administered with LD chemotherapy (and with daratumumab if applicable) after 1) loss of complete response (CR) within the first 2 years after initial infusion of CTX130, or 2) partial response (PR), stable disease (SD), or progressive disease (PD) with clinical benefit as determined by the investigator. Prior to the second (D35) infusion during the first course of treatment, or prior to an additional single infusion of CTX130 after the first course of treatment, all screening assessments must be repeated except for radiological (PET-CT or CT) disease assessments if performed within 28 days prior to next CTX130 infusion, bone marrow biopsy/aspirate unless clinically indicated, echocardiogram (unless new cardiac signs or symptoms), and brain MRI. In addition, not all central lab screening samples may be required (see Laboratory Manual for details). Local and central laboratory testing performed within 14 days of planned start of lymphodepletion may be used to confirm eligibility. After screening and LD regimen, subjects should be followed per the schedule of assessments consistent with the first infusion of CTX130 through Day 28, except that tumor biopsy will not be performed on Day 7 and Day 28 after CTX130 infusion.
Note
(follow-up schedule post-CTX130 infusion at D35 or after first course of treatment): Subjects who receive second (D35) CTX130 infusion should repeat screening through D28 assessments, and then continue with weekly (Q7 days ±2d) assessments, followed by Month 3 (post-initial CTX130 infusion) assessments, and then proceed to Table 11 for Month 4 through Month 24 assessments. Subjects receiving an additional single infusion of CTX130 (post-first course of treatment) should repeat screening through D28 assessments and then continue with the next scheduled visit assessments; if the next scheduled visit occurs within 28 days of previous disease response assessments, then disease response assessments for next scheduled visit do not need to be performed-note, however, that through Month 12 post-initial CTX130 infusion, there should not be more than a 3-month gap between disease response assessments, and after Month 12 (post-initial CTX130 infusion), there should be no more than a 6-month gap between disease response assessments.
Note:
the second (D35) CTX130 infusion during the first course of treatment or an additional single infusion of CTX130 after the first course of treatment may be administered without prior LD chemotherapy if subject is experiencing significant cytopenia.
Note:
Certain assessments for visits after 7 days post-CTX130 infusion may be performed as in-home or alternate-site visits. Assessments may include hospital utilization, changes in health and/or changes in medications, vital signs, weight, PRO questionnaire distribution, and blood sample
collections for local and central laboratory assessments.
¹Screening assessments to be completed within 14 days of informed consent. The screening period may be extended beyond 14 days to allow for COVID-19 testing only. Screening assessment of disease category/subtype will be reviewed via central pathology examination of collected tissue representative of patient’s disease. Subjects will be allowed a one-time rescreening, which may take place within 3 months of initial consent. Subjects who rescreen may use previous BM biopsy/aspirate samples for rescreening if no anticancer therapies have been administered in the interim. In addition, not all central lab samples may be required (see Laboratory Manual for details).
²All assessments must occur on the same day and prior to daratumumab administration.
³“X” indicates occurrence only on first day of LD chemo. “3X” indicates occurrence on each of the 3 days of LD chemo. Assessments scheduled on LD chemo days are to be performed pre-LD chemo unless otherwise specified.
⁴All baseline assessments on Day 1 are to be performed prior to CTX130 infusion unless otherwise specified; refer to the Laboratory Manual for details.
⁵Eligibility should be confirmed each time screening is completed. Eligibility should also be confirmed on day of daratumumab administration (Part A6 only), on first day of LD chemotherapy, and on day of CTX130 infusion. Eligibility should be reconfirmed after all assessments for that day are completed and before dosing.
⁶Subjects must have CD70-expressing tumors to receive DI CTX130 infusion during first course of treatment or single additional CTX130 infusion after first course of treatment. Tissue may be submitted and tested locally or centrally at any time prior to or during the 14-day window for screening, provided the subject has signed an appropriate consent. Tissue for CD70 testing should be representative of the subject’s disease but does not need to be collected within 3 months of CTX130 infusion or post progression after the last systemic therapy. To receive single additional infusion of CTX130 after first course of treatment, subjects must have CD70 expression of tumors confirmed from a new tumor sample.
⁷Includes complete surgical, neurological, and cardiac history.
⁸Includes assessment for signs and symptoms of GvHD: skin, oral mucosa, sclera, hands, and feet.
⁹Includes sitting blood pressure, heart rate, respiratory rate, pulse oximetry, and temperature.
¹⁰Height at initial screening only.
¹¹For female subjects of childbearing potential. Serum pregnancy test required at screening, within 72 hours of beginning LD chemotherapy (Part A5) or daratumumab dose (Part A6), and at Day 28, Day 56, and Month 3 visit. Will be assessed at a local laboratory.
¹²Prior to initiation of LD chemotherapy on each of the three days, ECOG performance status should be checked.
¹³12-lead ECG test should be conducted at screening, prior to daratumumab administration (Part A6 only), first day of LD chemotherapy, and CTX130 infusion, and on Day 28.
¹⁴On Day 1, prior to CTX130 administration. If CNS symptoms persist, ICE assessment should continue to be performed approximately every 2 days until symptom resolution to grade 1 or baseline.
¹⁵EORTC QLQ-30, EQ-5D-5L questionnaires for all indications; FACT-G, Skindex-29 questionnaire for SS and MF and for any subjects with skin lesions. PRO surveys should be administered before any visit-specific procedures are performed.
¹⁶All concomitant medications will be collected up to 3 months after each CTX130 infusion, after which only select concomitant medications will be collected
¹⁷Collect all AEs from informed consent to 3 months after each CTX130 infusion and collect only SAEs and AESIs from 3 months after last CTX130 infusion through Month 24 visit. After Month 24 to Month 60 or after a subject starts a new anticancer therapy, only CTX130-related SAEs, CTX130-related AESIs, and new malignancies will be reported.
¹⁸Part A6 only: All assessments on days of daratumumab administration must be completed prior to daratumumab dosing unless otherwise specified. One dose of daratumumab 16 mg/kg IV or 1800 mg SC administered at least 12 hours prior to starting LD chemotherapy and within 10 days prior to CTX130 infusion. To facilitate administration, the 16 mg/kg IV dose may be split (to 8 mg/kg IV) over 2 consecutive days per the daratumumab prescribing information. If a subject experiences disease progression or unacceptable adverse events related to daratumumab, repeat dosing with daratumumab will not be permitted.
¹⁹For first CTX130 infusion, start LD chemotherapy within 7 days of study enrollment. After completion of LD chemotherapy, ensure washout period of ≥ 48 hours (but ≤ 7 days) before CTX130 infusion. Physical exam, weight, and coagulation laboratories are performed prior to first dose of LD chemotherapy. Vital signs, CBC with differential, serum chemistry, ECOG status, and AEs/concomitant medications should be assessed and recorded daily (i.e., 3 times) during LD chemotherapy.
²⁰CTX130 will be administered 48 hours to 7 days after completion of LD chemotherapy.
²¹Subjects who achieve CR, PR, SD, or PD with clinical benefit at Day 28 scan (based on Lugano or Olsen criteria as appropriate) will receive a second infusion of CTX130 on Day 35 (−7 days/+21 days) with prior daratumumab (Part A6 only) and LD chemotherapy. Prior to the second CTX130 infusion, all screening assessments must be repeated, except for brain MRI, bone marrow biopsy unless clinically indicated, and radiological (PET-CT or CT) disease assessments if performed within 28 days of the screening period. In addition, not all central lab screening samples may be required (see Laboratory Manual for details). Subjects who receive second (D35) CTX130 infusion should repeat of screening through D28 assessments, and then continue with weekly (Q7 days ± 2d) assessments, followed by Month 3 (post-initial CTX130 infusion) assessments, and then proceed to Table 20 for Month 4 through Month 24 assessments. Note that the second CTX130 infusion may be administered without LD chemotherapy if subject is experiencing significant cytopenia
²²Whole body (including neck) PET/CT and MRI brain scan to be performed as part of or prior to screening (within 28 days prior to CTX130 infusion). Brain MRI is only required as part of screening for the first course of treatment, i.e., brain MRI does not need to be performed as part of screening prior to either the Day 35 CTX130 infusion or the single additional CTX130 infusion after the first course of treatment. Non FDG-avid lymphomas may be followed post-baseline by CT as clinically indicated. Whole body (including neck if involved at baseline) PET/CT or CT, as clinically indicated, to be performed per the schedule of assessments and upon suspected CR. Postinfusion scans will be performed per the schedule of assessments, per the protocol-defined response criteria and as clinically indicated for all baseline FDG-avid lymphomas. MRI with contrast may be used for the CT portion when CT is clinically contraindicated or as required by local regulation. If PET cannot be performed with diagnostic quality CT, a separate diagnostic CT must be performed. PET/CT will be evaluated locally and centrally.
²³Global response by ISCL response criteria and Overall response by Lugano criteria.
²⁴ Cutaneous assessment (mSWAT) to be evaluated locally but may also be evaluated centrally if indicated (i.e., skin punch biopsy).
²⁵Skin photographs and modified severity weight assessment tool (mSWAT) to be performed post LD chemotherapy day 3 and prior to CTX130 infusion (Day 1 pre-infusion is best).
²⁶Biopsy (including skin punch biopsy) to be performed at screening if postprogression biopsy tissue is not available/acceptable, Day 7 (+2 days), and Day 28 (±2 days) after the infusion of CTX130. For subjects undergoing a Day 35 infusion or a single additional infusion after the first course of treatment, tumor biopsy will not be performed on Day 7 and Day 28 after CTX130 infusion. Tumor biopsy to be evaluated locally and centrally. If a relapse occurs while a subject is on study, every attempt should be made to obtain biopsy of relapse tumor to send to central laboratory.
²⁷Day 7 tumor biopsy for Part A only
²⁸Perform peripheral blood tumor burden assessments, e.g., Sezary cell counts, ATLL cell counts, per institutional guidelines.
²⁹Bone marrow biopsy and aspirate collection will be performed if clinically indicated. For subjects with bone marrow involvement at screening, an additional bone marrow biopsy and aspirate collection will be performed to confirm CR. In the event a bone marrow biopsy or aspirate is performed, samples should be sent to central lab.
³⁰Hematocrit, hemoglobin, red blood cell count, white blood cell count, neutrophils, lymphocytes, monocytes, basophils, eosinophils, platelet count, absolute neutrophil count.
³¹For subjects experiencing grade ≥ 3 neutropenia, thrombocytopenia, or anemia that has not resolved within 28 days of CTX130 infusion, a CBC with differential must be performed weekly until resolution to grade ≤ 2.
³²Serum chemistries to include ALT (SGPT), AST (SGOT), bilirubin (total), albumin, alkaline phosphatase, bicarbonate, BUN, calcium, chloride, creatinine, eGFR, glucose, LDH, phosphorus, potassium, sodium, total protein, uric acid (uric acid should be included in serum chemistry assessments through Day 28). If acute renal tubular damage is suspected, additional tests should be conducted including urine sediment analysis and fractional excretion of sodium in urine, and consultation by a nephrologist should be initiated.
³³Include PTT, fibrinogen, INR, and d-dimer.
³⁴Viral serologies for HIV-1, HIV-2, HBV (HBsAg, HBsAb, HBcAb), HCV (HCV antibody and RNA), EBV (see EBV monitoring line item in schedule of assessments table), HHV-6, HHV-7, CMV at screening; however, historical results obtained within 60 days of enrollment may be used to determine eligibility.
³⁵Assessed using quantitative polymerase chain reaction (qPCR). Monitor as clinically indicated until CD4+ counts of 200/μl are reached. Suspicious or ambiguous lesions in the context of rising EBV DNA levels should be biopsied and examined by local pathology, EBER ISH.
³⁶ SARS-CoV-2 test will be performed at screening. Screening test does not need to be repeated if within 3-4 days prior to start of lymphodepletion regimen (LD chemotherapy with or without daratumumab).
³⁷Include IgA, IgG, IgM.
³⁸ Lymphocyte subset assessment at screening, before start of first day of LD chemo (Part A5) or pre daratumumab dose (Part A6), before CTX130 infusion, then all listed time points will be assessed at local laboratory and will include 6-color TBNK panel, or equivalent for T, B, and NK cells.
³⁹ sCD25 also to be assessed during suspected HLH.
⁴⁰Troponin, NT-proBNP, and BNP assessed at screening and in the event of a grade ≥ 2 CRS on day 1, 3, and 7 of CRS event or as clinically indicated (Maus et al., 2020).
⁴¹For CTX130 levels, 2 samples should be collected on Day 1: one pre-CTX130 infusion and one 20 minutes (±5 minutes) after the end of CTX130 infusion. If CRS occurs, samples for assessment of CTX130 levels will be collected every 48 hours (±5 hours) between scheduled visits until CRS resolves. Samples for CTX130 levels should be collected from any lumbar puncture, blood samples, BM aspirate samples and tissue biopsies performed following CTX130 infusion, including unscheduled visits. In subjects experiencing signs or symptoms of neurotoxicity and suspected HLH, additional blood samples should be drawn at intervals outlined in the laboratory manual. Sponsor may discontinue testing and request discontinuation of sample collection if consecutive tests are negative. Continue sample collection for all listed time points until otherwise instructed by sponsor.
⁴²In the event of grade ≥ 2 CRS, samples should be collected at the onset of symptoms. Additional cytokine samples should be collected every 24 hours (±5 hours) for the duration of any grade CRS. During neurotoxicity and suspected HLH, additional cytokine samples will be collected
⁴³Samples are to be collected at the same time of day (±2 hours) on the specified collection days
⁴⁴Samples for exploratory biomarkers should be sent from any LP or BM biopsy performed following CTX130 infusion. Subsequent LP and BM aspirate should only be collected when a screening sample is positive, and when follow-up samples are collected. If CRS occurs, samples for assessment of exploratory biomarkers will be collected every 48 hours (±5 hours) between scheduled visits until CRS resolves. If neurotoxicity or HLH occur, samples for assessment of exploratory biomarkers will be collected as instructed in the laboratory manual.

### B Cell and T Cell Lymphoma Disease and Response Assessments

Disease evaluations are based on assessments in accordance with the Lugano response criteria (Cheson et al., J Clin Oncol, 2014) for subjects with PTCL-NOS, ALCL, leukemic and lymphomatous ATLL, AITL, and DLBCL, and according to International Society for Cutaneous Lymphomas (ISCL) response criteria (Olsen et al., J Clin Oncol, 2011) for subjects with SS or MF.

Disease assessment in the brain is performed by MRI to rule out brain involvement in subjects during screening.

Per (Olsen et al., Blood, 2007), the subjects with SS must have:
Measurable disease per Lugano criteria; meeting the definition for SS with ≥80% of body surface area and blood involvement.
Erythroderma defined as erythema covering at least 80% body surface area.
A clonal TCR rearrangement in the blood identified by PCR or Southern blot analysis.
An absolute count of Sézary cells in blood of ≥1,000/μL or 1 of the following 2 criteria:
   Increased CD4+ or CD3+ cells with a CD4 to CD8 ratio of 10 or more.
   Increased CD4+ cells with an abnormal phenotype (such as a CD4+CD7-ratio ≥40% or a CD4+CD26− ratio ≥30%).

For efficacy analyses, disease outcome is graded using the Lugano response criteria for the following tumor subtype for PET/CT imaging or CT imaging for non-fluorodeoxyglucose (FDG)-avid disease:
PTCL-NOS
ALCL
Leukemic and lymphomatous ATLL
AITL
DLBCL

For subjects with ATLL hypercalcemia, flares are not considered PD as long as active disease persists and are treated symptomatically per institutional guidelines. Changes in peripheral blood levels of ATLL cells is monitored by immunophenotyping based on markers such as CD3, CD4, CD7, CD8, CD25, CD52, and HTLV-1 proviral load is an exploratory endpoint.

Increased lymphocytosis in the setting of a decrease in lymph node measurement is not considered PD, and response designation depends on lymph nodes and extranodal disease measurement.

Disease measurement for cutaneous lesions in non-CTCLs follows the guidelines for response assessment of cutaneous lesions per ISCL response criteria.

ISCL response criteria is used for subjects with SS or MF (or if indicated positron emission tomography [PET]/CT) imaging. Erythrodermic flare is not considered disease progression during the first 2 months.

T cell lymphoma disease and response evaluation is conducted per the schedule in Tables 20, 21, 40, and 41, and includes the assessments described below. All response categories (including progression) require 2 consecutive assessments made at least 1 week apart at any time before the institution of any new therapy.

### Pre-CTX130 Biopsy

Histopathological diagnosis of T cell lymphoma subtype is based on local and central laboratory assessment.

Subjects are required to undergo tumor biopsy at screening or, if a biopsy was performed within 3 months prior to enrollment and after the last systemic or targeted therapy, archival tissue may be provided. If archival tissue is of insufficient volume or quality to fulfill central laboratory requirements, a biopsy is performed during screening. Bone biopsies and other decalcified tissues are not acceptable due to interference with downstream assays. Portions of the tissue biopsy are submitted to a central laboratory for analysis. In some instances, subjects with MF or SS may not be required to provide a bone marrow biopsy at screening.

Tumor tissue samples may be analyzed for tumor intrinsic and TME-specific biomarkers, including analysis of DNA, RNA, protein, and metabolites.

### Whole Body PET/CT Radiographic Disease Assessment

Whole body (including neck) PET/CT to be performed as part of or prior to screening (i.e., within 28 days prior to first CTX130 infusion) and upon suspected CR. Postinfusion scans are conducted per the schedule of assessments in Tables 20, 21, 40, and 41, per the protocol-defined response criteria and as clinically indicated for all baseline FDG-avid lymphomas. PET/CT non-FDG-avid disease is followed post-baseline by CT. MRI with contrast may be used for the CT portion when CT is clinically contraindicated or as required by local regulation. If PET cannot be performed with diagnostic quality CT, a separate diagnostic CT must be performed.

Requirements for the acquisition, processing, and transfer of scans is outlined in the imaging manual. Whenever possible, the imaging modalities, machines, and scanning parameters used for radiographic disease assessment are kept consistent during the study. For efficacy analyses, radiographic disease assessments are performed by the Independent Review Committee (IRC) in accordance with protocol-defined response criteria.

### Cutaneous Assessment

Cutaneous assessment is performed as specified in Tables 20, 21, 40, and 41. Initial cutaneous disease assessment is performed post LD chemotherapy Day 3 and prior to CTX130 infusion (Day 1 pre-infusion is best). The prognosis of MF and SS depends on the type and extent of skin lesions and extracutaneous disease (Olsen et al., J Clin Oncol, 2011). The recommendations based on the consensus guidelines (ISCL, US Cutaneous Lymphoma Consortium); the Cutaneous Lymphoma Task Force of the EORTC including a scoring system for assessing tumor burden in skin, lymph nodes, blood, and viscera; the definition of response in skin, nodes, blood, and viscera; and a composite global response score are described herein. Response assessment should be supported by photographic documentation of representative areas.

### Bone Marrow Biopsy and Aspirate

Bone marrow biopsy and aspirate collection is performed, if clinically indicated. For subjects with bone marrow involvement at screening, an additional bone marrow biopsy and aspirate collection is performed to confirm CR. In the event a bone marrow biopsy or aspirate is performed, samples should be sent to central lab.

Samples for presence of CTX130 (detected via PCR) are sent for central laboratory evaluation at any point when BM analysis is performed. Samples from BM aspirate after CTX130 infusion are sent for CTX130 PK and exploratory biomarkers. Standard institutional guidelines for the BM biopsy are followed. Excess sample (if available) is stored for exploratory research.

### Tumor Biopsy

Subjects are required to undergo tumor biopsy (including skin punch biopsy) at screening or, if a postprogression biopsy was performed within 3 months prior to enrollment and after the last systemic or targeted therapy, archival tissue may be provided. If archival tissue is of insufficient volume or quality to fulfill central laboratory requirements, a biopsy must be performed during screening.

In Parts A1, A2, A5, and A6, tumor biopsy is performed on Day 7 (+2 days; or as soon as clinically feasible) and Day 28 (±2 days) after initial dosing only (i.e., first course of treatment); Day 7 tumor biopsy is not performed in Part B. In Parts A3 and A4 only, tumor biopsy is performed on Day 12 (+2 days) and Day 28 (±2 days) after initial dosing only; Day 12 tumor biopsy is not performed in Part B. If a relapse occurs while a subject is on study, every attempt should be made to obtain biopsy of relapse tumor and send to central laboratory.

Biopsies come from nontarget lesions. When multiple biopsies are taken, efforts are made to obtain them from similar tissues. Liver metastases are generally less desirable. Bone biopsies and other decalcified tissues are not acceptable due to interference with downstream assays. This sample is analyzed for presence of CTX130 as well as tumor-intrinsic and TME-specific biomarkers including analysis of DNA, RNA, protein, or metabolites.

### Lugano Response Criteria, 2014

The following is adapted from Cheson et al., J Clin Oncol, 2014, Recommendations for initial evaluation, staging, and response assessment of Hodgkin and non-Hodgkin lymphoma: The Lugano Classification. J Clin Oncol. 2014.

Diagnosis

A fine-needle aspirate is inadequate for initial diagnosis. An incisional or excisional biopsy is preferred to provide adequate tissue for these examinations. A core-needle biopsy can be considered when excisional biopsy is not possible and to document relapse; however, a nondiagnostic sample must be followed by an incisional or excisional biopsy.

Baseline Site Involvement

Criteria for site involvement are summarized in Table 22.

TABLE 22
Criteria for Involvement of Site

| Tissue Site | Clinical | FDG Avidity | Test | Positive Finding |
|---|---|---|---|---|
| Lymph nodes | Palpable | FDG-avid histologies | PET-CT | Increased FDG uptake |
| | | Nonavid disease | CT | Unexplained node |
| | | | | enlargement |
| Spleen | Palpable | FDG-avid histologies | PET-CT | Diffuse uptake, solitary |
| | | Nonavid disease | CT | mass, miliary lesions, |
| | | | | nodules |
| | | | | >13 cm in vertical |
| | | | | length, mass, nodules |
| Liver | Palpable | FDG-avid histologies | PET-CT | Diffuse uptake, mass |
| | | Nonavid disease | CT | Nodules |
| CNS | Signs, | | CT | Mass lesion(s) |
| | symptoms | | MRI | Leptomeningeal |
| | | | | infiltration, mass lesions |
| | | | CSF | Cytology, flow |
| | | | assessment | cytometry |
| Other (e.g., skin, lung, | Site | | PET-CT¹, | Lymphoma involvement |
| GI tract, bone, bone | dependent | | biopsy | |
| marrow) | | | | |

CSF: cerebrospinal fluid; CT: computed tomography FDG: fluorodeoxyglucose; GI: gastrointestinal; MRI: magnetic resonance imaging; PET: positron emission tomography.
¹PET-CT is adequate for determination of bone marrow involvement and can be considered highly suggestive for involvement of other extralymphatic sites. Biopsy confirmation of those sites can be considered if necessary.

Imaging

Positron emission tomography (PET)-computed tomography (CT) are used for staging of routinely fluorodeoxyglucose (FDG)-avid histologies. Scan are reported with visual assessment noting location of foci in nodal and extranodal sites. Images are scaled to a fixed standardized uptake value and color table; and distinguished from physiological uptake and other patterns of disease according to the distribution and/or CT characteristics.

PET-CT scans should be performed as follows:
As long as possible after the last chemotherapy administration for interim scans
6-8 weeks post chemotherapy at end of treatment ideally (but a minimum of 3 weeks)
≥3 months after radiotherapy

A contrast-enhanced CT scan may be included for a more accurate measurement of nodal size, and to more accurately distinguish bowel from lymphadenopathy; and in the setting of compression/thrombosis of central/mediastinal vessels. Contrast-enhanced CT is also preferred for radiation planning. Variably FDG-avid histologies should be staged with a CT scan.

For subjects staged with CT, disease should be evaluated according to Table 23.

TABLE 23
Disease Evaluation for CT-based Staging

| | Measurable Extranodal | Non-Measurable |
|---|---|---|
| Measurable Nodal Site | Disease Site | Disease Sites |
| LDi >1.5 cm | LDi >1.0 cm | All other disease sites: |
| | | Nodal |
| | | Extranodal |
| | | Assessable disease |

| | |
|---|---|
| Up to 6 measurable nodal/extranodal sites: | Examples: |
| Largest target nodes, nodal masses or other | skin, GI, bone, spleen, |
| lymphomatous lesions | liver, kidneys, effusions |
| Measurable extranodal disease | |
| Measurable in 2 diameters (LDi and SDi) | |
| Represent different body regions/overall disease | |
| burden | |
| Include mediastinal and retroperitoneal disease, if | |
| involved | |

GI: gastrointestinal; LDi: longest transverse diameter of a lesion; SDi: shortest axis perpendicular to LDi.

Tumor Bulk

A single nodal mass, in contrast to multiple smaller nodes, of 10 cm or greater than a third of the transthoracic diameter at any level of thoracic vertebrae as determined by CT is the definition of bulky disease for Hodgkin lymphoma (HL). A chest x-ray is not required to determine bulk. For HL and non-Hodgkin lymphoma (NHL) the longest measurement by CT scan should be recorded.

Measurements of total tumor volume should be explored as potential prognosticators with PET and CT.

Spleen Liver and Bone Marrow Involvement

Splenic and liver involvement are best determined by PET-CT as described in Table 24.

TABLE 24
Spleen and Liver Involvement

| Spleen | Liver |
|---|---|
| Use single measurement which | Liver size by physical examination or |
| correlates well with volume | CT scan not a reliable measure of |
| | hepatic involvement by lymphoma |
| Most studies use 10-12 cm for | Diffusely increased or focal uptake, |
| vertical length (cranial to caudal) | with or without focal or disseminated |
| Lugano recommendation: | nodules support liver involvement |
| Splenomegaly >13 cm | |

Bone marrow involvement may be determined as follows:
HL, if PET-CT is performed, bone marrow biopsy (BMB) is not required
DLBCL, BMB if the PET is negative and identifying a discordant histology is important for subject management
Other subtypes, ˜2.5 cm unilateral BMB is recommended, along with immunohistochemistry and flow cytometry at screening/baseline
If uninvolved at baseline, must be normal for CR and evidence of FDG-avid disease in marrow for complete metabolic response (CMR)

Staging System

A modified Ann Arbor staging system is used for anatomic description of disease extent (Table 25).

TABLE 25
Revised Staging System for Primary Nodal Lymphomas

| | | |
|---|---|---|
| Stage | Involvement | Extranodal Status |

Limited

| | | |
|---|---|---|
| Stage I | One node or group of adjacent | Single extranodal lesion without nodal |
| | nodes | involvement |
| Stage II | Two or more nodal groups on the | Stage I or II by nodal extent with |
| | same side of the diaphragm | limited, contiguous extranodal |
| | | involvement |
| Stage II bulky¹ | II as above with bulky disease | N/A |

Advanced

| | | |
|---|---|---|
| Stage III | Nodes on both sides of the | N/A |
| | diaphragm Nodes above the | |
| | diaphragm with spleen | |
| | involvement | |
| Stage IV | Additional noncontiguous | N/A |
| | extranodal involvement | |

NOTE.
Extent of disease is determined by positron emission tomography-computed tomography for avid lymphomas and computed tomography for nonavid histologies. Tonsils, Waldeyer's ring, and spleen are considered nodal tissue.
¹Whether Stage II bulky disease is treated as limited or advanced disease may be determined by histology and a number of prognostic factors.

Response Assessment

PET-CT is used for response assessment in FDG-avid histologies, using the 5-point scale; CT is preferred for low or variable FDG avidity.

Surveillance scans after remission are discouraged, especially for DLBCL and HL, although a repeat study may be considered after an equivocal finding after treatment. Judicious use of follow-up scans may be considered in indolent lymphomas with residual intra-abdominal or retroperitoneal disease.

To capture overall disease burden, disease extent at baseline and in subsequent assessments should be described as completely and clearly as feasible on a per lesion basis. Minimal information that should be captured per lesion as clinically appropriate are anatomical location, whether location is nodal or extra-nodal, the maximum SUV (SUVmax) by PET, and lesion dimensions by CT scan or MR To capture disease extent, 6 measurable target lesions should be selected if present, and measurable lesions in excess of 6 as well as non-measurable lesions should be captured as non-target lesions.

Criteria for response are summarized in Table 26.

TABLE 26
Revised Criteria for Response Assessment

| Response and | | CT-based Response |
|---|---|---|
| Site | PET/CT-based Response | Complete radiologic response (all of the |
| Complete | Complete metabolic response | following) |
| Lymph nodes & | Score 1, 2, or 3¹ with or without | Target nodes/nodal masses must regress to |
| extralymphatic | a residual mass on 5PS2 | ≤1.5 cm in LDi |
| sites | It is recognized that in | No extralymphatic sites of disease |
| | Waldeyer's ring or extranodal | |
| | sites with high physiologic | |
| | uptake or with activation within | |
| | spleen or marrow (eg, with | |
| | chemotherapy or myeloid | |
| | colony-stimulating factors), | |
| | uptake may be greater than | |
| | normal mediastinum and/or | |
| | liver. In this circumstance, | |
| | complete metabolic response | |
| | may be inferred if uptake at | |
| | sites of initial involvement is no | |
| | greater than surrounding normal | |
| | tissue even if the tissue has high | |
| | physiologic uptake | |
| Nonmeasured | Not applicable | Absent |
| lesion | | |
| Organ | Not applicable | Regress to normal |
| enlargement | | |
| New lesions | None | None |
| Bone marrow | No evidence of FDG-avid | Normal by morphology; if indeterminate, |
| | disease in marrow | IHC negative |
| Partial | Partial metabolic response | Partial remission (all of the following) |
| Lymph nodes & | Score 4 or 5² with reduced | ≥50% decrease in SPD of up to 6 target |
| extralymphatic | uptake compared with baseline | measurable nodes and extranodal sites |
| sites | and residual mass(es) of any | When a lesion is too small to measure on |
| | size | CT, assign 5 mm × 5 mm as the default value |
| | At interim, these findings | When no longer visible, 0 × 0 mm For a node |
| | suggest responding disease | >5 mm × 5 mm, but small |
| | At end of treatment, these | |
| | findings indicate residual | |
| | disease | |
| Nonmeasured | Not applicable | Absent/normal, regressed, but no increase |
| lesion | | |
| Organ | Not applicable | Spleen must have regressed by >50% in |
| enlargement | | length beyond normal |
| New lesions | None | None |
| Bone marrow | Residual uptake higher than | Not applicable |
| | uptake in normal marrow but | |
| | reduced compared with baseline | |
| | (diffuse uptake compatible with | |
| | reactive changes from | |
| | chemotherapy allowed). If there | |
| | are persistent focal changes in | |
| | the marrow in the context of a | |
| | nodal response, consideration | |
| | should be given to further | |
| | evaluation with MRI or biopsy | |
| | or an interval scan | |
| No response or | | |
| stable disease | No metabolic response | Stable disease |
| Target | Score 4 or 5 with no | <50% decrease from baseline in SPD of |
| nodes/nodal | significant change in FDG | up to 6 dominant, measurable nodes and |
| masses, | uptake from baseline at interim | extranodal sites; no criteria for |
| extranodal | or end of treatment | progressive disease are met |
| lesions | | |
| Nonmeasured | Not applicable | No increase consistent with progression |
| lesion | | |
| Organ | Not applicable | No increase consistent with progression |
| enlargement | | |
| New lesions | None | None |
| Bone marrow | No change from baseline | Not applicable |
| Progressive | | Progressive disease requires at |
| disease | Progressive metabolic disease | least 1 of the following |
| Individual target | Score 4 or 5 with an | PPD progression: |
| nodes/nodal | increase in intensity of | An individual node/lesion must be abnormal |
| masses | uptake from baseline and/or | with: |
| Extranodal | New FDG-avid foci | LDi >1.5cm and |
| lesions | consistent with lymphoma | Increase by ≥50% from PPD nadir and |
| | at interim or end-of- | An increase in LDi or SDi from nadir |
| | treatment assessment | 0.5 cm for lesions ≤2 cm |
| | | 1.0 cm for lesions >2 cm |
| | | In the setting of splenomegaly, the |
| | | splenic length must increase by >50% |
| | | of the extent of its prior increase |
| | | beyond baseline (e.g., a 15-cm spleen |
| | | must increase to >16 cm). If no prior |
| | | splenomegaly, must increase by at least |
| | | 2 cm from baseline |
| | | New or recurrent splenomegaly |
| Nonmeasured | None | New or clear progression of pre-existing |
| lesion | | nonmeasured lesions |
| New lesions | New FDG-avid foci | Regrowth of previously resolved lesions |
| | consistent with lymphoma | A new node >1.5 cm in any axis |
| | rather than another etiology | A new extranodal site >1.0 cm in any |
| | (eg, infection, | axis; if |
| | inflammation). If uncertain | <1.0 cm in any axis, its presence must |
| | regarding etiology of new | be unequivocal and must be attributable |
| | lesions, biopsy or interval | to lymphoma |
| | scan may be considered | Assessable disease of any size |
| | | unequivocally attributable to lymphoma |
| Bone | New or recurrent FDG- | New or recurrent involvement |
| marrow | avid foci | |

5PS: 5-point scale; CT: computed tomography; FDG: fluorodeoxyglucose; IHC: immunohistochemistry; LDi: longest transverse diameter of a lesion; MRI: magnetic resonance imaging; PET: positron emission tomography; PPD: cross product of the LDi and perpendicular diameter; SDi: shortest axis perpendicular to the LDi; SPD: sum of the product of the perpendicular diameters for multiple lesions.
¹A score of 3 in many subjects indicates a good prognosis with standard treatment, especially if at the time of an interim scan. However, in trials involving PET where de-escalation is investigated, it may be preferable to consider a score of 3 as inadequate response (to avoid undertreatment). Measured dominant lesions: Up to 6 of the largest dominant nodes, nodal masses, and extranodal lesions selected to be clearly measurable in 2 diameters. Nodes should preferably be from disparate regions of the body and should include, where applicable, mediastinal and retroperitoneal areas. Non-nodal lesions include those in solid organs (eg, liver, spleen, kidneys, lungs), GI involvement, cutaneous lesions, or those noted on palpation. Nonmeasured lesions: Any disease not selected as measured, dominant disease and truly assessable disease should be considered not measured. These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging. In Waldeyer's ring or in extranodal sites (e.g., GI tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
2PET 5-Point Scale: 1, no uptake above background; 2, uptake ≤mediastinum; 3, uptake ≥mediastinum but ≤liver; 4, uptake moderately >liver; 5, uptake markedly higher than liver and/or new lesions; X, new areas of uptake unlikely to be related to lymphoma.

International Society for Cutaneous Lymphoma Response Criteria, 2011

The following is adapted from Olsen et al., (2011) J Clin Oncol. 29, 18:2598-607.

TABLE 27
Modified ISCL/EORTC Revisions to the TNMB Classification of MF/SS

| TNMB | |
|---|---|
| Stages | Description of TNMB |
| Skin¹ | |
| T₁ | Limited patches, papules, and/or plaques covering <10% of the skin surface; |
| | may further stratify into T₁ₐ (patch only) v T_{1b} (plaque ± patch) |
| T₂ | Patches, papules, or plaques covering ≥10% of the skin surface; may further |
| | stratify into T₂ₐ (patch only) v T_{2b} (plaque ± patch) |
| T₃ | One or more tumors (≥1 cm diameter) |
| T₄ | Confluence of erythema covering ≥80% body surface area |
| Node² | |
| N₀ | No clinically abnormal lymph nodes; biopsy not required |
| N₁ | Clinically abnormal lymph nodes; histopathology Dutch grade 1 or NCI LN₀₋₂ |
| N₁ₐ | Clone negative |
| N_{1b} | Clone positive |
| N₂ | Clinically abnormal lymph nodes; histopathology Dutch grade 2 or NCI LN₃ |
| N₂ₐ | Clone negative |
| N_{2b} | Clone positive |
| N₃ | Clinically abnormal lymph nodes; histopathology Dutch grade 3-4 or NCI LN₄; |
| | clone positive or negative |
| Nₓ | Clinically abnormal lymph nodes without histologic confirmation or inability |
| | to fully characterize the histologic subcategories |
| Visceral | |
| M₀ | No visceral organ involvement |
| M₁ | Visceral involvement (must have pathology confirmation and organ involved |
| | should be specified) |
| Blood | |
| B₀ | Absence of significant blood involvement: ≤5% of peripheral blood |
| | lymphocytes are atypical (Sézary) cells |
| B₀ₐ | Clone negative |
| B_{0b} | Clone positive |
| B₁ | Low blood tumor burden: >5% of peripheral blood lymphocytes are atypical |
| | (Sézary) cells but does not meet the criteria of B2 |
| B₁ₐ | Clone negative |
| B_{1b} | Clone positive |
| B₂ | High blood tumor burden: ≥1,000/μL Sézary cells with positive clone³; 1 of the |
| | following can be substituted for Sézary cells: CD4/CD8 ≥10, CD4⁺CD7⁻ cells ≥40% |
| | or CD4⁺CD26⁻ cells ≥30% |

EORTC: European Organisation for Research and Treatment of Cancer; ISCL: International Society for Cutaneous Lymphomas; MF: mycosis fungoides; NCI: National Cancer Institute SS: Sézary syndrome; TNMB: tumor-node-metastasis-blood.
¹Patch = any size lesion without induration or significant elevation above the surrounding uninvolved skin: pokiloderma may be present. Plaque = any size lesion that is elevated or indurated: crusting or poikiloderma may be present. Tumor = any solid or nodular lesion ≥1 cm in diameter with evidence of deep infiltration in the skin and/or vertical growth.
²Lymph node classification has been modified from 2007 ISCL/EORTC consensus revisions to include central nodes. Lymph nodes are qualified as abnormal if >1.5 cm in diameter.
³The clone in the blood should match that of the skin. The relevance of an isolated clone in the blood or a clone in the blood that does not match the clone in the skin remains to be determined.

TABLE 42
Modified ISCL/EORTC Revisions to Staging of MF/SS

| Stage | T | N | M | B |
|---|---|---|---|---|
| IA | 1 | 0 | 0 | 0, 1 |
| IB | 2 | 0 | 0 | 0, 1 |
| IIA | 1-2 | 1, 2, X | 0 | 0, 1 |
| IIB | 3 | 0-2, X | 0 | 0, 1 |
| IIIA | 4 | 0-2, X | 0 | 0 |
| IIIB | 4 | 0-2, X | 0 | 1 |
| IVA₁ | 1-4 | 0-2, X | 0 | 2 |
| IVA₂ | 1-4 | 3 | 0 | 0-2 |
| IVB | 1-4 | 0-3, X | 1 | 0-2 |

EORTC: European Organisation for Research and Treatment of Cancer;
ISCL: International Society for Cutaneous Lymphomas;
MF: mycosis fungoides;
SS: Sézary syndrome;
X: clinically abnormal lymph nodes without histologic confirmation or inability to fully characterize histologic subcategories

Diagnosis

Histopathologic diagnosis is confirmed in a skin biopsy representative of current disease by a pathologist with expertise in cutaneous lymphoma. For Sézary syndrome (SS; defined as meeting T4 plus B2 criteria), where the biopsy of erythrodermic skin may only reveal suggestive but not diagnostic histopathologic features, the diagnosis may be based on either a node biopsy or fulfillment of B2 criteria including a clone in the blood that matches that of the skin. For early patch stage mycosis fungoides (MF) where the histological diagnosis by light microscopic examination is not confirmed, diagnostic criteria that have been recommended by the ISCL should be used.

Evaluation

Pretreatment evaluation and scoring of response parameters is done at baseline (day 1 of treatment), and not at screening.

All responses are at least 4 weeks in duration.

Skin Assessment, Scoring, and Definition of Response

The Severity Weighted Assessment Tool (SWAT) or the modified SWAT (mSWAT) is used for skin scoring.

The definition of response is presented in Table 28.

TABLE 28
Response in Skin

| Response | Definition |
|---|---|
| Complete | 100% clearance of skin lesions¹ |
| response | |
| Partial response | 50%-99% clearance of skin disease from baseline without new tumors (T3) |
| | in subjects with T1, T2 or T4 only skin disease |
| Stable disease | <25% increase to <50% clearance in skin disease from baseline without |
| | new tumors (T3) in subjects with T1, T2, or T4 only skin disease |
| Progressive | ≥25% increase in skin disease from baseline or New tumors (T3) in subjects |
| disease² | with T1, T2 or T4 only skin disease or Loss of response: in those with |
| | complete or partial response, increase of skin score of greater than the sum |
| | of nadir plus 50% baseline score |
| Relapse | Any disease recurrence in those with complete response |

NOTE
Based on modified Severity Weighted Assessment Tool score.
¹A biopsy of normal appearing skin is unnecessary to assign a complete response. However, a skin biopsy should be performed of a representative area of the skin if there is any question of residual disease (persistent erythema or pigmentary change) where otherwise a complete response would exist. If histologic features are suspicious or suggestive of mycosis fungoides/Sézary syndrome (see histologic criteria for early mycosis fungoides), the response should be considered a partial response only.
²Whichever criterion occurs first.

Lymph Node Assessment, Scoring, and Definition of Response

Peripheral lymph nodes: The full tumor-node-metastasis-blood (TNMB) status of participants is characterized, and computed tomography (CT) imaging is recommended, with the caveat that considerable inter-observer variability exists. Magnetic resonance imaging (MRI) is an alternative to CT.

Central lymph nodes: If there is evidence of enlarged central nodes (defined as >1.5 cm diameter in the long axis or >1.0 cm diameter in the short axis), and confirmation of involvement with MF/SS by biopsy (i.e., excisional, fine-needle aspirate, or core biopsy), then all central nodes are tracked thereafter in the same way as peripheral nodes (product of the longest bidimensional measurements of all enlarged nodes)

The definition of response is presented in Table 29.

TABLE 29
Response in Lymph Nodes

| Response | Definition¹ |
|---|---|
| Complete | All lymph nodes are now ≤1.5 cm in greatest transverse (long axis) diameter |
| response | by method used to assess lymph nodes at baseline or biopsy negative for |
| | lymphoma; in addition, lymph nodes that were N3 classification and ≤1.5 cm |
| | in their long axis and >1 cm in their short axis at baseline, must now be ≤1 |
| | cm in their short axis or biopsy negative for lymphoma |
| Partial response | Cumulative reduction ≥50% of the SPD of each abnormal lymph node at |
| | baseline and no new lymph node >1.5 cm in the diameter of the long axis or |
| | >1.0 cm in the diameter of the short axis if the long axis is 1-1.5 cm diameter |
| Stable disease | Fails to attain the criteria for CR, PR, and PD |
| Progressive | ≥50% increase in SPD from baseline of lymph nodes or |
| disease² | Any new node >1.5 cm in the long axis or >1 cm in the short axis if 1-1.5 cm |
| | in the long axis that is proven to be N3 histologically or |
| | Loss of response: >50% increase from nadir in SPD of lymph nodes in those |
| | with PR |
| Relapse | Any new lymph node >1.5 cm in the long axis in those with CR proven to be |
| | N3 histologically |

CR: complete response; PD: progressive disease; PR: partial response; SPD: sum of the maximum linear dimension (major axis) × longest perpendicular dimension (minor axis).
¹Peripheral and central lymph nodes.
²Whichever criterion occurs first.

Visceral Disease Assessment, Scoring, and Definition of Response

Biopsy confirmation at baseline is recommended for all forms of visceral disease except for liver and spleen involvement, which may be diagnosed by imaging studies. Of note, bone marrow aspirate/trephine biopsies are not considered obligatory for either evaluation or response assessment. There may be limitations in corroborating a CR in viscera by CT alone, and in those cases, a confirmatory biopsy may be necessary or lacking this, no CR assessment can be made.

The definition of response is presented in Table 30.

TABLE 30
Response in Viscera

| Response | Definition |
|---|---|
| Complete | Liver or spleen or any organ considered involved at baseline should not be |
| response | enlarged on physical exam and should be considered normal by imaging; no |
| | nodules should be present on imaging of liver or spleen; any post-treatment |
| | mass must be determined by biopsy to be negative for lymphoma |
| Partial response | ≥50% regression in any splenic or liver nodules, or in measurable disease |
| | (SPD) in any organs abnormal at baseline; no increase in size of liver or |
| | spleen and no new sites of involvement |
| Stable disease | Fails to attain the criteria for CR, PR, or PD |
| Progressive | >50% increase in size (SPD) of any organs involved at baseline or New |
| disease¹ | organ involvement or |
| | Loss of response: >50% increase from nadir in the size (SPD) of any |
| | previous organ involvement in those with PR |
| Relapse | New organ involvement in those with CR |

CR: complete response; PR: partial response; SPD: sum of the maximum linear dimension (major axis) × longest perpendicular dimension (minor axis); SD: stable disease; PD: progressive disease.
¹Whichever criterion occurs first.

Blood Assessment, Scoring, and Definition of Response

The absolute number of CD4⁺CD26⁻ cells determined by flow cytometry is the most reasonable, quantifiable measure of potential blood involvement in MF/SS. In CD26+ subjects, CD4⁺CD7⁻ T cells would be an alternate population to monitor.

Based on an upper limit of normal value of 1,600/μL for CD4 cells in the blood, an absolute count of lower than 250/μL CD4+/CD26⁻ or CD4+CD7⁻ cells would appear to be a normal value for these CD4 subsets and could also be used to define the absence of or normalization of blood involvement (B₀). Alternately, an absolute Sézary cell count is an optional method when good quality smears are interpreted by a single qualified reader with lower than 250/μL and higher than 1,000/μL of Sézary cells being reasonable determinants of B₀ and B₂.

The definition of response is presented in Table 31.

TABLE 31
Response in Blood¹

| Response | Definition |
|---|---|
| Complete response ² | B₀ |
| Partial response ³ | >50% decrease in quantitative measurements of blood tumor |
| | burden from baseline in those with high tumor burden at baseline |
| | (B₂) |
| Stable disease | Fails to attain criteria for CR, PR, or PD |
| Progressive disease ⁴ | B₀ to B₂ or |
| | >50% increase from baseline and at least 5,000 neoplastic |
| | cells/μL or |
| | Loss of response: in those with PR who were originally B2 at |
| | baseline, >50% increase from nadir and at least 5,000 neoplastic |
| | cells/μL |
| Relapse | Increase of neoplastic blood lymphocytes to ≥B₁ in those with CR |
| | CR: complete |

CR: complete response; PR: partial response; SD: stable disease; PD: progressive disease.
¹As determined by absolute numbers of neoplastic cells/μL.
² If a bone marrow biopsy was performed at baseline and determined to unequivocally be indicative of lymphomatous involvement, then to confirm a global CR where blood assessment now meets criteria for B₀, a repeat bone marrow biopsy must show no residual disease or the response should be considered a PR only.
³ There is no PR in those with B1 disease at baseline as the difference within the range of neoplastic cells that define B1 is not considered significant and should not affect determination of global objective response.
⁴ Whichever occurs first.

Global Response Score Definition

Consensus global response score for MF/SS is presented in Table 32.

TABLE 32
Global Response Score

| | | | | | |
|---|---|---|---|---|---|
| Global | | | | | |
| Score¹ | Definition | Skin | Nodes | Blood | Viscera |

| | | | |
|---|---|---|---|
| CR | Complete disappearance of | CR | All categories have CR/NI |
| | all clinical evidence of | | |
| | disease | | |
| PR | Regression of measurable | CR | All categories do not have a CR/NI and no |
| | disease | | category has a PD |
| | | PR | No category has a PD and if any category |
| | | | involved at baseline, at least 1 has a CR or PR |
| SD | Failure to attain CR, PR, or | PR | No category has a PD and if any category |
| | PD representative of all | SD | involved at baseline, no CR or PR in any |
| | disease | | CR/NI, PR, SD in any category and no category |
| | | | has a PD |
| PD | Progressive disease | PD | PD in any category |
| Relapse | Recurrence disease in prior | | Relapse in any category |
| | CR | | |

CR: complete response; NI: noninvolved; PR: partial response; PD: progressive disease; SD: stable disease.
¹It is recommended that not only the proportion of subjects who achieve a response or an unfavorable outcome be calculated but a life table account for the length of the interval during which each subject is under observation also be generated.

### 7. Treatment

Daratumumab Administration

Subjects in Part A2 (dose escalation with daratumumab added to the lymphodepletion regimen), in Part A4 (dose escalation with daratumumab added to the lymphodepletion regimen and with additional CTX130 infusion on Day 5 [+2 days]), and in Part A6 (dose escalation with daratumumab added to the lymphodepletion regimen and with Day 35 CTX130 consolidation) receive 1 dose of daratumumab (an anti-CD38 monoclonal antibody) 16 mg/kg by IV infusion or 1800 mg by subcutaneous injection at least 12 hours prior to starting LD chemotherapy and within 10 days prior to CTX130 infusion. In Parts A2 and A4, a second dose of daratumumab (16 mg/kg IV or 1800 mg SC) are administered on Day 21 and a third dose on Day 42, if the criteria for receiving additional daratumumab doses are met. In Part A6, daratumumab is not administered on Day 21 and Day 42. Subjects in Part A6 only receive daratumumab as part of the LD regimen prior to infusion of CTX130.

The first dose of daratumumab is delayed if any of the criteria described herein are present.

To be considered for any additional doses of daratumumab, subjects in Parts A2 and A4 must meet the following criteria at the time of daratumumab dosing:
No severe or unmanageable toxicity with prior daratumumab doses
No disease progression without significant clinical benefit
No ongoing uncontrolled infection
Platelet count ≥25,000 cells/μL (non-transfused)
No ≥grade 3 neutropenia
No CD4+ T cell count<100/μL

To be considered for additional courses of treatment with CTX130, subjects in Parts A2 and A4 must meet the criteria described herein, and daratumumab may be administered prior to LD chemotherapy if no unacceptable toxicity occurred with prior daratumumab doses.

Daratumumab administration (including pre- and postinfusion medications, preparation, infusion rates, and postinfusion monitoring) is performed according to the local prescribing information unless otherwise stated. To facilitate administration, the first 16 mg/kg IV dose may be split to 8 mg/kg IV over 2 consecutive days per daratumumab prescribing information (DARZALEX, USPI 2019).

After at least 3 subjects are treated at a specific CTX130 dose with daratumumab, additional subjects may be enrolled at a specific dose level with a lower dose of daratumumab (8 mg/kg IV).

Disease response is assessed in accordance with Lugano response criteria (Cheson et al., 2014) for subjects with PTCL-NOS, ALCL, leukemic and lymphomatous ATLL, AITL, and DLBCL, and ISCL response criteria (Olsen et al., 2011) for subjects with SS or MF before repeat dosing with daratumumab.

Daratumumab Administration Reactions

To reduce the risk of administration reactions with daratumumab IV or SC, 1 to 3 hours prior to infusion subjects are premedicated with corticosteroids (e.g., IV methylprednisolone 100 mg or equivalent; following the second infusion, the dose of corticosteroid may be reduced [oral or IV methylprednisolone 60 mg], antipyretics (e.g., oral acetaminophen [paracetamol] 650-1000 mg, or equivalent), and antihistamines (e.g., oral or IV diphenhydramine hydrochloride [or another H1-antihistamine] 25-50 mg, or equivalent). Use of any alternative prophylaxis regimen must be discussed and approved.

Subjects are monitored frequently during the entire administration of daratumumab. For infusion reactions of any grade/severity, infusion is interrupted immediately, and symptoms managed. If an anaphylactic reaction or life-threatening (grade 4) reaction occurs, therapy is permanently discontinued and appropriate emergency care administered. For subjects with grade 1, 2, or 3 reactions, after symptom resolution, the infusion rate is reduced when restarting the infusion, as described in the approved prescribing information or per site practice.

To reduce the risk of delayed infusion reactions, oral corticosteroids (20 mg methylprednisolone or equivalent dose of an intermediate-acting or long-acting corticosteroid in accordance with local standards) is administered to subjects following the daratumumab administration, per local prescribing information.

For the Day 21 or Day 42 dose of daratumumab, only intermediate-acting corticosteroids (i.e., prednisone, methylprednisone) are used to reduce the risk of interference with the CTX130.

If a subject has an unresolved event of infusion reaction after daratumumab treatment, the CTX130 infusion is delayed and discussed with the medical monitor prior to proceeding.

Additional Considerations

Daratumumab has been associated with herpes zoster (2%) and hepatitis B (1%) reactivation in patients with multiple myeloma.

Supportive care is provided according to the approved local prescribing information. Daratumumab binds to CD38 on red blood cells and results in a positive indirect antiglobulin test (indirect Coombs test). Typing and screening of blood occur per the approved prescribing information to prevent interference with blood compatibility testing.

Lymphodepleting Chemotherapy

All subjects receive LD chemotherapy prior to each infusion with CTX130 during each course of treatment in Parts A1 through A4, and prior to each CTX130 infusion in Parts A5 and A6. For Parts A5 and A6, the second (Day 35) CTX130 infusion as well as the single additional CTX130 infusion after the first course of treatment may be administered without LD chemotherapy if the subject is experiencing significant cytopenia.

LD chemotherapy consists of:
Fludarabine 30 mg/m2 IV daily for 3 doses AND
Cyclophosphamide 500 mg/m2 IV daily for 3 doses.

Adult subjects with moderate impairment of renal function (CrCl 50-70 mL/min/1.73 m²) receive a reduced dose of fludarabine by at least 20% or in accordance with local prescribing information.

Both agents are started on the same day and administered for 3 consecutive days. Prior to the initial infusion with CTX130, subjects in all Parts start LD chemotherapy within 7 days of study enrollment. Subjects in Parts A5 and A6, who achieve CR, PR, SD, or PD with clinical benefit at Day 28 scan (based on Lugano or Olsen criteria as appropriate), should complete LD chemo a maximum of 7 days and a minimum of 48 hours prior to receiving the second (Day 35) CTX130 infusion. An interruption/delay such that LD chemotherapy cannot be completed in 3 consecutive days may result in restart of LD chemotherapy from LD Day 1.

Reference the current full prescribing information for fludarabine and cyclophosphamide for guidance regarding the storage, preparation, administration, supportive care instructions, and toxicity management associated with LD chemotherapy.

LD chemotherapy, or the first daratumumab dose (for subjects in Parts A2, A4, or A6) is delayed if any of the following signs or symptoms are present on any of the planned dosing days (i.e., each of the 3 days of LD chemotherapy and the day of daratumumab dosing for subjects in Parts A2, A4, or A6):
Change in performance status to ECOG>1.
Significant worsening of clinical status that increases the potential risk of AEs associated with LD chemotherapy.
   Clinically significant worsening of any cytopenia
   Clinically significant increase of transaminase levels (e.g., >3×ULN)
   Clinically significant increase of total bilirubin (e.g., >2×ULN)
   Clinically significant increase in serum creatinine
Requirement for supplemental oxygen to maintain a saturation level of >92%.
New uncontrolled cardiac arrhythmia.
Hypotension requiring vasopressor support.
Active infection: Positive blood cultures for bacteria, fungus, or virus not responding to treatment, or negative culture but active infection.
Platelet count≤25,000/mm³, absolute neutrophil count≤500/mm³.
Any neurological toxicity

Cytopenia and Lymphodepleting Chemotherapy

During the first course of treatment, in subjects with significant cytopenias (ANC<500/mm3 and/or platelets <25,000 cells/μL), the investigator may omit LD chemotherapy prior to the Day 35 CTX130 infusion (in Parts A5 and A6), or prior to CTX130 infusion(s) occurring after the first course of treatment. Individual cases may be discussed with the medical monitor if there is strong evidence of cytopenia being due to alternative etiologies or expected recovery (including underlying malignancy).

Subjects who receive additional infusions with LD chemotherapy for prolonged cytopenia are continuously evaluated. After at least 6 subjects receive an additional infusion with LD chemotherapy in cohort expansion, if >50% of subjects have prolonged grade 3 or 4 cytopenia (i.e., lasting more than 28 days postinfusion), use of LD chemotherapy prior to additional infusions is reconsidered while current schema are reassessed and an alternate regimen is proposed.

In cohort expansion, in subjects who on Day 28 are eligible for and subsequently receive an additional CTX130 infusion without LD chemotherapy, if 8 subjects are infused and all have progressive disease or there is no improvement in response observed based on imaging 28 days after the last infusion, the option of subsequent infusions without LD chemotherapy is removed and subsequent subjects receive additional infusions of CTX130 with LD chemotherapy or do not receive additional infusions of CTX130.

Administration of CTX130

CTX130 consists of allogeneic T cells modified with CRISPR-Cas9, resuspended in cryopreservative solution (CryoStor CS5), and supplied in a 6-mL infusion vial. A flat dose of CTX130 (based on number of CAR⁺ T cells) is administered as a single IV infusion. A dose limit of 7×10⁴ TCR⁺ cells/kg per infusion is imposed for all dose levels, which determines the minimum weight for dosing. The total dose may be contained in multiple vials. Infusion should preferably occur through a central venous catheter. A leukocyte filter must not be used.

Prior to the start of CTX130 infusion, the site pharmacy must ensure that 2 doses of tocilizumab and emergency equipment are available for each specific subject treated. Subjects should be premedicated per the site standard of practice with acetaminophen orally (PO) (i.e., paracetamol or its equivalent per site formulary) and diphenhydramine hydrochloride IV or PO (or another H1-antihistamine per site formulary) approximately 30 to 60 minutes prior to CTX130 infusion.

Prophylactic systemic corticosteroids should not be administered, as they may interfere with the activity of CTX130.

For Parts A and B, CTX130 infusion is delayed if any of the following signs or symptoms are present:
Change in performance status to ECOG>1.
New active uncontrolled infection.
Significant worsening of clinical status that increases the potential risk of AEs associated with allogenic CAR T cell infusion, e.g.:
   Clinically significant increase of transaminase levels (e.g., >3×ULN)
   Clinically significant increase of total bilirubin (e.g., >2×ULN)
   Clinically significant increase in serum creatinine
Any neurological toxicity
   For Parts A3 and A4, in addition to the above criteria, the second CTX130 infusion (Day 5 [+2 days]) is not administered if any of the following signs or symptoms are present:
CRS following the first CTX130 infusion, except grade≤2 CRS (per American Society for Transplantation and Cellular Therapy [ASTCT] criteria) lasting <48 hours (the subject must be free of any symptoms for 48 hours prior to the second infusion)
Any medical condition that would put the subject at risk

CTX130 is administered at least 48 hours (but no more than 7 days) after the completion of LD chemotherapy. For subjects who are considered for an additional course of treatment with CTX130 (Parts A1 through A4), who meet the criteria for receiving a second/third infusion of CTX130 (Parts A5 and A6), who during a previous course of treatment experienced either a delay in LD regimen due to failure to meet the criteria described herein, or who experience a delay in CTX130 infusion due to failure to meet the criteria described herein, discussion with the medical monitor is required prior to initiation of screening for the CTX130.

Additional Courses of Treatment with CTX130: Parts A1 Through A4

In Parts A1 through A4 of this study, subjects may be considered for additional courses of treatment with CTX130. To be considered for additional courses of treatment, subjects must have either:
1. Achieved a CR after the previous course of treatment, and within 2 years of initial CTX130 infusion have an increase (compared with nadir) of at least 10% of the tumor burden by at least one of the following: 1) modified Severity Weighted Assessment Tool (mSWAT), or 2) radiology: sum of the product of the perpendicular diameters (SPD), or
3) blood tumor burden as determined by site investigators or formal progression by Lugano or ISCL criteria as appropriate.
or
2. Achieved PR, SD, or PD with clinical benefit

Decisions on whether subjects can receive additional courses of treatment with CTX130 is based upon local radiology scans and global or overall disease assessment appropriate to each subject's specific disease. Note that the first day of LD chemotherapy in the second (or third) course of treatment must be at least 28 days after the last day of LD chemotherapy in the previous course of treatment.

To receive additional courses of treatment, subjects in Parts A1 through A4 must meet the following criteria:
Confirmation that tumor is CD70+(based on local or central assessment) if a lesion is available that is amenable to biopsy
No prior DLT during dose escalation
No prior grade≥3 CRS without resolution to grade≤2 within 72 hours following CTX130 infusion
No prior grade≥1 GvHD following CTX130 infusion
No prior grade≥2 ICANS following CTX130 infusion
Meet initial study inclusion criteria (#1, #2, #7-12) and exclusion criteria (#4 [except prior treatment with CAR T cells]-19) as described herein
Meet criteria for daratumumab dosing (Parts A2 and A4 only), LD chemotherapy, and CTX130 infusion as described herein
   Subjects who receive additional courses of treatment with CTX130 receive 3 consecutive days of LD chemotherapy and should be followed per the schedule of assessments consistent with the first course of treatment, including the 7 days of hospitalization post CTX130 infusion. Note that additional courses of treatment may be administered without LD chemotherapy if subject is experiencing significant cytopenia (ANC<500/mm3 and/or platelets <25,000 cells/μL).

All procedures (screening through Day 28) must be repeated, with the following exceptions:
Not all central lab screening samples may be required; refer to Laboratory Manual for details. If additional CTX130 infusion is planned within 30 days of the Day 28 visit post prior infusion, no additional central collections at screening are required.
Local laboratory testing performed within 14 days of planned start of lymphodepletion may be used to confirm eligibility for subsequent CTX130 infusions.
Echocardiogram (unless new cardiac signs or symptoms) is not required.
Radiological (PET-CT or CT) disease assessments do not need to be repeated if performed within 28 days prior to next CTX130 infusion
Bone marrow biopsy/aspirate do not need to be repeated unless clinically indicated
Brain MRI does not need to be repeated

In Parts A2 and A4, daratumumab may be administered with additional courses of CTX130 treatment following the same administration schedule as described herein.

Additional considerations include the following:
If PD occurred prior to an additional course of treatment with CTX130, the most
recent PET/CT scan serves as the new baseline for disease response evaluation. CTX130 infusion during the additional course of treatment must occur within 28 days of that scan.
If SD or PR is the response assessment prior to an additional course of treatment with CTX130, the baseline scan for the SD or PR assessment ontinues to be used for disease response evaluation.
Subjects in the dose escalation cohorts who undergo additional courses of treatment with CTX130 receive a CTX130 dose that at time of administration has been deemed safe by the SRC (minimum n=3)
Subjects in the expansion cohorts will receive additional courses of treatment with
CTX130 per the RPBD regimen.

Parts A3 and A4: Second Infusion of CTX130

Subjects in Parts A3 and A4 receive a second CTX130 infusion without LD chemotherapy 4 days (+2 days) after the first CTX130 infusion at the same dose level as on Day 1. In the event of a dose delay outside the +2-day window, the timing of the second dose may be discussed, with the second dose to occur no later than Day 15.

Part A3 begins with CTX130 infusion at a dose level that has been deemed safe in Part A1, and Part A4 begins with CTX130 infusion at a dose level that has been deemed safe in Part A2. For both Part A3 and Part A4, sentinel dosing is implemented for the starting dose level only, i.e., the first 2 subjects are treated in a staggered manner, such that the second subject only receives CTX130 after the previous subject has completed the DLT evaluation period. The second and third subjects may be dosed concurrently. In subsequent dose levels or expansion of the same dose level, cohorts of up to 3 subjects may be enrolled and dosed concurrently.

The second CTX130 infusion (Day 5 [+2 days]) is delayed in Parts A3/A4 if any of the following signs or symptoms are present:
Change in performance status to ECOG>1.
New active uncontrolled infection.
Significant worsening of clinical status that, in the opinion of the investigator, increases the potential risk of AEs associated with allogeneic CAR T cell infusion e.g.:
   Clinically significant increase of transaminase levels (e.g., >3×ULN)
   Clinically significant increase of total bilirubin (e.g., >2×ULN)
   Clinically significant increase in serum creatinine
Any acute neurological toxicity

For Parts A3 and A4, in addition to the above criteria, the second CTX130 infusion (Day 5 [+2 days]) is not administered if any of the following signs or symptoms are present:
CRS following the first CTX130 infusion, except grade≤2 CRS (per American Society for Transplantation and Cellular Therapy [ASTCT] criteria) lasting <48 hours (the subject must be free of any symptoms for 48 hours prior to the second infusion)
Any medical condition that would put the subject at risk

Parts A5 and A6: Second Infusion of CTX130

In Parts A5 and A6, a second infusion of CTX130 on Day 35 (−7 days/+21 days) is administered to subjects who achieve CR, PR, SD, or PD with clinical benefit at Day 28 scan (based on Lugano or Olsen criteria as appropriate). In Part A5, subjects receive LD chemotherapy prior to the second infusion of CTX130 on Day 35, and in Part A6, subjects receive daratumumab+LD chemotherapy prior to the second infusion of CTX130 on Day 35. Note that the second CTX130 infusion may be administered without LD chemotherapy if subject is experiencing significant cytopenia.

To receive the second (Day 35) CTX130 infusion during the first course of treatment, subjects in Parts A5 and A6 must meet the following criteria:
No prior DLT during dose escalation
No prior grade≥3 CRS without resolution to grade≤2 within 72 hours following CTX130 infusion
No prior grade≥1 GvHD following CTX130 infusion
No prior grade≥2 ICANS following CTX130 infusion
Meet initial study inclusion criteria (#1, #2, #7-12) and exclusion criteria (#4 [except prior treatment with CAR T cells]-19) as described herein
Meet criteria for daratumumab dosing (Part A6 only), LD chemotherapy, and CTX130 infusion as described herein

Subjects who receive the second CTX130 infusion should be followed per the schedule of assessments consistent with the initial dosing. All procedures (screening through Day 28) must be repeated, with the following exceptions:
Not all central lab screening samples may be required. If additional CTX130 infusion is planned within 30 days of the Day 28 visit post prior infusion, no additional central collections at screening are required.
Local laboratory testing performed within 14 days of planned start of lymphodepletion may be used to confirm eligibility for subsequent CTX130 infusions.
Echocardiogram (unless new cardiac signs or symptoms) is not required.
Radiological (PET-CT or CT) disease assessments do not need to be repeated if performed within 28 days prior to next CTX130 infusion
Bone marrow biopsy/aspirate do not need to be repeated unless clinically indicated
Brain MRI does not need to be repeated

All subjects should meet the criteria specified in the protocol prior to both the initiation of LD chemotherapy (or daratumumab as applicable) and infusion of CTX130. Note that in Part A6, daratumumab is not administered on Day 21 and Day 42. Subjects in Part A6 only receive daratumumab as part of the LD regimen prior to infusion of CTX130.

Single Additional Infusion of CTX130 after First Course of Treatment

Subjects in Parts A5 and A6 undergo a first course of treatment, including CTX130 infusion on Day 1, and if eligible, CTX130 infusion on Day 35. Subjects in Parts A5 and A6 do not receive additional (second or third) courses of treatment; however, after the first course of treatment, an optional single additional infusion of CTX130 can be administered with LD chemotherapy (and with daratumumab if applicable) after 1) loss of complete response (CR) within the first 2 years after initial infusion of CTX130, or 2) partial response (PR), stable disease (SD), or progressive disease (PD) with clinical benefit as determined by the investigator. Note that the first day of LD chemotherapy prior to the single additional infusion of CTX130 must be at least 28 days after the last day of LD chemotherapy in the first course of treatment.

To receive the additional CTX130 infusion after the first course of treatment, subjects in Parts A5 and A6 must meet the following criteria:
Confirmation that tumor is CD70+(based on local or central assessment) if a lesion is available that is amenable to biopsy
No prior DLT during dose escalation
No prior grade≥3 CRS without resolution to grade≤2 within 72 hours following CTX130 infusion
No prior grade>1 GvHD following CTX130 infusion
No prior grade≥2 ICANS following CTX130 infusion
Meet initial study inclusion criteria (#1, #2, #7-12) and exclusion criteria (#4 [except prior treatment with CAR T cells]-19) as described herein
Meet criteria for daratumumab dosing (Part A6 only), LD chemotherapy, and CTX130 infusion as described herein

Subjects who receive the single additional infusion of CTX130 receive daratumumab if applicable, 3 consecutive days of LD chemotherapy, and should be followed per the schedule of assessments consistent with the first course of treatment, including the 7 days of hospitalization post CTX130 infusion.

The exceptions to (screening through Day 28) repeat assessment requirements are the same as those described herein for the second (Day 35) infusion of CTX130 in Parts A5/A6 and for additional courses of treatment for Parts A1 through A4.

The additional CTX130 infusion after the first course of treatment in Parts A5 and A6 is allowed at a CTX130 dose level that has been deemed safe and that is greater than or equal to the CTX130 dose level administered during the first course of treatment. Note that this additional CTX130 infusion may be administered without LD chemotherapy if subject is experiencing significant cytopenia.

CTX130 Postinfuion Monitoring

Following CTX130 infusion, subjects' vitals should be monitored every 30 minutes for 2 hours after infusion or until resolution of any potential clinical symptoms.

Subjects in Part A are hospitalized for a minimum of 7 days after CTX130 infusion, or longer if required. Postinfusion hospitalization in Part B is considered based on the safety information obtained during dose escalation and may be performed. In Parts A and B, the length of hospitalization may be extended where required by local regulation or site practice. In both Parts A and B, subjects must remain in proximity of the investigative site (i.e., 1-hour transit time) for at least 28 days after CTX130 infusion.

Management of acute CTX130-related toxicities should occur ONLY at the study site. Subjects are monitored for signs of CRS, TLS, neurotoxicity, GvHD, and other AEs according to the schedule of assessments (Tables 20, 21, 40, and 41). Subjects remain hospitalized until CTX130-related nonhematologic toxicities (e.g., fever, hypotension, hypoxia, ongoing neurological toxicity) return to grade 1. Subjects may remain hospitalized for longer periods.

Prior and Concomitant Medications

Allowed Medications and Procedures (Concomitant Treatments)

Necessary supportive measures for optimal medical care are given throughout the study, including IV antibiotics to treat infections, growth factors, blood components, etc., except for prohibited medications.

Medications to inhibit bone absorption such as bisphosphonates or RANKL inhibitor are allowed for symptomatic therapy including hypercalcemia.

All concurrent therapies, including prescription and nonprescription medication, and medical procedures are recorded from the date of signed informed consent through 3 months after each CTX130 infusion. Beginning 3 months post-CTX130 infusion, only the following selected concomitant medications are collected: vaccinations, anticancer treatments

(e.g., chemotherapy, radiation, immunotherapy), immunosuppressants (including steroids), and any investigational agents.

Prohibited/Restricted Medications and Procedures

The following medications are prohibited during certain periods of the study as specified below:
Prohibited Within 28 Days Prior to Enrollment to 3 Months After CTX130 Infusion
   Live vaccines
   Herbal medicine as part of traditional Chinese medicine or non-over-the-counter herbal remedies
Prohibited Throughout the Study Until the Start of New Anticancer Therapy
   Any immunosuppressive therapy unless recommended to treat CRS or ICANS or if previously discussed with and approved by the medical monitor.
   Corticosteroid therapy at a pharmacologic dose (>10 mg/day of prednisone or equivalent doses of other corticosteroids) and other immunosuppressive drugs are avoided after CTX130 administration unless medically indicated to treat new toxicity or as part of management of CRS or neurotoxicity associated with CTX130. Use of oral corticosteroids before and after daratumumab administration is permitted to prevent infusion reactions.
   Any anticancer therapy (e.g., chemotherapy, immunotherapy, targeted therapy, radiation, or other investigational agents) other than daratumumab (Part A2) or LD chemotherapy prior to disease progression. Palliative radiation therapy for symptom management is permitted prior to CTX130 infusion.
Prohibited Within the First Month After CTX130 Infusion
   Granulocyte-macrophage colony-stimulating factor (GM-CSF) following CTX130 infusion due to the potential to worsen symptoms of CRS. Care should be taken with administration of granulocyte colony-stimulating factor (G-CSF) following CTX130. G-CSF administration during and/or after LD chemotherapy should be discussed prior to administration and should be stopped 18 hours prior to CTX130 infusion if G-CSF is given IV and stopped 24 hours prior to CTX130 infusion if G-CSF is given subcutaneously.
   Within the first 28 days of CTX130 infusion: self-medication by the subject with antipyretics (e.g., acetaminophen, aspirin).
Prohibited 3 Months Prior and During the Treatment with CTX130, and up to 6 Months After CTX130 Infusion
   CCR-4-directed monoclonal antibodies, due to the increased risk of GvHD. Note that while other CCR-4-directed monoclonal antibodies are prohibited starting 3 months prior to CTX130 treatment, mogamulizumab is prohibited starting 50 days prior to CTX130 treatment.

### 8. Toxicity Management

General Guidance

Subjects are closely monitored for at least 28 days after CTX130 infusion. Significant toxicities have been reported with autologous CAR T cell therapies.

Although this is a first-in-human study and the clinical safety profile of CTX130 has not been described, the following general recommendations are provided based on prior experience with autologous CAR T cell therapies:
Fever is the most common early manifestation of CRS; however, subjects may also experience weakness, hypotension, or confusion as first presentation.
Diagnosis of CRS should be based on clinical symptoms and NOT laboratory values.
In subjects who do not respond to CRS-specific management, always consider sepsis and resistant infections. Subjects should be continually evaluated for resistant or emergent bacterial infections, as well as fungal or viral infections.
CRS, HLH, and TLS may occur at the same time following CAR T cell infusion. Subjects should be consistently monitored for signs and symptoms of all the conditions and managed appropriately.
ICANS may occur at the time of CRS, during CRS resolution, or following resolution of CRS. Grading and management of ICANS is performed separately from CRS.
Tocilizumab must be administered within 2 hours from the time of order.

In addition to toxicities observed with autologous CAR T cells, signs of GvHD are monitored closely due to the allogeneic nature of CTX130.

### Toxicity-Specific Guidance

CTX130 Infusion-Related Reactions

Infusion-related reactions have been reported in autologous CAR T cell trials, including transient fever, chills, and/or nausea most commonly occurring within 12 hours after administration.

CTX130 is formulated with CryoStor CS5, a well-established cryopreservant medium that contains 5% dimethyl sulfoxide (DMSO). Histamine release associated with DMSO can result in adverse effects such as nausea, vomiting, diarrhea, flushing, fevers, chills, headache, dyspnea, or rashes. In most severe cases, it can also cause bronchospasm, anaphylaxis, vasodilation and hypotension, and mental status changes.

If an infusion reaction occurs, acetaminophen (paracetamol) and diphenhydramine hydrochloride (or another H1-antihistamine) may be repeated every 6 hours after CTX130 infusion, as needed.

Nonsteroidal anti-inflammatory medications may be prescribed as needed if the subject continues to have fever not relieved by acetaminophen. Systemic steroids should NOT be administered except in cases of life-threatening emergency, as this intervention may have a deleterious effect on CAR T cells.

Infection Prophylaxis and Febrile Reaction

Infection prophylaxis should be managed according to the institutional standard of care. In the event of febrile reaction, an evaluation for infection should be initiated and the subject managed appropriately with antibiotics, fluids, and other supportive care as medically indicated and determined by the treating physician. Viral and fungal infections should be considered throughout a subject's medical management if fever persists. If a subject develops sepsis or systemic bacteremia following CTX130 infusion, appropriate cultures and medical management should be initiated. Additionally, consideration of CRS should be given in any instances of fever following CTX130 infusion within 28 days postinfusion.

For Parts A2, A4, and A6, prophylaxis for herpes zoster and hepatitis B reactivation in the setting of daratumumab treatment is strongly recommended, as per prescribing information. For subjects receiving multiple CTX130 infusions with LD chemotherapy, pneumocystis jirovecii prophylaxis is recommended.

Subjects undergoing CAR T therapy have an increased risk of infections due to underlying malignancy, prior antitumor therapies, daratumumab treatment, lymphodepleting chemotherapy, the specific target of CAR T cells (e.g., CD70), and/or complications of the procedure (e.g., CRS, ICANS) as well as treatment of these complications. Infection prophylaxis is recommended as detailed below in Table 43. These are guidelines only and should be applied based on individual subject circumstances (guidelines adapted from MD Anderson IEC Therapy Toxicity Assessment and Management recommendations).

TABLE 43
Infection Prophylaxis Guidelines

| | Preferred | Alternative | | | |
|---|---|---|---|---|---|
| Prophylaxis | Medication | Medication | Start | Stop | Comment |
| Viral | Valacyclovir¹ | Acyclovir¹ | CAR T | At least 1 year | Please note that the |
| Herpes simplex | 500 mg to 1000 | 400 mg PO BID to | infusion | post CAR T | varicella zoster |
| Varicella zoster | mg PO daily | 800 mg PO BID | day | infusion. May | prophylaxis covers |
| | | | | stop after 1 | the varicella zoster |
| | | | | year if CD4 | prophylaxis |
| | | | | >200/μL | recommendations |
| | | | | | for daratumumab |
| Hepatitis B | Entecavir¹ | Tenofovir | 2 weeks | 12-24 months | Monitor HBV titer |
| (only for subjects | 0.5 mg PO Daily | alafenamide¹ 25 | before | post CAR T | once a month while |
| who are positive | | mg PO daily or | CAR T | infusion | on prophylaxis and |
| for HBsAg or | | Tenofovir | infusion | | for a year after |
| HBcAb) | | disoproxil | | | stopping |
| | | fumarate¹ 300 mg | | | Consult Infectious |
| | | PO daily | | | Diseases if |
| | | | | | entecavir cannot be |
| | | | | | used |
| Bacterial | Levofloxacin¹ | Ciprofloxacin¹ | CAR T | 14 days after | Consult Infectious |
| (if neutropenia | 500 mg PO/IV | 500 mg PO BID | infusion | CAR T | Diseases if subject |
| with ANC | daily | —or— | day | infusion or | is allergic to |
| <1.0 K/μL is | | Any cephalosporin | | continue until | quinolones and |
| expected to last 7 | | with activity | | ANC >0.5 | cephalosporins. |
| days) | | against P. | | K/μL for 3 | |
| | | aeruginosa | | consecutive | |
| | | | | days without | |
| | | | | growth factor | |
| | | | | support | |
| | | | | (whichever is | |
| | | | | longer) | |
| Pneumocystis | Pentamidine | — | Within 1 | At least 1 year | Also has activity |
| jiroveci | inhaled or IV | | week | post CAR T | against toxoplasma |
| | recommended | | prior to | infusion. May | and nocardia |
| | within 1 week | | CAR T | stop after 1 | |
| | prior to CAR T | | infusion | year if CD4 | |
| | infusion and then | | | >200/μL | |
| | transition to | Pentamidine | Within 1 | At least 1 year | Albuterol nebulizer |
| | sulfamethoxazole/ | inhaled 300 mg | week | post CAR T | premed encouraged |
| | trimethoprim | flat dose every 28 | prior to | infusion. May | |
| | (SMZ/TMP; | days | CAR T | stop after 1 | |
| | preferred post | -or- | infusion | year if CD4 | |
| | CAR T infusion) | | | >200/μL | |
| | by 3-4 weeks if | Pentamidine IV | Within 1 | At least 1 year | Can cause |
| | counts have | 4 mg/kg (max | week | post CAR T | pancreatitis |
| | recovered: | 300 mg per | prior to | infusion. May | |
| | 1 DS tab every | dose) every 21 | CAR T | stop after 1 | |
| | M, W, F or | days | infusion | year if CD4 | |
| | 1 SS tab daily or | —or— | | >200/μL | |
| | 1 DS BID for 2 | Dapsone | 3-4 weeks | At least 1 year | Test for G6PD |
| | consecutive | 100 mg PO daily | post CAR | post CAR T | deficiency e.g., |
| | days/week | or 50 mg PO | T infusion | infusion. May | G6PD level |
| | | every 12 hours | | stop after 1 | Use caution if |
| | | —or— | | year if CD4 | subject has sulfa |
| | | | | >200/μL | allergy. |
| | | | | | Can cause |
| | | | | | hemolytic anemia |
| | | Atovaquone | Within 1 | At least 1 year | Must take with a |
| | | 1500 mg PO daily | week | post CAR T | fatty meal. |
| | | | prior to | infusion. May | Also has activity |
| | | | CAR T | stop after 1 | against toxoplasma, |
| | | | infusion | year if CD 4 | but inferior to |
| | | | | >200/μL | SMZ/TMP |
| Fungal | Fluconazole¹ | Caspofungin | CAR T | Continue until | |
| (Low risk)² | 200-400 mg | 50 mg IV | infusion | ANC >0.5 | |
| | PO/IV daily | daily | day | K/μL for 3 | |
| | | —or— | | consecutive | |
| | | Micafungin | | days without | |
| | | 50 mg IV Q 24 | | growth factor | |
| | | | | support | |
| Fungal | Posaconazole | Caspofungin | CAR T | Continue as | |
| including mold | 300 mg PO/IV | 50 mg IV | infusion | clinically | |
| prophylaxis | daily³ | daily | day or | indicated² | |
| (High risk)² | | —or-- | when | | |
| | | Micafungin | high-risk | | |
| | | 100 mg IV daily | criteria | | |
| | | | are met | | |

| | |
|---|---|
| CMV | Routine CMV prophylaxis is not required but CMV monitoring by PCR is recommended 1-2 times/week |
| | if neutropenia lasts 14 days or subject experiences grade 3 or 4 CRS/ICANS, or subject develops HLH. |
| | CMV monitoring is recommended for at least 30 days after completion of corticosteroids. |
| Immunoglobulin | Hypogammaglobulinemia may be observed after CAR T therapies that target B-cells and IgG levels |
| replacement | should be checked in such subjects when they develop respiratory infections. Immunoglobulin |
| therapy | replacement therapy and/or prophylaxis is only indicated for subjects who develop |
| | hypogammaglobulinemia and recurrent infections. |

ANC: absolute neutrophil count; BID: twice daily; CAR: chimeric antigen receptor; CMV: cytomegalovirus; CRS: cytokine release syndrome; DS: double strength; G6PD: glucose-6-phosphate dehydrogenase; HBcAb: hepatitis B core antibody; HBsAg: hepatitis B surface antigen; HBV: hepatitis B virus; HLH: hemophagocytic lymphohistiocytosis; ICANS: immune effector cell-associated neurotoxicity syndrome; IgG: immunoglobulin G; IV: intravenously; PCR: polymerase chain reaction; PO: by mouth; Q 24: every 24 hours; SMZ/TMP: sulfamethoxazole/trimethoprim; SS: single strength.
¹Adjust for renal function.
²Posaconazole prophylaxis is recommended for HIGH RISK subjects with leukemia, recent allogeneic stem cell therapy, prior history of mold infection, neutropenia lasting ≥14 days, subject experiences grade 3 or 4 CRS/ICANS, or if subject develops HLH. If corticosteroids are given, continue posaconazole for at least 1 month AFTER COMPLETION of corticosteroids. Do not stop posaconazole prophylaxis if ANC <1 K/μL. Voriconazole or isavuconazole may be used if the subject had previously been taking them or if posaconazole is not covered by insurance. If insurance only covers fluconazole, then use fluconazole for prophylaxis and consider aspergillus antigen testing 2 times a week for at least 1 month AFTER COMPLETION of steroids. Subjects not meeting the high-risk definition will be considered as LOW RISK for fungal infections and receive prophylaxis as detailed above.
³Loading dose of antifungals is not needed if it is being used for prophylaxis.

### Tumor Lysis Syndrome

Subjects receiving CAR T cell therapy may be at increased risk of TLS, which occurs when tumor cells release their contents into the bloodstream, either spontaneously or in response to therapy, leading to the characteristic findings of hyperuricemia, hyperkalemia, hyperphosphatemia, hypocalcemia, and elevated BUN. These electrolyte and metabolic disturbances can progress to clinical toxic effects, including renal insufficiency, cardiac arrhythmias, seizures, and death due to multiorgan failure (Howard et al., N Engl J Med, 2011). TLS has been reported in B cell malignancies with several drug products, including autologous CAR T cells, and there is significant familiarity with this syndrome and its management. In particular, leukemic forms such as ALL, acute myeloid leukemia, and CLL have a high (>5%) risk for TLS (Coiffier et al., J Clin Oncol, 2008). In the same high-risk group fall the noncutaneous T cell lymphomas, particularly ATLL as well as DLBCL (Coiffier et al., J Clin Oncol, 2008).

Subjects are closely monitored for TLS via laboratory assessments and symptoms from the start of LD chemotherapy until 28 days following each CTX130 infusion.

Subjects at increased risk of TLS receive prophylactic allopurinol (or a nonallopurinol alternative such as febuxostat) and/or rasburicase (Cortes et al., J Clin Oncol, 2010) and increased oral/IV hydration during screening and before initiation of LD chemotherapy. Prophylaxis can be stopped after 28 days following each CTX130 infusion or once the risk of TLS passes.

Sites monitor and treat TLS as per their institutional standard of care, or according to published guidelines (Cairo et al., Br J Haematol, 2004). TLS management, including administration of rasburicase, should be instituted promptly when clinically indicated.

Cytokine Release Syndrome

CRS is a toxicity associated with immune therapies, including CAR T cells, resulting from a release of cytokines, in particular IL-6 and IL-1 (Norelli et al., Nat Med, 2018). CRS is due to hyperactivation of the immune system in response to CAR engagement of the target antigen, resulting in multicytokine elevation from rapid T cell stimulation and proliferation (Frey et al., Blood, 2014; Maude et al., Cancer J, 2014). CRS has been observed in clinical trials irrespective of the antigen-targeted agents, including CD19-, BCMA-, CD123-, and mesothelin-directed CAR T cells, and anti-NY-ESO 1 and MART 1-targeted TCR-modified T cells (Frey et al., Blood, 2014; Hattori et al., Biol Blood Marrow Transplant, 2019; Maude et al., N Engl J Med, 2018; Neelapu et al., J Clin Oncol, 2018; Raje et al., N Engl J Med, 2019; Tanyi et al., J Immunother, 2017). CRS is a major toxicity reported with autologous CAR T cell therapy that has also been observed in early phase studies with allogeneic CAR T cell therapy (Benjamin et al., Lancet, 2018).

The clinical presentation of CRS may be mild and be limited to elevated temperatures or can involve 1 or multiple organ systems (e.g., cardiac, gastrointestinal [GI], respiratory, skin, central nervous) and multiple symptoms (e.g., high fevers, fatigue, anorexia, nausea, vomiting, rash, hypotension, hypoxia, headache, delirium, confusion). CRS may be life-threatening. Clinically, CRS can be mistaken for a systemic infection or, in severe cases, septic shock. Frequently the earliest sign is elevated temperature, which should prompt an immediate differential diagnostic work-up and timely initiation of appropriate treatment.

The goal of CRS management is to prevent life-threatening states and sequelae while preserving the potential for the anticancer effects of CTX130. Symptoms usually occur 1 to 14 days after autologous CAR T cell therapy in hematologic malignancies.

CRS should be identified and treated based on clinical presentation and not laboratory measurements. If CRS is suspected, grading should be applied according to the ASTCT (formerly known as American Society for Blood and Marrow Transplantation [ASBMT]) consensus recommendations (Table 33 (Lee et al., Biol Blood Marrow Transplant, 2019), and management should be performed according to the recommendations in Table 34, which are adapted from published guidelines (Lee et al., Blood, 2014; Lee et al., Biol Blood Marrow Transplant, 2019). Accordingly, grading of neurotoxicity is aligned with the ASTCT criteria for ICANS.

TABLE 33
Grading of CRS per ASTCT Consensus Criteria

| CRS | | | | |
|---|---|---|---|---|
| Parameter | Grade 1 | Grade 2 | Grade 3 | Grade 4 |
| Fever¹ | Temperature | Temperature | Temperature ≥38° C. | Temperature ≥38° C. |
| | ≥38° C. | ≥38° C. | | |
| With | None | Not requiring | Requiring a vasopressor | Requiring multiple |
| hypotension | | vasopressors | with or without | vasopressors (excluding |
| | | | vasopressin² | vasopressin)² |
| And/or³ | None | Requiring | Requiring high-flow | Requiring positive pressure |
| Hypoxia | | low-flow | nasal cannula⁴, | (e.g., CPAP, BiPAP, |
| | | nasal cannula⁴ | facemask, nonrebreather | intubation, and mechanical |
| | | or blow-by | mask, or Venturi mask | ventilation) |

ASTCT: American Society for Transplantation and Cellular Therapy; BiPAP: bilevel positive airway pressure; C: Celsius; CPAP: continuous positive airway pressure; CRS: cytokine release syndrome.
Note:
CRS grading based on ASTCT consensus criteria (Lee et al., Biol Blood Marrow Transplant, 2019)
Note:
Organ toxicides associated with CRS may be graded according to CTCAE v5.0 but they do not influence CRS grading.
¹Fever is defined as temperature ≥38° C. not attributable to any other cause. In patients who have CRS then receive antipyretics or anticytokine therapy such astocilizumab or steroids, fever is no longer required to grade subsequent CRS severity. In this case, CRS grading is driven by hypotension and/or hypoxia.
²See Table 35 for information on high-dose vasopressors.
³CRS grade is determined by the more severe event: hypotension or hypoxia not attributable to any other cause. For example, a patient with temperature of 39.5° C., hypotension requiring 1 vasopressor, and hypoxia requiring low-flow nasal cannula is classified as grade 3 CRS.
⁴Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 34
CRS Grading and Management Guidance

| CRS | | | Hypotension |
|---|---|---|---|
| Severity¹ | Tocilizumab | Corticosteroids | Management |
| Grade 1 | Tocilizumab2 may be considered | N/A | N/A |
| Grade 2 | Administer tocilizumab 8 mg/kg IV | Manage per institutional | Manage per |
| | over 1 hour (not to exceed 800 mg)² | guidelines. Continue | institutional |
| | Repeat tocilizumab every 8 hours as | corticosteroids use until | guidelines |
| | needed if not responsive to IV fluids | the event is grade ≤1, then | |
| | or increasing supplemental oxygen. | taper appropriately. | |
| | Limit to ≤3 doses in a 24-hour | | |
| | period; maximum total of 4 doses. | | |
| Grade 3 | Per grade 2 | Per grade 2 | Manage per |
| | | | institutional |
| | | | guidelines |
| Grade 4 | Per grade 2 | Per grade 2 | Manage per |
| | If no response to multiple doses of | | institutional |
| | tocilizumab and steroids, consider | | guidelines |
| | using other anticytokine therapies | | |
| | (e.g., anakinra). | | |

CRS: cytokine release syndrome; IV: intravenously; N/A: not applicable.
¹See (Lee et al., Biol Blood Marrow Transplant, 2019).
²Refer to tocilizumab prescribing information.

TABLE 35
High-dose Vasopressors

| Pressor | Dose* |
|---|---|
| Norepinephrine monotherapy | ≥20 μg/min |
| Dopamine monotherapy | ≥10 μg/kg/min |
| Phenylephrine monotherapy | ≥200 μg/min |
| Epinephrine monotherapy | ≥10 μg/min |
| If on vasopressin | Vasopressin + norepinephrine equivalent of ≥10 |
| | μg/min** |
| If on combination vasopressors | Norepinephrine equivalent of ≥20 μg/min** |
| (not vasopressin) | |

*All doses are required for ≥3 hours.
**VASST Trial vasopressor equivalent equation: norepinephrine equivalent dose = [norepinephrine (μg/min)] + [dopamine (μg/min)/2] + [epinephrine (μg/min)] + [phenylephrine (μg/min)/10].

Throughout the duration of CRS, subjects should be provided with supportive care consisting of antipyretics, IV fluids, and oxygen. In subjects experiencing signs or symptoms of CRS, initial blood sample collection for analysis of cytokines to occur at onset of symptoms, and additional samples should be drawn every 24 hours (±5 hours) until resolution. Troponin, N-terminal-pro hormone B-type natriuretic peptide (NT-proBNP), and B-type natriuretic peptide (BNP) should be assessed in the event of grade≥2 CRS on day 1, 3, and 7 of CRS event or as clinically indicated. Subjects who experience grade≥2 CRS should be monitored with continuous cardiac telemetry and pulse oximetry. For subjects experiencing grade 3 CRS, consider performing an echocardiogram to assess cardiac function. For grade 3 or 4 CRS, consider intensive care supportive therapy. The potential of an underlying infection in cases of severe CRS may be considered, as the presentation (fever, hypotension, hypoxia) is similar. Resolution of CRS is defined as resolution of fever (temperature≥38° C.), hypoxia, and hypotension (Lee et al., Biol Blood Marrow Transplant, 2019).

Hypotension and Renal Insufficiency

Hypotension and renal insufficiency have been reported with CAR T cell therapy and should be treated with IV administration of normal saline boluses according to institutional practice guidelines. Dialysis should be considered when appropriate.

Immune Effector Cell-associated Neurotoxicity Syndrome

Neurotoxicity has been documented in subjects with B cell malignancies treated with autologous CAR T cell therapies. Therefore, subjects are monitored for signs and symptoms of neurotoxicity associated with CAR T cell therapies in the current trial. Neurotoxicity may occur at the time of CRS, during the resolution of CRS, or following resolution of CRS, and its pathophysiology is unclear. The recent ASTCT (formerly known as ASBMT) consensus further defined ICANS as a disorder characterized by a pathologic process involving the CNS following any immune therapy that results in activation or engagement of endogenous or infused T cells and/or other immune effector cells (Lee et al., Biol Blood Marrow Transplant, 2019). The pathophysiology of neurotoxicity remains unclear; however, it is postulated that it may be due to a combination of cytokine release, trafficking of CAR T into CSF, and increased permeability of the blood-brain barrier (June et al., Science, 2018).

Signs and symptoms can be progressive and may include but are not limited to aphasia, altered level of consciousness, impairment of cognitive skills, motor weakness, seizures, and cerebral edema. ICANS grading (Table 35) was developed based on CAR T-Cell Therapy-Associated TOXicity (CARTOX) working group criteria used previously in autologous CAR T cell trials (Neelapu et al., J Clin Oncol, 2018). ICANS incorporates assessment of level of consciousness, presence/absence of seizures, motor findings, presence/absence of cerebral edema, and overall assessment of neurologic domains by using a modified tool called the immune effector cell-associated encephalopathy (ICE) assessment tool (Table 19).

Evaluation of any new onset neurotoxicity should include a neurological examination (including ICE assessment tool, Table 19), brain MRI, and examination of the CSF, as clinically indicated. If a brain MRI is not possible, all subjects should receive a noncontrast CT scan to rule out intracerebral hemorrhage. Electroencephalogram should also be considered as clinically indicated. Endotracheal intubation may be needed for airway protection in severe cases.

Lumbar puncture is required for any grade 3 or higher neurocognitive toxicity and is strongly recommended for grade 1 and grade 2 events, if clinically feasible. Lumbar puncture must be performed within 48 hours of symptom onset. Infectious etiology should be ruled out by performing a lumbar puncture whenever possible (especially for subjects with grade 3 or 4 ICANS).

Viral encephalitis (e.g., human herpesvirus 6 [HHV-6] encephalitis) must be considered in the differential diagnosis for subjects who experience neurocognitive symptoms after receiving CTX130. Whenever lumbar puncture is performed, the following viral panel needs to be performed in addition to standard panel performed at site (which should include cell count, gram stain, Neisseria meningitidis): CSF PCR analysis for herpes simplex virus 1 and 2, enterovirus, varicella-zoster virus, cytomegalovirus, EBV, and HHV-6, and HHV-7.

Results from the infectious disease panel must be available within 4 business days of the lumbar puncture to appropriately manage the subject.

In subjects diagnosed with HHV-6 encephalitis, treatment with ganciclovir or foscarnet should be initiated. Drug selection should be dictated by the drug's side effects, the subject's comorbidities and site clinical practice. The recommended duration of therapy is 3 weeks or as per site clinical practice (Hill et al., Curr Opin Virol, 2014; Ward et al., Haematologica, 2019). Once treatment is initiated, peripheral blood HHV-6 viral load should be checked weekly by PCR. An experienced bone marrow transplant physician and infectious disease expert in addition to the medical monitor need to be consulted.

CSF samples should be sent to a central laboratory for cytokine analysis and for presence of CTX130. Excess sample (if available) is retained for study-related exploratory research referenced herein.

Nonsedating, antiseizure prophylaxis (e.g., levetiracetam) should be considered, especially in subjects with a history of seizures, for at least 28 days following CTX130 infusion or upon resolution of neurological symptoms (unless it is considered the antiseizure medication to be contributing to the detrimental symptoms). Subjects who experience grade≥2 ICANS should be monitored with continuous pulse oximetry. For severe or life-threatening neurologic toxicities, intensive care supportive therapy should be provided. Neurology consultation should always be considered. Monitor platelets and for signs of coagulopathy, and transfuse blood products appropriately to diminish risk of intracerebral hemorrhage. Table 36 provides neurotoxicity grading and Table 37 provides management guidance.

For subjects who receive active steroid management for more than 3 days, antifungal and antiviral prophylaxis is recommended to mitigate a risk of severe infection with prolonged steroid use. Consideration for antimicrobial prophylaxis should also be given.

TABLE 36
ICANS Grading

| Neurotoxicity | | | | |
|---|---|---|---|---|
| Domain | Grade 1 | Grade 2 | Grade 3 | Grade 4 |
| ICE score¹ | 7-9 | 3-6 | 0-2 | 0 (subject is |
| | | | | unarousable and |
| | | | | unable to undergo ICE |
| | | | | assessment) |
| Depressed level | Awakens | Awakens | Awakens only to | Subject is unarousable |
| of | spontaneously | to voice | tactile stimulus | or requires vigorous or |
| consciousness² | | | | repetitive tactile |
| | | | | stimuli to arise; stupor |
| | | | | or coma |
| Seizure | N/A | N/A | Any clinical | Life-threatening |
| | | | seizure, focal or | prolonged seizure (>5 |
| | | | generalized, that | min) or repetitive |
| | | | resolves rapidly, or | clinical or electrical |
| | | | nonconvulsive | seizures without return |
| | | | seizures on EEG | to baseline in between |
| | | | that resolve with | |
| | | | intervention | |
| Motor findings³ | N/A | N/A | N/A | Deep focal motor |
| | | | | weakness such as |
| | | | | hemiparesis or |
| | | | | paraparesis |
| Elevated ICP/ | N/A | N/A | Focal/local edema | Diffuse cerebral |
| cerebral edema | | | on neuroimaging⁴ | edema on |
| | | | | neuroimaging, |
| | | | | decerebrate or |
| | | | | decorticate posturing, |
| | | | | cranial nerve VI palsy, |
| | | | | papilladema, or |
| | | | | Cushing's triad |

CTCAE: Common Terminology Criteria for Adverse Events; EEG: electroencephalogram; ICANS: immune effector cell-associated neurotoxicity syndrome; ICE: immune effector cell-associated encephalopathy (assessment tool); ICP: intracranial pressure; N/A: not applicable.
Note:
ICANS grade is determined by the most severe event (ICE score, level of consciousness, seizure, motor findings, raised ICP/cerebral edema) not attributable to any other cause.
¹A subject with an ICE score of 0 may be classified as grade 3 ICANS if awake with global aphasia, but a subject with an ICE score of 0 may be classified as grade 4 ICANS if unarousable (Table 19 for ICE assessment tool).
²Depressed level of consciousness should be attributable to no other cause (e.g., sedating medication).
³Tremors and myoclonus associated with immune effector therapies should be graded according to CTCAE v5.0 but do not influence ICANS grading.

TABLE 37
ICANS Management Guidance

| Severity | Management |
|---|---|
| Grade 1 | Provide supportive care per institutional practice. |
| Grade 2 | Consider administering dexamethasone 10 mg IV every 6 hours (or equivalent |
| | methylprednisolone) unless subject already on equivalent dose of steroids for CRS. |
| | Continue dexamethasone use until event is grade ≤1, then taper over 3 days. |
| Grade 3 | Administer dexamethasone 10 mg IV every 6 hours, unless subject already on |
| | equivalent dose of steroids for CRS. |
| | Continue dexamethasone use until event is grade ≤1, then taper over 3 days. |
| Grade 4 | Administer methylprednisolone 1000 mg IV per day for 3 days; if improves, then |
| | manage as above. |

CRS: cytokine release syndrome; ICANS: immune effector cell-associated neurotoxicity syndrome; IV: intravenously.

Headache, which may occur in a setting of fever or after chemotherapy, is a nonspecific symptom. Headache alone may not necessarily be a manifestation of ICANS and further evaluation should be performed. Weakness or balance problem resulting from deconditioning and muscle loss are excluded from definition of ICANS. Similarly, intracranial hemorrhage with or without associated edema may occur due to coagulopathies in these subjects and are also excluded from definition of ICANS. These and other neurotoxicities should be captured in accordance with CTCAE v5.0.

Hemophagocytic Lymphohistiocytosis

HLH has been reported after treatment with autologous CAR T cells (Barrett et al., Curr Opin Pediatr, 2014; Maude et al., N Engl J Med, 2014; Maude et al., Blood, 2015; Porter et al., Sci Transl Med, 2015; Teachey et al., Blood, 2013). HLH is a clinical syndrome that is a result of an inflammatory response following infusion of CAR T cells in which cytokine production from activated T cells leads to excessive macrophage activation. Signs and symptoms of HLH may include fevers, cytopenias, hepatosplenomegaly, hepatic dysfunction with hyperbilirubinemia, coagulopathy with significantly decreased fibrinogen, and marked elevations in ferritin and C-reactive protein (CRP). Neurologic findings have also been observed (Jordan et al., Blood, 2011; La Rosde, Hematology Am Soc Hematol Educ Program, 2015).

CRS and HLH may possess similar clinical syndromes with overlapping clinical features and pathophysiology. HLH will likely occur at the time of CRS or as CRS is resolving. HLH should be considered if there are unexplained elevated liver function tests or cytopenias with or without other evidence of CRS. Monitoring of CRP, ferritin, triglycerides, and fibrinogen may assist with diagnosis and define the clinical course. If these laboratory values further support a diagnosis of HLH, soluble CD25 blood levels should be determined in conjunction with a BM biopsy and aspirate if safe to conduct for further confirmation. Where feasible, excess BM samples should be sent to a central laboratory. Details of sample preparation and shipment are contained in the laboratory manual.

If HLH is suspected:
Frequently monitor coagulation parameters, including fibrinogen. These tests may be done more frequently than indicated in the schedule of assessments, and frequency should be driven based on laboratory findings.
Fibrinogen should be maintained ≥100 mg/dL to decrease risk of bleeding.
Coagulopathy should be corrected with blood products.
Given the overlap with CRS, manage according to grade 3 CRS with appropriate monitoring intensity. Follow institutional guidelines for additional treatment of HLH.
The IL-1 inhibitor anakinra or IFN-gamma inhibitor gamifant should be considered for management of HLH

Prolonged Cytopenias

Grade 3 neutropenia and thrombocytopenia, at times lasting more than 28 days after CAR T cell infusion, have been reported in subjects treated with autologous CAR T cell products (KYMRIAH [USPI], 2018; Raje et al., 2019; YESCARTA USPI, 2021). Therefore, subjects receiving CTX130 should be monitored for such toxicities and appropriately supported. Monitor platelets and for signs of coagulopathy and transfuse blood products appropriately to diminish risk of hemorrhage. Consideration should be given to antimicrobial and antifungal prophylaxis for any subject with prolonged neutropenia.

Due to the transient expression of CD70 on activated T and B lymphocytes, opportunistic infection such as viral reactivation may occur. Opportunistic infections may be considered when clinical symptoms arise.

During dose escalation, G-CSF may be considered in cases of grade 4 neutropenia post-CTX130 infusion. During cohort expansion G-CSF may be administered cautiously. For Parts A2, A4, and A6, daratumumab may increase neutropenia and/or thrombocytopenia induced by background therapy. Monitor complete blood cell counts periodically during treatment according to the manufacturer's prescribing information for background therapies. Monitor subjects with neutropenia for signs of infection. Daratumumab dose delay may be required to allow recovery of neutrophils and/or platelets, as per prescribing information. Consider supportive care with growth factors for neutropenia or transfusions for thrombocytopenia.

### Graft Versus Host Disease

GvHD is seen in the setting of allogeneic SCT and is the result of immunocompetent donor T cells (the graft) recognizing the recipient (the host) as foreign. The subsequent immune response activates donor T cells to attack the recipient to eliminate foreign antigen-bearing cells. GvHD is divided into acute, chronic, and overlap syndromes based on both the time from allogeneic SCT and clinical manifestations. Signs of acute GvHD may include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser. Et al., N Engl J Med, 2017).

To support the proposed clinical study, a nonclinical GLP-compliant GvHD and tolerability study was performed in immunocompromised mice treated at 2 IV doses: a high dose of 4×10⁷ CTX130 cells per mouse (approximately 1.6×10⁹ cells/kg) and a low dose of 2×10⁷ cells per mouse (approximately 0.8×10⁹ cells/kg). Both dose levels exceed the proposed highest clinical dose by more than 10-fold when normalized for body weight. No mice treated with CTX130 developed fatal GvHD during the course of the 12-week study. At necropsy, mononuclear cell infiltration was observed in some animals in the mesenteric lymph node and the thymus.

Minimal to mild perivascular inflammation was also observed in the lungs of some animals. These findings are consistent with mild GvHD, but did not manifest in clinical symptoms in these mice.

Further, due to the specificity of CAR insertion at the TRAC locus, it is highly unlikely for a T cell to be both CAR⁺ and TCR⁺. Remaining TCR⁺ cells are removed during the manufacturing process by immunoaffinity chromatography on an anti-TCR antibody column to achieve ≤0.4% TCR⁺ cells in the final product. A dose limit of 7×10⁴ TCR⁺ cells/kg is imposed for all dose levels. This is based on published reports on the number of allogeneic cells capable of causing severe GvHD during SCT with haploidentical donors (Bertaina et al., Blood, 2014). Through this specific editing, purification, and strict product release criteria, the risk of GvHD following CTX130 should be low, although the true incidence is unknown. However, given that CAR T cell expansion is antigen-driven and likely occurs only in TCR⁻ cells, it is unlikely that the number of TCR+ cells appreciably increase above the number infused.

Diagnosis and grading of GvHD is performed according to MAGIC criteria (Harris et al., Biol Blood Marrow Transplant, 2016), as outlined in Table 38.

TABLE 38
Criteria for Grading Acute GvHD

| | Skin (active | Liver | | Lower GI (stool |
|---|---|---|---|---|
| Stage | erythema only) | (bilirubin) | Upper GI | output/day) |
| 0 | No active | <2 mg/dL | No or intermittent | <500 mL/day or |
| | (erythematous) | | nausea, vomiting, | <3 episodes/day |
| | GvHD rash | | or anorexia | |
| 1 | Maculopapular rash | 2-3 mg/dL | Persistent nausea, | 500-999 mL/day or |
| | <25% BSA | | vomiting, or anorexia | 3-4 episodes/day |
| 2 | Maculopapular rash | 3.1-6 mg/dL | — | 1000-1500 mL/day or |
| | 25-50% BSA | | | 5-7 episodes/day |
| 3 | Maculopapular rash | 6.1-15 mg/dL | — | >1500 mL/day or |
| | >50% BSA | | | >7 episodes/day |
| 4 | Generalized | >15 mg/dL | — | Severe abdominal pain with |
| | erythroderma (>50% | | | or without ileus, or grossly |
| | BSA) plus bullous | | | bloody stool (regardless of |
| | formation and | | | stool volume) |
| | desquamation | | | |
| | >5% BSA | | | |

BSA: body surface area; GI: gastrointestinal; GvHD: graft versus host disease.

Overall GvHD grade is determined based on most severe target organ involvement.
Grade 0: No Stage 1-4 of any organ.
Grade 1: Stage 1-2 skin without liver, upper GI, or lower GI involvement.
Grade 2: Stage 3 rash and/or Stage 1 liver and/or Stage 1 upper GI and/or Stage 1 lower GI.
Grade 3: Stage 2-3 liver and/or Stage 2-3 lower GI, with Stage 0-3 skin and/or Stage 0-1 upper GI.
Grade 4: Stage 4 skin, liver, or lower GI involvement, with Stage 0-1 upper GI.

Potential confounding factors that may mimic GvHD such as infections and reactions to medications should be ruled out. Skin and/or GI biopsy should be obtained for confirmation before or soon after treatment has been initiated. In instance of liver involvement, liver biopsy should be attempted if clinically feasible. Sample(s) of all biopsies will also be sent to a central laboratory for pathology assessment. Details of sample preparation and shipment are contained in the laboratory manual.

Recommendations for management of acute GvHD are outlined in Table 39. To allow for intersubject comparability at the end of the trial, these recommendations shall be followed except in specific clinical scenarios in which following them could put the subject at risk.

TABLE 39
Acute GvHD Management

| Grade | Management |
|---|---|
| 1 | Skin: Topical steroids or immunosuppressants; if Stage 2: Prednisone 1 mg/kg (or |
| | equivalent dose). |
| 2-4 | Initiate methylprednisolone 2 mg/kg daily (or equivalent dose). |
| | IV form of steroid such as methylprednisolone should be considered if there are |
| | concerns with malabsorption. |
| | Steroid taper may begin after improvement is seen after ≥3 days of steroids. Taper |
| | should be 50% decrease of total daily steroid dose every 5 days. |
| | GI: in addition to steroids, start antidiarrheal agents as per standard practice. |

GI: gastrointestinal; IV: intravenous.

Decisions to initiate second-line GvHD therapy should be made sooner for subjects with more severe GvHD. For example, secondary therapy may be indicated after 3 days with progressive manifestations of GvHD, after 1 week with persistent grade 3 GvHD, or after 2 weeks with persistent grade 2 GvHD. Second-line systemic therapy may be indicated earlier in subjects who cannot tolerate high-dose glucocorticoid treatment (Martin et al., Biol Blood Marrow Transplant, 2012).

Management of refractory acute GvHD or chronic GvHD is per institutional guidelines. Anti-infective prophylaxis measures should be instituted per local guidelines when treating subjects with immunosuppressive agents (including steroids).

### On-Target Off-Tumor Toxicities

Activity of CTX130 Against Activated T and B Lymphocytes, Dendritic Cells

As described previously, activated T and B lymphocytes express CD70 transiently, and dendritic cells, as well as thymic epithelial cells express CD70 to a certain degree. Thus, these cells might become a target for activated CTX130.

Activity of CTX130 Against Osteoblasts

Activity of CTX130 was detected in nonclinical studies in cell culture of human primary osteoblasts. Hence, bone turnover is monitored via calcium levels as well as 2 osteoblast-specific markers: amino-terminal propeptide of type I procollagen (PINP) and bone-specific alkaline phosphatase (BSAP), which are considered the most useful markers in the assessment of bone formation (Fink et al., Osteoporosis, 2000). Standardized assays for assessment of both markers in serum are available. The concentration of these peptide markers reflects the activity of osteoblasts and the formation of new bone collagen.

PINP and BSAP is measured through a central laboratory assessment at screening, baseline, Days 7, 14, 21, and 28, and Months 3, 6, and 12 of the study (Table 20). Samples are collected at the same time of day (±2 hours) on the specified collection days because of the strong effect of circadian rhythm on bone turnover.

Activity of CTX130 Against Renal Tubular-Like Epithelium

Activity of CTX130 against renal tubular-like epithelial cells was detected in nonclinical studies of CTX130 in primary human kidney epithelium. Hence, subjects should be monitored for acute tubular damage by monitoring for an increase in serum creatinine of at least 0.3 mg/dL (26.5 μmol/L) over a 48-hour period and/or ≥1.5 times the baseline value within the previous 7 days. Serum creatinine is assessed at all scheduled visits during the study (Table 20). If acute renal tubular damage is suspected, additional tests should be conducted, including urine sediment analysis and fractional excretion of sodium in urine, and consultation by a nephrologist should be initiated.

Uncontrolled T Cell Proliferation

Upon recognition of target tumor antigen, in vivo activation and expansion have been observed with CAR T cells (Grupp et al., N Engl J Med, 2013). Autologous CAR T cells have been detected in peripheral blood, bone marrow, CSF, ascites, and other compartments (Badbaran et al., Cancers (Basel), 2020). If a subject develops signs of uncontrolled T cell proliferation, a sample from the clinical investigation should be submitted to the central laboratory to determine the origin of the proliferating T cells.

Special Consideration During COVID-19 Pandemic

Subjects enrolled in this study undergo LD chemotherapy, are immunocompromised, and at increased risk of infections. Hence, the clinical study protocol requires exclusion of subjects in the case of any ongoing active infection during screening, prior to LD chemotherapy, and prior to CTX130 infusion, or delayed infusions. This measure will include subjects with active infection with Severe Acute Respiratory Syndrome Coronavirus-2 (SARS-CoV-2), the causal agent of COVID-19 (coronavirus disease-2019).

Due to the rapidly changing evidence as well as locoregional differences, Additionally, the minimal requirements regarding COVID-19 infection and vaccinations were defined in a memorandum to the study centers that is periodically updated as evidence and guidelines evolve.

### 9. Statistical Methods

Sample Size

The exact number of subjects in each part of the study during dose escalation depends upon the number of dose levels evaluated and the occurrence of DLTs.

Part A1 (dose escalation) sample size is approximately 6 to 36 DLT-evaluable subjects.

Part A2 (dose escalation with daratumumab added to the lymphodepletion regimen) sample size is approximately 6 to 36 DLT-evaluable subjects.

Part A3 (dose escalation with additional CTX130 infusion on Day 5) sample size is approximately 6 to 12 DLT-evaluable subjects.

Part A4 (dose escalation with daratumumab added to the lymphodepletion regimen and with additional CTX130 infusion on Day 5) sample size is approximately 6 to 12 subjects.

Part A5 (dose escalation with Day 35 CTX130 consolidation) sample size is approximately 6 to 18 subjects.

Part A6 (dose escalation with daratumumab added to the lymphodepletion regimen and with Day 35 CTX130 consolidation) sample size is approximately 6 to 18 subjects.

For Part B (cohort expansion), each of the MF/SS and PTCL arms have an interim analysis to assess futility and early efficacy when approximately 50% of subjects have been enrolled and have completed at least their Month 3 visit or discontinued earlier, followed by a final analysis.

For the MF/SS arm, a sample size of 56 subjects have over 91% power (α=0.025, 1-sided test) to reject that ORR is less than or equal to a response rate of 20%, if assuming the true ORR with CTX130 is 40%.

For the PTCL arm, a sample size of 117 subjects have over 91% power (α=0.025, 1-sided test) to reject that ORR is less than or equal to a response rate of 30%, if assuming the true ORR with CTX130 is 45%.

Analysis Sets

The following analysis sets are evaluated and used for presentation of the data: Part A (Dose Escalation)

DLT-evaluable set: All subjects who receive CTX130 and either have completed the DLT evaluation period following the initial infusion (and second infusion in Parts A3/A4) or have discontinued earlier after experiencing a DLT.

### Part A+Part B

Safety analysis set (SAS): All subjects who were enrolled and received at least 1 dose of CTX130. Subjects are classified according to the treatment received, where treatment received is defined as the assigned dose level/schedule if it was received at least once, or the first dose level/schedule received if assigned treatment was never received. The SAS is the primary set for the analysis of safety data.

Full analysis set (FAS): All subjects who were enrolled and received CTX130 infusion. The FAS is the primary analysis set for clinical activity assessment.

### Study Endpoints

Primary Endpoints
Part A (Dose Escalation): Incidence of AEs defined as DLTs
Part B (Cohort Expansion): ORR, defined as the proportion of subjects who have achieved a best overall response of CR or PR, according to the Lugano response criteria (Cheson et al., J Clin Oncol, 2014) for subjects with DLBCL, and according to the ISCL criteria (Olsen et al., 2011) for subjects with MF/SS.

Secondary Endpoints
Efficacy
   Part A: The following efficacy endpoints per Lugano response criteria for subjects with PTCL-NOS, ALCL, leukemic and lymphomatous ATLL, AITL, and DLBCL, and per ISCL response criteria for subjects with SS or MF;
   Part B: The following efficacy endpoints per Lugano response criteria for subjects with PTCL, and per ISCL response criteria for subjects with SS or MF:
Best overall response (CR, PR, SD, PD, or not evaluable)
Objective Response Rate (ORR), defined as the proportion of subjects who achieved a best overall response of CR or PR (in Part B, as assessed by the investigator only)
Duration of Response (DOR), defined as the longest time interval between the date of an objective response of PR/CR and the date of first disease progression or death due to any cause following the OR. Reported only for subjects who have had PR/CR events
Duration of Response by BOR (DOR by BOR), defined as the longest time interval between the date of an objective response of PR/CR and the date of first disease progression or death due to any cause following the OR. Reported separately for responders by best overall response
Treatment Failure Free Survival (TFFS): TFFS is calculated as the time between the date of initial CTX130 infusion and the date of last documented relapse/progression following a response of SD or better, or death, whichever occurs first. Subjects without relapse/progression or death are censored at the last adequate response assessment that is SD or better prior to the date of alternative anticancer therapy.
Duration of Clinical Benefit (DOCB): Among subjects who have a response of CR or PR, DOCB is calculated as the time between the date of the first occurrence of any response and the date of last progression following a response of SD or better, or death. Responders without disease progression, relapse or death are censored at the date of last adequate response assessment that is SD or better prior to the date of alternative anticancer therapy.
Progression-Free Survival (PFS), defined as the time between the date of initial CTX130 infusion and the date of disease progression or death due to any cause
Overall Survival (OS), defined as the time between the date of initial CTX130 infusion and the date of death due to any cause
Time to Response (TTR), defined as the time between the date of initial CTX130 infusion and the date of first documented response (PR/CR)
Response Rate by Compartment (RR by Compartment; for subjects with MF/SS), defined as the proportion of subjects who achieved a best overall response of CR or PR, in each of the following compartments: Blood, Skin, Lymph Nodes, and Viscera

Safety

The incidence and severity of AEs and clinically significant laboratory abnormalities is summarized and reported according to CTCAE v5.0, except for CRS, which is graded according to ASTCT criteria (Lee et al., Biol Blood Marrow Transplant, 2019), neurotoxicity, which is graded according to ICANS criteria (Lee et al., Biol Blood Marrow Transplant, 2019) and CTCAE v5.0, and GvHD, which is graded according to MAGIC criteria (Harris et al., Biol Blood Marrow Transplant, 2016).

Pharmacokinetics

The levels of CTX130 in blood over time are assessed using a PCR-based assay.

Exploratory Endpoints
Levels of CTX130 in tissues.
Levels of cytokines in blood and other tissues.
Incidence of anti-CTX130 antibodies.
Incidence of autologous or allogeneic HSCT following CTX130 therapy.
Incidence and type of subsequent anticancer therapy.
Changes in peripheral blood levels of ATLL cells as monitored by immunophenotyping based on markers such as CD3, CD4, CD7, CD8, CD25, CD52, and HTLV-1 proviral load.
First subsequent therapy-free survival, defined as the time between date of CTX130 infusion and date of first subsequent therapy or PFS events.
Change from baseline in cognitive outcome, as assessed by ICE.
Other genomic, protein, metabolic, or pharmacodynamic endpoints.

### Results

To date, all subjects that participated in this study have completed Stage 1 (eligibility screening) within 14 days. After having met the eligibility criteria, two subjects started lymphodepleting therapy within 24 hours of completing Stage 1. All eligible subjects have completed the screening period (stage 1) and started LD chemotherapy in less than 8 days, with one subject completing screening and starting an LD chemo dose within 72 hrs. One subject receiving LD chemotherapy has already progressed to receiving the DL1 dose of CTX130 within 5 days following completion of the LD chemotherapy. Table 44 below summarizes patients subject to the treatment disclosed herein.

TABLE 44
Summary of CTX130 Exposure in Instant Study

| | | | Number of | Number of |
|---|---|---|---|---|
| | | | Subjects who | Subjects who |
| | | CTX130 Dose | Received a | Received |
| | Treatment | Level (Total | Single Infusion | 2 Infusions |
| Population | Cohort | CAR+ T Cells) | of CTX 130 | of CTX130 |
| Relapsed/Refractory | Part A1 (LD | DL1 (3 × 10⁷) | 4 (80.0) | 1 (20.0) |
| T or B cell | chemotherapy + | N = 5 | | |
| Malignancies | CTX130 | DL2 (1 × 10⁸) | 4 (100.0) | 0 |
| | | N = 4 | | |
| | | DL3 (3 × 10⁸) | 6 (100.0) | 0 |
| | | N = 6 | | |
| | | DL4 (9 × 108) | 2 (100.0) | 0 |
| | | N = 2 | | |
| | | Total Part A1 | 16 (94.1) | 1 (5.9) |
| | | N = 17 | | |

None of the treated subjects in this study exhibited any DLTs so far. Similarly, no DTLs were observed in a parallel study using CTX130 to treat subjects with RCC. See, e.g., International Patent Application No. PCT/IB2020/060718, the relevant discloses of which are incorporated by reference for the subject matter and purpose referenced herein. Further, the allogeneic CAR T cell therapy exhibited desired pharmacokinetic features in the treated human subjects, including CAR T cell expansion and persistence after infusion. Significant CAR T cell distribution, expansion and persistence has been observed as early as DL1. Up to 20-fold expansion of CTX130 in peripheral blood over T₀ has been observed in one T-cell lymphoma subject evaluated to date and persistence of CTX130 cells were detected in DL1 subjects up to 14 days post-infusion. Similar patterns of CAR T cell distribution, expansion and persistence are observed in the corresponding CTX130 RCC study, where 87-fold expansion of CTX130 has been observed and CTX130 cells have been detected for at least 28 days following infusion.

The eligible subjects in this study have MF with large cell transformation. Results obtained from the first T-cell lymphoma subject are summarized below.

The subject receiving the DL1 dose experienced significant reduction of the skin lesions as documented per photography according to the Olson/ISCL criteria for cutaneous T-cell lymphoma response assessment. Furthermore, a PET/CT scan 4 weeks following CTX130 infusion in the same subject revealed a drastic decrease in nodal and cutaneous lesions with most lesions entirely disappeared qualifying for a formal partial metabolic response.

Based on the available response assessments for 16 subjects in this study, the overall response was PD in 3 subjects, SD in 2 subjects, PR in 4 subjects, and CR in 3 subjects. A summary is provided in Table 45 below.

TABLE 45
Summary of Overall Responses

| | Part A1 | | | |
|---|---|---|---|---|
| | DL1 | DL2 | DL3 | DL4 |
| | N = 5 | N = 4 | N = 5 | N = 2 |
| Complete Response | 1 (20.0) | 0 | 2 (40.0) | 0 |
| n (%) | | | | |
| Partial Response | 2 (40.0) | 0 | 1 (20.0) | 0 |
| n (%) | | | | |
| Stable Disease | 1 (20.0) | 2 (50.0) | 0 | 0 |
| n (%) | | | | |
| Progressive Disease | 1 (20.0) | 1 (25.0) | 1 (20.0) | 1 (50) |
| n (%) | | | | |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method for treating a hematopoietic cancer, the method comprising:
(i) administering to a human patient having a hematopoietic cancer, which optionally is a CD70+ hematopoietic cancer, one or more doses of an anti-CD38 antibody,
(ii) performing a first lymphodepletion treatment to the human patient after the first dose of the anti-CD38 antibody; and
(iii) administering to the human patient a first dose of a population of genetically engineered T cells, which expresses a chimeric antigen receptor (CAR) that binds CD70 (anti-CD70 CAR-T cells) and is deficient in MHC Class I expression.

2. The method of claim 1, wherein step (i) comprises administering to the human patient a first dose of the anti-CD38 antibody at least 12 hours prior to the lymphodepletion treatment in step (ii) and within 10 days of the administration of the genetically engineered T cells in step (iii).

3. The method of claim 2, wherein step (i) further comprises administering to the human patient a second dose of the anti-CD38 antibody about three weeks after the first dose of the anti-CD70 CAR-T cells.

4. The method of claim 3, wherein step (i) further comprises administering to the human patient a third dose of the anti-CD83 antibody about six weeks after the first dose of the anti-CD70 CAR-T cells.

5. The method of claim 1, wherein step (iii) further comprises administering to the human patient a second dose of the population of anti-CD70 CAR-T cells.

6. The method of claim 5, wherein the second dose of the anti-CD70 CAR-T cells is performed about 4-15 days, optionally 4-6 days or 5-7 days, after the first dose of the anti-CD70 CAR-T cells.

7. The method of claim 5, wherein the second dose of the anti-CD70 CAR-T cells is not accompanied with a second lymphodepletion treatment.

8. The method of claim 1, wherein the method further comprises repeating steps (ii)-(iii), optionally step (i), when the human patient (a) loses complete response within 2 years after the first dose of the anti-CD70 CAR-T cells, or (b) show partial response, stable disease or progressive disease with clinical benefit.

9. The method of claim 8, wherein steps (ii)-(iii), optionally step (i), are repeated once.

10. The method of claim 8, wherein steps (ii)-(iii), optionally step (i), are repeated twice.

11. The method of claim 5, wherein the second dose of the anti-CD70 CAR-T cells is performed about 4-8 weeks after the first dose of the anti-CD70 CAR-T cells.

12. The method of claim 11, wherein the second dose of the anti-CD70 CAR-T cells is accompanied with a second lymphodepletion treatment, and optionally treatment with the anti-CD83 antibody.

13. The method of claim 12, wherein the human patient achieves complete response, partial response, stable disease, or progressive disease with clinical benefit about 4 weeks after the first dose of the anti-CD70 CAR-T cells.

14. The method of claim 11, wherein the second dose of the anti-CD70 CAR-T cells is not accompanied with a second lymphodepletion treatment when the human patient experiences significant cytopenia.

15. The method of claim 11, the method further comprises (iv) administering to the human patient a third dose of the anti-CD70 CAR-T cells when the human patient (a) loses complete response within 2 years after the first dose of the anti-CD70 CAR-T cells, or (b) show partial response, stable disease or progressive disease with clinical benefit.

16. The method of claim 15, wherein the third dose of the anti-CD70 CAR-T cells is greater than or equal to the first dose and/or the second dose of the anti-CD70 CAR-T cells.

17. The method of claim 15, wherein the third dose of the anti-CD70 CAR-T cells is accompanied with a third lymphodepletion treatment, and optionally a further treatment with the anti-CD38 antibody.

18. The method of claim 15, wherein the third dose of the anti-CD70 CAR-T cells is not accompanied with a third lymphodepletion treatment when the human patient experiences significant cytopenia.

19. The method of claim 1, wherein the anti-CD38 antibody is daratumumab.

20. The method of claim 1, wherein the one or more doses of the anti-CD38 antibody are about 8 mg/kg to about 16 mg/kg via intravenous infusion or 1800 mg via subcutaneous injection.

21. The method of claim 20, wherein the one or more doses of the anti-CD38 antibody are 16 mg/kg via intravenous infusion, and optionally wherein each dose is split evenly into two portions, which are administered to the human patient over two consecutive days.

22. The method of claim 20, wherein the one or more doses of the anti-CD38 antibody are 8 mg/kg via intravenous infusion.

23. The method of claim 1, wherein the human patient has one or more of the following features prior to a subsequent dose of the anti-CD38 antibody:
(a) no severe or unmanageable toxicity with prior doses of the anti-CD38 antibody,
(b) no disease progression,
(c) no ongoing uncontrolled infection,
(d) no grade≥3 neutropenia;
(e) no CD4+ T cell count<100/μl; and
(f) platelet count ≥25,000 cells/μl.

24. A method for treating a hematopoietic cancer, the method comprising:
(i) performing a first lymphodepletion treatment to a human patient having a hematopoietic cancer, which optionally is a CD70+ hematopoietic cancer;
(ii) administering to the human patient a first dose of a population of genetically engineered T cells, which expresses a chimeric antigen receptor (CAR) that binds CD70 (anti-CD70 CAR-T cells); and
(iii) administering to the human patient a second dose of the anti-CD70 CAR-T cells.

25-45. (canceled)

46. A method for treating a hematopoietic cancer, the method comprising:
(i) performing a lymphodepletion treatment to the human patient; and
(ii) administering to the human patient an effective amount of a population of genetically engineered T cells, which expresses a chimeric antigen receptor (CAR) that binds CD70 (anti-CD70 CAR-T cells), wherein the effective amount of the anti-CD70 CAR-T cell ranges from about 9×10⁸ CAR⁺ T cells to about 1.8×10⁹ CAR⁺ T cells.

47-74. (canceled)

75. The method of claim 1, wherein the human patient has a B cell malignancy, which optionally is diffuse large B cell lymphoma (DLBCL), follicular lymphoma, or mantle cell lymphoma (MCL).

76. The method of claim 75, wherein the human patient has DLBCL and has received up to 4 lines of prior anti-cancer therapy, optionally wherein one line of the prior anti-cancer therapy is a systemic therapy.

77. The method of claim 76, wherein the DLBCL patient failed a prior anti-CD19 CAR-T cell therapy.

78. The method of claim 1, wherein the human patient has a myeloid cell malignancy, which optionally is acute myeloid leukemia (AML).

79. The method of claim 1, wherein the human patient is free of mogamulizumab treatment at least 50 days prior to the first dose of the anti-CD70 CAR-T cells.

80. The method of claim 1, wherein the human patient has at least 10% CD70⁺ tumor cells in a biological sample obtained from the human patient.

81. The method of claim 80, wherein the biological sample is a tumor tissue sample and the level of CD70+ tumor cells is measured by immunohistochemistry (IHC).

82. The method of claim 80, wherein the biological sample is a blood sample or a bone marrow sample and the level of CD70+ tumor cells is determined by flow cytometry.

83. The method of claim 80, the method further comprising, prior to step (i), identifying a human patient having CD70+ tumor cells involved in a hematopoietic cell malignancy, which optionally is a T cell malignancy, a B cell malignancy, or a myeloid cell malignancy.

84. The method of claim 1, wherein the human patient has one or more of the following features:
(a) adequate organ function,
(b) measurable disease, peripheral blood tumor burden, or last one measurable lesion by imaging;
(c) free of a prior stem cell transplantation (SCT),
(d) free of a prior anti-CD70 agent or adoptive T cell or NK cell therapy,
(e) free of known contraindication to a lymphodepletion therapy,
(f) free of T cell or B cell lymphomas with a present or a past malignant effusion that is or was symptomatic,
(g) free of hemophagocytic lymphohistiocytosis (HLH),
(h) free of central nervous system malignancy or disorders,
(i) free of unstable angina, arrhythmia, and/or myocardial infarction,
(j) free of diabetes mellitus,
(k) free of uncontrolled infections,
(l) free of immunodeficiency disorders or autoimmune disorders that require immunosuppressive therapy, and
(m) free of solid organ transplantation.

85. The method of claim 1, further comprising monitoring development of acute toxicity after each administration of the population of genetically engineered T cells.

86. The method of claim 85, wherein the acute toxicity comprises cytokine release syndrome (CRS), ICAN, tumor lysis syndrome, GvHD, on target off-tumor toxicity, viral encephalitis, and/or uncontrolled T cell proliferation.

87. The method of claim 86, further comprising subjecting the human patient to toxicity management when acute toxicity is observed.

88. (canceled)

89. The method of claim 1, wherein the population of genetically engineered T cells comprise ≥30% CAR⁺ T cells, ≤0.5% TCR+ T cells, ≤30% B2M+ T cells, and ≤20% CD70+ T cells.

90. (canceled)


SEQUENCE LISTING (81 sequences)

SEQ ID NO: 1 - protein, 1368 aa, Artificial Sequence [sequence omitted]

SEQ ID NO: 2 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuuuggucccauuggucgcguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence; identical to SEQ ID NO: 2
  Synthetic

SEQ ID NO: 4 - RNA, 20 nt, Artificial Sequence
  Synthetic
  misc_feature (1)..(4): modified with 2′-O-methyl phosphorothioate
gcuuuggucccauuggucgc

SEQ ID NO: 5 - RNA, 20 nt, Artificial Sequence
  Synthetic
gcuuuggucccauuggucgc

SEQ ID NO: 6 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
agagcaacagugcuguggccguuuuagagcuagaaauagcaaguuaaaauaaggcuaguccguuaucaacuugaaaaaguggcaccgagucggugcuuuu

SEQ ID NO: 7 - RNA, 100 nt, Artificial Sequence; identical to SEQ ID NO: 6
  Synthetic

SEQ ID NO: 8 - RNA, 20 nt, Artificial Sequence
  Synthetic
  misc_feature (1)..(4): modified with 2′-O-methyl phosphorothioate
agagcaacagugcuguggcc

SEQ ID NO: 9 - RNA, 20 nt, Artificial Sequence
  Synthetic
agagcaacagugcuguggcc

SEQ ID NO: 10 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuacucucucuuucuggccguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence; identical to SEQ ID NO: 10
  Synthetic

SEQ ID NO: 12 - RNA, 20 nt, Artificial Sequence
  Synthetic
  misc_feature (1)..(4): modified with 2′-O-methyl phosphorothioate
gcuacucucucuuucuggcc

SEQ ID NO: 13 - RNA, 20 nt, Artificial Sequence
  Synthetic
gcuacucucucuuucuggcc

SEQ ID NO: 14 - DNA, 23 bp, Artificial Sequence
  Synthetic
gctttggtcccattggtcgcggg

SEQ ID NO: 15 - DNA, 20 bp, Artificial Sequence
  Synthetic
gctttggtcccattggtcgc

SEQ ID NO: 16 - DNA, 23 bp, Artificial Sequence
  Synthetic
agagcaacagtgctgtggcctgg

SEQ ID NO: 17 - DNA, 20 bp, Artificial Sequence
  Synthetic
agagcaacagtgctgtggcc

SEQ ID NO: 18 - DNA, 23 bp, Artificial Sequence
  Synthetic
gctactctctctttctggcctgg

SEQ ID NO: 19 - DNA, 20 bp, Artificial Sequence
  Synthetic
gctactctctctttctggcc

SEQ ID NO: 20 - RNA, 100 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 21 - RNA, 96 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 22 - RNA, 111 nt, Artificial Sequence
  Synthetic
  misc_feature (1)..(17): n is a, c, g, or u
  misc_feature (18)..(30): may be absent
  misc_feature (105)..(111): may be absent
nnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 23 - DNA, 19 bp, Artificial Sequence
  Synthetic
aagagcaacaaatctgact

SEQ ID NO: 24 - DNA, 39 bp, Artificial Sequence
  Synthetic
aagagcaacagtgctgtgcctggagcaacaaatctgact

SEQ ID NO: 25 - DNA, 33 bp, Artificial Sequence
  Synthetic
aagagcaacagtgctggagcaacaaatctgact

SEQ ID NO: 26 - DNA, 34 bp, Artificial Sequence
  Synthetic
aagagcaacagtgcctggagcaacaaatctgact

SEQ ID NO: 27 - DNA, 19 bp, Artificial Sequence
  Synthetic
aagagcaacagtgctgact

SEQ ID NO: 28 - DNA, 41 bp, Artificial Sequence
  Synthetic
aagagcaacagtgctgtgggcctggagcaacaaatctgact

SEQ ID NO: 29 - DNA, 38 bp, Artificial Sequence
  Synthetic
aagagcaacagtgctggcctggagcaacaaatctgact

SEQ ID NO: 30 - DNA, 41 bp, Artificial Sequence
  Synthetic
aagagcaacagtgctgtgtgcctggagcaacaaatctgact

SEQ ID NO: 31 - DNA, 79 bp, Artificial Sequence
  Synthetic
cgtggccttagctgtgctcgcgctactctctctttctgcctggaggctatccagcgtgagtctctcctaccctcccgct

SEQ ID NO: 32 - DNA, 78 bp, Artificial Sequence
  Synthetic
cgtggccttagctgtgctcgcgctactctctctttcgcctggaggctatccagcgtgagtctctcctaccctcccgct

SEQ ID NO: 33 - DNA, 75 bp, Artificial Sequence
  Synthetic
cgtggccttagctgtgctcgcgctactctctctttctggaggctatccagcgtgagtctctcctaccctcccgct

SEQ ID NO: 34 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 35 - DNA, 55 bp, Artificial Sequence
  Synthetic
cgtggccttagctgtgctcgcgctatccagcgtgagtctctcctaccctcccgct

SEQ ID NO: 36 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 37 - DNA, 58 bp, Artificial Sequence
  Synthetic
cacaccacgaggcagatcaccaagcccgcgcaatgggaccaaagcagcccgcaggacg

SEQ ID NO: 38 - DNA, 61 bp, Artificial Sequence
  Synthetic
cacaccacgaggcagatcaccaagcccgcgaaccaatgggaccaaagcagcccgcaggacg

SEQ ID NO: 39 - DNA, 48 bp, Artificial Sequence
  Synthetic
cacaccacgaggcagatcaccaatgggaccaaagcagcccgcaggacg

SEQ ID NO: 40 - DNA, 59 bp, Artificial Sequence
  Synthetic
cacaccacgaggcagatcaccaagcccgcgccaatgggaccaaagcagcccgcaggacg

SEQ ID NO: 41 - DNA, 59 bp, Artificial Sequence
  Synthetic
cacaccacgaggcagatcaccaagcccgcaccaatgggaccaaagcagcccgcaggacg

SEQ ID NO: 42 - DNA, 35 bp, Artificial Sequence
  Synthetic
cacaccacgaggcagatcaccaagcccgcaggacg

SEQ ID NO: 43 - DNA, 4688 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 45 - DNA, 1527 bp, Artificial Sequence
  Synthetic
atggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataa

SEQ ID NO: 46 - protein, 508 aa, Artificial Sequence
  Synthetic
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 735 bp, Artificial Sequence
  Synthetic
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 48 - protein, 245 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 49 - protein, 118 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 50 - protein, 111 aa, Artificial Sequence
  Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 51 - protein, 16 aa, Artificial Sequence
  Synthetic
GGGGSGGGGSGGGGSG

SEQ ID NO: 52 - protein, 22 aa, Artificial Sequence
  Synthetic
MLLLVTSLLLCELPHPAFLLIP

SEQ ID NO: 53 - protein, 21 aa, Artificial Sequence
  Synthetic
MALPVTALLLPLALLLHAARP

SEQ ID NO: 54 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 55 - protein, 23 aa, Artificial Sequence
  Synthetic
IYIWAPLAGTCGVLLLSLVITLY

SEQ ID NO: 56 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 57 - protein, 42 aa, Artificial Sequence
  Synthetic
KRGRKKLLYIFKQPFMRPVQTTQEEDGCSCRFPEEEEGGCEL

SEQ ID NO: 58 - DNA, 120 bp, Artificial Sequence
  Synthetic
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 59 - protein, 40 aa, Artificial Sequence
  Synthetic
SKRSRLLHSDYMNMTPRRPGPTRKHYQPYAPPRDFAAYRS

SEQ ID NO: 60 - DNA, 336 bp, Artificial Sequence
  Synthetic
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 61 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 800 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 63 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 64 - DNA, 49 bp, Artificial Sequence
  Synthetic
aataaaatcgctatccatcgaagatggatgtgtgttggttttttgtgtg

SEQ ID NO: 65 - DNA, 804 bp, Artificial Sequence
  Synthetic
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 66 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcccgcaggacgcacccauaguuuuagagcuagaaauagcaaguuaaaauaaggcuaguccguuaucaacuugaaaaaguggcaccgagucggugcuuuu

SEQ ID NO: 67 - RNA, 100 nt, Artificial Sequence; identical to SEQ ID NO: 66
  Synthetic

SEQ ID NO: 68 - RNA, 20 nt, Artificial Sequence
  Synthetic
  misc_feature (1)..(4): modified with 2′-O-methyl phosphorothioate
gcccgcaggacgcacccaua

SEQ ID NO: 69 - RNA, 20 nt, Artificial Sequence
  Synthetic
gcccgcaggacgcacccaua

SEQ ID NO: 70 - protein, 300 aa, Artificial Sequence
  Synthetic
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 71 - protein, 452 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 124 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSAS

SEQ ID NO: 73 - protein, 213 aa, Artificial Sequence
  Synthetic
EIVLTQSPATLSLSPGERATLSCRASQSVSSYLAWYQQKPQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 107 aa, Artificial Sequence [sequence omitted]

SEQ ID NO: 75 - protein, 5 aa, Artificial Sequence
  Synthetic
SFAMS

SEQ ID NO: 76 - protein, 17 aa, Artificial Sequence
  Synthetic
AISGSGGGTYYADSVKG

SEQ ID NO: 77 - protein, 13 aa, Artificial Sequence
  Synthetic
DKILWFGEPVFDY

SEQ ID NO: 78 - protein, 11 aa, Artificial Sequence
  Synthetic
RASQSVSSYLA

SEQ ID NO: 79 - protein, 7 aa, Artificial Sequence
  Synthetic
DASNRAT

SEQ ID NO: 80 - protein, 9 aa, Artificial Sequence
  Synthetic
QQRSNWPPT

SEQ ID NO: 81 - protein, 487 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR